(19) United States
(12) Patent Application Publication
(10) Pub. No.: US 2026/0356846 A1
(43) Pub. Date: Nov. 12, 2026

(54) STATIN DERIVATIVES AND METHODS OF USING THE SAME

(71) Applicants: Clotilde LAGIER-TOURENNE, Charlestown, MA (US); Matthew NOLAN, Charlestown, MA (US); Robert D. HUBBARD, Cambridge, MA (US); The General Hospital Corporation, Boston, MA (US)

(72) Inventors: Clotilde LAGIER-TOURENNE, Charlestown, MA (US); Matthew NOLAN, Charlestown, MA (US); Robert D. HUBBARD, San Diego, CA (US)

(21) Appl. No.: 19/491,467

(22) PCT Filed: Jun. 12, 2024
(86) PCT No.: PCT/US2024/033626

§ 371 Date: Dec. 9, 2025

Related U.S. Application Data
(60) Provisional application No. 63/472,434, filed on Jun. 12, 2023.
(60) Provisional application No. 63/611,822, filed on Dec. 19, 2023.

(51) Int. Cl. C07D 495/04; A61K 31/428; A61K 31/436; A61K 31/4365; A61K 31/437; A61K 31/4418; A61K 31/444; A61K 31/4545; C07D 213/55; C07D 277/62; C07D 277/64; C07D 491/052; C07D 513/04

(52) U.S. Cl. CPC C07D 495/04; A61K 31/428; A61K 31/436; A61K 31/4365; A61K 31/437; A61K 31/4418; A61K 31/444; A61K 31/4545; C07D 213/55; C07D 277/62; C07D 277/64; C07D 491/052; C07D 513/04

(57) ABSTRACT

The present application relates to compounds of Formula (I′) or Formula (II′) and to their pharmaceutically acceptable salts, pharmaceutical compositions, methods of use, and methods for their preparation. The compounds disclosed herein are useful for modulating stathmin-2 (STMN2) activity and may be used in the treatment of disorders in which STMN2 activity is implicated, such as neurodegenerative diseases and disorders or axonopathy.

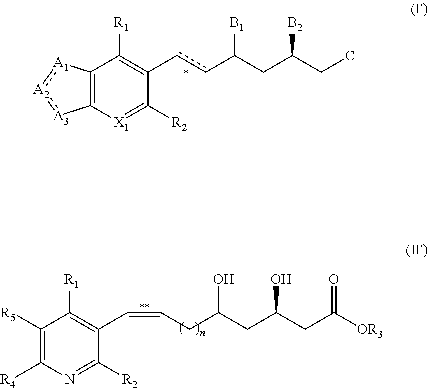

### RELATED APPLICATIONS

This application claims priority to, and the benefit of, U.S. Provisional Application No. 63/472,434, filed on Jun. 12, 2023; and U.S. Provisional Application No. 63/611,822, filed on Dec. 19, 2023, the entire contents of which are incorporated herein by reference.

### BACKGROUND

The present disclosure relates to statin derivatives and their use as modulators of stathmin (e.g., stathmin-2) for the treatment of a disease or disorder (e.g., neurodegenerative diseases).

About 97% of amyotrophic lateral sclerosis (ALS) cases and almost half of patients with frontotemporal dementia (FTD) are pathologically associated with cytoplasmic mislocalization and aggregation of the RNA binding protein, TDP-43, within neurons and glia (ALS-TDP and FTD-TDP). Additionally, mutations in its encoding gene, TARDBP, are a rare cause of both ALS and FTD, indicating that TDP-43 may be central to ALS/FTD pathogenesis. It has been identified that human mRNAs are affected by a reduction in TDP-43, such as neuronal growth-associated protein Stathmin-2 (STMN2; also known as SCG10). Aberrant splicing (with inclusion of an abnormal ‘cryptic’ exon 2a) and premature polyadenylation of STMN2 leads to a reduction of STMN2 protein level in response to nuclear loss of TDP-43 in cultured neurons, with this cryptic exon signature also detectable in tissues from patients with sporadic and familial forms of ALS and FTD, and patients with Alzheimer's disease associated with TDP-43 pathology. STMN2 is a binding partner of tubulin heterodimers, which is related to neurite outgrowth and axonal regeneration. Thus, modulation of STMN2 is a therapeutic target applicable for the treatment of ALS.

Currently available therapies for ALS are non-target driven and of limited efficacy. There is a need for effective therapies targeted at specific elements of disease pathophysiology, wherein STMN2 is a target that has demonstrated its tractability for therapeutic modulation. HMG-COA reductase inhibitors (e.g., statins) are compounds capable of increasing STMN2. The disclosure arises from a need to provide further compounds for the modulation of STMN2 activity with improved therapeutic potential. In particular, compounds with improved physicochemical, pharmacological and/or pharmaceutical properties.

### SUMMARY

In some aspects, the present disclosure provides a compound of Formula (I′):

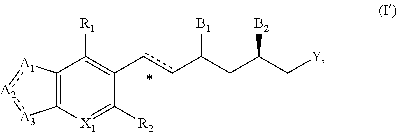

or a pharmaceutically acceptable salt thereof, wherein:
   [symbol] is a single or double bond, as valency allows;
   [symbol] is a single or double bond, wherein the double bond is the (E) isomer;
   X₁ is CH or N;
   A₁ is CR_{A1}, N, O, or S;
   A₂ is CR_{A2}, N, O, or S;
   A₃ is CR_{A3}, N, O, or S, wherein at least one A₁, A₂, or A₃ is S;
   R₁ is C₆-C₁₀ aryl or 5- to 10-membered heteroaryl, wherein the aryl or heteroaryl is optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl;
   R₂ is C₃-C₁₀ cycloalkyl;
   B₁ is H or —OH;
   B₂ is H or —OH;
   Y is H, —C(O)OR₃, —C(O)N(R₃)₂,
   each R₃ independently is H, C₁-C₆ alkyl, C₂-C₆ alkenyl, C₂-C₆ alkynyl, C₁-C₆ alkoxyl, or C₁-C₆ haloalkyl;
   R_{A1} is H, C₁-C₆ alkyl, C₂-C₆ alkenyl, C₂-C₆ alkynyl, C₁-C₆ alkoxyl, C₁-C₆ haloalkyl, or C₃-C₁₀ cycloalkyl;
   R_{A2} is H, C₁-C₆ alkyl, C₂-C₆ alkenyl, C₂-C₆ alkynyl, C₁-C₆ alkoxyl, C₁-C₆ haloalkyl, C₃-C₁₀ cycloalkyl, C₆-C₁₀ aryl, or 5- to 10-membered heteroaryl; and
   R_{A3} is H, C₁-C₆ alkyl, C₂-C₆ alkenyl, C₂-C₆ alkynyl, C₁-C₆ alkoxyl, C₁-C₆ haloalkyl, C₃-C₁₀ cycloalkyl, 3- to 10-membered heterocyclyl optionally substituted with one or more C₁-C₆ alkyl, C₆-C₁₀ aryl, or 5- to 10-membered heteroaryl optionally substituted with one or more C₁-C₆ alkyl,
provided that:
   (a) when R₂ is cyclopropyl, X₁ is N, A₁ is CR_{A1}, and A₂ is CR_{A2}, then at least one of R_{A1} and R_{A2} is not H; and
   (b) when R₂ is cyclopropyl, X₁ is N, A₁ is CR_{A1}, and A₂ is CH, then R_{A1} is not ethyl.

In some aspects, the present disclosure provides a compound of Formula (II′):

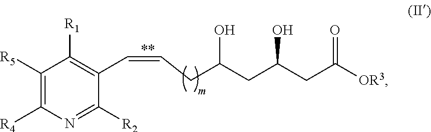

or a pharmaceutically acceptable salt thereof, wherein:
   [symbol] is a double bond, wherein the double bond is the (E) or (Z) isomer;
   R₁ is C₆-C₁₀ aryl or 5- to 10-membered heteroaryl, wherein the aryl or heteroaryl is optionally substituted with one or more R₁ₐ;
   each R₁ₐ independently is halo, —CN, —OH, —NH₂, C₁-C₆ alkyl, C₂-C₆ alkenyl, C₂-C₆ alkynyl, C₁-C₆ alkoxyl, or C₁-C₆ haloalkyl;
   R₂ is C₃-C₁₀ cycloalkyl or methyl;
   R₃ is H, C₁-C₆ alkyl, C₂-C₆ alkenyl, C₂-C₆ alkynyl, C₁-C₆ alkoxyl, or C₁-C₆ haloalkyl;
   R₄ is H, C₃-C₁₀ cycloalkyl, 3- to 10-membered heterocyclyl, C₁-C₆ alkyl, C₂-C₆ alkenyl, C₂-C₆ alkynyl, C₁-C₆ alkoxyl, or C₁-C₆ haloalkyl;
   R₅ is H, C₁-C₆ alkyl, C₂-C₆ alkenyl, C₂-C₆ alkynyl, C₁-C₆ alkoxyl, C₁-C₆ haloalkyl, or C₃-C₁₀ cycloalkyl, wherein the alkyl, alkenyl, alkynyl, alkoxyl, haloalkyl or C₃-C₁₀ cycloalkyl is optionally substituted with one or more C₁-C₆ alkoxy or —O(C₃-C₁₀ cycloalkyl); or
   R₅ and one R₁ₐ, together with the intervening atoms, form 3- to 10-membered heterocyclyl; and
   m is 0 or 1,
provided that:
   (a) when R₂ is cyclopropyl, and R₅ is C₁ alkyl substituted by C₁ alkoxy, then R₄ is not isopropyl; and
   (b) when R₁ is monosubstituted C₆ aryl substituted by one fluoro, R₂ is cyclopropyl, and R₅ is C₁ alkyl substituted by C₁ alkoxy, then R₄ is not cyclopropyl; and
   (c) when R₂ is methyl, then R₄ is not C₁-C₆ alkyl.

In some aspects, the present disclosure provides a compound obtainable by, or obtained by, a method for preparing a compound as described herein (e.g., a method comprising one or more steps described in Schemes 1-7).

In some aspects, the present disclosure provides a pharmaceutical composition comprising a compound of the present disclosure, or a pharmaceutically acceptable salt thereof, and one or more pharmaceutically acceptable excipient, diluent, or carrier, or a combination thereof.

In some aspects, the present disclosure provides an intermediate as described herein, being suitable for use in a method for preparing a compound as described herein (e.g., the intermediate is selected from the intermediates described in Examples 1-2).

In some aspects, the present disclosure provides a method of modulating stathmin-2 (STMN2) activity (e.g., in vitro or in vivo) with aa compound of the present disclosure or a pharmaceutically acceptable salt thereof.

In some aspects, the present disclosure provides a method of treating or preventing a disease or disorder disclosed herein in a subject in need thereof, comprising administering to the subject a compound of the present disclosure or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject a compound of the present disclosure or a pharmaceutically acceptable salt thereof, or a pharmaceutical composition of the present disclosure.

In some aspects, the present disclosure provides a compound of the present disclosure or a pharmaceutically acceptable salt thereof for use in modulating STMN2 activity (e.g., in vitro or in vivo).

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides use of a compound of the present disclosure or a pharmaceutically acceptable salt thereof in the manufacture of a medicament for modulating STMN2 activity (e.g., in vitro or in vivo).

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating a disease or disorder disclosed herein.

In some embodiments, the disease or disorder is a neurodegenerative disease (e.g., Amyotrophic Lateral Sclerosis (ALS)).

In some embodiments, the disease or disorder is associated with axonal degeneration, axonal damage, or axonopathy.

In some embodiments, the neurodegenerative disease is associated with axonal degeneration, axonal damage, or axonopathy.

In some aspects, the present disclosure provides a method of preparing a compound of the present disclosure.

In some aspects, the present disclosure provides a method of preparing a compound, comprising one or more steps described herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### BRIEF DESCRIPTION OF DRAWINGS

FIG. 1 depicts compound activity in WT (left) and TDP-43 mutant (right) SH-SY5Y cells, wherein darker grey correlates to higher relative light unit (RLU) per cell.

FIG. 2 depicts compounds of the present disclosure activity across mutant TDP-43 cell lines as compared to known controls.

### DETAILED DESCRIPTION

The present disclosure relates to statin derivatives, prodrugs, and pharmaceutically acceptable salts thereof, which may modulate stathmin-2 (STMN2) expression or activity and are accordingly useful in methods of treatment of the human or animal body. The present disclosure also relates to processes for the preparation of these compounds, to pharmaceutical compositions comprising them and to their use in the treatment of disorders in which STMN2 is implicated, such as neurodegenerative diseases or disorders or diseases or disorders associated with axonopathy.

### DEFINITIONS

Unless otherwise stated, the following terms used in the specification and claims have the following meanings set out below.

Without wishing to be limited by this statement, it is understood that, while various options for variables are described herein, the disclosure intends to encompass operable embodiments having combinations of the options. The disclosure may be interpreted as excluding the non-operable embodiments caused by certain combinations of the options. For example, while various options for variables are described herein, the disclosure may be interpreted as excluding structures for non-operable compound caused by certain combinations of variables.

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intends to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl, or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), (including alkylcarbonylamino, acylamino arylcarbonylamino, carbamoyl and ureido), amidino, imino, sulfhydryl, alkylthio, arylthio, thiocarboxylate, sulfates, sulfamoyl, sulfonamido, nitro, trifluoromethyl, cyano, azido, heterocyclyl, alkylaryl, or an aromatic or heteroaromatic moiety.

As used herein, the term “alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

As used herein, the term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

As used herein, the term “alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms. As used herein, “C₂-C₆ alkenylene linker” or “C₂-C₆ alkynylene linker” is intended to include C₂, C₃, C₄, C₅ or C₆ chain (linear or branched) divalent unsaturated aliphatic hydrocarbon groups. For example, C₂-C₆ alkenylene linker is intended to include C₂, C₃, C₄, C₅ and C₆ alkenylene linker groups.

As used herein, the term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Other optionally substituted moieties (such as optionally substituted cycloalkyl, heterocyclyl, aryl, or heteroaryl) include both the unsubstituted moieties and the moieties having one or more of the designated substituents. For example, substituted heterocyclyl includes those substituted with one or more alkyl groups, such as 2,2,6,6-tetramethyl-piperidinyl and 2,2,6,6-tetramethyl-1,2,3,6-tetrahydropyridinyl.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic.

As used herein, the term “heterocyclyl” refers to a saturated or partially unsaturated 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur, unless specified otherwise. Examples of heterocyclyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, 5,6-dihydro-4H-cyclopenta[b]thiophenyl, and the like. In the case of multicyclic heterocyclyl, only one of the rings in the heterocyclyl needs to be non-aromatic (e.g., 4,5,6,7-tetrahydrobenzo[c]isoxazolyl).

It is understood that when a variable has two attachments to the rest of the formula of the compound, the two attachments could be at the same atom or different atoms of the variable. For example, when a variable (e.g., variable X) is cycloalkyl or heterocyclyl, and has two attachments to the rest of the formula of the compound, the two attachments could be at the same atom or different atoms of the cycloalkyl or heterocyclyl.

As used herein, the term “aryl” includes groups with aromaticity, including “conjugated,” or multicyclic systems with one or more aromatic rings and do not contain any heteroatom in the ring structure. The term aryl includes both monovalent species and divalent species. Examples of aryl groups include, but are not limited to, phenyl, biphenyl, naphthyl and the like.

As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidised (i.e., N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, isothiazole, pyridine, pyrazine, pyridazine, pyrimidine, and the like. Heteroaryl groups can also be fused or bridged with alicyclic or heterocyclic rings, which are not aromatic so as to form a multicyclic system (e.g., 4,5,6,7-tetrahydrobenzo[c]isoxazolyl).

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthrydine, indole, benzofuran, purine, benzofuran, deazapurine, or indolizine.

The cycloalkyl, heterocyclyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, the term “about” refers to a recited amount, value, or duration±10% or less of said amount, value, or duration. In some embodiments, “about” refers to a recited amount, value, or duration±10%, ±8%, ±6%, ±5%, ±4%, ±2%, ±1%, or +0.5%. In other embodiments, “about” refers to a recited amount, value, or duration±10%, ±8%, ±6%, ±5%, ±4%, or ±2%. In other embodiments, “about” refers to a recited amount, value, or duration±5%. In some embodiments, “about” refers to a listed amount, value, or duration ±2% or ±1%. For example, in some embodiments, when the term “about” is used when reciting a temperature or temperature range, these terms refer to the recited temperature or temperature range±5 °C, ±2 °C, or ±1 °C. In other embodiments, the term “about” refers to the recited temperature or temperature range±2 °C.

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

As used herein, the term “hydroxy” or “hydroxyl” includes groups with an —OH or —O⁻.

As used herein, the term “halo” or “halogen” refers to fluoro, chloro, bromo and iodo.

The term “haloalkyl” or “haloalkoxyl” refers to an alkyl or alkoxyl substituted with one or more halogen atoms.

As used herein, the term “optionally substituted haloalkyl” refers to unsubstituted haloalkyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more C₅, or any combination thereof, unless indicated otherwise.

It is to be understood that the present disclosure provides methods for the synthesis of the compounds of any of the Formulae described herein. The present disclosure also provides detailed methods for the synthesis of various disclosed compounds of the present disclosure according to the following schemes as well as those shown in the Examples.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps order for performing certain actions is immaterial so long as the invention remains operable. Moreover, two or more steps or actions can be conducted simultaneously.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for organic Synthesis, John Wiley and Sons (1994); and L. Paquette, ed., Encyclopedia of Reagents for organic Synthesis, John Wiley and Sons (1995), incorporated by reference herein, are useful and recognized reference textbooks of organic synthesis known to those in the art.

One of ordinary skill in the art will note that, during the reaction sequences and synthetic schemes described herein, the order of certain steps may be changed, such as the introduction and removal of protecting groups. One of ordinary skill in the art will recognize that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

It is to be understood that, unless otherwise stated, any description of a method of treatment or prevention includes use of the compounds to provide such treatment or prevention as is described herein. It is to be further understood, unless otherwise stated, any description of a method of treatment or prevention includes use of the compounds to prepare a medicament to treat or prevent such condition. The treatment or prevention includes treatment or prevention of human or non-human animals including rodents and other disease models.

It is to be understood that, unless otherwise stated, any description of a method of treatment includes use of the compounds to provide such treatment as is described herein. It is to be further understood, unless otherwise stated, any description of a method of treatment includes use of the compounds to prepare a medicament to treat such condition. The treatment includes treatment of human or non-human animals including rodents and other disease models.

As used herein, the term “subject” includes human and non-human animals, as well as cell lines, cell cultures, tissues, and organs. In some embodiments, the subject is a mammal. The mammal can be e.g., a human or appropriate non-human mammal, such as primate, mouse, rat, dog, cat, cow, horse, goat, camel, sheep or a pig. The subject can also be a bird or fowl. In some embodiments, the subject is a human.

As used herein, the term “subject in need thereof” refers to a subject having a disease or having an increased risk of developing the disease. In some embodiments, the subject in need thereof has a pathologic deficiency in STMN2. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

It is to be understood that a compound of the present disclosure, or a pharmaceutically acceptable salt, polymorph or solvate thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18^{th} edition (1990). These texts can, of course, also be referred to in making or using an aspect of the disclosure.

It is to be understood that the present disclosure also provides pharmaceutical compositions comprising any compound described herein in combination with one or more pharmaceutically acceptable excipient, diluent, adjuvant, carrier, or a combination thereof.

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., a disease or disorder disclosed herein) and the health of the patient should preferably be closely monitored during and for a reasonable period after treatment.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), cyclodextrins and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, capsules or sachets. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays, powders or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

It is to be understood that the pharmaceutical compositions can be included in a container, pack, or dispenser together with instructions for administration.

It is to be understood that, for the compounds of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. Examples of pharmaceutically acceptable salts include, but are not limited to, mineral organic acid salts of basic residues such as amines, alkali organic salts of acidic residues such as carboxylic acids, and the like. The pharmaceutically acceptable salts include the conventional non-toxic salts or the quaternary ammonium salts of the parent compound formed, for example, from non-toxic inorganic organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed.

Techniques for formulation and administration of the disclosed compounds of the disclosure can be found in Remington: the Science and Practice of Pharmacy, 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

As use herein, the phrase “compound of the disclosure” refers to those compounds which are disclosed herein, both generically and specifically.

### Compounds of the Present Disclosure

### Formula (I′)

In some aspects, the present disclosure provides a compound of Formula (I′):

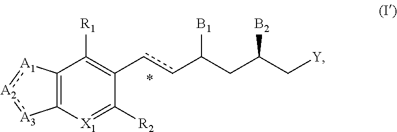

or a pharmaceutically acceptable salt thereof, wherein:
   [symbol] is a single or double bond, as valency allows;
   [symbol] is a single or double bond, wherein the double bond is the (E) isomer;
   X₁ is CH or N;
   A₁ is CR_{A1}, N, O, or S;
   A₂ is CR_{A2}, N, O, or S;
   A₃ is CR_{A3}, N, O, or S, wherein at least one A₁, A₂, or A₃ is S;
   R₁ is C₆-C₁₀ aryl or 5- to 10-membered heteroaryl, wherein the aryl or heteroaryl is optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl;
   R₂ is C₃-C₁₀ cycloalkyl;
   B₁ is H or —OH;
   B₂ is H or —OH;
   Y is H, —C(O)OR₃, —C(O)N(R₃)₂,
   each R₃ independently is H, C₁-C₆ alkyl, C₂-C₆ alkenyl, C₂-C₆ alkynyl, C₁-C₆ alkoxyl, or C₁-C₆ haloalkyl;
   R_{A1} is H, C₁-C₆ alkyl, C₂-C₆ alkenyl, C₂-C₆ alkynyl, C₁-C₆ alkoxyl, C₁-C₆ haloalkyl, or C₃-C₁₀ cycloalkyl;
   R_{A2} is H, C₁-C₆ alkyl, C₂-C₆ alkenyl, C₂-C₆ alkynyl, C₁-C₆ alkoxyl, C₁-C₆ haloalkyl, C₃-C₁₀ cycloalkyl, C₆-C₁₀ aryl, or 5- to 10-membered heteroaryl; and
   R_{A3} is H, C₁-C₆ alkyl, C₂-C₆ alkenyl, C₂-C₆ alkynyl, C₁-C₆ alkoxyl, C₁-C₆ haloalkyl, C₃-C₁₀ cycloalkyl, 3- to 10-membered heterocyclyl optionally substituted with one or more C₁-C₆ alkyl, C₆-C₁₀ aryl, or 5- to 10-membered heteroaryl optionally substituted with one or more C₁-C₆ alkyl,
provided that:
   (a) when R₂ is cyclopropyl, X₁ is N, A₁ is CR_{A1}, and A₂ is CR_{A2}, then at least one of R_{A1} and R_{A2} is not H; and
   (b) when R₂ is cyclopropyl, X₁ is N, A₁ is CR_{A1}, and A₂ is CH, then R_{A1} is not ethyl.

In some aspects, the present disclosure provides a compound of Formula (I′), or a pharmaceutically acceptable salt thereof, wherein:
[symbol] is a single or double bond, as valency allows;
[symbol] is a single or double bond, wherein the double bond is the (E) isomer;
X₁ is CH or N;
A₁ is CR_{A1}, N, O, or S;
A₂ is CR_{A2}, N, O, or S;
A₃ is CR_{A3}, N, O, or S, wherein at least one A₁, A₂, or A₃ is S;
R₁ is C₆-C₁₀ aryl or 5- to 10-membered heteroaryl, wherein the aryl or heteroaryl is optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl;
R₂ is C₃-C₁₀ cycloalkyl;
B₁ is H or —OH;
B₂ is H or —OH;
Y is H, —C(O)OR₃, —C(O)N(R₃)₂,
each R₃ independently is H, C₁-C₆ alkyl, C₂-C₆ alkenyl, C₂-C₆ alkynyl, C₁-C₆ alkoxyl, or C₁-C₆ haloalkyl;
R_{A1} is H, C₁-C₆ alkyl, C₂-C₆ alkenyl, C₂-C₆ alkynyl, C₁-C₆ alkoxyl, C₁-C₆ haloalkyl, or C₃-C₁₀ cycloalkyl;
R_{A2} is H, C₁-C₆ alkyl, C₂-C₆ alkenyl, C₂-C₆ alkynyl, C₁-C₆ alkoxyl, C₁-C₆ haloalkyl, C₃-C₁₀ cycloalkyl, C₆-C₁₀ aryl, or 5- to 10-membered heteroaryl; and
R_{A3} is H, C₁-C₆ alkyl, C₂-C₆ alkenyl, C₂-C₆ alkynyl, C₁-C₆ alkoxyl, C₁-C₆ haloalkyl, C₃-C₁₀ cycloalkyl, 3- to 10-membered heterocyclyl optionally substituted with one or more C₁-C₆ alkyl, C₆-C₁₀ aryl, or 5- to 10-membered heteroaryl optionally substituted with one or more C₁-C₆ alkyl,
   provided that:
(a) when R₂ is cyclopropyl, X₁ is N, A₁ is CR_{A1}, and A₂ is CR_{A2}, then at least one of R_{A1} and R_{A2} is not H.

In some aspects, the present disclosure provides a compound of Formula (I′), or a pharmaceutically acceptable salt thereof, wherein:
[symbol] is a single or double bond, as valency allows;
[symbol] is a single or double bond, wherein the double bond is the (E) isomer;
X₁ is CH or N;
A₁ is CR_{A1}, N, O, or S;
A₂ is CR_{A2}, N, O, or S;
A₃ is CR_{A3}, N, O, or S, wherein at least one A₁, A₂, or A₃ is S;
R₁ is C₆-C₁₀ aryl or 5- to 10-membered heteroaryl, wherein the aryl or heteroaryl is optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl;
R₂ is C₃-C₁₀ cycloalkyl;
B₁ is H or —OH;
B₂ is H or —OH;
Y is H, —C(O)OR₃, —C(O)N(R₃)₂,
each R₃ independently is H, C₁-C₆ alkyl, C₂-C₆ alkenyl, C₂-C₆ alkynyl, C₁-C₆ alkoxyl, or C₁-C₆ haloalkyl;
R_{A1} is H, C₁-C₆ alkyl, C₂-C₆ alkenyl, C₂-C₆ alkynyl, C₁-C₆ alkoxyl, C₁-C₆ haloalkyl, or C₃-C₁₀ cycloalkyl;
R_{A2} is H, C₁-C₆ alkyl, C₂-C₆ alkenyl, C₂-C₆ alkynyl, C₁-C₆ alkoxyl, C₁-C₆ haloalkyl, C₃-C₁₀ cycloalkyl, C₆-C₁₀ aryl, or 5- to 10-membered heteroaryl; and
R_{A3} is H, C₁-C₆ alkyl, C₂-C₆ alkenyl, C₂-C₆ alkynyl, C₁-C₆ alkoxyl, C₁-C₆ haloalkyl, C₃-C₁₀ cycloalkyl, 3- to 10-membered heterocyclyl optionally substituted with one or more C₁-C₆ alkyl, C₆-C₁₀ aryl, or 5- to 10-membered heteroaryl optionally substituted with one or more C₁-C₆ alkyl,
   provided that:
(b) when R₂ is cyclopropyl, X₁ is N, A₁ is CR_{A1}, and A₂ is CH, then R_{A1} is not ethyl.

In some aspects, the present disclosure provides a compound of Formula (I):

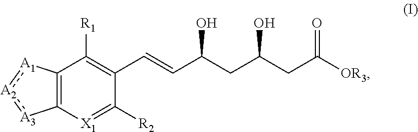

or a pharmaceutically acceptable salt thereof, wherein:
   [symbol] is a single or double bond, as valency allows;
   X₁ is CH or N;
   A₁ is CR_{A1}, N, O, or S;
   A₂ is CR_{A2}, N, O, or S;
   A₃ is CR_{A3}, N, O, or S, wherein at least one A₁, A₂, or A₃ is S;
   R₁ is C₆-C₁₀ aryl or 5- to 10-membered heteroaryl optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl;
   R₂ is C₃-C₁₀ cycloalkyl;
   R₃ is H, C₁-C₆ alkyl, C₂-C₆ alkenyl, C₂-C₆ alkynyl, C₁-C₆ alkoxyl, or C₁-C₆ haloalkyl;
   R_{A1} is H, C₁-C₆ alkyl, C₂-C₆ alkenyl, C₂-C₆ alkynyl, C₁-C₆ alkoxyl, or C₁-C₆ haloalkyl;
   R_{A2} is H, C₁-C₆ alkyl, C₂-C₆ alkenyl, C₂-C₆ alkynyl, C₁-C₆ alkoxyl, C₁-C₆ haloalkyl, C₆-C₁₀ aryl, or 5- to 10-membered heteroaryl; and
   R_{A3} is H, C₁-C₆ alkyl, C₂-C₆ alkenyl, C₂-C₆ alkynyl, C₁-C₆ alkoxyl, or C₁-C₆ haloalkyl,
provided that:
   (a) when R₂ is cyclopropyl, X₁ is N, A₁ is CR_{A1}, and A₂ is CR_{A2}, then at least one of R_{A1} and R_{A2} is not H; and
   (b) when R₂ is cyclopropyl, X₁ is N, A₁ is CR_{A1}, and A₂ is CH, then R_{A1} is not ethyl.

In some embodiments, the compound of Formula (I′) is a compound of Formula (I).

In some embodiments, the compound is of Formula (I), or a pharmaceutically acceptable salt thereof, wherein:
[symbol] is a single or double bond, as valency allows;
X₁ is CH or N;
A₁ is CR_{A1}, N, O, or S;
A₂ is CR_{A2}, N, O, or S;
A₃ is CR_{A3}, N, O, or S, wherein at least one A₁, A₂, or A₃ is S;
R₁ is C₆-C₁₀ aryl or 5- to 10-membered heteroaryl optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl;
R₂ is C₃-C₁₀ cycloalkyl;
R₃ is H, C₁-C₆ alkyl, C₂-C₆ alkenyl, C₂-C₆ alkynyl, C₁-C₆ alkoxyl, or C₁-C₆ haloalkyl;
R_{A1} is H, C₁-C₆ alkyl, C₂-C₆ alkenyl, C₂-C₆ alkynyl, C₁-C₆ alkoxyl, or C₁-C₆ haloalkyl;
R_{A2} is H, C₁-C₆ alkyl, C₂-C₆ alkenyl, C₂-C₆ alkynyl, C₁-C₆ alkoxyl, or C₁-C₆ haloalkyl; and
R_{A3} is H, C₁-C₆ alkyl, C₂-C₆ alkenyl, C₂-C₆ alkynyl, C₁-C₆ alkoxyl, or C₁-C₆ haloalkyl,
   provided that:
(a) when R₂ is cyclopropyl, X₁ is N, A₁ is CR_{A1}, and A₂ is CR_{A2}, then at least one of R_{A1} and R_{A2} is not H; and
(b) when R₂ is cyclopropyl, X₁ is N, A₁ is CR_{A1}, and A₂ is CH, then R_{A1} is not ethyl.

In some aspects, the present disclosure provides a compound of Formula (I):

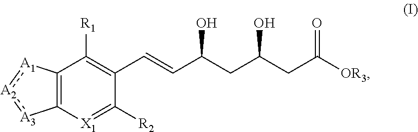

or a pharmaceutically acceptable salt thereof, wherein:
   [symbol] is a single or double bond, as valency allows;
   X₁ is CH or N;
   A₁ is CR_{A1}, N, O, or S;
   A₂ is CR_{A2}, N, O, or S;
   A₃ is CR_{A3}, N, O, or S, wherein at least one A₁, A₂, or A₃ is S;
   R₁ is C₆-C₁₀ aryl or 5- to 10-membered heteroaryl optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl;
   R₂ is C₃-C₁₀ cycloalkyl;
   R₃ is H, C₁-C₆ alkyl, C₂-C₆ alkenyl, C₂-C₆ alkynyl, C₁-C₆ alkoxyl, or C₁-C₆ haloalkyl;
   R_{A1} is C₁-C₆ alkyl, C₂-C₆ alkenyl, C₂-C₆ alkynyl, C₁-C₆ alkoxyl, or C₁-C₆ haloalkyl;
   R_{A2} is H, C₁-C₆ alkyl, C₂-C₆ alkenyl, C₂-C₆ alkynyl, C₁-C₆ alkoxyl, C₁-C₆ haloalkyl, C₆-C₁₀ aryl, or 5- to 10-membered heteroaryl; and
   R_{A3} is H, C₁-C₆ alkyl, C₂-C₆ alkenyl, C₂-C₆ alkynyl, C₁-C₆ alkoxyl, or C₁-C₆ haloalkyl,
provided that:
   (b) when R₂ is cyclopropyl, X₁ is N, A₁ is CR_{A1}, and A₂ is CH, then R_{A1} is not ethyl.

In some aspects, the present disclosure provides a compound of Formula (I):

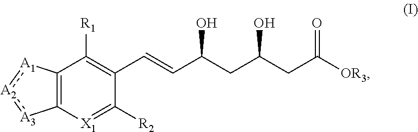

or a pharmaceutically acceptable salt thereof, wherein:
   [symbol] is a single or double bond, as valency allows;
   X₁ is CH or N;
   A₁ is CR_{A1}, N, O, or S;
   A₂ is CR_{A2}, N, O, or S;
   A₃ is CR_{A3}, N, O, or S, wherein at least one A₁, A₂, or A₃ is S;
   R₁ is C₆-C₁₀ aryl or 5- to 10-membered heteroaryl optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl;
   R₂ is C₃-C₁₀ cycloalkyl;
   R₃ is H, C₁-C₆ alkyl, C₂-C₆ alkenyl, C₂-C₆ alkynyl, C₁-C₆ alkoxyl, or C₁-C₆ haloalkyl;
   R_{A1} is H, C₁-C₆ alkyl, C₂-C₆ alkenyl, C₂-C₆ alkynyl, C₁-C₆ alkoxyl, or C₁-C₆ haloalkyl;
   R_{A2} is C₁-C₆ alkyl, C₂-C₆ alkenyl, C₂-C₆ alkynyl, C₁-C₆ alkoxyl, C₁-C₆ haloalkyl, C₆-C₁₀ aryl, or 5- to 10-membered heteroaryl; and
   R_{A3} is H, C₁-C₆ alkyl, C₂-C₆ alkenyl, C₂-C₆ alkynyl, C₁-C₆ alkoxyl, or C₁-C₆ haloalkyl,
provided that:
   (b) when R₂ is cyclopropyl, X₁ is N, A₁ is CR_{A1}, and A₂ is CH, then R_{A1} is not ethyl.

In some aspects, the present disclosure provides a compound of Formula (I):

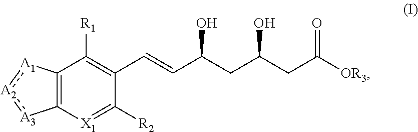

or a pharmaceutically acceptable salt thereof, wherein:
   [symbol] is a single or double bond, as valency allows;
   X₁ is CH or N;
   A₁ is CR_{A1}, N, O, or S;
   A₂ is CR_{A2}, N, O, or S;
   A₃ is CR_{A3}, N, O, or S, wherein at least one A₁, A₂, or A₃ is S;
   R₁ is C₆-C₁₀ aryl or 5- to 10-membered heteroaryl optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl;
   R₂ is C₃-C₁₀ cycloalkyl;
   R₃ is H, C₁-C₆ alkyl, C₂-C₆ alkenyl, C₂-C₆ alkynyl, C₁-C₆ alkoxyl, or C₁-C₆ haloalkyl;
   R_{A1} is H, C₁ or C₃-C₆ alkyl, C₂-C₆ alkenyl, C₂-C₆ alkynyl, C₁-C₆ alkoxyl, or C₁-C₆ haloalkyl;
   R_{A2} is H, C₁-C₆ alkyl, C₂-C₆ alkenyl, C₂-C₆ alkynyl, C₁-C₆ alkoxyl, C₁-C₆ haloalkyl, C₆-C₁₀ aryl, or 5- to 10-membered heteroaryl; and
   R_{A3} is H, C₁-C₆ alkyl, C₂-C₆ alkenyl, C₂-C₆ alkynyl, C₁-C₆ alkoxyl, or C₁-C₆ haloalkyl,
provided that:
   (a) when R₂ is cyclopropyl, X₁ is N, A₁ is CR_{A1}, and A₂ is CR_{A2}, then at least one of R_{A1} and R_{A2} is not H.

In some aspects, the present disclosure provides a compound of Formula (I):

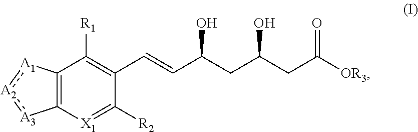

or a pharmaceutically acceptable salt thereof, wherein:
   [symbol] is a single or double bond, as valency allows;
   X₁ is CH or N;
   A₁ is CR_{A1}, N, O, or S;
   A₂ is CR_{A2}, N, O, or S;
   A₃ is CR_{A3}, N, O, or S, wherein at least one A₁, A₂, or A₃ is S;
   R₁ is C₆-C₁₀ aryl or 5- to 10-membered heteroaryl optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl;
   R₂ is C₃-C₁₀ cycloalkyl;
   R₃ is H, C₁-C₆ alkyl, C₂-C₆ alkenyl, C₂-C₆ alkynyl, C₁-C₆ alkoxyl, or C₁-C₆ haloalkyl;
   R_{A1} is C₁ or C₃-C₆ alkyl, C₂-C₆ alkenyl, C₂-C₆ alkynyl, C₁-C₆ alkoxyl, or C₁-C₆ haloalkyl;
   R_{A2} is H, C₁-C₆ alkyl, C₂-C₆ alkenyl, C₂-C₆ alkynyl, C₁-C₆ alkoxyl, C₁-C₆ haloalkyl, C₆-C₁₀ aryl, or 5- to 10-membered heteroaryl; and
   R_{A3} is H, C₁-C₆ alkyl, C₂-C₆ alkenyl, C₂-C₆ alkynyl, C₁-C₆ alkoxyl, or C₁-C₆ haloalkyl.

In some aspects, the present disclosure provides a compound of Formula (I):

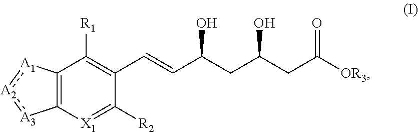

or a pharmaceutically acceptable salt thereof, wherein:
   [symbol] is a single or double bond, as valency allows;
   X₁ is CH or N;
   A₁ is CR_{A1}, N, O, or S;
   A₂ is CR_{A2}, N, O, or S;
   A₃ is CR_{A3}, N, O, or S, wherein at least one A₁, A₂, or A₃ is S;
   R₁ is C₆-C₁₀ aryl or 5- to 10-membered heteroaryl optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl;
   R₂ is C₃-C₁₀ cycloalkyl;
   R₃ is H, C₁-C₆ alkyl, C₂-C₆ alkenyl, C₂-C₆ alkynyl, C₁-C₆ alkoxyl, or C₁-C₆ haloalkyl;
   R_{A1} is H, C₁ or C₃-C₆ alkyl, C₂-C₆ alkenyl, C₂-C₆ alkynyl, C₁-C₆ alkoxyl, or C₁-C₆ haloalkyl;
   R_{A2} is C₁-C₆ alkyl, C₂-C₆ alkenyl, C₂-C₆ alkynyl, C₁-C₆ alkoxyl, C₁-C₆ haloalkyl, C₆-C₁₀ aryl, or 5- to 10-membered heteroaryl; and
   R_{A3} is H, C₁-C₆ alkyl, C₂-C₆ alkenyl, C₂-C₆ alkynyl, C₁-C₆ alkoxyl, or C₁-C₆ haloalkyl.

In some aspects, the present disclosure provides a compound of Formula (I″):

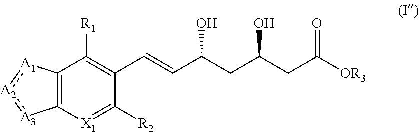

or a pharmaceutically acceptable salt thereof, wherein:
   [symbol] is a single or double bond, as valency allows;
   X₁ is CH or N;
   A₁ is CR_{A1}, N, O, or S;
   A₂ is CR_{A2}, N, O, or S;
   A₃ is CR_{A3}, N, O, or S, wherein at least one A₁, A₂, or A₃ is S;
   R₁ is C₆-C₁₀ aryl or 5- to 10-membered heteroaryl optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl;
   R₂ is C₃-C₁₀ cycloalkyl;
   R₃ is H, C₁-C₆ alkyl, C₂-C₆ alkenyl, C₂-C₆ alkynyl, C₁-C₆ alkoxyl, or C₁-C₆ haloalkyl;
   R_{A1} is H, C₁-C₆ alkyl, C₂-C₆ alkenyl, C₂-C₆ alkynyl, C₁-C₆ alkoxyl, or C₁-C₆ haloalkyl;
   R_{A2} is H, C₁-C₆ alkyl, C₂-C₆ alkenyl, C₂-C₆ alkynyl, C₁-C₆ alkoxyl, C₁-C₆ haloalkyl, C₆-C₁₀ aryl, or 5- to 10-membered heteroaryl; and
   R_{A3} is H, C₁-C₆ alkyl, C₂-C₆ alkenyl, C₂-C₆ alkynyl, C₁-C₆ alkoxyl, or C₁-C₆ haloalkyl,
provided that:
   (a) when R₂ is cyclopropyl, X₁ is N, A₁ is CR_{A1}, and A₂ is CR_{A2}, then at least one of R_{A1} and R_{A2} is not H; and
   (b) when R₂ is cyclopropyl, X₁ is N, A₁ is CR_{A1}, and A₂ is CH, then R_{A1} is not ethyl.

In some aspects, the present disclosure provides a compound of Formula (I′″):

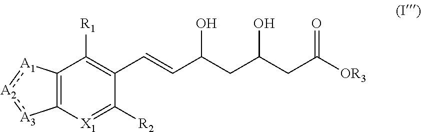

or a pharmaceutically acceptable salt thereof, wherein:
   [symbol] is a single or double bond, as valency allows;
   X₁ is CH or N;
   A₁ is CR_{A1}, N, O, or S;
   A₂ is CR_{A2}, N, O, or S;
   A₃ is CR_{A3}, N, O, or S, wherein at least one A₁, A₂, or A₃ is S;
   R₁ is C₆-C₁₀ aryl or 5- to 10-membered heteroaryl optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl;
   R₂ is C₃-C₁₀ cycloalkyl;
   R₃ is H, C₁-C₆ alkyl, C₂-C₆ alkenyl, C₂-C₆ alkynyl, C₁-C₆ alkoxyl, or C₁-C₆ haloalkyl;
   R_{A1} is H, C₁-C₆ alkyl, C₂-C₆ alkenyl, C₂-C₆ alkynyl, C₁-C₆ alkoxyl, or C₁-C₆ haloalkyl;
   R_{A2} is H, C₁-C₆ alkyl, C₂-C₆ alkenyl, C₂-C₆ alkynyl, C₁-C₆ alkoxyl, C₁-C₆ haloalkyl, C₆-C₁₀ aryl, or 5- to 10-membered heteroaryl; and
   R_{A3} is H, C₁-C₆ alkyl, C₂-C₆ alkenyl, C₂-C₆ alkynyl, C₁-C₆ alkoxyl, or C₁-C₆ haloalkyl,
provided that:
   (a) when R₂ is cyclopropyl, X₁ is N, A₁ is CR_{A1}, and A₂ is CR_{A2}, then at least one of R_{A1} and R_{A2} is not H; and
   (b) when R₂ is cyclopropyl, X₁ is N, A₁ is CR_{A1}, and A₂ is CH, then R_{A1} is not ethyl.

It is understood that, for a compound of the present disclosure, variables Y, X₁, A₁, A₂, A₃, B₁, B₂, R₁, R₂, R₃, R_{A1}, R_{A2}, and R_{A3} can each be, where applicable, selected from the groups described herein, and any group described herein for any of variables Y, X₁, A₁, A₂, A₃, B₁, B₂, R₁, R₂, R₃, R_{A1}, R_{A2}, and R_{A3} can be combined, where applicable, with any group described herein for one or more of the remainder of variables Y, X₁, A₁, A₂, A₃, B₁, B₂, R₁, R₂, R₃, R_{A1}, R_{A2}, and R_{A3}.

In some embodiments, [symbol] is a single bond, as valency allows.

In some embodiments, [symbol] is a double bond, as valency allows.

In some embodiments, [symbol] is a single bond.

In some embodiments, [symbol] is a double bond, wherein the double bond is the (E) isomer.

In some embodiments, X₁ is CH or N.

In some embodiments, X₁ is CH. In some embodiments, X₁ is N.

In some embodiments, A₁ is CR_{A1}, N, O, or S.

In some embodiments, A₁ is CR_{A1}. In some embodiments, A₁ is CH.

In some embodiments, A₁ is N. In some embodiments, A₁ is O. In some embodiments, A₁ is S.

In some embodiments, A₂ is CR_{A2}, N, O, or S.

In some embodiments, A₂ is CR_{A2}. In some embodiments, A₂ is CH.

In some embodiments, A₂ is N. In some embodiments, A₂ is O. In some embodiments, A₂ is S.

In some embodiments, A₃ is CR_{A3}, N, O, or S.

In some embodiments, A₃ is CR_{A3}. In some embodiments, A₃ is CH.

In some embodiments, A₃ is N. In some embodiments, A₃ is O. In some embodiments, A₃ is S.

In some embodiments, at least one A₁, A₂, or A₃ is S.

In some embodiments, B₁ is H.

In some embodiments, B₁ is —OH.

In some embodiments, B₂ is H.

In some embodiments, B₂ is —OH.

In some embodiments, Y is H.

In some embodiments, Y is —C(O)OR₃. In some embodiments, Y is —C(O)OH. In some embodiments, Y is —C(O)OMe.

In some embodiments, Y is —C(O)N(R₃)₂. In some embodiments, Y is —C(O)NH₂. In some embodiments, Y is —C(O)NH (Me). In some embodiments, Y is —C(O)N (Me) 2.

In some embodiments, R₁ is C₆-C₁₀ aryl or 5- to 10-membered heteroaryl.

In some embodiments, R₁ is C₆-C₁₀ aryl or 5- to 10-membered heteroaryl optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl.

In some embodiments, R₁ is C₆-C₁₀ aryl or 5- to 10-membered heteroaryl substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl.

In some embodiments, R₁ is C₆-C₁₀ aryl or 5- to 10-membered heteroaryl substituted with one or more halo.

In some embodiments, R₁ is C₆-C₁₀ aryl or 5- to 10-membered heteroaryl substituted with one halo.

In some embodiments, R₁ is C₆-C₁₀ aryl.

In some embodiments, R₁ is C₆-C₁₀ aryl optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl.

In some embodiments, R₁ is C₆-C₁₀ aryl substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl.

In some embodiments, R₁ is C₆-C₁₀ aryl substituted with one or more halo.

In some embodiments, R₁ is C₆-C₁₀ aryl substituted with one halo.

In some embodiments, R₁ is C₆ aryl.

In some embodiments, R₁ is C₆ aryl optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl.

In some embodiments, R₁ is C₆ aryl substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl.

In some embodiments, R₁ is C₆ aryl substituted with one or more halo.

In some embodiments, R₁ is C₆ aryl substituted with one halo.

In some embodiments, R₁ is 5- to 10-membered heteroaryl.

In some embodiments, R₁ is 5- to 10-membered heteroaryl optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl.

In some embodiments, R₁ is 5- to 10-membered heteroaryl substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl.

In some embodiments, R₁ is 5- to 10-membered heteroaryl substituted with one or more halo.

In some embodiments, R₁ is 5- to 10-membered heteroaryl substituted with one halo.

In some embodiments, R₁ is 6-membered heteroaryl.

In some embodiments, R₁ is 6-membered heteroaryl optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl.

In some embodiments, R₁ is 6-membered heteroaryl substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl.

In some embodiments, R₁ is 6-membered heteroaryl substituted with one or more halo.

In some embodiments, R₁ is 6-membered heteroaryl substituted with one halo.

In some embodiments, R₁ is 6-membered heteroaryl substituted with two halo.

In some embodiments, R₁ is 6-membered heteroaryl substituted with one halo and one methyl.

In some embodiments, R₁ is

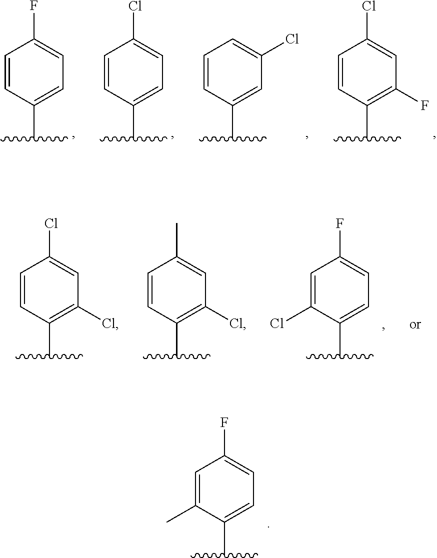

In some embodiments, R₁ is

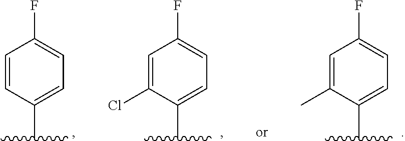

In some embodiments, R₁ is

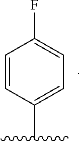 [SMILES: *c1ccc(F)cc1]

In some embodiments, R₁ is substituted, at least, at the 2-position.

In some embodiments, R₁ is substituted, at least, at the 3-position.

In some embodiments, R₁ is substituted, at least, at the 4-position.

In some embodiments, R₂ is C₃-C₁₀ cycloalkyl.

In some embodiments, R₂ is C₃ cycloalkyl (cyclopropyl).

In some embodiments, R₂ is C₄ cycloalkyl (cyclobutyl).

In some embodiments, R₂ is C₈ cycloalkyl. In some embodiments, R₂ is C₆ cycloalkyl. In some embodiments, R₂ is C₇ cycloalkyl. In some embodiments, R₂ is C₈ cycloalkyl. In some embodiments, R₂ is C₉ cycloalkyl. In some embodiments, R₂ is C₁₀ cycloalkyl.

In some embodiments, R₂ is C₅-C₁₀ cycloalkyl. In some embodiments, R₂ is bridged C₅-C₁₀ cycloalkyl. In some embodiments, R₂ is bicyclic C₅-C₁₀ cycloalkyl.

In some embodiments, R₂ is

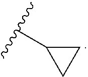 [SMILES: *C1CC1]

In some embodiments, R₃ is H, C₁-C₆ alkyl, C₂-C₆ alkenyl, C₂-C₆ alkynyl, C₁-C₆ alkoxyl, or C₁-C₆ haloalkyl.

In some embodiments, R₃ is H.

In some embodiments, R₃ is C₁-C₆ alkyl, C₂-C₆ alkenyl, C₂-C₆ alkynyl, C₁-C₆ alkoxyl, or C₁-C₆ haloalkyl.

In some embodiments, R₃ is C₁-C₆ alkyl.

In some embodiments, R₃ is methyl. In some embodiments, R₃ is ethyl. In some embodiments, R₃ is propyl. In some embodiments, R₃ is butyl. In some embodiments, R₃ is pentyl. In some embodiments, R₃ is hexyl. In some embodiments, R₃ is isopropyl. In some embodiments, R₃ is isobutyl. In some embodiments, R₃ is isopentyl. In some embodiments, R₃ is isohexyl. In some embodiments, R₃ is secbutyl. In some embodiments, R₃ is secpentyl. In some embodiments, R₃ is sechexyl. In some embodiments, R₃ is tertbutyl.

In some embodiments, R₃ is C₂-C₆ alkenyl (e.g., ethenyl, propenyl, butenyl).

In some embodiments, R₃ is C₂-C₆ alkynyl (e.g., ethynyl, propynyl, butynyl).

In some embodiments, R₃ is C₁-C₆ alkoxyl.

In some embodiments, R₃ is methoxyl. In some embodiments, R₃ is ethoxyl. In some embodiments, R₃ is propoxyl. In some embodiments, R₃ is butoxyl. In some embodiments, R₃ is pentoxyl. In some embodiments, R₃ is hexoxyl.

In some embodiments, R₃ is C₁-C₆ haloalkyl.

In some embodiments, R₃ is halomethyl. In some embodiments, R₃ is haloethyl. In some embodiments, R₃ is halopropyl. In some embodiments, R₃ is halobutyl. In some embodiments, R₃ is halopentyl. In some embodiments, R₃ is halohexyl.

In some embodiments, at least one R₃ is C₁-C₆ alkyl, C₂-C₆ alkenyl, C₂-C₆ alkynyl, C₁-C₆ alkoxyl, or C₁-C₆ haloalkyl.

In some embodiments, at least one R₃ is C₁-C₆ alkyl.

In some embodiments, at least one R₃ is methyl. In some embodiments, at least one R₃ is ethyl. In some embodiments, at least one Rₐ is propyl. In some embodiments, at least one R₃ is butyl. In some embodiments, at least one R₅ is pentyl. In some embodiments, at least one R₃ is hexyl. In some embodiments, at least one R₃ is isopropyl. In some embodiments, at least one R₃ is isobutyl. In some embodiments, at least one R₃ is isopentyl. In some embodiments, at least one R₃ is isohexyl. In some embodiments, at least one R₃ is secbutyl. In some embodiments, at least one R₃ is secpentyl. In some embodiments, at least one R₃ is sechexyl. In some embodiments, at least one R₃ is tertbutyl.

In some embodiments, at least one R₃ is C₂-C₆ alkenyl (e.g., ethenyl, propenyl, butenyl).

In some embodiments, at least one R₃ is C₂-C₆ alkynyl (e.g., ethynyl, propynyl, butynyl).

In some embodiments, at least one R₃ is C₁-C₆ alkoxyl.

In some embodiments, at least one R₃ is methoxyl. In some embodiments, at least one R₃ is ethoxyl. In some embodiments, at least one R₃ is propoxyl. In some embodiments, at least one R₃ is butoxyl. In some embodiments, at least one R₃ is pentoxyl. In some embodiments, at least one R₃ is hexoxyl.

In some embodiments, at least one R₃ is C₁-C₆ haloalkyl.

In some embodiments, at least one R₃ is halomethyl. In some embodiments, at least one R₃ is haloethyl. In some embodiments, at least one R₃ is halopropyl. In some embodiments, at least one R₃ is halobutyl. In some embodiments, at least one R₃ is halopentyl. In some embodiments, at least one R₅ is halohexyl.

In some embodiments, R₃ is H or methyl.

In some embodiments, R_{A1} is H, C₁-C₆ alkyl, C₂-C₆ alkenyl, C₂-C₆ alkynyl, C₁-C₆ alkoxyl, C₁-C₆ haloalkyl, or C₃-C₁₀ cycloalkyl.

In some embodiments, R_{A1} is H, C₁-C₆ alkyl, C₂-C₆ alkenyl, C₂-C₆ alkynyl, C₁-C₆ alkoxyl, or C₁-C₆ haloalkyl.

In some embodiments, R_{A1} is H.

In some embodiments, R_{A1} is C₁-C₆ alkyl, C₂-C₆ alkenyl, C₂-C₆ alkynyl, C₁-C₆ alkoxyl, or C₁-C₆ haloalkyl.

In some embodiments, R_{A1} is C₁-C₆ alkyl.

In some embodiments, R_{A1} is methyl. In some embodiments, R_{A1} is ethyl. In some embodiments, R_{A1} is propyl. In some embodiments, R_{A1} is butyl. In some embodiments, R_{A1} is pentyl. In some embodiments, R_{A1} is hexyl. In some embodiments, R_{A1} is isopropyl. In some embodiments, R_{A1} is isobutyl. In some embodiments, R_{A1} is isopentyl. In some embodiments, R_{A1} is isohexyl. In some embodiments, R_{A1} is secbutyl. In some embodiments, R_{A1} is secpentyl. In some embodiments, R_{A1} is sechexyl. In some embodiments, R_{A1} is tertbutyl.

In some embodiments, R_{A1} is C₂-C₆ alkenyl (e.g., ethenyl, propenyl, butenyl).

In some embodiments, R_{A1} is C₂-C₆ alkynyl (e.g., ethynyl, propynyl, butynyl). In some embodiments, R_{A1} is C₁-C₆ alkoxyl.

In some embodiments, R_{A1} is methoxyl. In some embodiments, R_{A1} is ethoxyl. In some embodiments, R_{A1} is propoxyl. In some embodiments, R_{A1} is butoxyl. In some embodiments, R_{A1} is pentoxyl. In some embodiments, R_{A1} is hexoxyl.

In some embodiments, R_{A1} is C₁-C₆ haloalkyl.

In some embodiments, R_{A1} is halomethyl. In some embodiments, R_{A1} is haloethyl. In some embodiments, R_{A1} is halopropyl. In some embodiments, R_{A1} is halobutyl. In some embodiments, R_{A1} is halopentyl. In some embodiments, R_{A1} is halohexyl.

In some embodiments, R_{A1} is C₃-C₁₀ cycloalkyl.

In some embodiments, R_{A1} is C₃ cycloalkyl (cyclopropyl).

In some embodiments, R_{A1} is C₄ cycloalkyl (cyclobutyl).

In some embodiments, R_{A1} is C₈ cycloalkyl. In some embodiments, R_{A1} is C₆ cycloalkyl. In some embodiments, R_{A1} is C₇ cycloalkyl. In some embodiments, R_{A1} is C₈ cycloalkyl. In some embodiments, R_{A1} is C₉ cycloalkyl. In some embodiments, R_{A1} is C₁₀ cycloalkyl.

In some embodiments, R_{A1} is C₅-C₁₀ cycloalkyl. In some embodiments, R_{A1} is bridged C₅-C₁₀ cycloalkyl. In some embodiments, R_{A1} is bicyclic C₅-C₁₀ cycloalkyl.

In some embodiments, R_{A1} is H, methyl, or cyclopropyl.

In some embodiments, R_{A1} is H or methyl.

In some embodiments, R_{A2} is H, C₁-C₆ alkyl, C₂-C₆ alkenyl, C₂-C₆ alkynyl, C₁-C₆ alkoxyl, C₁-C₆ haloalkyl, C₃-C₁₀ cycloalkyl, C₆-C₁₀ aryl, or 5- to 10-membered heteroaryl.

In some embodiments, R_{A2} is H, C₁-C₆ alkyl, C₂-C₆ alkenyl, C₂-C₆ alkynyl, C₁-C₆ alkoxyl, C₁-C₆ haloalkyl, C₆-C₁₀ aryl, or 5- to 10-membered heteroaryl.

In some embodiments, R_{A2} is H, C₁-C₆ alkyl, C₂-C₆ alkenyl, C₂-C₆ alkynyl, C₁-C₆ alkoxyl, or C₁-C₆ haloalkyl.

In some embodiments, R_{A2} is H.

In some embodiments, R_{A2} is C₁-C₆ alkyl, C₂-C₆ alkenyl, C₂-C₆ alkynyl, C₁-C₆ alkoxyl, C₁-C₆ haloalkyl, C₆-C₁₀ aryl, or 5- to 10-membered heteroaryl.

In some embodiments, R_{A2} is C₁-C₆ alkyl, C₂-C₆ alkenyl, C₂-C₆ alkynyl, C₁-C₆ alkoxyl, or C₁-C₆ haloalkyl.

In some embodiments, R_{A2} is C₆-C₁₀ aryl.

In some embodiments, R_{A2} is C₆ aryl.

In some embodiments, R_{A2} is C₈ aryl. In some embodiments, R_{A2} is C₁₀ aryl.

In some embodiments, R_{A2} is 5- to 10-membered heteroaryl.

In some embodiments, R_{A2} is 5-membered heteroaryl. In some embodiments, R_{A2} is 6-membered heteroaryl. In some embodiments, R_{A2} is 7-membered heteroaryl. In some embodiments, R_{A2} is 8-membered heteroaryl. In some embodiments, R_{A2} is 9-membered heteroaryl. In some embodiments, R_{A2} is 10-membered heteroaryl.

In some embodiments, R_{A2} is C₁-C₆ alkyl.

In some embodiments, R_{A2} is methyl. In some embodiments, R_{A2} is ethyl. In some embodiments, R_{A2} is propyl. In some embodiments, R_{A2} is butyl. In some embodiments, R_{A2} is pentyl. In some embodiments, R_{A2} is hexyl. In some embodiments, R_{A2} is isopropyl. In some embodiments, R_{A2} is isobutyl. In some embodiments, R_{A2} is isopentyl. In some embodiments, R_{A2} is isohexyl. In some embodiments, R_{A2} is secbutyl. In some embodiments, R_{A2} is secpentyl. In some embodiments, R_{A2} is sechexyl. In some embodiments, R_{A2} is tertbutyl.

In some embodiments, R_{A2} is C₂-C₆ alkenyl (e.g., ethenyl, propenyl, butenyl).

In some embodiments, R_{A2} is C₂-C₆ alkynyl (e.g., ethynyl, propynyl, butynyl).

In some embodiments, R_{A2} is C₁-C₆ alkoxyl.

In some embodiments, R_{A2} is methoxyl. In some embodiments, R_{A2} is ethoxyl. In some embodiments, R_{A2} is propoxyl. In some embodiments, R_{A2} is butoxyl. In some embodiments, R_{A2} is pentoxyl. In some embodiments, R_{A2} is hexoxyl.

In some embodiments, R_{A2} is C₁-C₆ haloalkyl.

In some embodiments, R_{A2} is halomethyl. In some embodiments, R_{A2} is haloethyl. In some embodiments, R_{A2} is halopropyl. In some embodiments, R_{A2} is halobutyl. In some embodiments, R_{A2} is halopentyl. In some embodiments, R_{A2} is halohexyl.

In some embodiments, R_{A2} is C₃-C₁₀ cycloalkyl.

In some embodiments, R_{A2} is C₃ cycloalkyl (cyclopropyl).

In some embodiments, R_{A2} is C₄ cycloalkyl (cyclobutyl).

In some embodiments, R_{A2} is C₈ cycloalkyl. In some embodiments, R_{A2} is C₆ cycloalkyl. In some embodiments, R_{A2} is C₇ cycloalkyl. In some embodiments, R_{A2} is C₈ cycloalkyl. In some embodiments, R_{A2} is C₉ cycloalkyl. In some embodiments, R_{A2} is C₁₀ cycloalkyl.

In some embodiments, R_{A2} is C₅-C₁₀ cycloalkyl. In some embodiments, R_{A2} is bridged C₅-C₁₀ cycloalkyl. In some embodiments, R_{A2} is bicyclic C₅-C₁₀ cycloalkyl.

In some embodiments, R_{A2} is H, methyl, or cyclopropyl.

In some embodiments, R_{A2} is H or methyl.

In some embodiments, R_{A3} is H, C₁-C₆ alkyl, C₂-C₆ alkenyl, C₂-C₆ alkynyl, C₁-C₆ alkoxyl, C₁-C₆ haloalkyl, C₃-C₁₀ cycloalkyl, 3- to 10-membered heterocyclyl optionally substituted with one or more C₁-C₆ alkyl, C₆-C₁₀ aryl, or 5- to 10-membered heteroaryl optionally substituted with one or more C₁-C₆ alkyl.

In some embodiments, R_{A3} is H, C₁-C₆ alkyl, C₂-C₆ alkenyl, C₂-C₆ alkynyl, C₁-C₆ alkoxyl, or C₁-C₆ haloalkyl.

In some embodiments, R_{A3} is H.

In some embodiments, R_{A3} is C₁-C₆ alkyl, C₂-C₆ alkenyl, C₂-C₆ alkynyl, C₁-C₆ alkoxyl, or C₁-C₆ haloalkyl.

In some embodiments, R_{A3} is C₁-C₆ alkyl.

In some embodiments, R_{A3} is methyl. In some embodiments, R_{A3} is ethyl. In some embodiments, R_{A3} is propyl. In some embodiments, R_{A3} is butyl. In some embodiments, R_{A3} is pentyl. In some embodiments, R_{A3} is hexyl. In some embodiments, R_{A3} is isopropyl. In some embodiments, R_{A3} is isobutyl. In some embodiments, R_{A3} is isopentyl. In some embodiments, R_{A3} is isohexyl. In some embodiments, R_{A3} is secbutyl. In some embodiments, R_{A3} is secpentyl. In some embodiments, R_{A3} is sechexyl. In some embodiments, R_{A3} is tertbutyl.

In some embodiments, R_{A3} is C₂-C₆ alkenyl (e.g., ethenyl, propenyl, butenyl).

In some embodiments, R_{A3} is C₂-C₆ alkynyl (e.g., ethynyl, propynyl, butynyl).

In some embodiments, R_{A3} is C₁-C₆ alkoxyl.

In some embodiments, R_{A3} is methoxyl. In some embodiments, R_{A3} is ethoxyl. In some embodiments, R_{A3} is propoxyl. In some embodiments, R_{A3} is butoxyl. In some embodiments, R_{A3} is pentoxyl. In some embodiments, R_{A3} is hexoxyl.

In some embodiments, R_{A3} is C₁-C₆ haloalkyl.

In some embodiments, R_{A3} is halomethyl. In some embodiments, R_{A3} is haloethyl. In some embodiments, R_{A3} is halopropyl. In some embodiments, R_{A3} is halobutyl. In some embodiments, R_{A3} is halopentyl. In some embodiments, R_{A3} is halohexyl.

In some embodiments, R_{A3} is C₃-C₁₀ cycloalkyl.

In some embodiments, R_{A3} is C₃ cycloalkyl (cyclopropyl).

In some embodiments, R_{A3} is C₄ cycloalkyl (cyclobutyl).

In some embodiments, R_{A3} is C₈ cycloalkyl. In some embodiments, R_{A3} is C₆ cycloalkyl. In some embodiments, R_{A3} is C₇ cycloalkyl. In some embodiments, R_{A3} is C₈ cycloalkyl. In some embodiments, R_{A3} is C₉ cycloalkyl. In some embodiments, R_{A3} is C₁₀ cycloalkyl.

In some embodiments, R_{A3} is C₅-C₁₀ cycloalkyl. In some embodiments, R_{A3} is bridged C₅-C₁₀ cycloalkyl. In some embodiments, R_{A3} is bicyclic C₅-C₁₀ cycloalkyl.

In some embodiments, R_{A3} is 3- to 10-membered heterocyclyl.

In some embodiments, R_{A3} is 3-membered heterocyclyl. In some embodiments, R_{A3} is 4-membered heterocyclyl. In some embodiments, R_{A3} is 5-membered heterocyclyl. In some embodiments, R_{A3} is 6-membered heterocyclyl. In some embodiments, R_{A3} is 7-membered heterocyclyl. In some embodiments, R_{A3} is 8-membered heterocyclyl. In some embodiments, R_{A3} is 9-membered heterocyclyl. In some embodiments, R_{A3} is 10-membered heterocyclyl.

In some embodiments, R_{A3} is 3- to 10-membered heterocyclyl optionally substituted with one or more C₁-C₆ alkyl.

In some embodiments, R_{A3} is 3- to 10-membered heterocyclyl substituted with one or more C₁-C₆ alkyl.

In some embodiments, R_{A3} is C₆-C₁₀ aryl.

In some embodiments, R_{A3} is C₆ aryl.

In some embodiments, R_{A3} is 5- to 10-membered heteroaryl.

In some embodiments, R_{A3} is 5- to 10-membered heteroaryl optionally substituted with one or more C₁-C₆ alkyl.

In some embodiments, R_{A3} is 5- to 10-membered heteroaryl substituted with one or more C₁-C₆ alkyl.

In some embodiments, R_{A3} is 5- to 6-membered heteroaryl.

In some embodiments, R_{A3} is 5- to 6-membered heteroaryl optionally substituted with one or more C₁-C₆ alkyl.

In some embodiments, R_{A3} is 5- to 6-membered heteroaryl substituted with one or more C₁-C₆ alkyl.

In some embodiments, R_{A3} is H, methyl, phenyl, cyclopropyl, cyclobutyl, cyclohexyl,

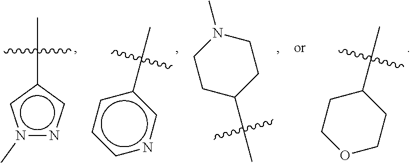

In some embodiments, R_{A3} is H or methyl.

In some embodiments, when R₂ is cyclopropyl, X₁ is N, A₁ is CR_{A1}, and A₂ is CR_{A2}, then at least one of R_{A1} and R_{A2} is not H.

In some embodiments, when R₂ is cyclopropyl, X₁ is N, A₁ is CR_{A1}, and A₂ is CR_{A2}, then R_{A1} is not H.

In some embodiments, when R₂ is cyclopropyl, X₁ is N, A₁ is CR_{A1}, and A₂ is CR_{A2}, then R_{A2} is not H.

In some embodiments, when R₂ is cyclopropyl, X₁ is N, A₁ is CR_{A1}, and A₂ is CH, then R_{A1} is not ethyl.

In some embodiments, the compound of Formula (I) or Formula (I′) is of Formula (I′):

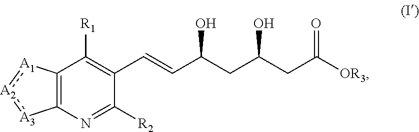

or a pharmaceutically acceptable salt thereof.

In some embodiments, the compound of Formula (I) or Formula (I′) is of Formula (I-a), (I-b), (I-c), or (I-d):

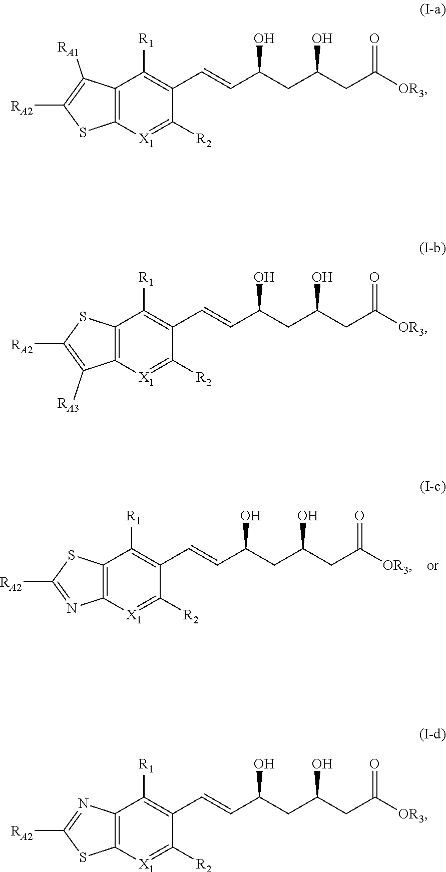

or a pharmaceutically acceptable salt thereof.

In some embodiments, the compound of Formula (I) or Formula (I′) is of Formula (I′-a), (I′-b), (I′-c), or (I′-d):

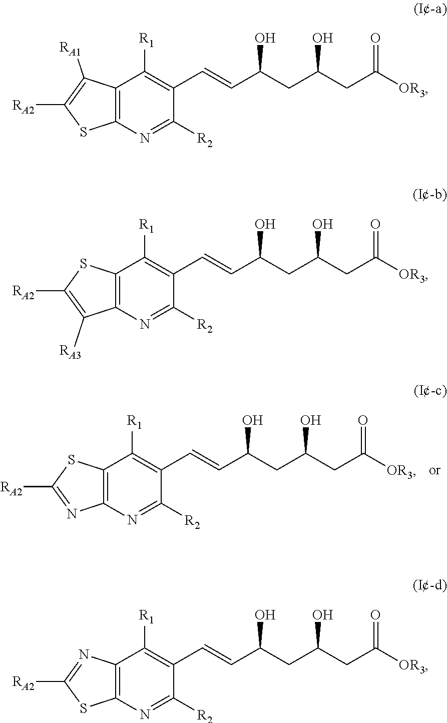

or a pharmaceutically acceptable salt thereof.

In some embodiments, the compound of Formula (I) or Formula (I′) is of Formula (I-d), (I-e), (I-f), or (I-g):

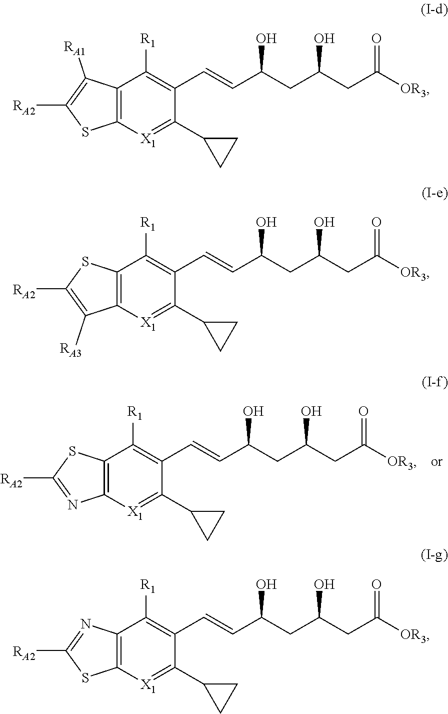

or a pharmaceutically acceptable salt thereof.

In some embodiments, the compound of Formula (I) or Formula (I′) is of Formula (I′-d), (I′-e), (I′-f), or (I′-g):

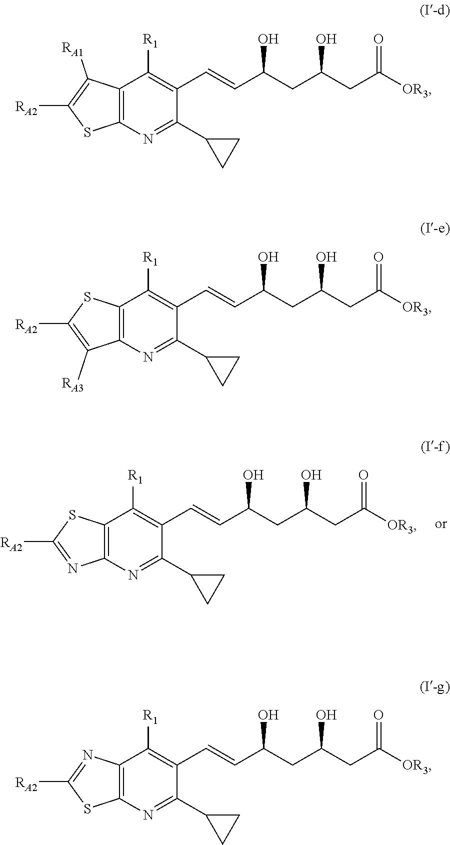

or a pharmaceutically acceptable salt thereof.

In some embodiments, the compound of Formula (I) or Formula (I′) is of Formula (I-h), (I-i), (I-j), or (I-k):

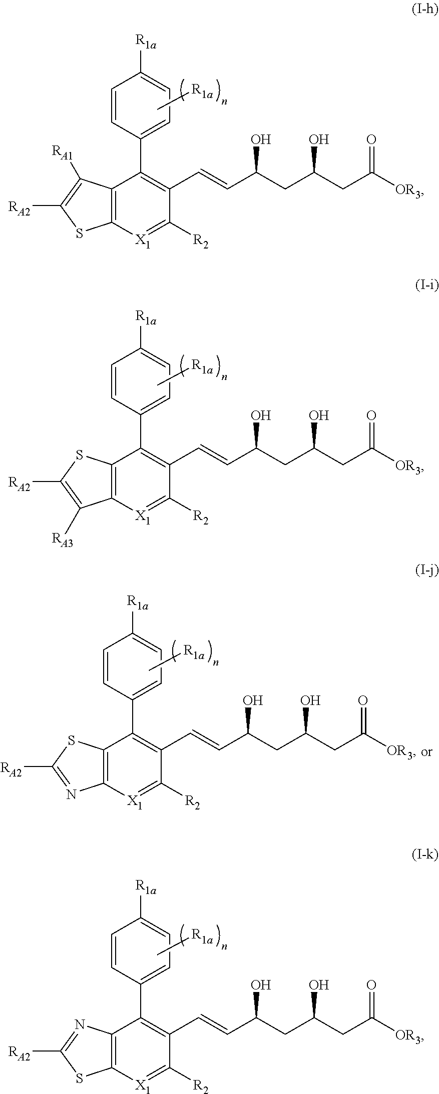

or a pharmaceutically acceptable salt thereof, wherein R₁ₐ is halo, —CN, —OH, —NH₂, C₁-C₆ alkyl, C₂-C₆ alkenyl, C₂-C₆ alkynyl, C₁-C₆ alkoxyl, or C₁-C₆ haloalkyl, and n is 0, 1, 2, 3, or 4.

In some embodiments, the compound of Formula (I) or Formula (I′) is of Formula (I′-h), (I′-i), (I′-j), or (I′-k):

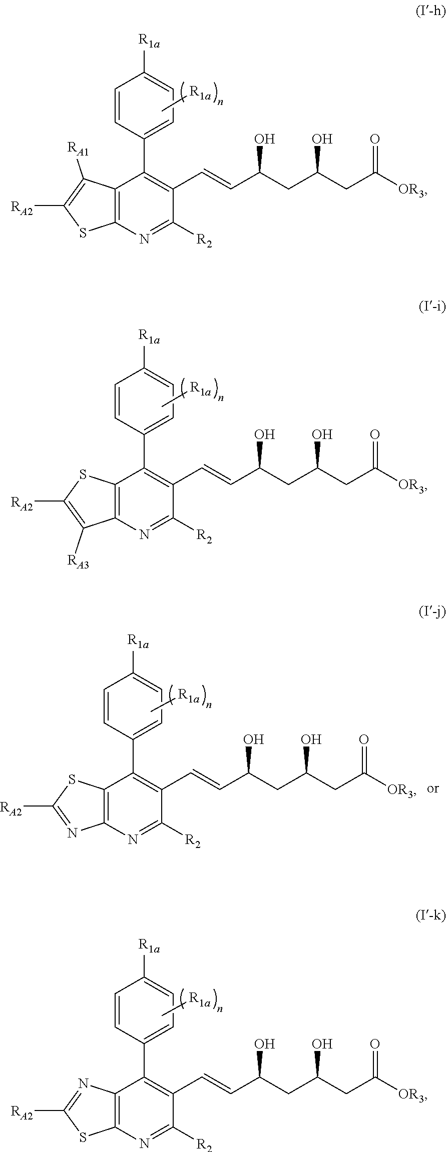

or a pharmaceutically acceptable salt thereof, wherein R₁ₐ is halo, —CN, —OH, —NH₂, C₁-C₆ alkyl, C₂-C₆ alkenyl, C₂-C₆ alkynyl, C₁-C₆ alkoxyl, or C₁-C₆ haloalkyl, and n is 0, 1, 2, 3, or 4.

In some embodiments, the compound of Formula (I) or Formula (I′) is of Formula (I-1), (I-m), (I-n), or (I-o):

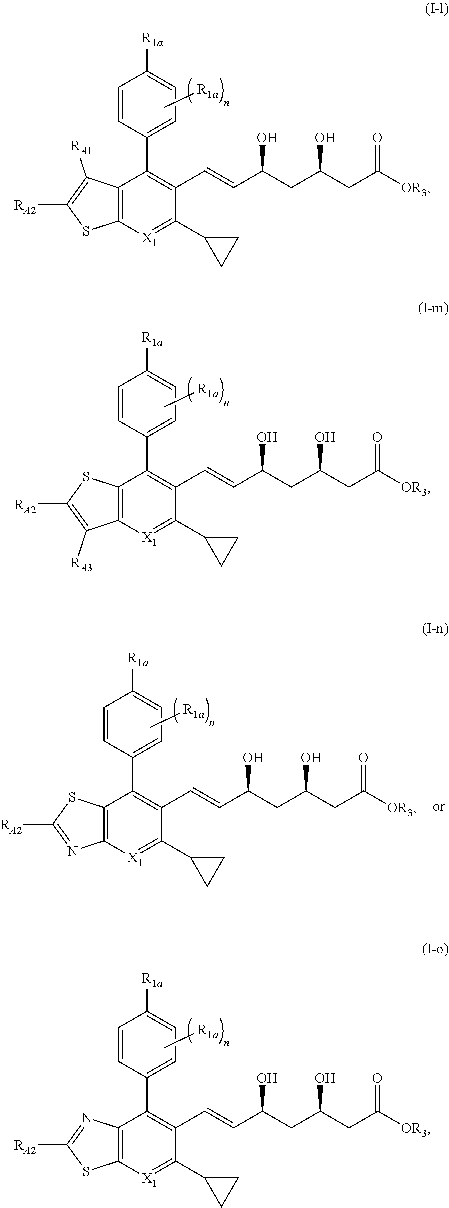

or a pharmaceutically acceptable salt thereof, wherein R₁ₐ is halo, —CN, —OH, —NH₂, C₁-C₆ alkyl, C₂-C₆ alkenyl, C₂-C₆ alkynyl, C₁-C₆ alkoxyl, or C₁-C₆ haloalkyl, and n is 0, 1, 2, 3, or 4.

In some embodiments, the compound of Formula (I) or Formula (I′) is of Formula (I′-1), (I′-m), (I′-n), or (I′-o):

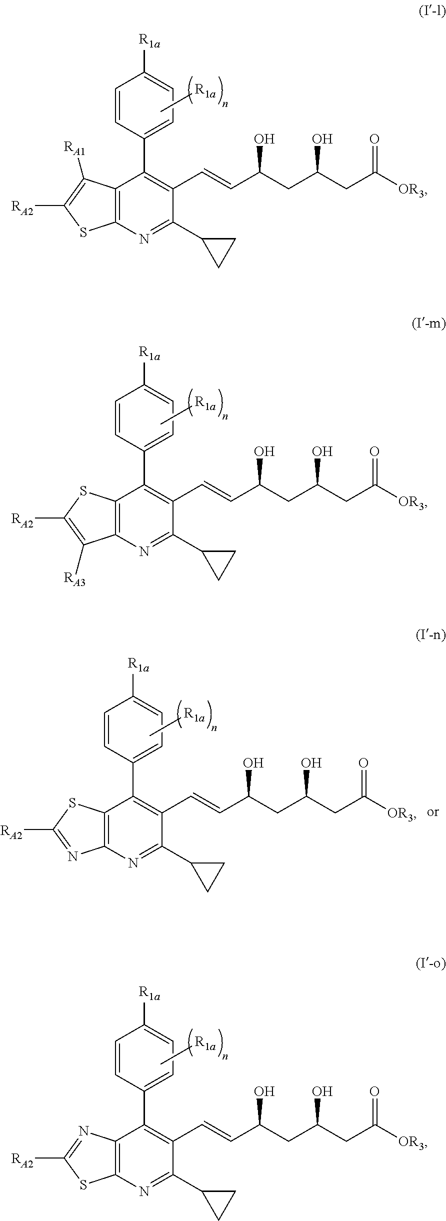

or a pharmaceutically acceptable salt thereof, wherein R₁ₐ is halo, —CN, —OH, —NH₂, C₁-C₆ alkyl, C₂-C₆ alkenyl, C₂-C₆ alkynyl, C₁-C₆ alkoxyl, or C₁-C₆ haloalkyl, and n is 0, 1, 2, 3, or 4.

### Formula (II′)

In some aspects, the present disclosure provides a compound of Formula (II′):

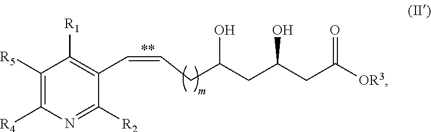

or a pharmaceutically acceptable salt thereof, wherein:
   [symbol] is a double bond, wherein the double bond is the (E) or (Z) isomer;
   R₁ is C₆-C₁₀ aryl or 5- to 10-membered heteroaryl, wherein the aryl or heteroaryl is optionally substituted with one or more R₁ₐ;
   each R₁ₐ independently is halo, —CN, —OH, —NH₂, C₁-C₆ alkyl, C₂-C₆ alkenyl, C₂-C₆ alkynyl, C₁-C₆ alkoxyl, or C₁-C₆ haloalkyl;
   R₂ is C₃-C₁₀ cycloalkyl or methyl;
   R₃ is H, C₁-C₆ alkyl, C₂-C₆ alkenyl, C₂-C₆ alkynyl, C₁-C₆ alkoxyl, or C₁-C₆ haloalkyl;
   R₄ is H, C₃-C₁₀ cycloalkyl, 3- to 10-membered heterocyclyl, C₁-C₆ alkyl, C₂-C₆ alkenyl, C₂-C₆ alkynyl, C₁-C₆ alkoxyl, or C₁-C₆ haloalkyl;
   R₅ is H, C₁-C₆ alkyl, C₂-C₆ alkenyl, C₂-C₆ alkynyl, C₁-C₆ alkoxyl, C₁-C₆ haloalkyl, or C₃-C₁₀ cycloalkyl, wherein the alkyl, alkenyl, alkynyl, alkoxyl, haloalkyl or C₃-C₁₀ cycloalkyl is optionally substituted with one or more C₁-C₆ alkoxy or —O(C₃-C₁₀ cycloalkyl), or
   R₅ and one R₁ₐ, together with the intervening atoms, form 3- to 10-membered heterocyclyl; and
   m is 0 or 1,
provided that:
   (a) when R₂ is cyclopropyl, and R₅ is C₁ alkyl substituted by C₁ alkoxy, then R₄ is not isopropyl; and
   (b) when R₁ is monosubstituted C₆ aryl substituted by one fluoro, R₂ is cyclopropyl, and R₅ is C₁ alkyl substituted by C₁ alkoxy, then R₄ is not cyclopropyl; and
   (c) when R₂ is methyl, then R₄ is not C₁-C₆ alkyl.

In some aspects, the present disclosure provides a compound of Formula (II′), or a pharmaceutically acceptable salt thereof, wherein:
[symbol] is a double bond, wherein the double bond is the (E) or (Z) isomer;
R₁ is C₆-C₁₀ aryl or 5- to 10-membered heteroaryl, wherein the aryl or heteroaryl is optionally substituted with one or more R₁ₐ;
each R₁ₐ independently is halo, —CN, —OH, —NH₂, C₁-C₆ alkyl, C₂-C₆ alkenyl, C₂-C₆ alkynyl, C₁-C₆ alkoxyl, or C₁-C₆ haloalkyl;
R₂ is C₃-C₁₀ cycloalkyl or methyl;
R₃ is H, C₁-C₆ alkyl, C₂-C₆ alkenyl, C₂-C₆ alkynyl, C₁-C₆ alkoxyl, or C₁-C₆ haloalkyl;
R₄ is H, C₃-C₁₀ cycloalkyl, 3- to 10-membered heterocyclyl, C₁-C₆ alkyl, C₂-C₆ alkenyl, C₂-C₆ alkynyl, C₁-C₆ alkoxyl, or C₁-C₆ haloalkyl;
R₅ is H, C₁-C₆ alkyl, C₂-C₆ alkenyl, C₂-C₆ alkynyl, C₁-C₆ alkoxyl, C₁-C₆ haloalkyl, or C₃-C₁₀ cycloalkyl, wherein the alkyl, alkenyl, alkynyl, alkoxyl, haloalkyl or C₃-C₁₀ cycloalkyl is optionally substituted with one or more C₁-C₆ alkoxy or —O(C₃-C₁₀ cycloalkyl), or
R₅ and one R₁ₐ, together with the intervening atoms, form 3- to 10-membered heterocyclyl; and
m is 0 or 1,
   provided that:
(a) when R₂ is cyclopropyl, and R₅ is C₁ alkyl substituted by C₁ alkoxy, then R₄ is not isopropyl; and
(b) when R₁ is monosubstituted C₆ aryl substituted by one fluoro, R₂ is cyclopropyl, and R₅ is C₁ alkyl substituted by C₁ alkoxy, then R₄ is not cyclopropyl.

In some aspects, the present disclosure provides a compound of Formula (II′), or a pharmaceutically acceptable salt thereof, wherein:
[symbol] is a double bond, wherein the double bond is the (E) or (Z) isomer;
R₁ is C₆-C₁₀ aryl or 5- to 10-membered heteroaryl, wherein the aryl or heteroaryl is optionally substituted with one or more R₁ₐ,
each R₁ₐ independently is halo, —CN, —OH, —NH₂, C₁-C₆ alkyl, C₂-C₆ alkenyl, C₂-C₆ alkynyl, C₁-C₆ alkoxyl, or C₁-C₆ haloalkyl;
R₂ is C₃-C₁₀ cycloalkyl or methyl;
R₃ is H, C₁-C₆ alkyl, C₂-C₆ alkenyl, C₂-C₆ alkynyl, C₁-C₆ alkoxyl, or C₁-C₆ haloalkyl;
R₄ is H, C₃-C₁₀ cycloalkyl, 3- to 10-membered heterocyclyl, C₁-C₆ alkyl, C₂-C₆ alkenyl, C₂-C₆ alkynyl, C₁-C₆ alkoxyl, or C₁-C₆ haloalkyl;
R₅ is H, C₁-C₆ alkyl, C₂-C₆ alkenyl, C₂-C₆ alkynyl, C₁-C₆ alkoxyl, C₁-C₆ haloalkyl, or C₃-C₁₀ cycloalkyl, wherein the alkyl, alkenyl, alkynyl, alkoxyl, haloalkyl or C₃-C₁₀ cycloalkyl is optionally substituted with one or more C₁-C₆ alkoxy or —O(C₃-C₁₀ cycloalkyl), or
R₅ and one R₁ₐ, together with the intervening atoms, form 3- to 10-membered heterocyclyl; and
m is 0 or 1,
   provided that:
(a) when R₂ is cyclopropyl, and R₅ is C₁ alkyl substituted by C₁ alkoxy, then R₄ is not isopropyl.

In some aspects, the present disclosure provides a compound of Formula (II′), or a pharmaceutically acceptable salt thereof, wherein:
[symbol] is a double bond, wherein the double bond is the (E) or (Z) isomer;
R₁ is C₆-C₁₀ aryl or 5- to 10-membered heteroaryl, wherein the aryl or heteroaryl is optionally substituted with one or more R₁ₐ;
each R₁ₐ independently is halo, —CN, —OH, —NH₂, C₁-C₆ alkyl, C₂-C₆ alkenyl, C₂-C₆ alkynyl, C₁-C₆ alkoxyl, or C₁-C₆ haloalkyl;
R₂ is C₃-C₁₀ cycloalkyl or methyl;
R₃ is H, C₁-C₆ alkyl, C₂-C₆ alkenyl, C₂-C₆ alkynyl, C₁-C₆ alkoxyl, or C₁-C₆ haloalkyl;
R₄ is H, C₃-C₁₀ cycloalkyl, 3- to 10-membered heterocyclyl, C₁-C₆ alkyl, C₂-C₆ alkenyl, C₂-C₆ alkynyl, C₁-C₆ alkoxyl, or C₁-C₆ haloalkyl;
R₅ is H, C₁-C₆ alkyl, C₂-C₆ alkenyl, C₂-C₆ alkynyl, C₁-C₆ alkoxyl, C₁-C₆ haloalkyl, or C₃-C₁₀ cycloalkyl, wherein the alkyl, alkenyl, alkynyl, alkoxyl, haloalkyl or C₃-C₁₀ cycloalkyl is optionally substituted with one or more C₁-C₆ alkoxy or —O(C₃-C₁₀ cycloalkyl), or
R₅ and one R₁ₐ, together with the intervening atoms, form 3- to 10-membered heterocyclyl; and
m is 0 or 1,
   provided that:
(b) when R₁ is monosubstituted C₆ aryl substituted by one fluoro, R₂ is cyclopropyl.

In some aspects, the present disclosure provides a compound of Formula (II):

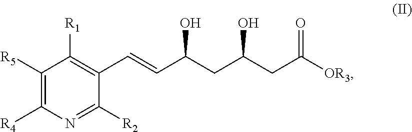

or a pharmaceutically acceptable salt thereof, wherein:
   R₁ is C₆-C₁₀ aryl or 5- to 10-membered heteroaryl optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl;
   R₂ is C₃-C₁₀ cycloalkyl or methyl;
   R₃ is H, C₁-C₆ alkyl, C₂-C₆ alkenyl, C₂-C₆ alkynyl, C₁-C₆ alkoxyl, or C₁-C₆ haloalkyl;
   R₄ is C₃-C₁₀ cycloalkyl, 3- to 10-membered heterocyclyl, C₁-C₆ alkyl, C₂-C₆ alkenyl, C₂-C₆ alkynyl, C₁-C₆ alkoxyl, or C₁-C₆ haloalkyl; and
   R₅ is H, C₁-C₆ alkyl, C₂-C₆ alkenyl, C₂-C₆ alkynyl, C₁-C₆ alkoxyl, or C₁-C₆ haloalkyl, wherein the alkyl, alkenyl, alkynyl, alkoxyl, or haloalkyl is optionally substituted with one or more C₁-C₆ alkoxy or —O(C₃-C₁₀ cycloalkyl),
provided that:
   (a) when R₂ is cyclopropyl, and R₅ is C₁ alkyl substituted by C₁ alkoxy, then R₄ is not isopropyl; and
   (b) when R₁ is monosubstituted C₆ aryl substituted by one fluoro, R₂ is cyclopropyl, and R₅ is C₁ alkyl substituted by C₁ alkoxy, then R₄ is not cyclopropyl; and
   (c) when R₂ is methyl, then R₄ is not C₁-C₆ alkyl.

In some embodiments, the compound of Formula (II′) is a compound of Formula (II).

In some aspects, the present disclosure provides a compound of Formula (II):

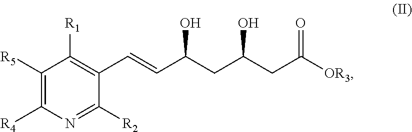

or a pharmaceutically acceptable salt thereof, wherein:
   R₁ is C₆-C₁₀ aryl or 5- to 10-membered heteroaryl optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl;
   R₂ is C₃-C₁₀ cycloalkyl;
   R₃ is H, C₁-C₆ alkyl, C₂-C₆ alkenyl, C₂-C₆ alkynyl, C₁-C₆ alkoxyl, or C₁-C₆ haloalkyl;
   R₄ is C₃-C₁₀ cycloalkyl, 3- to 10-membered heterocyclyl, C₁-C₆ alkyl, C₂-C₆ alkenyl, C₂-C₆ alkynyl, C₁-C₆ alkoxyl, or C₁-C₆ haloalkyl; and
   R₅ is H, C₁-C₆ alkyl, C₂-C₆ alkenyl, C₂-C₆ alkynyl, C₁-C₆ alkoxyl, or C₁-C₆ haloalkyl, wherein the alkyl, alkenyl, alkynyl, alkoxyl, or haloalkyl is optionally substituted with one or more C₁-C₆ alkoxy,
provided that:
   (a) when R₂ is cyclopropyl, and R₅ is C₁ alkyl substituted by C₁ alkoxy, then R₄ is not isopropyl; and
   (b) when R₁ is monosubstituted C₆ aryl substituted by one fluoro, R₂ is cyclopropyl, and R₅ is C₁ alkyl substituted by C₁ alkoxy, then R₄ is not cyclopropyl.

In some aspects, the present disclosure provides a compound of Formula (II):

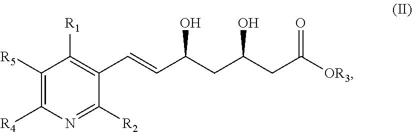

or a pharmaceutically acceptable salt thereof, wherein:
   R₁ is C₆-C₁₀ aryl or 5- to 10-membered heteroaryl optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl;
   R₂ is C₃-C₁₀ cycloalkyl;
   R₃ is H, C₁-C₆ alkyl, C₂-C₆ alkenyl, C₂-C₆ alkynyl, C₁-C₆ alkoxyl, or C₁-C₆ haloalkyl;
   R₄ is C₄-C₁₀ cycloalkyl, 3- to 10-membered heterocyclyl, C₁-C₆ alkyl, C₂-C₆ alkenyl, C₂-C₆ alkynyl, C₁-C₆ alkoxyl, or C₁-C₆ haloalkyl; and
   R₅ is H, C₁-C₆ alkyl, C₂-C₆ alkenyl, C₂-C₆ alkynyl, C₁-C₆ alkoxyl, or C₁-C₆ haloalkyl, wherein the alkyl, alkenyl, alkynyl, alkoxyl, or haloalkyl is optionally substituted with one or more C₁-C₆ alkoxy,
provided that:
   (a) when R₂ is cyclopropyl, and R₅ is C₁ alkyl substituted by C₁ alkoxy, then R₄ is not isopropyl.

In some aspects, the present disclosure provides a compound of Formula (II):

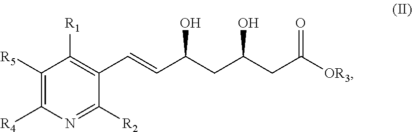

or a pharmaceutically acceptable salt thereof, wherein:
   R₁ is C₆-C₁₀ aryl or 5- to 10-membered heteroaryl optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl;
   R₂ is C₃-C₁₀ cycloalkyl;
   R₃ is H, C₁-C₆ alkyl, C₂-C₆ alkenyl, C₂-C₆ alkynyl, C₁-C₆ alkoxyl, or C₁-C₆ haloalkyl;
   R₄ is C₃-C₁₀ cycloalkyl, 3- to 10-membered heterocyclyl, C₁-C₂ or C₄-C₆ alkyl, C₂-C₆ alkenyl, C₂-C₆ alkynyl, C₁-C₆ alkoxyl, or C₁-C₆ haloalkyl; and
   R₅ is H, C₁-C₆ alkyl, C₂-C₆ alkenyl, C₂-C₆ alkynyl, C₁-C₆ alkoxyl, or C₁-C₆ haloalkyl, wherein the alkyl, alkenyl, alkynyl, alkoxyl, or haloalkyl is optionally substituted with one or more C₁-C₆ alkoxy,
provided that:
   (b) when R₁ is monosubstituted C₆ aryl substituted by one fluoro, R₂ is cyclopropyl, and R₅ is C₁ alkyl substituted by C₁ alkoxy, then R₄ is not cyclopropyl.

In some aspects, the present disclosure provides a compound of Formula (II):

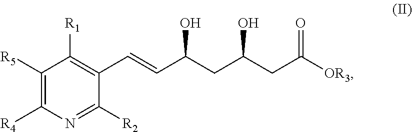

or a pharmaceutically acceptable salt thereof, wherein:
   R₁ is C₆-C₁₀ aryl or 5- to 10-membered heteroaryl optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl;
   R₂ is C₃-C₁₀ cycloalkyl;
   R₃ is H, C₁-C₆ alkyl, C₂-C₆ alkenyl, C₂-C₆ alkynyl, C₁-C₆ alkoxyl, or C₁-C₆ haloalkyl;
   R₄ is C₄-C₁₀ cycloalkyl, 3- to 10-membered heterocyclyl, C₁-C₂ or C₄-C₆ alkyl, C₂-C₆ alkenyl, C₂-C₆ alkynyl, C₁-C₆ alkoxyl, or C₁-C₆ haloalkyl; and
   R₅ is H, C₁-C₆ alkyl, C₂-C₆ alkenyl, C₂-C₆ alkynyl, C₁-C₆ alkoxyl, or C₁-C₆ haloalkyl, wherein the alkyl, alkenyl, alkynyl, alkoxyl, or haloalkyl is optionally substituted with one or more C₁-C₆ alkoxy.

It is understood that, for a compound of the present disclosure, variables R₁, R₁a, R₂, R₃, R₄, and R₅ can each be, where applicable, selected from the groups described herein, and any group described herein for any of variables R₁, R₁ₐ, R₂, R₃, R₄, and R₅ can be combined, where applicable, with any group described herein for one or more of the remainder of variables R₁, R₁ₐ, R₂, R₃, R₄, and R₅.

In some embodiments, [symbol] is a double bond, wherein the double bond is the (E) isomer.

In some embodiments, [symbol] is a double bond, wherein the double bond is the (Z) isomer.

In some embodiments, R₁ is C₆-C₁₀ aryl or 5- to 10-membered heteroaryl.

In some embodiments, R₁ is C₆-C₁₀ aryl or 5- to 10-membered heteroaryl, wherein the aryl or heteroaryl is optionally substituted with one or more R₁ₐ.

In some embodiments, R₁ is C₆-C₁₀ aryl or 5- to 10-membered heteroaryl, wherein the aryl or heteroaryl is substituted with one or more R₁ₐ.

In some embodiments, R₁ is C₆-C₁₀ aryl or 5- to 10-membered heteroaryl optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl.

In some embodiments, R₁ is C₆-C₁₀ aryl or 5- to 10-membered heteroaryl substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl.

In some embodiments, R₁ is C₆-C₁₀ aryl or 5- to 10-membered heteroaryl optionally substituted with one or more halo, —CN, —OH, or —NH₂.

In some embodiments, R₁ is C₆-C₁₀ aryl or 5- to 10-membered heteroaryl substituted with one or more halo, —CN, —OH, or —NH₂.

In some embodiments, R₁ is C₆-C₁₀ aryl or 5- to 10-membered heteroaryl substituted with one or more halo.

In some embodiments, R₁ is C₆-C₁₀ aryl or 5- to 10-membered heteroaryl substituted with one halo.

In some embodiments, R₁ is C₆-C₁₀ aryl.

In some embodiments, R₁ is C₆-C₁₀ aryl optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl.

In some embodiments, R₁ is C₆-C₁₀ aryl substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl.

In some embodiments, R₁ is C₆-C₁₀ aryl substituted with one or more halo.

In some embodiments, R₁ is C₆-C₁₀ aryl substituted with one halo.

In some embodiments, R₁ is C₆ aryl.

In some embodiments, R₁ is C₆ aryl optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl.

In some embodiments, R₁ is C₆ aryl substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl.

In some embodiments, R₁ is C₆ aryl optionally substituted with one or more halo, —CN, —OH, or —NH₂.

In some embodiments, R₁ is C₆ aryl substituted with one or more halo, —CN, —OH, or —NH₂.

In some embodiments, R₁ is C₆ aryl substituted with one or more halo.

In some embodiments, R₁ is C₆ aryl substituted with one halo.

In some embodiments, R₁ is 5- to 10-membered heteroaryl.

In some embodiments, R₁ is 5- to 10-membered heteroaryl optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl.

In some embodiments, R₁ is 5- to 10-membered heteroaryl substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl.

In some embodiments, R₁ is 5- to 10-membered heteroaryl substituted with one or more halo.

In some embodiments, R₁ is 5- to 10-membered heteroaryl substituted with one halo.

In some embodiments, R₁ is 6-membered heteroaryl.

In some embodiments, R₁ is 6-membered heteroaryl optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl.

In some embodiments, R₁ is 6-membered heteroaryl substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl.

In some embodiments, R₁ is 6-membered heteroaryl substituted with one or more halo.

In some embodiments, R₁ is 6-membered heteroaryl substituted with one halo.

In some embodiments, R₁ is

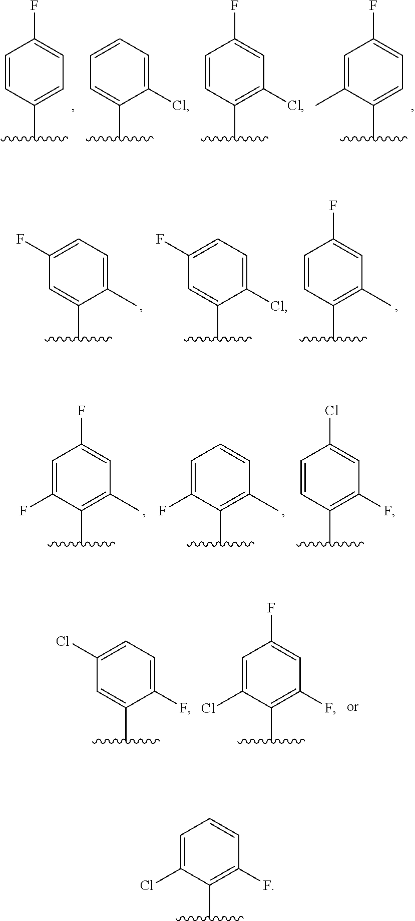

In some embodiments, R₁ is

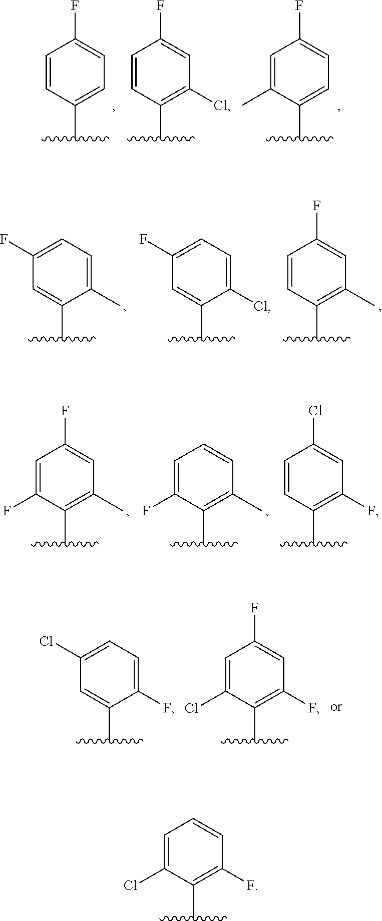

In some embodiments, R₁ is

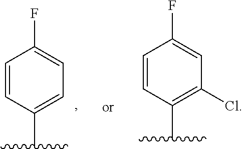 [SMILES: *c1ccc(F)cc1 ; *c1ccc(F)cc1Cl]

In some embodiments, R₁ is substituted, at least, at the 2-position.

In some embodiments, R₁ is substituted, at least, at the 3-position.

In some embodiments, R₁ is substituted, at least, at the 4-position.

In some embodiments, each R₁ₐ independently is halo, —CN, —OH, —NH₂, C₁-C₆ alkyl, C₂-C₆ alkenyl, C₂-C₆ alkynyl, C₁-C₆ alkoxyl, or C₁-C₆ haloalkyl.

In some embodiments, at least one R₁ₐ is halo (e.g., C₁, F, I, Br).

In some embodiments, at least one R₁ₐ is —CN.

In some embodiments, at least one R₁ₐ is —OH.

In some embodiments, at least one R₁ₐ is-NH₂.

In some embodiments, at least one R₁ₐ is C₁-C₆ alkyl.

In some embodiments, R₁ₐ is methyl. In some embodiments, R₁ₐ is ethyl. In some embodiments, R₁ₐ is propyl. In some embodiments, R₁ₐ is butyl. In some embodiments, R₁ₐ is pentyl. In some embodiments, R₁ₐ is hexyl. In some embodiments, R₁ₐ is isopropyl. In some embodiments, R₁ₐ is isobutyl. In some embodiments, R₁ₐ is isopentyl. In some embodiments, R₁ₐ is isohexyl. In some embodiments, R₁ₐ is secbutyl. In some embodiments, R₁ₐ is secpentyl. In some embodiments, R₁ₐ is sechexyl. In some embodiments, R₁ₐ is tertbutyl.

In some embodiments, R₁ₐ is C₂-C₆ alkenyl (e.g., ethenyl, propenyl, butenyl).

In some embodiments, R₁ₐ is C₂-C₆ alkynyl (e.g., ethynyl, propynyl, butynyl).

In some embodiments, R₁ₐ is C₁-C₆ alkoxyl.

In some embodiments, R₁ₐ is methoxyl. In some embodiments, R₁ₐ is ethoxyl. In some embodiments, R₁ₐ is propoxyl. In some embodiments, R₁ₐ is butoxyl. In some embodiments, R₁ₐ is pentoxyl. In some embodiments, R₁ₐ is hexoxyl.

In some embodiments, R₁ₐ is C₁-C₆ haloalkyl.

In some embodiments, R₁ₐ is halomethyl. In some embodiments, R₁ₐ is haloethyl. In some embodiments, R₁ₐ is halopropyl. In some embodiments, R₁ₐ is halobutyl. In some embodiments, R₁ₐ is halopentyl. In some embodiments, R₁ₐ is halohexyl.

In some embodiments, R₂ is methyl.

In some embodiments, R₂ is C₃-C₁₀ cycloalkyl.

In some embodiments, R₂ is C₃ cycloalkyl (cyclopropyl).

In some embodiments, R₂ is C₄ cycloalkyl (cyclobutyl).

In some embodiments, R₂ is C₈ cycloalkyl. In some embodiments, R₂ is C₆ cycloalkyl. In some embodiments, R₂ is C₇ cycloalkyl. In some embodiments, R₂ is C₈ cycloalkyl. In some embodiments, R₂ is C₉ cycloalkyl. In some embodiments, R₂ is C₁₀ cycloalkyl.

In some embodiments, R₂ is C₅-C₁₀ cycloalkyl. In some embodiments, R₂ is bridged C₅-C₁₀ cycloalkyl. In some embodiments, R₂ is bicyclic C₅-C₁₀ cycloalkyl.

In some embodiments, R₂ is methyl,

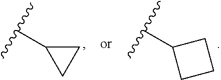

In some embodiments, R₂ is

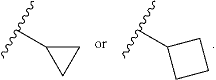

In some embodiments, R₃ is H, C₁-C₆ alkyl, C₂-C₆ alkenyl, C₂-C₆ alkynyl, C₁-C₆ alkoxyl, or C₁-C₆ haloalkyl.

In some embodiments, R₃ is H.

In some embodiments, R₃ is C₁-C₆ alkyl, C₂-C₆ alkenyl, C₂-C₆ alkynyl, C₁-C₆ alkoxyl, or C₁-C₆ haloalkyl.

In some embodiments, R₃ is C₁-C₆ alkyl.

In some embodiments, R₃ is methyl. In some embodiments, R₃ is ethyl. In some embodiments, R₃ is propyl. In some embodiments, R₃ is butyl. In some embodiments, R₃ is pentyl. In some embodiments, R₃ is hexyl. In some embodiments, R₃ is isopropyl. In some embodiments, R₃ is isobutyl. In some embodiments, R₃ is isopentyl. In some embodiments, R₃ is isohexyl. In some embodiments, R₃ is secbutyl. In some embodiments, R₃ is secpentyl. In some embodiments, R₃ is sechexyl. In some embodiments, R₃ is tertbutyl.

In some embodiments, R₃ is C₂-C₆ alkenyl (e.g., ethenyl, propenyl, butenyl).

In some embodiments, R₃ is C₂-C₆ alkynyl (e.g., ethynyl, propynyl, butynyl).

In some embodiments, R₃ is C₁-C₆ alkoxyl.

In some embodiments, R₃ is methoxyl. In some embodiments, R₃ is ethoxyl. In some embodiments, R₃ is propoxyl. In some embodiments, R₃ is butoxyl. In some embodiments, R₃ is pentoxyl. In some embodiments, R₃ is hexoxyl.

In some embodiments, R₃ is C₁-C₆ haloalkyl.

In some embodiments, R₃ is halomethyl. In some embodiments, R₃ is haloethyl. In some embodiments, R₃ is halopropyl. In some embodiments, R₃ is halobutyl. In some embodiments, R₃ is halopentyl. In some embodiments, R₃ is halohexyl.

In some embodiments, R₄ is H, C₃-C₁₀ cycloalkyl, 3- to 10-membered heterocyclyl, C₁-C₆ alkyl, C₂-C₆ alkenyl, C₂-C₆ alkynyl, C₁-C₆ alkoxyl, or C₁-C₆ haloalkyl.

In some embodiments, R₄ is H.

In some embodiments, R₄ is C₃-C₁₀ cycloalkyl, 3- to 10-membered heterocyclyl, C₁-C₆ alkyl, C₂-C₆ alkenyl, C₂-C₆ alkynyl, C₁-C₆ alkoxyl, or C₁-C₆ haloalkyl.

In some embodiments, R₄ is C₃-C₁₀ cycloalkyl, 3- to 10-membered heterocyclyl, C₂-C₆ alkenyl, C₂-C₆ alkynyl, C₁-C₆ alkoxyl, or C₁-C₆ haloalkyl.

In some embodiments, R₄ is C₃-C₁₀ cycloalkyl.

In some embodiments, R₄ is C₃ cycloalkyl (cyclopropyl).

In some embodiments, R₄ is C₄ cycloalkyl (cyclobutyl).

In some embodiments, R₄ is C₈ cycloalkyl. In some embodiments, R₄ is C₆ cycloalkyl. In some embodiments, R₄ is C₇ cycloalkyl. In some embodiments, R₄ is C₈ cycloalkyl. In some embodiments, R₄ is C₉ cycloalkyl. In some embodiments, R₄ is C₁₀ cycloalkyl.

In some embodiments, R₄ is C₅-C₁₀ cycloalkyl. In some embodiments, R₄ is bridged C₅-C₁₀ cycloalkyl. In some embodiments, R₄ is bicyclic C₅-C₁₀ cycloalkyl.

In some embodiments, R₄ is 3- to 10-membered heterocyclyl.

In some embodiments, R₄ is 3-membered heterocyclyl. In some embodiments, R₄ is 4-membered heterocyclyl. In some embodiments, R₄ is 5-membered heterocyclyl. In some embodiments, R₄ is 6-membered heterocyclyl. In some embodiments, R₄ is 7-membered heterocyclyl. In some embodiments, R₄ is 8-membered heterocyclyl. In some embodiments, R₄ is 9-membered heterocyclyl. In some embodiments, R₄ is 10-membered heterocyclyl.

In some embodiments, R₄ is C₁-C₆ alkyl, C₂-C₆ alkenyl, C₂-C₆ alkynyl, C₁-C₆ alkoxyl, or C₁-C₆ haloalkyl.

In some embodiments, R₄ is C₁-C₆ alkyl.

In some embodiments, R₄ is methyl. In some embodiments, R₄ is ethyl. In some embodiments, R₄ is propyl. In some embodiments, R₄ is butyl. In some embodiments, R₄ is pentyl. In some embodiments, R₄ is hexyl. In some embodiments, R₄ is isopropyl. In some embodiments, R₄ is isobutyl. In some embodiments, R₄ is isopentyl. In some embodiments, R₄ is isohexyl. In some embodiments, R₄ is secbutyl. In some embodiments, R₄ is secpentyl. In some embodiments, R₄ is sechexyl. In some embodiments, R₄ is tertbutyl.

In some embodiments, R₄ is C₂-C₆ alkenyl (e.g., ethenyl, propenyl, butenyl).

In some embodiments, R₄ is C₂-C₆ alkynyl (e.g., ethynyl, propynyl, butynyl).

In some embodiments, R₄ is C₁-C₆ alkoxyl.

In some embodiments, R₄ is methoxyl. In some embodiments, R₄ is ethoxyl. In some embodiments, R₄ is propoxyl. In some embodiments, R₄ is butoxyl. In some embodiments, R₄ is pentoxyl. In some embodiments, R₄ is hexoxyl.

In some embodiments, R₄ is C₁-C₆ haloalkyl.

In some embodiments, R₄ is halomethyl. In some embodiments, R₄ is haloethyl. In some embodiments, R₄ is halopropyl. In some embodiments, R₄ is halobutyl. In some embodiments, R₄ is halopentyl. In some embodiments, R₄ is halohexyl.

In some embodiments, R₄ is H,

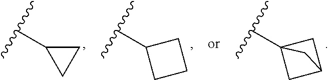

In some embodiments, R₄ is

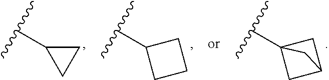

In some embodiments, R₅ is H, C₁-C₆ alkyl, C₂-C₆ alkenyl, C₂-C₆ alkynyl, C₁-C₆ alkoxyl, C₁-C₆ haloalkyl, or C₃-C₁₀ cycloalkyl.

In some embodiments, R₅ is H, C₁-C₆ alkyl, C₂-C₆ alkenyl, C₂-C₆ alkynyl, C₁-C₆ alkoxyl, C₁-C₆ haloalkyl, or C₃-C₁₀ cycloalkyl, wherein the alkyl, alkenyl, alkynyl, alkoxyl, haloalkyl or C₃-C₁₀ cycloalkyl is optionally substituted with one or more C₁-C₆ alkoxy or —O(C₃-C₁₀ cycloalkyl).

In some embodiments, R₅ is H, C₁-C₆ alkyl, C₂-C₆ alkenyl, C₂-C₆ alkynyl, C₁-C₆ alkoxyl, C₁-C₆ haloalkyl, or C₃-C₁₀ cycloalkyl, wherein the alkyl, alkenyl, alkynyl, alkoxyl, haloalkyl or C₃-C₁₀ cycloalkyl is substituted with one or more C₁-C₆ alkoxy or —O(C₃-C₁₀ cycloalkyl).

In some embodiments, R₅ is H, C₁-C₆ alkyl, C₂-C₆ alkenyl, C₂-C₆ alkynyl, C₁-C₆ alkoxyl, or C₁-C₆ haloalkyl.

In some embodiments, R₅ is H, C₁-C₆ alkyl, C₂-C₆ alkenyl, C₂-C₆ alkynyl, C₁-C₆ alkoxyl, or C₁-C₆ haloalkyl, wherein the alkyl, alkenyl, alkynyl, alkoxyl, or haloalkyl is optionally substituted with one or more C₁-C₆ alkoxy.

In some embodiments, R₅ is H, C₁-C₆ alkyl, C₂-C₆ alkenyl, C₂-C₆ alkynyl, C₁-C₆ alkoxyl, or C₁-C₆ haloalkyl, wherein the alkyl, alkenyl, alkynyl, alkoxyl, or haloalkyl is substituted with one or more C₁-C₆ alkoxy or —O(C₃-C₁₀ cycloalkyl).

In some embodiments, R₅ is H, C₁-C₆ alkyl, C₂-C₆ alkenyl, C₂-C₆ alkynyl, C₁-C₆ alkoxyl, or C₁-C₆ haloalkyl, wherein the alkyl, alkenyl, alkynyl, alkoxyl, or haloalkyl is substituted with one or more C₁-C₆ alkoxy.

In some embodiments, R₅ is H.

In some embodiments, R₅ is C₁-C₆ alkyl.

In some embodiments, R₅ is C₁-C₆ alkyl optionally substituted with one or more C₁-C₆ alkoxy or —O(C₃-C₁₀ cycloalkyl).

In some embodiments, R₅ is C₁-C₆ alkyl substituted with one or more C₁-C₆ alkoxy or —O(C₃-C₁₀ cycloalkyl).

In some embodiments, R₅ is C₁-C₆ alkyl optionally substituted with one or more C₁-C₆ alkoxy.

In some embodiments, R₅ is C₁-C₆ alkyl substituted with one or more C₁-C₆ alkoxy. In some embodiments, R₅ is methyl. In some embodiments, R₅ is ethyl. In some embodiments, R₅ is propyl. In some embodiments, R₅ is butyl. In some embodiments, R₅ is pentyl. In some embodiments, R₅ is hexyl. In some embodiments, R₅ is isopropyl. In some embodiments, R₅ is isobutyl. In some embodiments, R₅ is isopentyl. In some embodiments, R₅ is isohexyl. In some embodiments, R₅ is secbutyl. In some embodiments, R₅ is secpentyl. In some embodiments, R₅ is sechexyl. In some embodiments, R₅ is tertbutyl.

In some embodiments, R₅ is methyl optionally substituted with one or more C₁-C₆ alkoxy. In some embodiments, R₅ is ethyl optionally substituted with one or more C₁-C₆ alkoxy. In some embodiments, R₅ is propyl optionally substituted with one or more C₁-C₆ alkoxy. In some embodiments, R₅ is butyl optionally substituted with one or more C₁-C₆ alkoxy. In some embodiments, R₅ is pentyl optionally substituted with one or more C₁-C₆ alkoxy. In some embodiments, R₅ is hexyl optionally substituted with one or more C₁-C₆ alkoxy. In some embodiments, R₅ is isopropyl optionally substituted with one or more C₁-C₆ alkoxy. In some embodiments, R₅ is isobutyl optionally substituted with one or more C₁-C₆ alkoxy. In some embodiments, R₅ is isopentyl optionally substituted with one or more C₁-C₆ alkoxy. In some embodiments, R₅ is isohexyl optionally substituted with one or more C₁-C₆ alkoxy. In some embodiments, R₅ is secbutyl optionally substituted with one or more C₁-C₆ alkoxy. In some embodiments, R₅ is secpentyl optionally substituted with one or more C₁-C₆ alkoxy. In some embodiments, R₅ is sechexyl optionally substituted with one or more C₁-C₆ alkoxy. In some embodiments, R₅ is tertbutyl optionally substituted with one or more C₁-C₆ alkoxy.

In some embodiments, R₅ is methyl substituted with one or more C₁-C₆ alkoxy. In some embodiments, R₅ is ethyl substituted with one or more C₁-C₆ alkoxy. In some embodiments, R₅ is propyl substituted with one or more C₁-C₆ alkoxy. In some embodiments, R₅ is butyl substituted with one or more C₁-C₆ alkoxy. In some embodiments, R₅ is pentyl substituted with one or more C₁-C₆ alkoxy. In some embodiments, R₅ is hexyl substituted with one or more C₁-C₆ alkoxy. In some embodiments, R₅ is isopropyl substituted with one or more C₁-C₆ alkoxy. In some embodiments, R₅ is isobutyl substituted with one or more C₁-C₆ alkoxy. In some embodiments, R₅ is isopentyl substituted with one or more C₁-C₆ alkoxy. In some embodiments, R₅ is isohexyl substituted with one or more C₁-C₆ alkoxy. In some embodiments, R₅ is secbutyl substituted with one or more C₁-C₆ alkoxy. In some embodiments, R₅ is secpentyl substituted with one or more C₁-C₆ alkoxy. In some embodiments, R₅ is sechexyl substituted with one or more C₁-C₆ alkoxy. In some embodiments, R₅ is tertbutyl substituted with one or more C₁-C₆ alkoxy.

In some embodiments, R₅ is methyl optionally substituted with one or more —O(C₃-C₁₀ cycloalkyl). In some embodiments, R₅ is ethyl optionally substituted with one or more —O(C₃-C₁₀ cycloalkyl). In some embodiments, R₅ is propyl optionally substituted with one or more —O(C₃-C₁₀ cycloalkyl). In some embodiments, R₅ is butyl optionally substituted with one or more —O(C₃-C₁₀ cycloalkyl). In some embodiments, R₅ is pentyl optionally substituted with one or more —O(C₃-C₁₀ cycloalkyl). In some embodiments, R₅ is hexyl optionally substituted with one or more —O(C₃-C₁₀ cycloalkyl). In some embodiments, R₅ is isopropyl optionally substituted with one or more —O(C₃-C₁₀ cycloalkyl). In some embodiments, R₅ is isobutyl optionally substituted with one or more —O(C₃-C₁₀ cycloalkyl). In some embodiments, R₅ is isopentyl optionally substituted with one or more —O(C₃-C₁₀ cycloalkyl). In some embodiments, R₅ is isohexyl optionally substituted with one or more —O(C₃-C₁₀ cycloalkyl). In some embodiments, R₅ is secbutyl optionally substituted with one or more —O(C₃-C₁₀ cycloalkyl). In some embodiments, R₅ is secpentyl optionally substituted with one or more —O(C₃-C₁₀ cycloalkyl). In some embodiments, R₅ is sechexyl optionally substituted with one or more —O(C₃-C₁₀ cycloalkyl). In some embodiments, R₅ is tertbutyl optionally substituted with one or more —O(C₃-C₁₀ cycloalkyl).

In some embodiments, R₅ is methyl substituted with one or more —O(C₃-C₁₀ cycloalkyl). In some embodiments, R₅ is ethyl substituted with one or more —O(C₃-C₁₀ cycloalkyl). In some embodiments, R₅ is propyl substituted with one or more —O(C₃-C₁₀ cycloalkyl). In some embodiments, R₅ is butyl substituted with one or more —O(C₃-C₁₀ cycloalkyl). In some embodiments, R₅ is pentyl substituted with one or more —O(C₃-C₁₀ cycloalkyl). In some embodiments, R₅ is hexyl substituted with one or more —O(C₃-C₁₀ cycloalkyl). In some embodiments, R₅ is isopropyl substituted with one or more —O(C₃-C₁₀ cycloalkyl). In some embodiments, R₅ is isobutyl substituted with one or more —O(C₃-C₁₀ cycloalkyl). In some embodiments, R₅ is isopentyl substituted with one or more —O(C₃-C₁₀ cycloalkyl). In some embodiments, R₅ is isohexyl substituted with one or more —O(C₃-C₁₀ cycloalkyl). In some embodiments, R₅ is secbutyl substituted with one or more —O(C₃-C₁₀ cycloalkyl). In some embodiments, R₅ is secpentyl substituted with one or more —O(C₃-C₁₀ cycloalkyl). In some embodiments, R₅ is sechexyl substituted with one or more —O(C₃-C₁₀ cycloalkyl). In some embodiments, R₅ is tertbutyl substituted with one or more —O(C₃-C₁₀ cycloalkyl).

In some embodiments, R₅ is C₂-C₆ alkenyl (e.g., ethenyl, propenyl, butenyl).

In some embodiments, R₅ is C₂-C₆ alkenyl optionally substituted with one or more C₁-C₆ alkoxy.

In some embodiments, R₅ is C₂-C₆ alkenyl substituted with one or more C₁-C₆ alkoxy.

In some embodiments, R₅ is C₂-C₆ alkynyl (e.g., ethynyl, propynyl, butynyl).

In some embodiments, R₅ is C₂-C₆ alkynyl optionally substituted with one or more C₁-C₆ alkoxy.

In some embodiments, R₅ is C₂-C₆ alkynyl substituted with one or more C₁-C₆ alkoxy.

In some embodiments, R₅ is C₁-C₆ alkoxyl.

In some embodiments, R₅ is C₁-C₆ alkoxyl optionally substituted with one or more C₁-C₆ alkoxy.

In some embodiments, R₅ is C₁-C₆ alkoxyl substituted with one or more C₁-C₆ alkoxy.

In some embodiments, R₅ is methoxyl. In some embodiments, R₅ is ethoxyl. In some embodiments, R₅ is propoxyl. In some embodiments, R₅ is butoxyl. In some embodiments, R₅ is pentoxyl. In some embodiments, R₅ is hexoxyl.

In some embodiments, R₅ is methoxyl optionally substituted with one or more C₁-C₆ alkoxy. In some embodiments, R₅ is ethoxyl optionally substituted with one or more C₁-C₆ alkoxy. In some embodiments, R₅ is propoxyl optionally substituted with one or more C₁-C₆ alkoxy. In some embodiments, R₅ is butoxyl optionally substituted with one or more C₁-C₆ alkoxy. In some embodiments, R₅ is pentoxyl optionally substituted with one or more C₁-C₆ alkoxy. In some embodiments, R₅ is hexoxyl optionally substituted with one or more C₁-C₆ alkoxy.

In some embodiments, R₅ is methoxyl substituted with one or more C₁-C₆ alkoxy. In some embodiments, R₅ is ethoxyl substituted with one or more C₁-C₆ alkoxy. In some embodiments, R₅ is propoxyl substituted with one or more C₁-C₆ alkoxy. In some embodiments, R₅ is butoxyl substituted with one or more C₁-C₆ alkoxy. In some embodiments, R₅ is pentoxyl substituted with one or more C₁-C₆ alkoxy. In some embodiments, R₅ is hexoxyl substituted with one or more C₁-C₆ alkoxy.

In some embodiments, R₅ is C₁-C₆ haloalkyl.

In some embodiments, R₅ is C₁-C₆ haloalkyl optionally substituted with one or more C₁-C₆ alkoxy.

In some embodiments, R₅ is C₁-C₆ haloalkyl substituted with one or more C₁-C₆ alkoxy.

In some embodiments, R₅ is halomethyl. In some embodiments, R₅ is haloethyl. In some embodiments, R₅ is halopropyl. In some embodiments, R₅ is halobutyl. In some embodiments, R₅ is halopentyl. In some embodiments, R₅ is halohexyl.

In some embodiments, R₅ is halomethyl optionally substituted with one or more C₁-C₆ alkoxy. In some embodiments, R₅ is haloethyl optionally substituted with one or more C₁-C₆ alkoxy. In some embodiments, R₅ is halopropyl optionally substituted with one or more C₁-C₆ alkoxy. In some embodiments, R₅ is halobutyl optionally substituted with one or more C₁-C₆ alkoxy. In some embodiments, R₅ is halopentyl optionally substituted with one or more C₁-C₆ alkoxy. In some embodiments, R₅ is halohexyl optionally substituted with one or more C₁-C₆ alkoxy.

In some embodiments, R₅ is halomethyl substituted with one or more C₁-C₆ alkoxy. In some embodiments, R₅ is haloethyl substituted with one or more C₁-C₆ alkoxy. In some embodiments, R₅ is halopropyl substituted with one or more C₁-C₆ alkoxy. In some embodiments, R₅ is halobutyl substituted with one or more C₁-C₆ alkoxy. In some embodiments, R₅ is halopentyl substituted with one or more C₁-C₆ alkoxy. In some embodiments, R₅ is halohexyl substituted with one or more C₁-C₆ alkoxy.

In some embodiments, R₅ and one R₁ₐ, together with the intervening atoms, form 3- to 10-membered heterocyclyl.

In some embodiments, R₅ and one R₁ₐ, together with the intervening atoms, form tetrahydropyranyl.

In some embodiments, R₅ is —(CH₂)—OCH₃, —(CH₂)—O-cyclopropyl, cyclopropyl, or H.

In some embodiments, R₅ is —(CH₂)—OCH₃, —(CH₂)—O-cyclopropyl, or H.

In some embodiments, R₅ is —(CH₂)—OCH₃ or H.

In some embodiments, when R₂ is cyclopropyl, and R₅ is C₁ alkyl substituted by C₁ alkoxy, then R₄ is not isopropyl.

In some embodiments, when R₁ is monosubstituted C₆ aryl substituted by one fluoro, R₂ is cyclopropyl, and R₅ is C₁ alkyl substituted by C₁ alkoxy, then R₄ is not cyclopropyl.

In some embodiments, when R₂ is methyl, then R₄ is not C₁-C₆ alkyl.

In some embodiments, the compound of Formula (II) or Formula (II′) is of Formula (II-a) or (II-b):

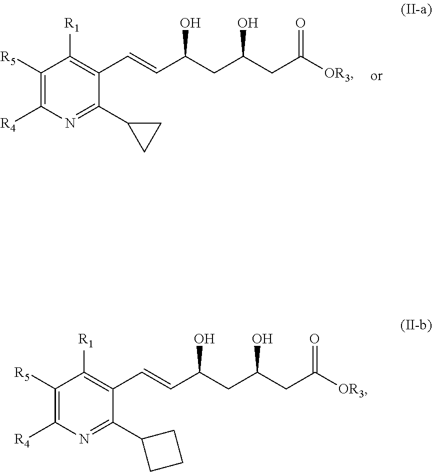

or a pharmaceutically acceptable salt thereof.

In some embodiments, the compound of Formula (II) or Formula (II′) is of Formula (II-c):

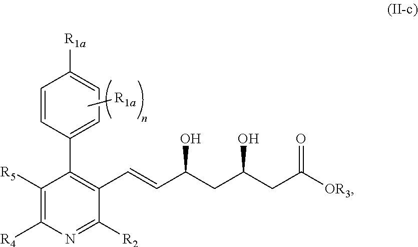

or a pharmaceutically acceptable salt thereof, wherein R₁ₐ is halo, —CN, —OH, —NH₂, C₁-C₆ alkyl, C₂-C₆ alkenyl, C₂-C₆ alkynyl, C₁-C₆ alkoxyl, or C₁-C₆ haloalkyl, and n is 0, 1, 2, 3, or 4.

In some embodiments, the compound of Formula (II) or Formula (II′) is of Formula (II-d) or (II-e):

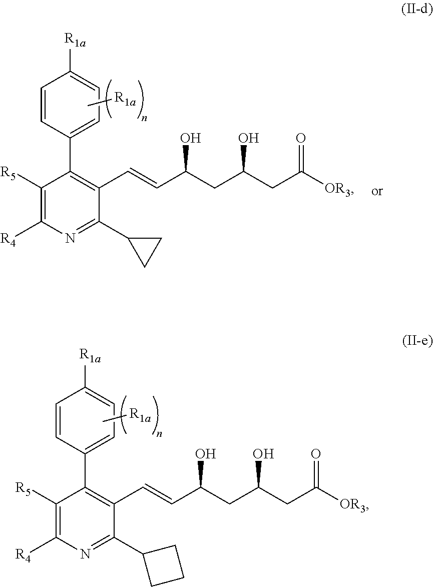

or a pharmaceutically acceptable salt thereof, wherein R₁ₐ is halo, —CN, —OH, —NH₂, C₁-C₆ alkyl, C₂-C₆ alkenyl, C₂-C₆ alkynyl, C₁-C₆ alkoxyl, or C₁-C₆ haloalkyl, and n is 0, 1, 2, 3, or 4.

In some embodiments, the compound of Formula (II) or Formula (II′) is of Formula (II-f) or (II-g):

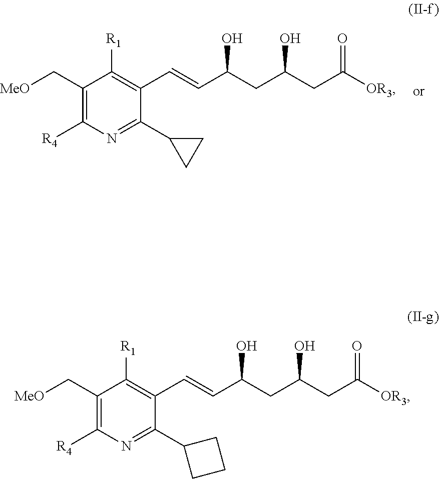

or a pharmaceutically acceptable salt thereof.

In some embodiments, the compound of Formula (II) or Formula (II′) is of Formula (II-h):

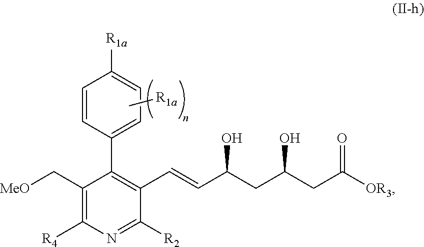

or a pharmaceutically acceptable salt thereof, wherein R₁ₐ is halo, —CN, —OH, —NH₂, C₁-C₆ alkyl, C₂-C₆ alkenyl, C₂-C₆ alkynyl, C₁-C₆ alkoxyl, or C₁-C₆ haloalkyl, and n is 0, 1, 2, 3, or 4.

In some embodiments, the compound of Formula (II) or Formula (II′) is of Formula (II-i) or (II-j):

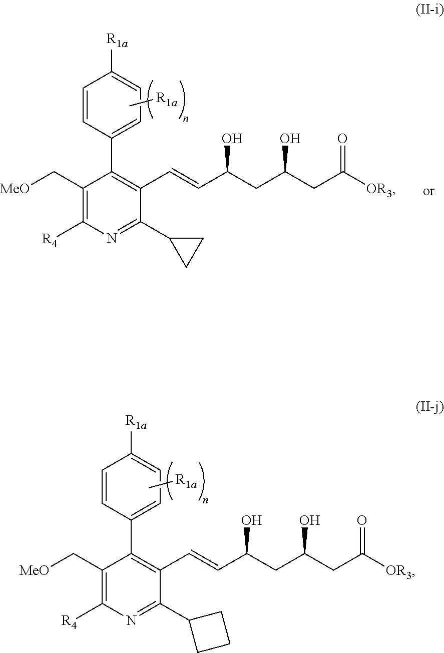

or a pharmaceutically acceptable salt thereof, wherein R₁ₐ is halo, —CN, —OH, —NH₂, C₁-C₆ alkyl, C₂-C₆ alkenyl, C₂-C₆ alkynyl, C₁-C₆ alkoxyl, or C₁-C₆ haloalkyl, and n is 0, 1, 2, 3, or 4.

### Compounds

In some embodiments, the compound is selected from the compounds described in Table 1, or a prodrug or pharmaceutically acceptable salt thereof.

In some embodiments, the compound is selected from the compounds described in Table 1, or a pharmaceutically acceptable salt thereof.

In some embodiments, the compound is selected from the prodrugs of compounds described in Table 1, or a pharmaceutically acceptable salt thereof.

In some embodiments, the compound is selected from the compounds described in Table 1.

In some embodiments, the compound is selected from the compounds described in Table 1A, or a prodrug or pharmaceutically acceptable salt thereof.

In some embodiments, the compound is selected from the compounds described in Table 1A, or a pharmaceutically acceptable salt thereof.

In some embodiments, the compound is selected from the prodrugs of compounds described in Table 1A, or a pharmaceutically acceptable salt thereof.

In some embodiments, the compound is selected from the compounds described in Table 1A.

In some embodiments, the compound is selected from the compounds described in Table 2, or a prodrug or pharmaceutically acceptable salt thereof.

In some embodiments, the compound is selected from the compounds described in Table 2, or a pharmaceutically acceptable salt thereof.

In some embodiments, the compound is selected from the prodrugs of compounds described in Table 2, or a pharmaceutically acceptable salt thereof.

In some embodiments, the compound is selected from the compounds described in Table 2.

In some embodiments, the compound is selected from the compounds described in Table 2A, or a prodrug or pharmaceutically acceptable salt thereof.

In some embodiments, the compound is selected from the compounds described in Table 2A, or a pharmaceutically acceptable salt thereof.

In some embodiments, the compound is selected from the prodrugs of compounds described in Table 2A, or a pharmaceutically acceptable salt thereof.

In some embodiments, the compound is selected from the compounds described in Table 2A.

In some embodiments, the compound is selected from the compounds described in Table 3, or a prodrug or pharmaceutically acceptable salt thereof.

In some embodiments, the compound is selected from the compounds described in Table 3, or a pharmaceutically acceptable salt thereof.

In some embodiments, the compound is selected from the prodrugs of compounds described in Table 3, or a pharmaceutically acceptable salt thereof.

In some embodiments, the compound is selected from the compounds described in Table 3.

TABLE 1

| Compound | | |
|---|---|---|
| No. | Compound Name | Structure |
| I-1 | (3R,5S,E)-7-(5-cyclopropyl-7- (4-fluorophenyl)-3- methylthieno[3,2-b]pyridin-6- yl)-3,5-dihydroxyhept-6-enoic acid | 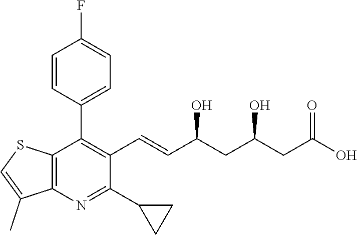 [SMILES: Cc1csc2c(-c3ccc(F)cc3)c(/C=C/[C@@H](O)C[C@@H](O)CC(=O)O)c(C3CC3)nc12] |
| I-2 | (3R,5S,E)-7-(5-cyclopropyl-7- (4-fluorophenyl)thiazolo[5,4- b]pyridin-6-yl)-3,5- dihydroxyhept-6-enoic acid | 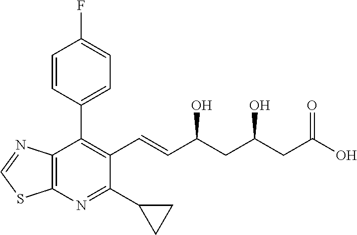 [SMILES: O=C(O)C[C@H](O)C[C@H](O)/C=C/c1c(C2CC2)nc2scnc2c1-c1ccc(F)cc1] |
| I-3 | (3R,5S,E)-7-(5-cyclopropyl-7- (4-fluorophenyl)-2- methylthiazolo[5,4-b]pyridin-6- yl)-3,5-dihydroxyhept-6-enoic acid | 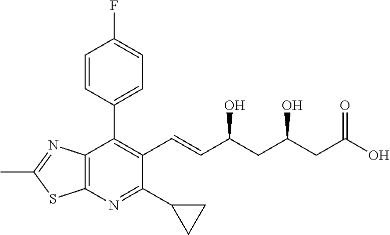 [SMILES: Cc1nc2c(-c3ccc(F)cc3)c(/C=C/[C@@H](O)C[C@@H](O)CC(=O)O)c(C3CC3)nc2s1] |
| I-4 | (3R,5S,E)-7-(5-cyclopropyl-7- (4-fluorophenyl)thieno[3,2- b]pyridin-6-yl)-3,5- dihydroxyhept-6-enoic acid | 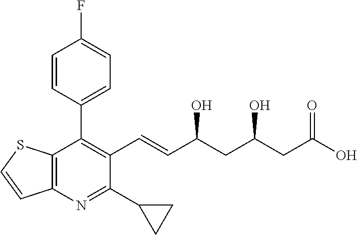 [SMILES: O=C(O)C[C@H](O)C[C@H](O)/C=C/c1c(C2CC2)nc2ccsc2c1-c1ccc(F)cc1] |
| I-5 | (3R,5S,E)-7-(6-cyclopropyl-4- (4-fluorophenyl)-2- methylthieno[2,3-b]pyridin-5- yl)-3,5-dihydroxyhept-6-enoic acid | 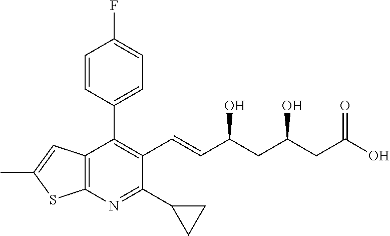 [SMILES: Cc1cc2c(-c3ccc(F)cc3)c(/C=C/[C@@H](O)C[C@@H](O)CC(=O)O)c(C3CC3)nc2s1] |
| I-5a | (3R,5S,E)-7-(6-cyclopropyl-4- (4-fluorophenyl)-2- methylthieno[2,3-b]pyridin-5- yl)-3,5-dihydroxyhept-6-enoic acid (single unknown atropoisomer) | 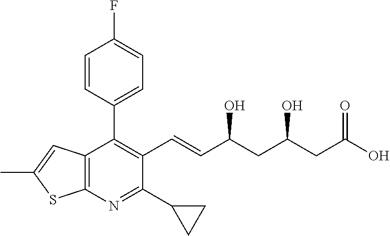 [SMILES: Cc1cc2c(-c3ccc(F)cc3)c(/C=C/[C@@H](O)C[C@@H](O)CC(=O)O)c(C3CC3)nc2s1] |
| I-5b | (3R,5S,E)-7-(6-cyclopropyl-4- (4-fluorophenyl)-2- methylthieno[2,3-b]pyridin-5- yl)-3,5-dihydroxyhept-6-enoic acid (single unknown atropoisomer) | 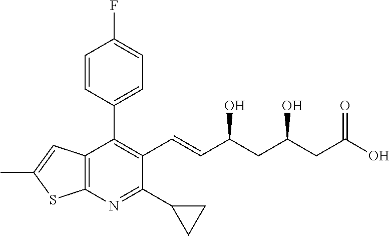 [SMILES: Cc1cc2c(-c3ccc(F)cc3)c(/C=C/[C@@H](O)C[C@@H](O)CC(=O)O)c(C3CC3)nc2s1] |
| I-5c | (3R,5S,E)-7-(6-cyclopropyl-4- (4-fluorophenyl)-2- methylthieno[2,3-b]pyridin-5- yl)-3,5-dihydroxyhept-6-enoic acid (mixture of atropoisomers I-5a and I-5b) | 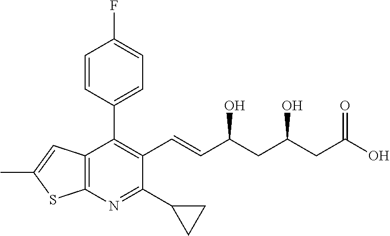 [SMILES: Cc1cc2c(-c3ccc(F)cc3)c(/C=C/[C@@H](O)C[C@@H](O)CC(=O)O)c(C3CC3)nc2s1] |
| I-6 | (3R,5S,E)-7-(5-cyclopropyl-7- (4-fluorophenyl)-2- methylthieno[3,2-b]pyridin-6- yl)-3,5-dihydroxyhept-6-enoic acid | 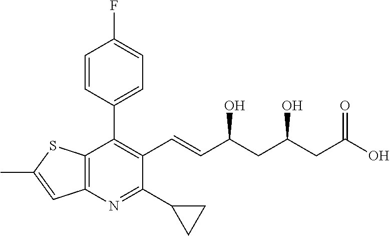 [SMILES: Cc1cc2nc(C3CC3)c(/C=C/[C@@H](O)C[C@@H](O)CC(=O)O)c(-c3ccc(F)cc3)c2s1] |
| I-7 | (3R,5S,E)-7-(6-cyclopropyl-4- (4-fluorophenyl)-2- methylbenzo[d]thiazol-5-yl)- 3,5-dihydroxyhept-6-enoic acid | 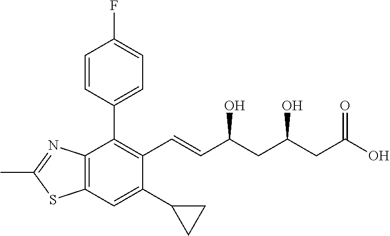 [SMILES: Cc1nc2c(-c3ccc(F)cc3)c(/C=C/[C@@H](O)C[C@@H](O)CC(=O)O)c(C3CC3)cc2s1] |
| I-8 | (3R,5S,E)-7-(5-cyclopropyl-7- (4-fluorophenyl)-2- methylbenzo[d]thiazol-6-yl)- 3,5-dihydroxyhept-6-enoic acid | 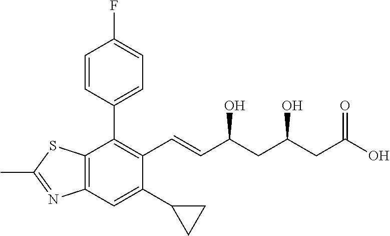 [SMILES: Cc1nc2cc(C3CC3)c(/C=C/[C@@H](O)C[C@@H](O)CC(=O)O)c(-c3ccc(F)cc3)c2s1] |
| I-9 | (3R,5S,E)-7-(6-cyclopropyl-4- (4- fluorophenyl)benzo[d]thiazol-5- yl)-3,5-dihydroxyhept-6-enoic acid | 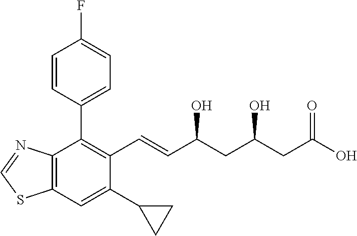 [SMILES: O=C(O)C[C@H](O)C[C@H](O)/C=C/c1c(C2CC2)cc2scnc2c1-c1ccc(F)cc1] |
| I-10 | (3R,5S,E)-7-(6-cyclopropyl-4- (4-fluorophenyl)-3- methylthieno[2,3-b]pyridin-5- yl)-3,5-dihydroxyhept-6-enoic acid | 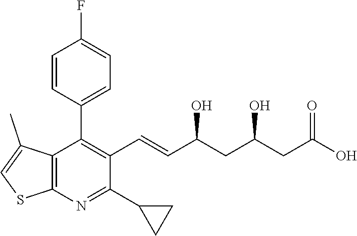 [SMILES: Cc1csc2nc(C3CC3)c(/C=C/[C@@H](O)C[C@@H](O)CC(=O)O)c(-c3ccc(F)cc3)c12] |
| I-11 | (3R,5S,E)-7-(5-cyclopropyl-7- (4- fluorophenyl)benzo[d]thiazol-6- yl)-3,5-dihydroxyhept-6-enoic acid | 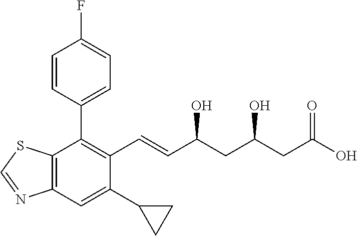 [SMILES: O=C(O)C[C@H](O)C[C@H](O)/C=C/c1c(C2CC2)cc2ncsc2c1-c1ccc(F)cc1] |

TABLE 1A

| Compound | | |
|---|---|---|
| No. | Compound Name | Structure |
| I-12 | (3R,5S,E)-7-(5- cyclopropyl-7-(4-fluoro- 2-methylphenyl)-2- methylbenzo[d]thiazol- 6-yl)-3,5- dihydroxyhept-6-enoic acid | 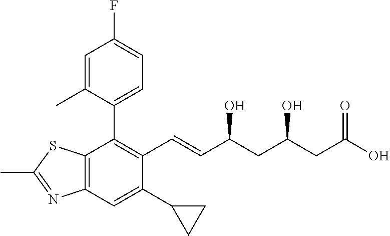 [SMILES: Cc1nc2cc(C3CC3)c(/C=C/[C@@H](O)C[C@@H](O)CC(=O)O)c(-c3ccc(F)cc3C)c2s1] |
| I-13 | (3R,5S,E)-7-(5- cyclopropyl-7-(4-fluoro- 2-methylphenyl)-3- methylthieno[3,2- b]pyridin-6-yl)-3,5- dihydroxyhept-6-enoic acid | 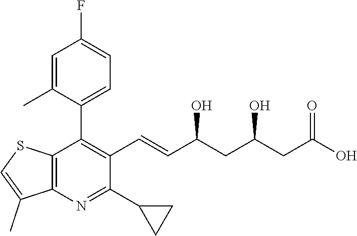 [SMILES: Cc1cc(F)ccc1-c1c(/C=C/[C@@H](O)C[C@@H](O)CC(=O)O)c(C2CC2)nc2c(C)csc12] |
| I-14 | (3R,5S,E)-7-(7-(2- chloro-4-fluorophenyl)- 5-cyclopropyl-2- methylbenzo[d]thiazol- 6-yl)-3.5- dihydroxyhept-6-enoic acid | 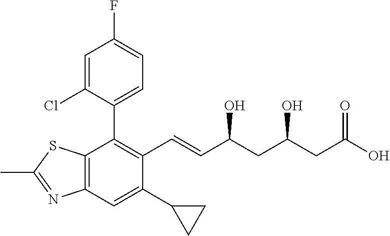 [SMILES: Cc1nc2cc(C3CC3)c(/C=C/[C@@H](O)C[C@@H](O)CC(=O)O)c(-c3ccc(F)cc3Cl)c2s1] |
| I-15 | (3R,5S,E)-7-(7-(2- chloro-4-fluorophenyl)- 5-cyclopropyl-3- methylthieno[3,2- b]pyridin-6-yl)-3,5- dihydroxyhept-6-enoic acid | 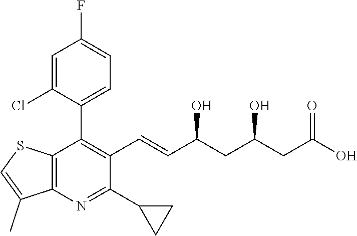 [SMILES: Cc1csc2c(-c3ccc(F)cc3Cl)c(/C=C/[C@@H](O)C[C@@H](O)CC(=O)O)c(C3CC3)nc12] |
| I-16 | (3R,5S,E)-7-(5- cyclopropyl-7-(4- fluorophenyl)-3-(1- methyl-1H-pyrazol-4- yl)thieno[3,2-b]pyridin- 6-yl)-3,5- dihydroxyhept-6-enoic acid | 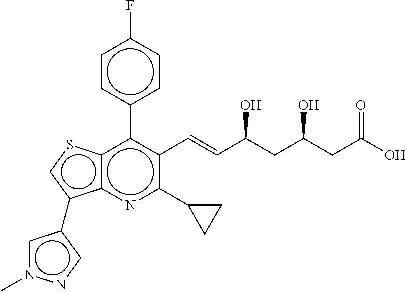 |
| I-17 | (3R,5S,E)-7-(5- cyclopropyl-7-(4- fluorophenyl)-3- (pyridin-3-yl)thieno[3,2- b]pyridin-6-yl)-3,5- dihydroxyhept-6-enoic acid | 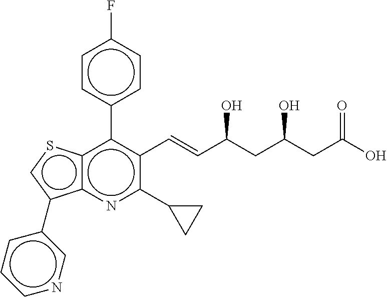 |
| I-18 | (3R,5S,E)-7-(5- cyclopropyl-7-(4- fluorophenyl)-3- phenylthieno[3,2- b]pyridin-6-yl)-3,5- dihydroxyhept-6-enoic acid (single unknown atropoisomer) | 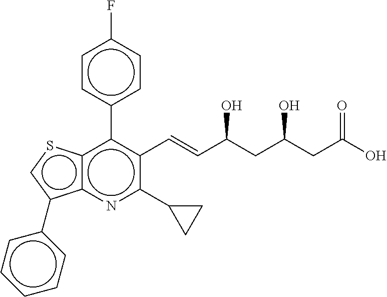 |
| I-18a | (3R,5S,E)-7-(5- cyclopropyl-7-(4- fluorophenyl)-3- phenylthieno[3,2- b]pyridin-6-yl)-3,5- dihydroxyhept-6-enoic acid (single unknown atropoisomer) | 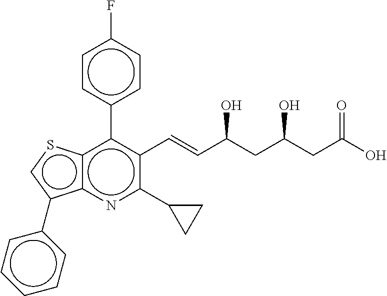 |
| I-19 | (3R,5S,E)-7-(5- cyclopropyl-3-ethyl-7- (4- fluorophenyl)thieno[3,2- b]pyridin-6-yl)-3,5- dihydroxyhept-6-enoic acid | 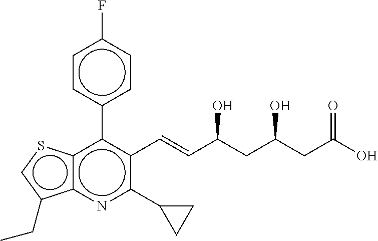 |
| I-20 | (3R,5S,E)-7-(5- cyclopropyl-7-(4- fluorophenyl)-3-(1- methylpiperidin-4- yl)thieno[3,2-b]pyridin- 6-yl)-3,5- dihydroxyhept-6-enoic acid | 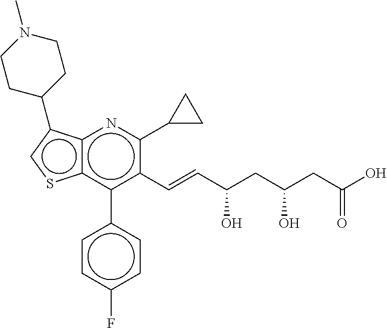 |
| I-21 | (3R,5S,E)-7-(5- cyclopropyl-7-(4- fluorophenyl)-3- (tetrahydro-2H-pyran-4- yl)thieno[3,2-b]pyridin- 6-yl)-3,5- dihydroxyhept-6-enoic acid | 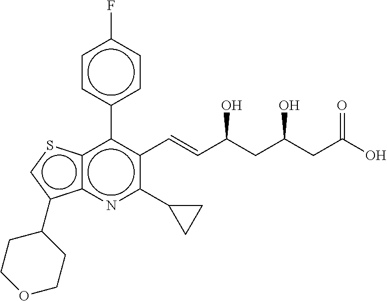 |
| I-22 | (3R,5S,E)-7-(3- cyclohexyl-5- cyclopropyl-7-(4- fluorophenyl)thieno[3,2- b]pyridin-6-yl)-3,5- dihydroxyhept-6-enoic acid | 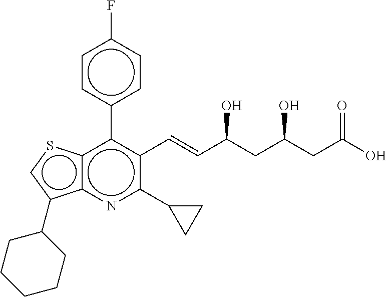 |
| I-23 | (3R,5S,E)-7-(5- cyclopropyl-7-(4- fluorophenyl)-3- isopropylthieno[3,2- b]pyridin-6-yl)-3,5- dihydroxyhept-6-enoic acid | 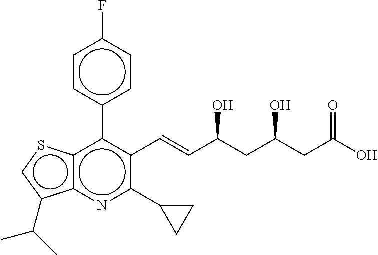 |
| I-24 | (3R,5S,E)-7-(3- cyclobutyl-5- cyclopropyl-7-(4- fluorophenyl)thieno[3,2- b]pyridine-6-yl)-3,5- dihydroxyhept-6-enoic acid | 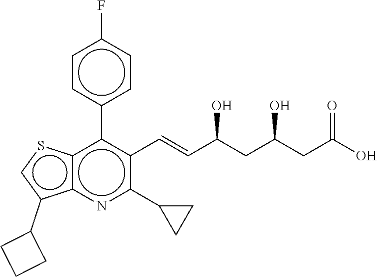 |
| I-25 | (3R,5S,E)-7-(3,5- dicyclopropyl-7-(4- fluorophenyl)thieno[3,2- b]pyridin-6-yl)-3,5- dihydroxyhept-6-enoic acid | 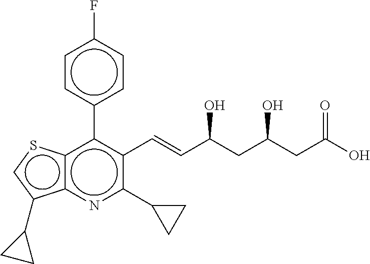 |
| I-26 | (3R,5S,E)-7-(5- cyclopropyl-7-(4- fluorophenyl)-3- methylthieno[3,2- b]pyridin-6-yl)-3,5- dihydroxy-N,N- dimethylhept-6-enamide | 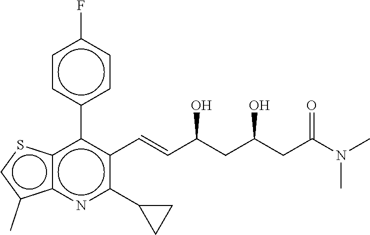 |
| I-27 | (3R,5S,E)-7-(5- cyclopropyl-7-(4- fluorophenyl)-3- methylthieno[3,2- b]pyridin-6-yl)-3,5- dihydroxy-N- methylhept-6-enamide | 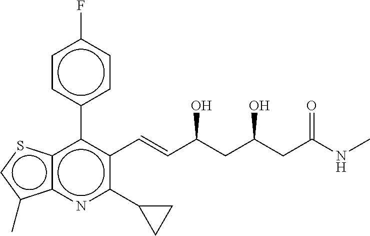 |
| I-28 | (3R,5S,E)-7-(5- cyclopropyl-7-(4- fluorophenyl)-3- methylthieno[3,2- b]pyridin-6-yl)-3,5- dihydroxyhept-6- enamide | 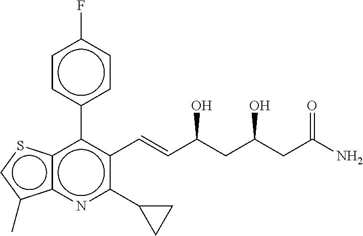 |
| I-29 | (R,E)-7-(5-cyclopropyl- 7-(4-fluorophenyl)-3- methylthieno[3,2- b]pyridin-6-yl)-3- hydroxyhept-6-enoic acid | 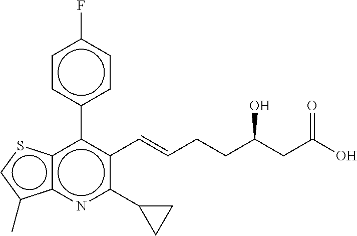 |
| I-30 | (S,E)-7-(5-cyclopropyl- 7-(4-fluorophenyl)-3- methylthieno[3,2- b]pyridin-6-yl)-5- hydroxyhept-6-enoic acid | 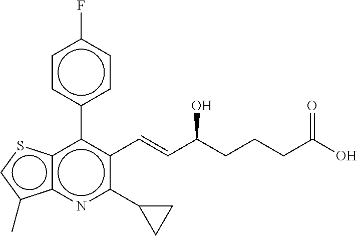 |
| I-31 | (2S,4S,E)-6-(5- cyclopropyl-7-(4- fluorophenyl)-3- methylthieno[3,2- b]pyridin-6-yl)hex-5- ene-2,4-diol | 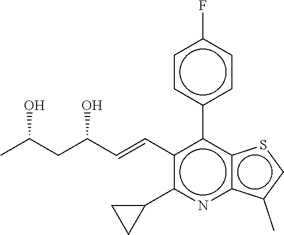 |
| I-32 | (3R,5S)-7-(6- cyclopropyl-4-(4- fluorophenyl)-3- methylthieno[2,3- b]pyridin-5-yl)-3,5- dihydroxyheptanoic acid | 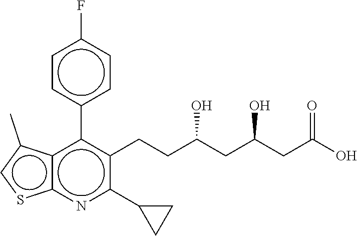 |
| I-33 | (3R,5S)-7-(5- cyclopropyl-7-(4- fluorophenyl)-3- methylthieno[3,2- b]pyridin-6-yl)-3,5- dihydroxyheptanoic acid | 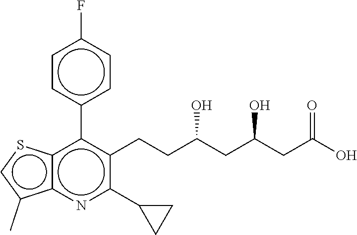 |
| I-34 | (3R,5S,E)-7-(4-(4- chlorophenyl)-6- cyclopropyl-3- methylthieno[2,3- b]pyridin-5-yl)-3,5- dihydroxyhept-6-enoic acid | 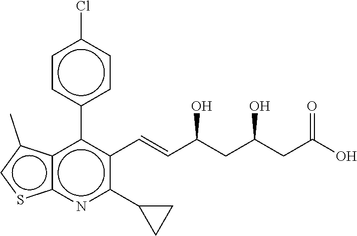 |
| I-35 | (3R,5S,E)-7-(4-(3- chlorophenyl)-6- cyclopropyl-3- methylthieno[2,3- b]pyridin-5-yl)-3,5- dihydroxyhept-6-enoic acid | 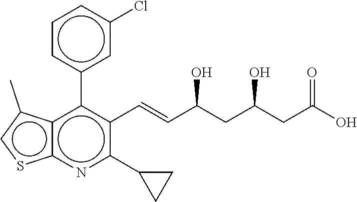 |
| I-36 | (3R,5S,E)-7-(6- cyclopropyl-4-(4-fluoro- 2-methylphenyl)-3- methylthieno[2,3- b]pyridin-5-yl)-3,5- dihydroxyhept-6-enoic acid (single unknown atropisomer) | 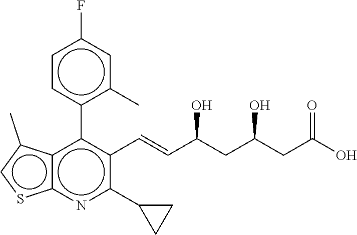 |
| I-37 | (3R,5S,E)-7-(6- cyclopropyl-4-(4-fluoro- 2-methylphenyl)-3- methylthieno[2,3- b]pyridin-5-yl)-3,5- dihydroxyhept-6-enoic acid (single unknown atropisomer) | 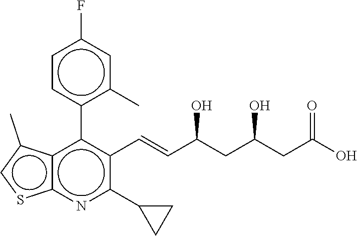 |
| I-38 | (3R,5S,E)-7-(3,6- dicyclopropyl-4-(4- fluorophenyl)thieno[2,3- b]pyridin-5-yl)-3,5- dihydroxyhept-6-enoic acid | 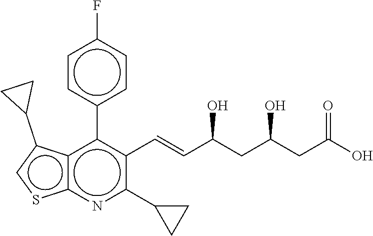 |
| I-39 | (3R,5S,E)-7-(2,6- dicyclopropyl-4-(4- fluorophenyl)thieno[2,3- b]pyridin-5-yl)-3,5- dihydroxyhept-6-enoic acid | 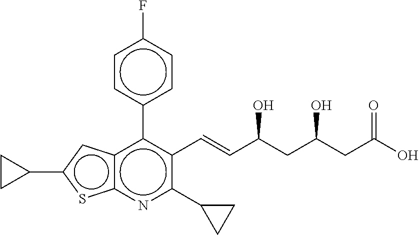 |
| I-40 | methyl (3R,5S,E)-7-(7- (4-chloro-2- fluorophenyl)-5- cyclopropyl-2- methylthieno[3,2- b]pyridin-6-yl)-3,5- dihydroxyhept-6-enoate | 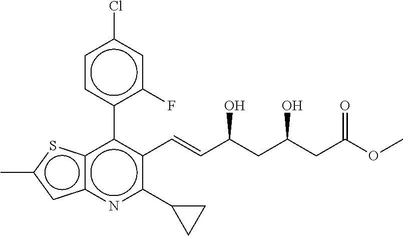 |
| I-41 | methyl (3R,5S,E)-7-(5- cyclopropyl-7-(2,4- dichlorophenyl)-2- methylthieno[3,2- b]pyridin-6-yl)-3,5- dihydroxyhept-6-enoate | 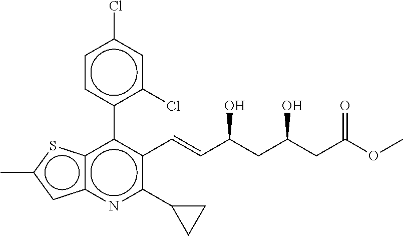 |
| I-42 | methyl (3R,5S,E)-7-(7- (2-chloro-4- methylphenyl)-5- cyclopropyl-2- methylthieno[3,2- b]pyridin-6-yl)-3,5- dihydroxyhept-6-enoate | 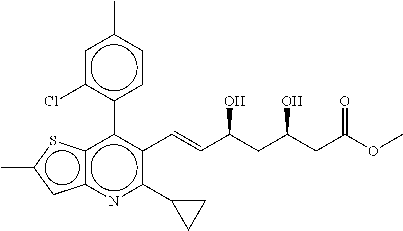 |
| I-43 | methyl (3R,5S,E)-7-(7- (2-chloro-4- fluorophenyl)-5- cyclopropyl-2- methylthieno[3,2- b]pyridin-6-yl)-3,5- dihydroxyhept-6-enoate | 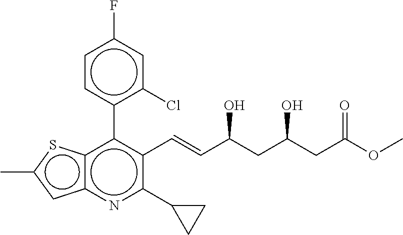 |
| I-44 | methyl (3R,5S,E)-7-(5- cyclopropyl-7-(4- fluorophenyl)-2- methylthieno[3,2- b]pyridin-6-yl)-3,5- dihydroxyhept-6-enoate | 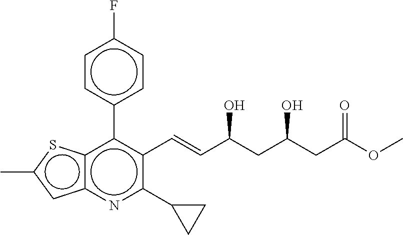 |
| I-45 | (3R,5S,E)-7-(2,5- dicyclopropyl-7-(4- fluorophenyl)thieno[3,2- b]pyridin-6-yl)-3,5- dihydroxyhept-6-enoic acid | 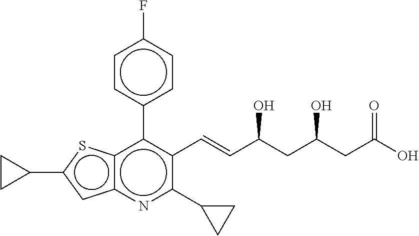 |

TABLE 2

| Compound | | |
|---|---|---|
| No. | Compound Name | Structure |
| II-1 | (3R,5S,E)-7-(6-(bicyclo[1.1.1]pentan-1- yl)-4-(2-chloro-4-fluorophenyl)-2- cyclopropyl-5-(methoxymethyl)pyridin-3- yl)-3,5-dihydroxyhept-6-enoic acid | 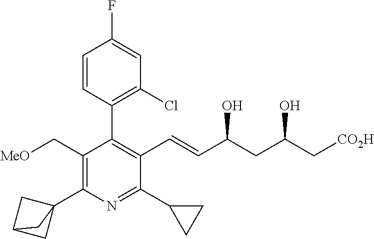 [SMILES: COCc1c(C23CC(C2)C3)nc(C2CC2)c(/C=C/[C@@H](O)C[C@@H](O)CC(=O)O)c1-c1ccc(F)cc1Cl] |
| II-2 | (3R,5S,E)-7-(6-(bicyclo[1.1.1]pentan-1- yl)-2-cyclopropyl-4-(4-fluorophenyl)-5- (methoxymethyl)pyridin-3-yl)-3,5- dihydroxyhept-6-enoic acid | 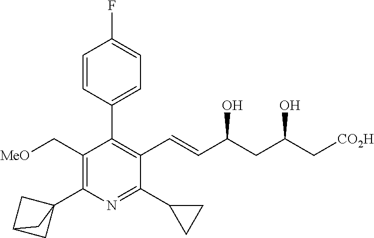 [SMILES: COCc1c(C23CC(C2)C3)nc(C2CC2)c(/C=C/[C@@H](O)C[C@@H](O)CC(=O)O)c1-c1ccc(F)cc1] |
| II-3 | (3R,5S,E)-7-(4-(2-chloro-4-fluorophenyl)- 2,6-dicyclobutyl-5- (methoxymethyl)pyridin-3-yl)-3,5- dihydroxyhept-6-enoic acid | 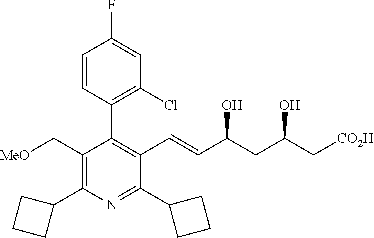 [SMILES: COCc1c(C2CCC2)nc(C2CCC2)c(/C=C/[C@@H](O)C[C@@H](O)CC(=O)O)c1-c1ccc(F)cc1Cl] |
| II-4 | (3R,5S,E)-7-(4-(2-chloro-4-fluorophenyl)- 6-cyclobutyl-2-cyclopropyl-5- (methoxymethyl)pyridin-3-yl)-3,5- dihydroxyhept-6-enoic acid | 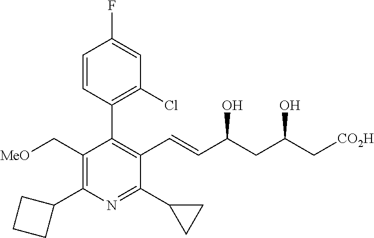 [SMILES: COCc1c(C2CCC2)nc(C2CC2)c(/C=C/[C@@H](O)C[C@@H](O)CC(=O)O)c1-c1ccc(F)cc1Cl] |
| II-5 | (3R,5S,E)-7-(6-cyclobutyl-2-cyclopropyl- 4-(4-fluorophenyl)-5- (methoxymethyl)pyridin-3-yl)-3,5- dihydroxy hept-6-enoic acid | 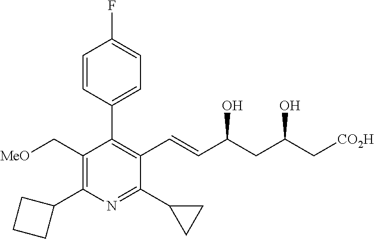 [SMILES: COCc1c(C2CCC2)nc(C2CC2)c(/C=C/[C@@H](O)C[C@@H](O)CC(=O)O)c1-c1ccc(F)cc1] |
| II-6 | (3R,5S,E)-7-(2,6-dicyclobutyl-4-(4- fluorophenyl)-5-(methoxymethyl)pyridin- 3-yl)-3,5-dihydroxyhept-6-enoic acid | 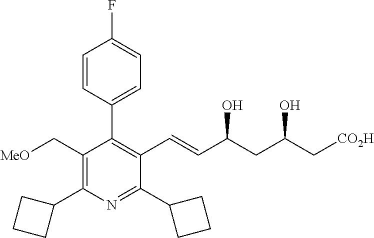 [SMILES: COCc1c(C2CCC2)nc(C2CCC2)c(/C=C/[C@@H](O)C[C@@H](O)CC(=O)O)c1-c1ccc(F)cc1] |
| II-7 | (3R,5S,E)-7-(4-(2-chloro-4-fluorophenyl)- 6-cyclobutyl-2-cyclopropylpyridin-3-yl)- 3,5-dihydroxyhept-6-enoic acid | 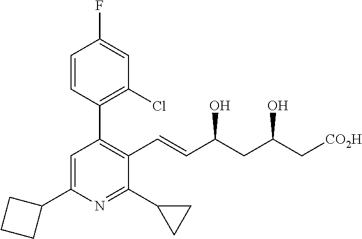 [SMILES: O=C(O)C[C@H](O)C[C@H](O)/C=C/c1c(-c2ccc(F)cc2Cl)cc(C2CCC2)nc1C1CC1] |
| II-8 | (3R,5S,E)-7-(4-(2-chloro-4-fluorophenyl)- 2,6-dicyclopropylpyridin-3-yl)-3,5- dihydroxyhept-6-enoic acid | 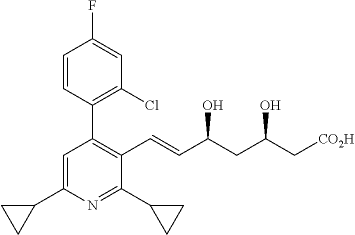 [SMILES: O=C(O)C[C@H](O)C[C@H](O)/C=C/c1c(-c2ccc(F)cc2Cl)cc(C2CC2)nc1C1CC1] |
| II-9 | (3R,5S,E)-7-(4-(2-chloro-4-fluorophenyl)- 2,6-dicyclopropyl-5- (methoxymethyl)pyridin-3-yl)-3,5- dihydroxyhept-6-enoic acid | 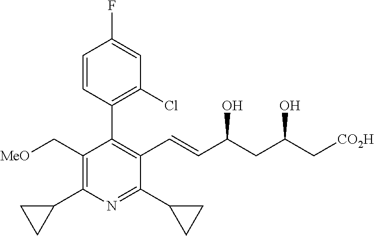 [SMILES: COCc1c(C2CC2)nc(C2CC2)c(/C=C/[C@@H](O)C[C@@H](O)CC(=O)O)c1-c1ccc(F)cc1Cl] |
| II-10 | (3R,5S,E)-7-(2,6-dicyclopropyl-4-(4- fluoro-2-methylphenyl)-5- (methoxymethyl)pyridin-3-yl)-3,5- dihydroxyhept-6-enoic acid | 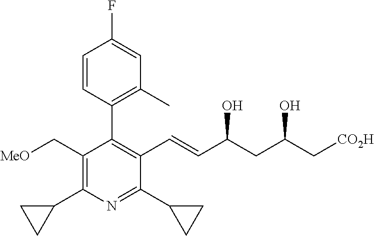 [SMILES: COCc1c(C2CC2)nc(C2CC2)c(/C=C/[C@@H](O)C[C@@H](O)CC(=O)O)c1-c1ccc(F)cc1C] |

TABLE 2A

| Compound | | |
|---|---|---|
| No. | Compound Name | Structure |
| II-12 | (3R,5S,E)-7-(2,6- dicyclopropyl-4-(4-fluoro- 2-methylphenyl)pyridin- 3-yl)-3,5-dihydroxyhept- 6-enoic acid | 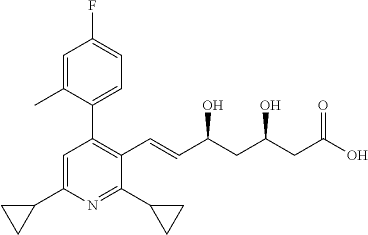 [SMILES: Cc1cc(F)ccc1-c1cc(C2CC2)nc(C2CC2)c1/C=C/[C@@H](O)C[C@@H](O)CC(=O)O] |
| II-13 | (3R,5S,E)-7-(4-(2-chloro- 5-fluorophenyl)-2,6- dicyclopropyl-5- (methoxymethyl)pyridin- 3-yl)-3,5-dihydroxyhept- 6-enoic acid | 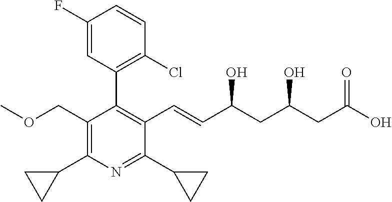 [SMILES: COCc1c(C2CC2)nc(C2CC2)c(/C=C/[C@@H](O)C[C@@H](O)CC(=O)O)c1-c1cc(F)ccc1Cl] |
| II-14 | (3R,5S,E)-7-(6- (bicyclo[1.1.1]pentan-1- yl)-2-cyclopropyl-4-(4- fluoro-2- methylphenyl)pyridin-3- yl)-3,5-dihydroxyhept-6- enoic acid | 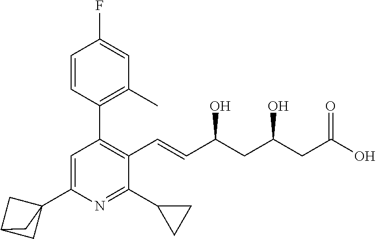 [SMILES: Cc1cc(F)ccc1-c1cc(C23CC(C2)C3)nc(C2CC2)c1/C=C/[C@@H](O)C[C@@H](O)CC(=O)O] |
| II-15 | (3R,5S,E)-7-(6- (bicyclo[1.1.1]pentan-1- yl)-4-(2-chloro-4- fluorophenyl)-2- cyclopropylpyridin-3-yl)- 3,5-dihydroxyhept-6- enoic acid | 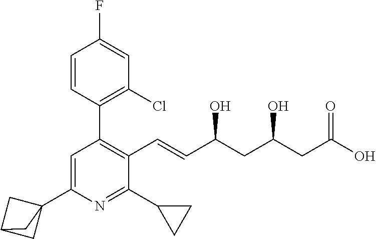 [SMILES: O=C(O)C[C@H](O)C[C@H](O)/C=C/c1c(-c2ccc(F)cc2Cl)cc(C23CC(C2)C3)nc1C1CC1] |
| II-16 | (3R,5S,E)-7-(6- (bicyclo[1.1.1]pentan-1- yl)-2-cyclopropyl-4-(4- fluorophenyl)pyridin-3- yl)-3,5-dihydroxyhept-6- enoic acid | 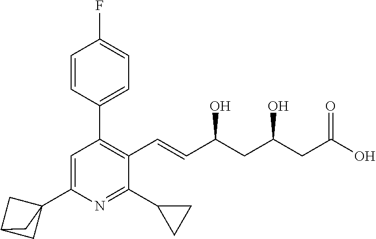 [SMILES: O=C(O)C[C@H](O)C[C@H](O)/C=C/c1c(-c2ccc(F)cc2)cc(C23CC(C2)C3)nc1C1CC1] |
| II-17 | (3R,5S,E)-7-(2,6- dicyclopropyl-4-(2,4- difluoro-6-methylphenyl)- 5- (methoxymethyl)pyridin- 3-yl)-3,5-dihydroxyhept- 6-enoic acid | 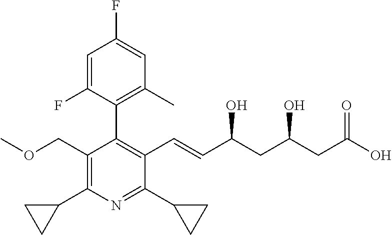 [SMILES: COCc1c(C2CC2)nc(C2CC2)c(/C=C/[C@@H](O)C[C@@H](O)CC(=O)O)c1-c1c(C)cc(F)cc1F] |
| II-18 | (3R,5S,E)-7-(2,6- dicyclopropyl-4-(2-fluoro- 6-methylphenyl)-5- (methoxymethyl)pyridin- 3-yl)-3,5-dihydroxyhept- 6-enoic acid | 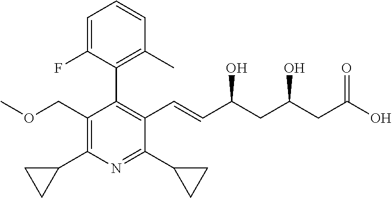 [SMILES: COCc1c(C2CC2)nc(C2CC2)c(/C=C/[C@@H](O)C[C@@H](O)CC(=O)O)c1-c1c(C)cccc1F] |
| II-19 | (3R,5S,E)-7-(4-(4-chloro- 2-fluorophenyl)-2,6- dicyclopropyl-5- (methoxymethyl)pyridin- 3-yl)-3,5-dihydroxyhept- 6-enoic acid | 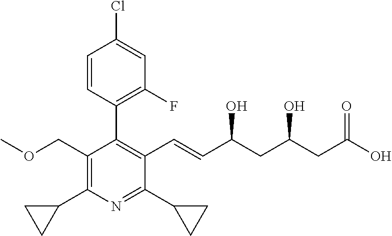 [SMILES: COCc1c(C2CC2)nc(C2CC2)c(/C=C/[C@@H](O)C[C@@H](O)CC(=O)O)c1-c1ccc(Cl)cc1F] |
| II-20 | (3R,5S,E)-7-(4-(5-chloro- 2-fluorophenyl)-2,6- dicyclopropyl-5- (methoxymethyl)pyridin- 3-yl)-3,5-dihydroxyhept- 6-enoic acid | 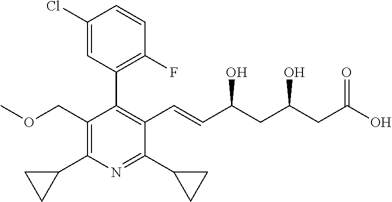 [SMILES: COCc1c(C2CC2)nc(C2CC2)c(/C=C/[C@@H](O)C[C@@H](O)CC(=O)O)c1-c1cc(Cl)ccc1F] |
| II-21 | (3R,5S,E)-7-(4-(2-chloro- 4,6-difluorophenyl)-2,6- dicyclopropyl-5- (methoxymethyl)pyridin- 3-yl)-3,5-dihydroxyhept- 6-enoic acid | 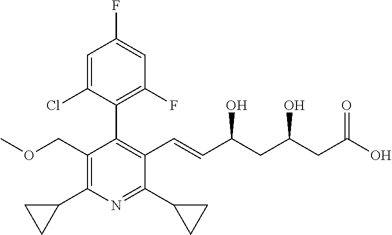 [SMILES: COCc1c(C2CC2)nc(C2CC2)c(/C=C/[C@@H](O)C[C@@H](O)CC(=O)O)c1-c1c(F)cc(F)cc1Cl] |
| II-22 | (3R,5S,E)-7-(4-(2-chloro- 6-fluorophenyl)-2,6- dicyclopropyl-5- (methoxymethyl)pyridin- 3-yl)-3,5-dihydroxyhept- 6-enoic acid | 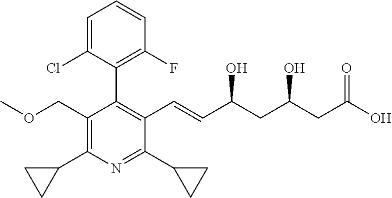 [SMILES: COCc1c(C2CC2)nc(C2CC2)c(/C=C/[C@@H](O)C[C@@H](O)CC(=O)O)c1-c1c(F)cccc1Cl] |
| II-23 | (3R,5S,E)-7-(4-(4-chloro- 2-fluorophenyl)-5- (cyclopropoxymethyl)- 2,6-dicyclopropylpyridin- 3-yl)-3,5-dihydroxyhept- 6-enoic acid | 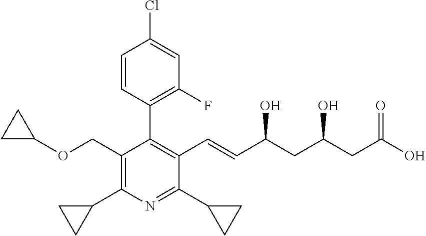 [SMILES: O=C(O)C[C@H](O)C[C@H](O)/C=C/c1c(C2CC2)nc(C2CC2)c(COC2CC2)c1-c1ccc(Cl)cc1F] |
| II-24 | (3R,5S,E)-7-(2,6- dicyclopropyl-4-(5-fluoro- 2-methylphenyl)-5- (methoxymethyl)pyridin- 3-yl)-3,5-dihydroxyhept- 6-enoic acid | 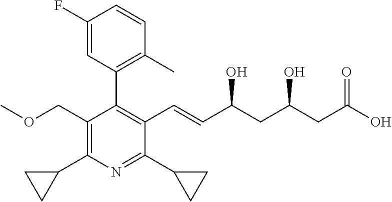 [SMILES: COCc1c(C2CC2)nc(C2CC2)c(/C=C/[C@@H](O)C[C@@H](O)CC(=O)O)c1-c1cc(F)ccc1C] |
| II-25 | (3R,5S,E)-7-(4-(2- chlorophenyl)-2,6- dicyclopropyl-5- (methoxymethyl)pyridin- 3-yl)-3,5-dihydroxyhept- 6-enoic acid | 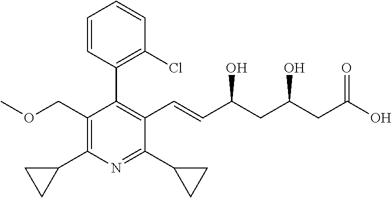 [SMILES: COCc1c(C2CC2)nc(C2CC2)c(/C=C/[C@@H](O)C[C@@H](O)CC(=O)O)c1-c1ccccc1Cl] |
| II-26 | (3R,5S,E)-7-(6- cyclopropyl-4-(4-fluoro- 2-methylphenyl)-5- (methoxymethyl)-2- methylpyridin-3-yl)-3,5- dihydroxyhept-6-enoic acid | 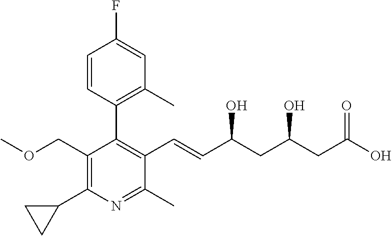 [SMILES: COCc1c(C2CC2)nc(C)c(/C=C/[C@@H](O)C[C@@H](O)CC(=O)O)c1-c1ccc(F)cc1C] |
| II-27 | (3R,5S,E)-7-(4-(2-chloro- 4-fluorophenyl)-2- cyclobutyl-6- cyclopropylpyridin-3-yl)- 3,5-dihydroxyhept-6- enoic acid | 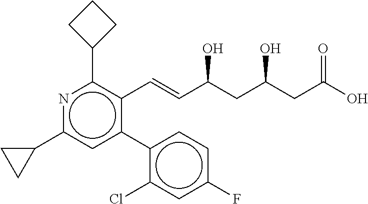 |
| II-28 | (3R,5S,E)-7-(2,6- dicyclopropyl-4-(5-fluoro- 2-methylphenyl)pyridin- 3-yl)-3,5-dihydroxyhept- 6-enoic acid | 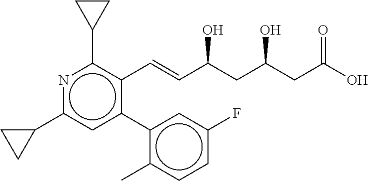 |
| II-29 | (3R,5S,E)-7-(4-(2-chloro- 5-fluorophenyl)-2,6- dicyclopropylpyridin-3- yl)-3,5-dihydroxyhept-6- enoic acid | 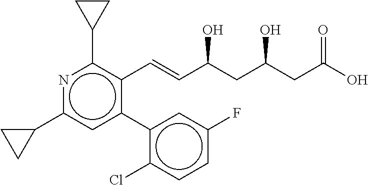 |
| II-30 | (3R,5S,E)-7-(2,6- dicyclopropyl-4-(4- fluorophenyl)pyridin-3- yl)-3,5-dihydroxyhept-6- enoic acid | 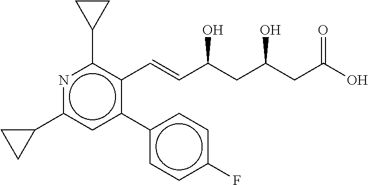 |
| II-31 | (3R,5S,E)-7-(4-(2-chloro- 4-fluorophenyl)-2,5- dicyclopropylpyridin-3- yl)-3,5-dihydroxyhept-6- enoic acid | 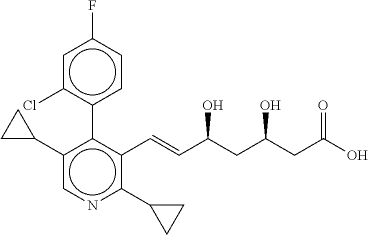 |
| II-32 | (3R,5S,E)-7-(2,5- dicyclopropyl-4-(4- fluorophenyl)pyridin-3- yl)-3,5-dihydroxyhept-6- enoic acid | 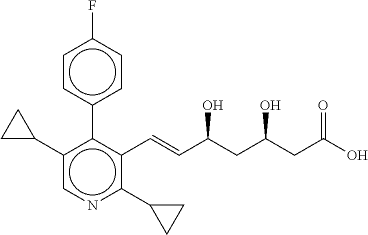 |

TABLE 3

| Compound | | |
|---|---|---|
| No. | Compound Name | Structure |
| III-1 | (3R,5S,E)-7-(8-chloro-2,4- dicyclopropyl-5H-chromeno[3,4- c]pyridin-1-yl)-3,5- dihydroxyhept-6-enoic acid | 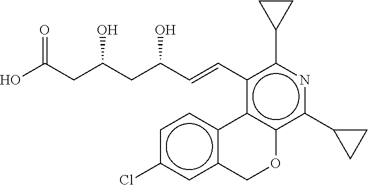 |
| III-2 | (3R,5S,Z)-8-(8-chloro-2,4- dicyclopropyl-5H-chromeno[3,4- c]pyridin-1-yl)-3,5-dihydroxyoct- 7-enoic acid | 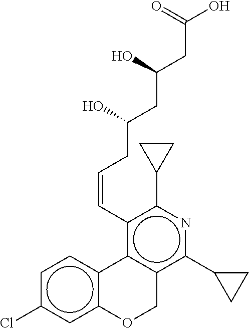 |

In some embodiments, the compound is a pharmaceutically acceptable salt of any one of the compounds described in Table 1.

In some embodiments, the compound is a pharmaceutically acceptable salt of any one of the compounds described in Table 1A.

In some embodiments, the compound is a pharmaceutically acceptable salt of any one of the compounds described in Table 2.

In some embodiments, the compound is a pharmaceutically acceptable salt of any one of the compounds described in Table 2A.

In some embodiments, the compound is a pharmaceutically acceptable salt of any one of the compounds described in Table 3.

In some aspects, the present disclosure provides a compound being an isotopic derivative (e.g., isotopically labeled compound) of any one of the compounds of the Formulae disclosed herein.

In some embodiments, the compound is an isotopic derivative of any one of the compounds described in Table 1, or a prodrug or pharmaceutically acceptable salt thereof.

In some embodiments, the compound is an isotopic derivative of any one of the compounds described in Table 1, or a pharmaceutically acceptable salt thereof.

In some embodiments, the compound is an isotopic derivative of any one of prodrugs of the compounds described in Table 1, or a pharmaceutically acceptable salt thereof.

In some embodiments, the compound is an isotopic derivative of any one of the compounds described in Table 1.

In some embodiments, the compound is an isotopic derivative of any one of the compounds described in Table 1A, or a prodrug or pharmaceutically acceptable salt thereof.

In some embodiments, the compound is an isotopic derivative of any one of the compounds described in Table 1A, or a pharmaceutically acceptable salt thereof.

In some embodiments, the compound is an isotopic derivative of any one of prodrugs of the compounds described in Table 1A, or a pharmaceutically acceptable salt thereof.

In some embodiments, the compound is an isotopic derivative of any one of the compounds described in Table 1A.

In some embodiments, the compound is an isotopic derivative of any one of the compounds described in Table 2, or a prodrug or pharmaceutically acceptable salt thereof.

In some embodiments, the compound is an isotopic derivative of any one of the compounds described in Table 2, or a pharmaceutically acceptable salt thereof.

In some embodiments, the compound is an isotopic derivative of any one of prodrugs of the compounds described in Table 2, or a pharmaceutically acceptable salt thereof.

In some embodiments, the compound is an isotopic derivative of any one of the compounds described in Table 2.

In some embodiments, the compound is an isotopic derivative of any one of the compounds described in Table 2A, or a prodrug or pharmaceutically acceptable salt thereof.

In some embodiments, the compound is an isotopic derivative of any one of the compounds described in Table 2A, or a pharmaceutically acceptable salt thereof.

In some embodiments, the compound is an isotopic derivative of any one of prodrugs of the compounds described in Table 2A, or a pharmaceutically acceptable salt thereof.

In some embodiments, the compound is an isotopic derivative of any one of the compounds described in Table 2A.

In some embodiments, the compound is an isotopic derivative of any one of the compounds described in Table 3, or a prodrug or pharmaceutically acceptable salt thereof.

In some embodiments, the compound is an isotopic derivative of any one of the compounds described in Table 3, or a pharmaceutically acceptable salt thereof.

In some embodiments, the compound is an isotopic derivative of any one of prodrugs of the compounds described in Table 3, or a pharmaceutically acceptable salt thereof.

In some embodiments, the compound is an isotopic derivative of any one of the compounds described in Table 3.

In some embodiments, the pharmaceutically acceptable salt is a sodium salt.

It is understood that the isotopic derivative can be prepared using any of a variety of art-recognized techniques. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

The term “isotopic derivative”, as used herein, refers to a derivative of a compound in which one or more atoms are isotopically enriched or labelled. For example, an isotopic derivative of a compound of Formula (I) or Formula (II) is isotopically enriched with regard to, or labelled with, one or more isotopes as compared to the corresponding compound of Formula (I) or Formula (II). In some embodiments, the isotopic derivative is enriched with regard to, or labelled with, one or more atoms selected from ²H, ¹³C, ¹⁴C, ¹⁵N, ¹⁸O, ²⁹Si, ³¹P, and ³⁴S. In some embodiments, the isotopic derivative is a deuterium labeled compound (i.e., being enriched with ²H with regard to one or more atoms thereof). In some embodiments, the compound is a ¹⁸F labeled compound. In some embodiments, the compound is a ¹²³I labeled compound, a ¹²⁴I labeled compound, a ¹²⁵I labeled compound, a ¹²⁹I labeled compound, a ¹³¹I labeled compound, a ¹³⁵I labeled compound, or any combination thereof. In some embodiments, the compound is a ³³S labeled compound, a ³⁴S labeled compound, a ³⁵S labeled compound, a ³⁶S labeled compound, or any combination thereof.

It is understood that the ¹⁸F, ¹²³I, ¹²⁴I, ¹²⁵I, ¹²⁹I, ¹³¹I, ¹³⁵I, ³²S, ³⁴S, ³⁵S, and/or ³⁶S labeled compound, can be prepared using any of a variety of art-recognized techniques. For example, the deuterium labeled compound can generally be prepared by carrying out the procedures disclosed in the Schemes and/or in the Examples described herein, by substituting a ¹⁸F, ¹²³I, ¹²⁴I, ¹²⁵I, ¹²⁹I, ¹³¹I, ¹³⁵I, ³S, ³⁴S, ³⁵S, and/or ³⁶S labeled reagent for a non-isotope labeled reagent.

A compound of the invention or a pharmaceutically acceptable salt or solvate thereof that contains one or more of the aforementioned ¹⁸F, ¹²³I, ¹²⁴I, ¹²⁵I, ¹²⁹I, ¹³¹I, ¹³⁵I, ³²S, ³⁴S, ³⁵S, and ³⁶S atom(s) is within the scope of the invention. Further, substitution with isotope (e.g., ¹⁸F, ¹²³I, ¹²⁴I, ¹²⁵I, ¹²⁹I, ¹³¹I, ¹³⁵I, ³S, ³⁴S, ³⁵S, and/or ³⁶S) may afford certain therapeutic advantages resulting from greater metabolic stability, e.g., increased in vivo half-life or reduced dosage requirements.

For the avoidance of doubt it is to be understood that, where in this specification a group is qualified by “described herein”, the said group encompasses the first occurring and broadest definition as well as each and all of the particular definitions for that group.

The various functional groups and substituents making up the compounds of the Formula (I) or Formula (II) are typically chosen such that the molecular weight of the compound does not exceed 1000 daltons. More usually, the molecular weight of the compound will be less than 900, for example less than 800, or less than 750, or less than 700, or less than 650 daltons. More conveniently, the molecular weight is less than 600 and, for example, is 550 daltons or less.

A suitable pharmaceutically acceptable salt of a compound of the disclosure is, for example, an acid-addition salt of a compound of the disclosure which is sufficiently basic, for example, an acid-addition salt with, for example, an inorganic organic acid, for example hydrochloric, hydrobromic, sulfuric, phosphoric, trifluoroacetic, formic, citric methane sulfonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl)amine.

It will be understood that the compounds of any one of the Formulae disclosed herein and any pharmaceutically acceptable salts thereof, comprise stereoisomers, mixtures of stereoisomers, polymorphs of all isomeric forms of said compounds.

It will be understood that while compounds disclosed herein may be presented in one particular configuration. Such particular configuration is not to be construed as limiting the disclosure to one or another isomer, tautomer, regioisomer or stereoisomer, nor does it exclude mixtures of isomers, tautomers, regioisomers or stereoisomers. In some embodiments, the presentation of a compound herein in a particular configuration intends to encompass, and to refer to, each of the available isomers, tautomers, regioisomers, and stereoisomers of the compound, or any mixture thereof; while the presentation further intends to refer to the specific configuration of the compound.

It will be understood that while compounds disclosed herein may be presented without specified configuration (e.g., without specified stereochemistry). Such presentation intends to encompass all available isomers, tautomers, regioisomers, and stereoisomers of the compound. In some embodiments, the presentation of a compound herein without specified configuration intends to refer to each of the available isomers, tautomers, regioisomers, and stereoisomers of the compound, or any mixture thereof.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral center” refers to a carbon atom bonded to four nonidentical substituents.

As used herein, the term “chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterised by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4^{th} edition J. March, John Wiley and Sons, New York, 2001), for example by synthesis from optically active starting materials or by resolution of a racemic form. Some of the compounds of the disclosure may have geometric isomeric centers (E- and Z-isomers). It is to be understood that the present disclosure encompasses all optical, diastereoisomers and geometric isomers and mixtures thereof that possess STMN2 modulatory activity.

The present disclosure also encompasses compounds of the disclosure as defined herein which comprise one or more isotopic substitutions.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water, the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonamides, tetrazoles, sulfonates and phosphonates. See. e.g., Patani and LaVoie, Chem. Rev. 96, 3147-3176, 1996.

It is also to be understood that certain compounds of any one of the Formulae disclosed herein may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate. It is to be understood that the disclosure encompasses all such solvated forms that possess STMN2 activity.

It is also to be understood that certain compounds of any one of the Formulae disclosed herein may exhibit polymorphism, and that the disclosure encompasses all such forms, or mixtures thereof, which possess STMN2 activity. It is generally known that crystalline materials may be analysed using conventional techniques such as X-Ray Powder Diffraction analysis, Differential Scanning calorimetry, Thermal Gravimetric Analysis, Diffuse Reflectance Infrared Fourier Transform (DRIFT) spectroscopy, Near Infrared (NIR) spectroscopy, solution and/or solid state nuclear magnetic resonance spectroscopy. The water content of such crystalline materials may be determined by Karl Fischer analysis.

Compounds of any one of the Formulae disclosed herein may exist in a number of different tautomeric forms and references to compounds of Formula (I) or Formula (II) include all such forms. For the avoidance of doubt, where a compound can exist in one of several tautomeric forms, and only one is specifically described or shown, all others are nevertheless embraced by Formula (I) or Formula (II). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

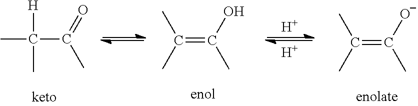

Compounds of any one of the Formulae disclosed herein containing an amine function may also form N-oxides. A reference herein to a compound of Formula (I) or Formula (II) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-oxides can be formed by treatment of the corresponding amine with an oxidizing agent such as hydrogen peroxide or a peracid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with meta-chloroperoxybenzoic acid (mCPBA), for example, in an inert solvent such as dichloromethane.

The compounds of any one of the Formulae disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at the ester or amide group in any one of the Formulae disclosed herein.

Accordingly, the present disclosure includes those compounds of any one of the Formulae disclosed herein as defined hereinbefore when made available by organic synthesis and when made available within the human or animal body by way of cleavage of a prodrug thereof. Accordingly, the present disclosure includes those compounds of any one of the Formulae disclosed herein that are produced by organic synthetic means and also such compounds that are produced in the human or animal body by way of metabolism of a precursor compound, that is a compound of any one of the Formulae disclosed herein may be a synthetically-produced compound or a metabolically-produced compound.

A suitable pharmaceutically acceptable prodrug of a compound of any one of the Formulae disclosed herein is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of any one of the Formulae disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl) 2carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl) piperazin-1-ylmethyl. Suitable pharmaceutically acceptable ether forming groups for a hydroxy group include «-acyloxyalkyl groups such as acetoxymethyl and pivaloyloxymethyl groups.

A suitable pharmaceutically acceptable prodrug of a compound of any one of the Formulae disclosed herein that possesses a carboxy group is, for example, an in vivo cleavable amide thereof, for example an amide formed with an amine such as ammonia, a C₁-4alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of any one of the Formulae disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl) piperazin-1-ylmethyl.

The in vivo effects of a compound of any one of the Formulae disclosed herein may be exerted in part by one or more metabolites that are formed within the human or animal body after administration of a compound of any one of the Formulae disclosed herein. As stated hereinbefore, the in vivo effects of a compound of any one of the Formulae disclosed herein may also be exerted by way of metabolism of a precursor compound (a prodrug).

Suitably, the present disclosure excludes any individual compounds not possessing the biological activity defined herein.

### Methods of Synthesis

In some aspects, the present disclosure provides a method of preparing a compound of the present disclosure.

In some aspects, the present disclosure provides a method of a compound, comprising one or more steps as described herein.

In some aspects, the present disclosure provides a compound obtainable by, or obtained by, or directly obtained by a method for preparing a compound as described herein.

In some aspects, the present disclosure provides an intermediate as described herein, being suitable for use in a method for preparing a compound as described herein.

The compounds of the present disclosure can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilized.

It will be appreciated that during the synthesis of the compounds of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl, or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium on carbon.

Once a compound of Formula (I) or Formula (II) has been synthesized by any one of the processes defined herein, the processes may then further comprise the additional steps of: (i) removing any protecting groups present; (ii) converting the compound Formula (I) or Formula (II) into another compound of Formula (I) or Formula (II); (iii) forming a pharmaceutically acceptable salt, hydrate or solvate thereof; and/or (iv) forming a prodrug thereof.

The resultant compounds of Formula (I) or Formula (II) can be isolated and purified using techniques well known in the art.

In some embodiments, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF), 2-methyltetrahydrofuran, cyclopentylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutylketone (MIBK) or butanone; amides, such as acetamide, dimethylacetamide, dimethylformamide (DMF) or N-methylpyrrolidinone (NMP); nitriles, such as acetonitrile; sulfoxides, such as dimethyl sulfoxide (DMSO); nitro compounds, such as nitromethane or nitrobenzene; esters, such as ethyl acetate or methyl acetate, or mixtures of the said solvents or mixtures with water.

The reaction temperature is suitably between about-100 °C and 300 °C, depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, by utilizing the procedures described herein, in conjunction with ordinary skills in the art, additional compounds of the present disclosure can be readily prepared. Those skilled in the art will readily understand that known variations of the conditions and processes of the following preparative procedures can be used to prepare these compounds.

As will be understood by the person skilled in the art of organic synthesis, compounds of the present disclosure are readily accessible by various synthetic routes, some of which are exemplified in the accompanying examples. The skilled person will easily recognise which kind of reagents and reactions conditions are to be used and how they are to be applied and adapted in any particular instance—wherever necessary or useful—in order to obtain the compounds of the present disclosure. Furthermore, some of the compounds of the present disclosure can readily be synthesized by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person. Likewise, the skilled person will apply-whenever necessary or useful-synthetic protecting (or protective) groups; suitable protecting groups as well as methods for introducing and removing them are well-known to the person skilled in the art of chemical synthesis and are described, in more detail, in, e.g., P. G. M. Wuts, T. W. Greene, “Greene's Protective Groups in Organic Synthesis”, 4^{th} edition (2006) (John Wiley & Sons).

General routes for the preparation of a compound of the application are described in Schemes 1-7 herein.

In some embodiments, the compound described in Schemes 1-7 is the sodium salt of the compound.

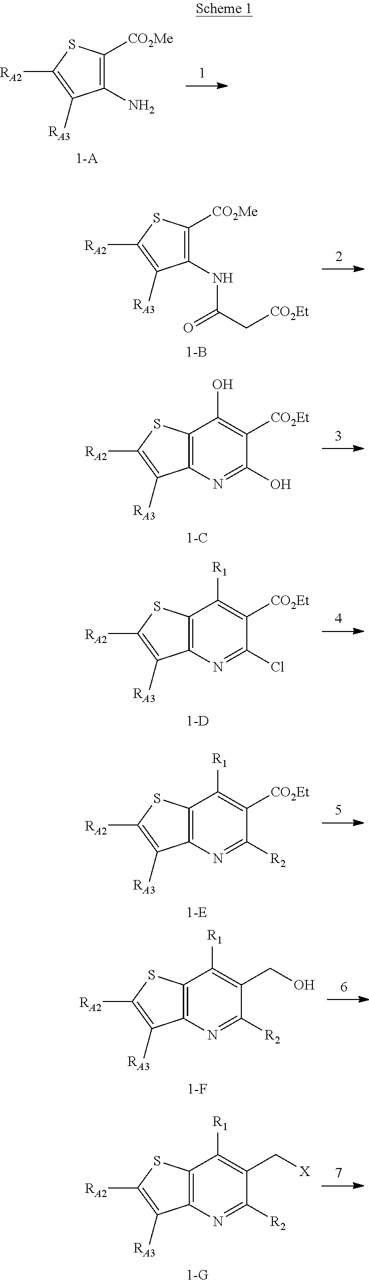

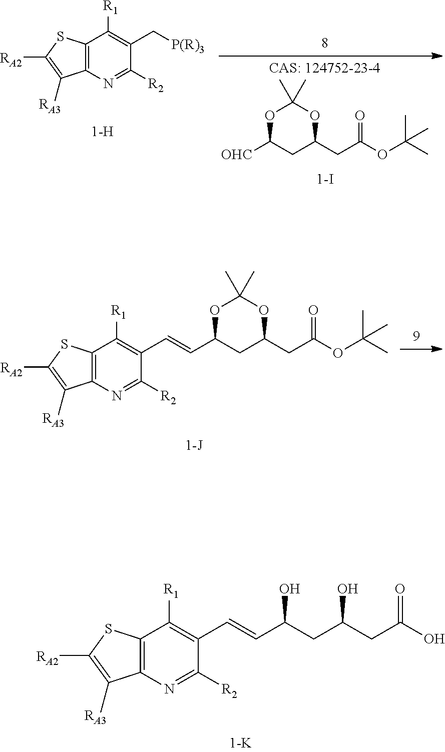

In Scheme 1, the amine can be acylated (e.g., with ethyl malonyl chloride) under standard conditions (e.g., with a tertiary amine base such as Et₃N, DIPEA, or pyridine) at room temperature in an anhydrous solvent (e.g., DCM). Ring closure (i.e., step 2) may occur under both basic (e.g., NaOEt in EtOH) conditions and with an amine base (e.g., DBU). Alternatively, aqueous acid (e.g., HCl) at reflux followed by neutralization with base may afford the desired product. Chlorination of 1-C (step 3) may afford 1-D. Sequential Suzuki coupling conditions followed by the reduction of the ester (step 4) may provide 1-E. Conversion to the halide (1-G, step 6) may occur via mesylation and Finkelstein reaction. The halide may then be treated with a phosphine source (e.g., PPh₃, POR) to provide the corresponding phosphine or phosphonate (step 7). Olefination may occur with the commercial aldehyde with 1-H (step 8) followed by Global deprotection of 1-J may afford 1-K.

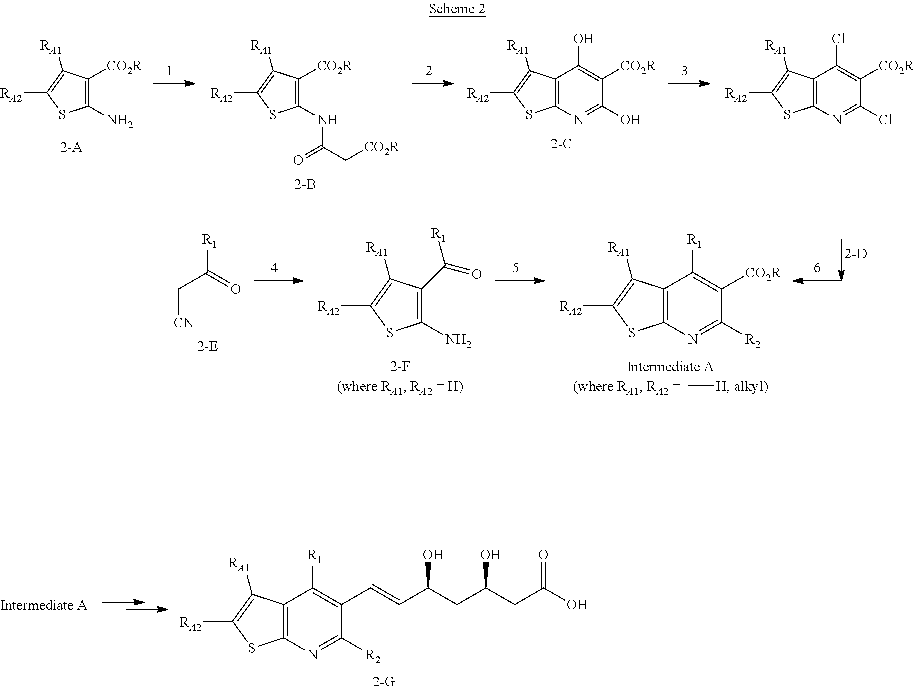

Starting with commercially available thiophene-anilines, the reactions 1, 2, 3, and 6 to Intermediate A are described in Scheme 1. Further, processing Intermediate A to the desired analogs is also described in Scheme 1.

Commercially available substituted benzoylacetonitriles and 1,4-dithiane-2,5-diol may be stirred in an alcoholic solvent in the presence of an amine (e.g., DIPEA) (step 4). The aniline may be treated with a ketone to install R₁ and R₂ in acidic conditions to provide cyclized product—Intermediate A.

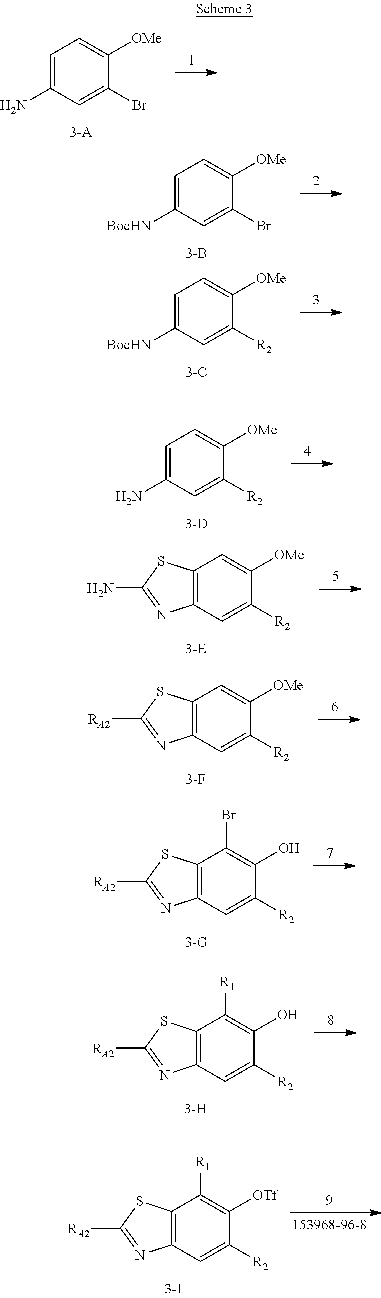

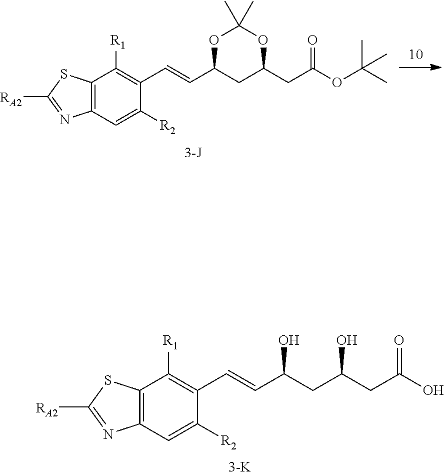

Starting with anilino-bromide, the aniline may be protected (e.g., Boc-protected) (step 1). Suzuki coupling conditions of bromide with boronic acids or boronic esters used in the presence of a Pd catalyst, aqueous based (carbonate) may be utilized in a mixed solvent systems (step 2). Deprotection (step 3) followed be thiazole formation (step 4) may generate the thiazole ring. Sandmeyer reduction of the aniline followed by Suzuki reaction may produce 3-F (step 5). Bromination and deprotection (step 6) followed by Suzuki coupling (step 7) may provide 3-H. Phenol conversion to the triflate may provide 3-I (step 8). Coupling the triflate with commercially available olefin in the presence of combinations of the following: palladium salt/base (e.g., K₂CO₃, triethylamine, or N,N-dicyclohexylmethylamine), phosphine ligand, in solvents may produce 3-J. Global deprotection may afford 3-K (step 10).

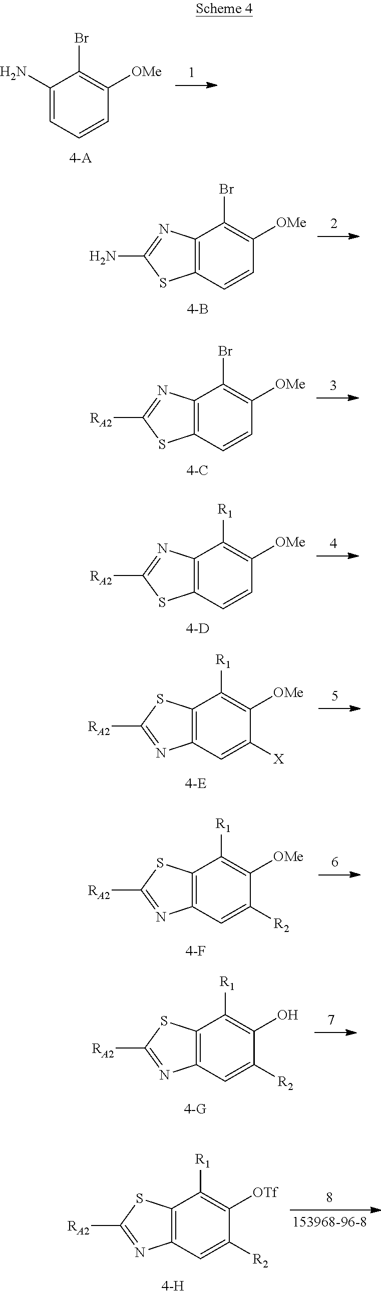

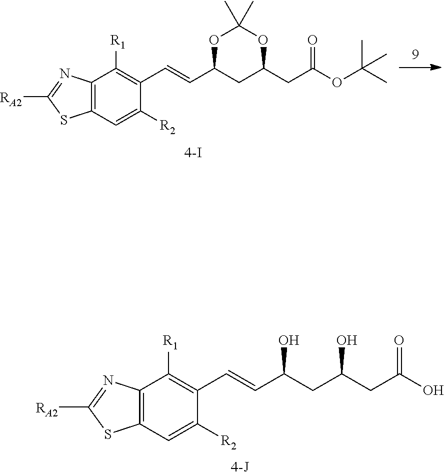

Thiazole formation (step 1) followed by Sandmeyer reduction of the aniline and Suzuki reaction (step 2) may provide 4-C. Alternatively, the Sandmeyer intermediate may be reduced to provide scaffold, wherein R_{A2} is —H. A subsequent Suzuki reaction may be completed to install R₁ (step 3). Halogenation (X=I or Br) (step 4) followed by a Suzuki reaction (step 5) provides 4-F. Deprotection of the phenol (step 6) and phenol conversion to the triflate (step 7) provides 4-H. Coupling triflate with the commercially available olefin followed by global deprotection may afford 4-J (step 9).

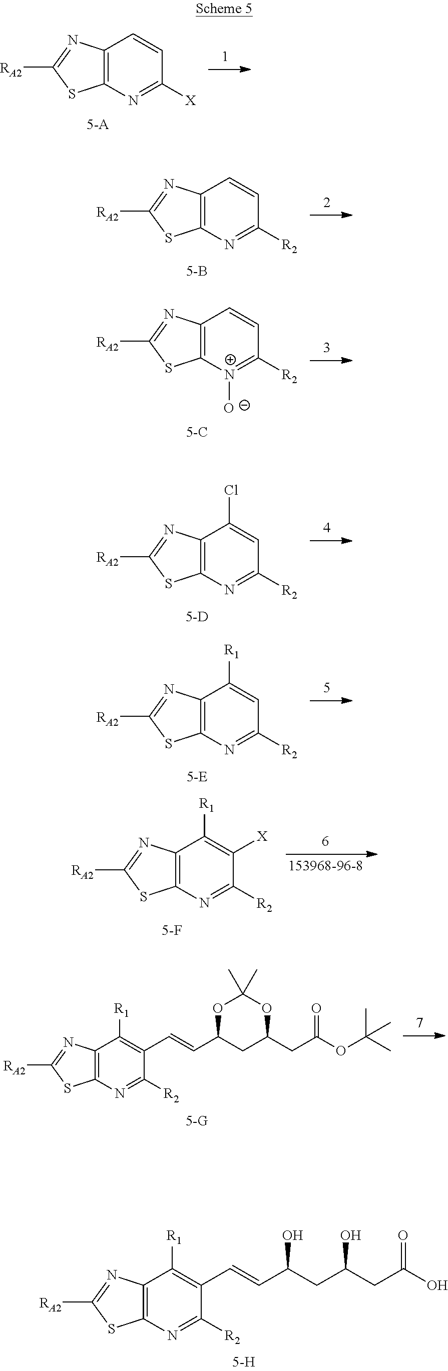

Under Suzuki coupling conditions, installation of R₂ may proceed (step 1). Oxidation of the pyridine (step 2) and subsequent halogenation (step 3) may provide 5-D. Suzuki coupling (step 4) with the desired boronic ester or boronic acid (reaction 5) followed by halogenation and coupling with the commercially available olefin (step 6) provides 5-G. Global deprotection may afford 5-H (step 7).

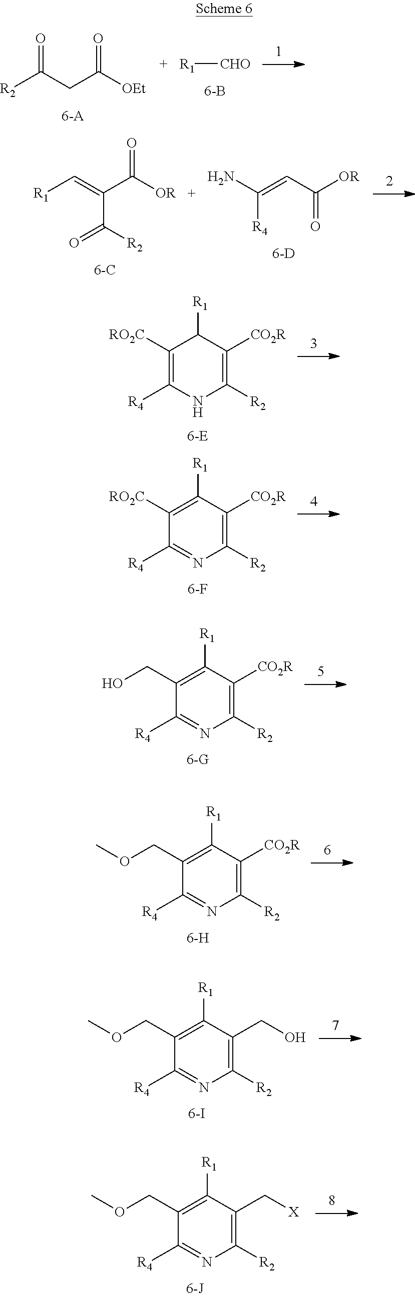

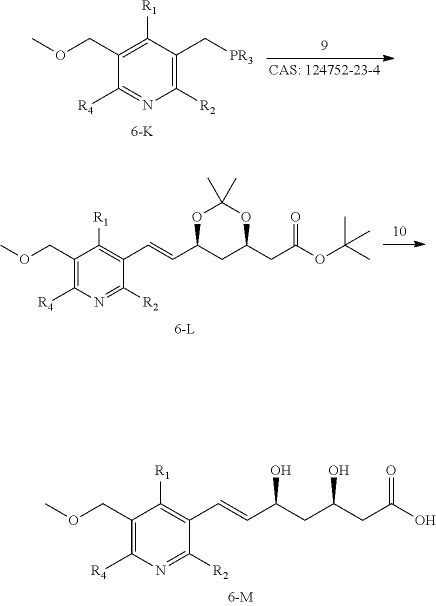

Keto-ester condensation with an aldehyde in the presence of a secondary amine base may provide 6-C (step 1). Amine mediated cyclization with the olefin (6-D) provides the dihydropyridine ring (step 2). The ring system may then be oxidized (step 3) followed by selective reduction of one ester (step 4) and methylation may provide the methyl ether (6-h; step 5). Reduction of the ester (step 6) followed by conversion to the halide (6-J, step 7) and Finkelstein reaction. The halide may then be treated with a phosphine source to provide the corresponding phosphine or phosphonate (step 8). Olefination with commercially available aldehyde with 6-K (step 9) and global deprotection of 6-L may afford 6-M (step 10).

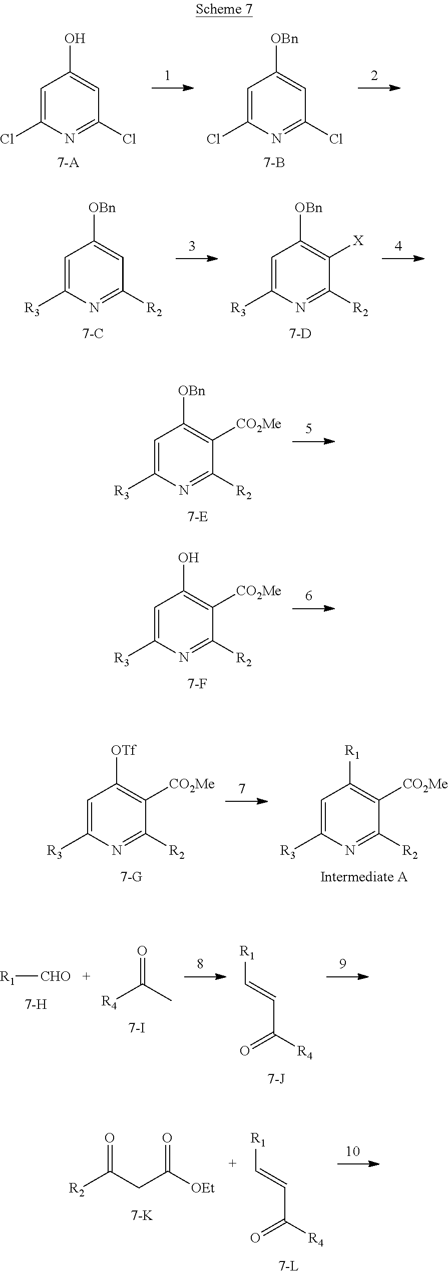

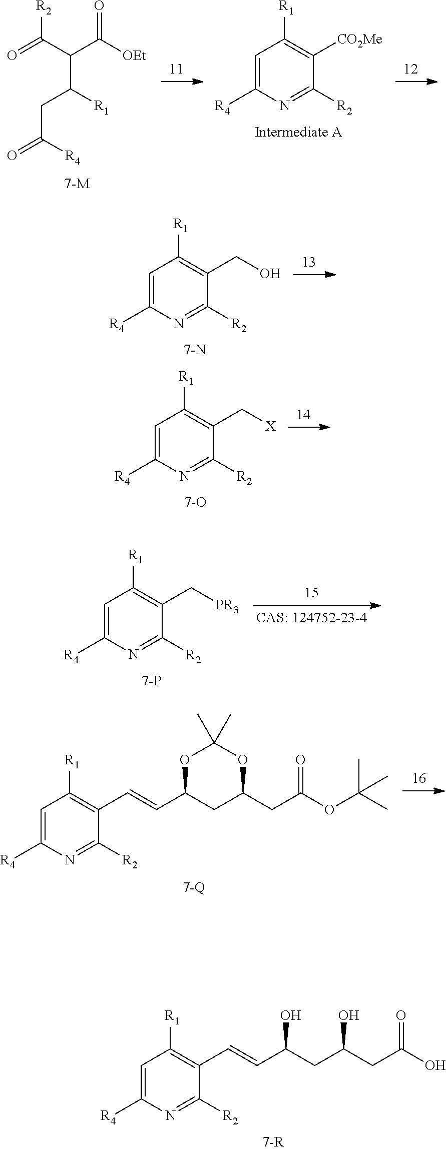

Phenolic protection (step 1) and a sequential set of Suzuki reactions (step 2) may provide 7-C. Halogenation of the pyridine (step 3) and carbonylation of the pyridine ring may provide the ester (7-E; step 4). Deprotection of the benzyl-group may afford a phenol (step 5). Phenol conversion to the triflate (step 6) followed by Suzuki coupling of the triflate to install R₁ (step 7) may yield Intermediate A. From Intermediate A, ester reduction (step 12) and conversion to the halide (7-O, step 13) may be followed by treatment with a phosphine source to provide the corresponding phosphine or phosphonate (step 14). Olefination with commercially available aldehyde (step 15) followed by global deprotection of 7-Q may afford 7-R.

Alternatively, preparation of Intermediate A may begin with aqueous base mediated condensation of an aldehyde and ketone (step 8) followed by a Michael reaction with a keto-ester (step 10) followed by cyclization may yield the Intermediate A.

### Biological Assays

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to accelerate analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays are may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

In some embodiments, the biological assay is described in the Examples herein.

STMN2 has been shown to be implicated not only for axonal regeneration, but also for the maintenance of neuromuscular junctions. Constitutive knock-out of Stmn2 in mice leads to a motor and sensory axonopathy and chronic suppression of Stmn2 in the central nervous system of otherwise wild-type adult mice results in a progressive muscle denervation and structural collapse of motor axons. Reduced level of Stmn2 leads to shrinkage of axonal diameter of motor axons and severing of myelin layers. In vitro, lentivirus-mediated replacement of STMN2 in TDP-43 deficient iPSC-neurons can restore axonal re-growth following axotomy, demonstrating that supplementing STMN2 levels may result in axonal regeneration despite TDP-43 loss-of-function causing the mis-splicing of thousands of other genes.

In some embodiments, a compound of the instant disclosure inhibits HMG-COA reductase.

In some embodiments, inhibition of HMG-COA reductase correlates to increased STMN2 expression.

In some embodiments, the compounds of the present disclosure may be screened and validated using a nanoluciferase assay.

In some embodiments, cell (e.g., TDP-mut STMN2-NLuc SH-SY5Y cells) may be used to screen compounds of Formula (I′), Formula (I), Formula (II′), or Formula (II).

In some embodiments, the compounds of the present disclosure may be screened using a neurite extension assay (e.g., in SH-SY5Y cells).

### Pharmaceutical Compositions

In some aspects, the present disclosure provides a pharmaceutical composition comprising a compound of the present disclosure as an active ingredient. In some embodiments, the present disclosure provides a pharmaceutical composition comprising at least one compound of each of the formulae described herein, or a pharmaceutically acceptable salt or solvate thereof, and one or more pharmaceutically acceptable carrier, diluent, adjuvant, excipient, or a combination thereof. In some embodiments, the present disclosure provides a pharmaceutical composition comprising a compound described in Table 1, Table 1A, Table 2, Table 2A, or Table 3. In some embodiments, the present disclosure provides a pharmaceutical composition comprising at least one compound selected from Table 1, Table 1A, Table 2, Table 2A, or Table 3.

As used herein, the term “composition” is intended to encompass a product comprising the specified ingredients in the specified amounts, as well as any product which results, directly or indirectly, from combination of the specified ingredients in the specified amounts.

The compounds of present disclosure can be formulated for oral administration in forms such as tablets, capsules (each of which includes sustained release or timed release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio) propyl-β-cyclodextrin, glucosyl-β-cyclodextrin, sulfated β-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols-such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base-depending on the formulation, the addition of one of the acid or base may be sufficient to bring the mixture to the desired pH range.

The aqueous vehicle may also contain a buffering agent to stabilize the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and ¿-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, orange flavoring.

According to a further aspect of the disclosure there is provided a pharmaceutical composition which comprises a compound of the disclosure as defined hereinbefore, or a pharmaceutically acceptable salt, hydrate or solvate thereof, in association with a pharmaceutically acceptable diluent or carrier.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more coloring, sweetening, flavoring and/or preservative agents.

An effective amount of a compound of the present disclosure for use in therapy is an amount sufficient to treat or prevent a STMN2 related condition referred to herein, slow its progression and/or reduce the symptoms associated with the condition.

An effective amount of a compound of the present disclosure for use in therapy is an amount sufficient to treat a STMN2 related condition referred to herein, slow its progression and/or reduce the symptoms associated with the condition.

The size of the dose for therapeutic or prophylactic purposes of a compound of Formula (I) or Formula (II) will naturally vary according to the nature and severity of the conditions, the age and sex of the animal or patient and the route of administration, according to well-known principles of medicine.

### Methods of Use

In some aspects, the present disclosure provides a method of modulating STMN2 expression with a compound of the present disclosure or a pharmaceutically acceptable salt thereof.

In some aspects, the present disclosure provides a method of modulating STMN2 expression (e.g., in vitro or in vivo) with an effective amount of a compound of the present disclosure or a pharmaceutically acceptable salt thereof.

In some aspects, the present disclosure provides a method of modulating STMN2 expression (e.g., in vitro or in vivo) with a compound of the present disclosure or a pharmaceutically acceptable salt thereof.

In some aspects, the present disclosure provides a method of modulating STMN2 expression (e.g., in vitro or in vivo) with an effective amount of a pharmaceutical composition comprising a compound of the present disclosure or a pharmaceutically acceptable salt thereof.

In some aspects, the present disclosure provides a method of modulating STMN2 expression (e.g., in vitro or in vivo) with a pharmaceutical composition comprising a compound of the present disclosure or a pharmaceutically acceptable salt thereof.

In some aspects, the present disclosure provides a method of increasing neurite outgrowth in a subject in need thereof, comprising administering to the subject a therapeutically effective amount of a compound of the present disclosure or a pharmaceutically acceptable salt thereof.

In some aspects, the present disclosure provides a method of increasing neurite outgrowth in a subject in need thereof, comprising administering to the subject a compound of the present disclosure or a pharmaceutically acceptable salt thereof.

In some aspects, the present disclosure provides a method of increasing axon growth in a subject in need thereof, comprising administering to the subject a therapeutically effective amount of a compound of the present disclosure or a pharmaceutically acceptable salt thereof.

In some aspects, the present disclosure provides a method of increasing axon growth in a subject in need thereof, comprising administering to the subject a compound of the present disclosure or a pharmaceutically acceptable salt thereof.

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable salt thereof.

In some aspects, the present disclosure provides a method of treating a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a disease or disorder disclosed herein in a subject in need thereof, comprising administering to the subject a compound of the present disclosure or a pharmaceutically acceptable salt thereof.

In some aspects, the present disclosure provides a method of treating a disease or disorder disclosed herein in a subject in need thereof, comprising administering to the subject a compound of the present disclosure or a pharmaceutically acceptable salt thereof, or a pharmaceutical composition of the present disclosure.

In some embodiments, the disease or disorder is associated with an implicated STMN2 expression. In some embodiments, the disease or disorder is a disease or disorder in which STMN2 expression is implicated.

In some embodiments, a modulation in expression is a modulation in activity.

In some embodiments, the disease or disorder is a neurodegenerative disease or disorder.

In some embodiments, the disease or disorder is associated with axonal degeneration, axonal damage, or axonopathy.

In some embodiments, the disease or disorder is axonal degeneration, axonal damage, or axonopathy.

In some embodiments, the disease or disorder is axonal degeneration.

In some embodiments, the disease or disorder is axonal damage.

In some embodiments, the disease or disorder is axonopathy.

In some embodiments, the neurodegenerative disease is associated with axonal degeneration, axonal damage, or axonopathy.

In some embodiments, the neurodegenerative disease is associated with axonal degeneration.

In some embodiments, the neurodegenerative disease is associated with axonal damage.

In some embodiments, the neurodegenerative disease is associated with axonopathy.

In some aspects, the present disclosure provides a method of treating or preventing a neurodegenerative disease or disorder in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a neurodegenerative disease or disorder in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a neurodegenerative disease or disorder in a subject in need thereof, comprising administering to the subject a compound of the present disclosure or a pharmaceutically acceptable salt thereof, or a pharmaceutical composition of the present disclosure.

In some aspects, the present disclosure provides a method of treating a neurodegenerative disease or disorder in a subject in need thereof, comprising administering to the subject a compound of the present disclosure or a pharmaceutically acceptable salt thereof, or a pharmaceutical composition of the present disclosure.

In some aspects, the present disclosure provides a method of treating or preventing a axonopathy in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating axonopathy in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing axonopathy in a subject in need thereof, comprising administering to the subject a compound of the present disclosure or a pharmaceutically acceptable salt thereof, or a pharmaceutical composition of the present disclosure.

In some aspects, the present disclosure provides a method of treating axonopathy in a subject in need thereof, comprising administering to the subject a compound of the present disclosure or a pharmaceutically acceptable salt thereof, or a pharmaceutical composition of the present disclosure.

In some aspects, the present disclosure provides a compound of the present disclosure or a pharmaceutically acceptable salt thereof, or a pharmaceutical composition of the present disclosure for use in modulating STMN2 expression.

In some aspects, the present disclosure provides a compound of the present disclosure or a pharmaceutically acceptable salt thereof for use in modulating STMN2 expression (e.g., in vitro or in vivo).

In some aspects, the present disclosure provides a compound of the present disclosure or a pharmaceutically acceptable salt thereof for use in modulating STMN2 expression (e.g., in vitro or in vivo).

In some aspects, the present disclosure provides a compound of the present disclosure or a pharmaceutically acceptable salt thereof for use in the treatment and/or prevention of a disease in a patient, wherein the disease is characterized by a loss of STMN2 expression.

In some aspects, the present disclosure provides a compound of the present disclosure or a pharmaceutically acceptable salt thereof, or a pharmaceutical composition of the present disclosure for use in increasing neurite outgrowth.

In some aspects, the present disclosure provides a compound of the present disclosure or a pharmaceutically acceptable salt thereof for use in increasing neurite outgrowth.

In some aspects, the present disclosure provides a compound of the present disclosure or a pharmaceutically acceptable salt thereof, or a pharmaceutical composition of the present disclosure for use in increasing axon growth.

In some aspects, the present disclosure provides a compound of the present disclosure or a pharmaceutically acceptable salt thereof for use in increasing axon growth.

In some aspects, the present disclosure provides a compound of the present disclosure or a pharmaceutically acceptable salt thereof, or a pharmaceutical composition of the present disclosure for use in treating or preventing a disease or disorder.

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides a compound of the present disclosure or a pharmaceutically acceptable salt thereof for use in treating or preventing a neurodegenerative disease or disorder in a subject in need thereof.

In some aspects, the present disclosure provides a compound of the present disclosure or a pharmaceutically acceptable salt thereof for use in treating a neurodegenerative disease or disorder in a subject in need thereof.

In some aspects, the present disclosure provides a compound of the present disclosure or a pharmaceutically acceptable salt thereof for use in treating or preventing axonopathy in a subject in need thereof.

In some aspects, the present disclosure provides a compound of the present disclosure or a pharmaceutically acceptable salt thereof for use in treating axonopathy in a subject in need thereof.

In some aspects, the present disclosure provides use of a compound of the present disclosure or a pharmaceutically acceptable salt thereof in the manufacture of a medicament for modulating STMN2 expression (e.g., in vitro or in vivo).

In some aspects, the present disclosure provides use of a compound of the present disclosure or a pharmaceutically acceptable salt thereof in the manufacture of a medicament for increasing neurite outgrowth.

In some aspects, the present disclosure provides use of a compound of the present disclosure or a pharmaceutically acceptable salt thereof in the manufacture of a medicament for increasing axon growth.

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating a disease or disorder disclosed herein.

In some aspects, the present disclosure provides use of a compound of the present disclosure or a pharmaceutically acceptable salt thereof in the manufacture of a medicament for treating or preventing a neurodegenerative disease or disorder in a subject in need thereof.

In some aspects, the present disclosure provides use of a compound of the present disclosure or a pharmaceutically acceptable salt thereof in the manufacture of a medicament for treating a neurodegenerative disease or disorder in a subject in need thereof.

In some aspects, the present disclosure provides use of a compound of the present disclosure or a pharmaceutically acceptable salt thereof in the manufacture of a medicament for treating or preventing axonopathy in a subject in need thereof.

In some aspects, the present disclosure provides use of a compound of the present disclosure or a pharmaceutically acceptable salt thereof in the manufacture of a medicament for treating axonopathy in a subject in need thereof.

In some embodiments, the present disclosure provides compounds that function as modulators of STMN2 activity.

In some embodiments, modulation results in an increase of STMN2 expression.

In some embodiments, modulation results in an increase of STMN2 expression via inhibition of HMG-COA-reductase.

In some embodiments, the present disclosure provides compounds that function as inhibitors of HMG-COA-reductase.

Effectiveness of compounds of the disclosure can be determined by industry-accepted assays/disease models according to standard practices of elucidating the same as described in the art and are found in the current general knowledge.

The present disclosure also provides a method of treating a disease or disorder in which STMN2 activity is implicated in a subject in need of such treatment, said method comprising administering to said subject a compound, or a pharmaceutically acceptable salt thereof, or a pharmaceutical composition as defined herein.

In some embodiments, the disease or disorder is associated with a TDP43-induced STMN2 deficiency.

In some embodiments, the disease or disorder is a neurodegenerative disease or disorder associated with a TDP43-induced STMN2 deficiency.

In some embodiments, the disease or disorder is associated with phenotypic axonopathy.

In some embodiments, the phenotypic axonopathy is caused by injury, insult, or aging.

In some embodiments, the phenotypic axonopathy is caused by brain injury or brain insult.

In some embodiments, the disease or disorder is associated with decreased axon growth.

In some embodiments, the disease or disorder is associate with decreased neurite outgrowth.

In some embodiments, the disease or disorder is a synaptic disorder.

In some embodiments, the synaptic disorder is associated with a synaptic deficiency.

In some embodiments, the synaptic deficiency is associated with a neurological disease or disorder.

In some embodiments, the disease or disorder has a deficiency in synaptic function.

In some embodiments, the disorder is a spinal injury.

In some embodiments, the disorder is a nerve injury.

In some embodiments, the neurodegenerative disease or disorder is selected from Amyotrophic Lateral Sclerosis (ALS), Parkinson's Disease (PD), Alzheimer's Disease (AD), Frontotemporal Dementia (FTD), Inclusion Body Myopathies (IBM), Rett Syndrome, Alexander Syndrome, Perry Syndrome, Limbic-predominant Age-related TDP-43 Encephalopathy Neuropathologic Change (LATE-NC), Lewy Body Dementia (LBD), Peripheral neuropathies (chemotherapy induced neuropathy, injury induced), and Autism spectrum disorder.

In some embodiments, an axonopathy associated with Amyotrophic Lateral Sclerosis (ALS), Parkinson's Disease (PD), Alzheimer's Disease (AD), Frontotemporal Dementia (FTD), Inclusion Body Myopathies (IBM), Rett Syndrome, Alexander Syndrome, Perry Syndrome, Limbic-predominant Age-related TDP-43 Encephalopathy Neuropathologic Change (LATE-NC), Lewy Body Dementia (LBD), Peripheral neuropathies (chemotherapy induced neuropathy, injury induced), and Autism spectrum disorder.

In some embodiments, the neurodegenerative disease or disorder is Amyotrophic Lateral Sclerosis (ALS).

In some embodiments, the neurodegenerative disease or disorder is Parkinson's Disease (PD).

In some embodiments, the neurodegenerative disease or disorder is Alzheimer's Disease (AD).

In some embodiments, the neurodegenerative disease or disorder is Frontotemporal Dementia (FTD).

In some embodiments, the neurodegenerative disease or disorder is Inclusion Body Myopathies (IBM).

In some embodiments, the neurodegenerative disease or disorder is Rett Syndrome.

In some embodiments, the neurodegenerative disease or disorder is Alexander Syndrome.

In some embodiments, the neurodegenerative disease or disorder is Perry Syndrome.

In some embodiments, the neurodegenerative disease or disorder is Limbic-predominant Age-related TDP-43 Encephalopathy Neuropathologic Change (LATE-NC).

In some embodiments, the neurodegenerative disease or disorder is Lewy Body Dementia (LBD).

In some embodiments, the neurodegenerative disease or disorder is a peripheral neuropathy (e.g., chemotherapy induced neuropathy or injury induced neuropathy).

In some embodiments, the neurodegenerative disease or disorder is Autism spectrum disorder.

### Routes of Administration

Compounds of the present disclosure, or pharmaceutically acceptable salts thereof, may be administered alone as a sole therapy or can be administered in addition with one or more other substances and/or treatments. Such conjoint treatment may be achieved by way of the simultaneous, sequential or separate administration of the individual components of the treatment.

For example, therapeutic effectiveness may be enhanced by administration of an adjuvant (i.e., by itself the adjuvant may only have minimal therapeutic benefit, but in combination with another therapeutic agent, the overall therapeutic benefit to the individual is enhanced). Alternatively, by way of example only, the benefit experienced by an individual may be increased by administering the compound of Formula (I′), Formula (I), Formula (II′), or Formula (II) with another therapeutic agent (which also includes a therapeutic regimen) that also has therapeutic benefit.

In the instances where the compound of the present disclosure is administered in combination with other therapeutic agents, the compound of the disclosure need not be administered via the same route as other therapeutic agents, and may, because of different physical and chemical characteristics, be administered by a different route. For example, the compound of the disclosure may be administered orally to generate and maintain good blood levels thereof, while the other therapeutic agent may be administered intravenously. The initial administration may be made according to established protocols known in the art, and then, based upon the observed effects, the dosage, modes of administration and times of administration can be modified by the skilled clinician.

The particular choice of other therapeutic agent will depend upon the diagnosis of the attending physicians and their judgment of the condition of the individual and the appropriate treatment protocol. According to this aspect of the disclosure there is provided a combination for use in the treatment of a disease in which STMN2 activity is implicated comprising a compound of the disclosure as defined hereinbefore, or a pharmaceutically acceptable salt thereof, and another suitable agent.

According to a further aspect of the disclosure there is provided a pharmaceutical composition which comprises a compound of the disclosure, or a pharmaceutically acceptable salt thereof, in combination with a suitable, in association with a pharmaceutically acceptable diluent or carrier.

In addition to its use in therapeutic medicine, compounds of Formula (I′), Formula (I), Formula (II′), or Formula (II) and pharmaceutically acceptable salts thereof are also useful as pharmacological tools in the development and standardization of in vitro and in vivo test systems for the evaluation of the effects of modulators of STMN2 activity in laboratory animals such as dogs, rabbits, monkeys, mini-pigs, rats and mice, as part of the search for new therapeutic agents.

In any of the above-mentioned pharmaceutical composition, process, method, use, medicament, and manufacturing features of the instant disclosure, any of the alternate embodiments of macromolecules of the present disclosure described herein also apply.

The compounds of the disclosure or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray or powder); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### EXAMPLES

For exemplary purpose, neutral compounds of Formula (I′), Formula (I), Formula (II′), or Formula (II) are synthesized and tested in the examples. It is understood that the neutral compounds of Formula (I) or Formula (II) may be converted to the corresponding pharmaceutically acceptable salts of the compounds using routine techniques in the art (e.g., by saponification of an ester to the carboxylic acid salt, or by hydrolyzing an amide to form a corresponding carboxylic acid and then converting the carboxylic acid to a carboxylic acid salt).

In some embodiments, the compound of Formula (I′), Formula (I), Formula (II′), or Formula (II) is the sodium salt of the compound.

Abbreviations

| | |
|---|---|
| ACN | Acetonitrile |
| CDCl₃ | Chloroform-d |
| DCM | Dichloromethane |
| DMSO-d₆ | Dimethyl sulfoxide-d6 |
| eq. | Equivalents |
| ESI | Electrospray Ionisation |
| EtOH | Ethanol |
| hrs | Hour(s) |
| HPLC | High-performance Liquid Chromatography |
| LCMS | Liquid Chromatography-Mass Spectrometry |
| MeOH-d₄ | Deuterated methanol |
| MeOH | Methanol |
| min | Minute(s) |
| NMR | Nuclear Magnetic Resonance |
| RP | Reverse phase |
| RT | Retention time |
| TLC | Thin layer chromatography |

### Example 1. Synthesis of (3R,5S,E)-7-(5-cyclopropyl-7-(4-fluorophenyl)-3-methylthieno[3,2-b]pyridin-6-yl)-3,5-dihydroxyhept-6-enoic acid

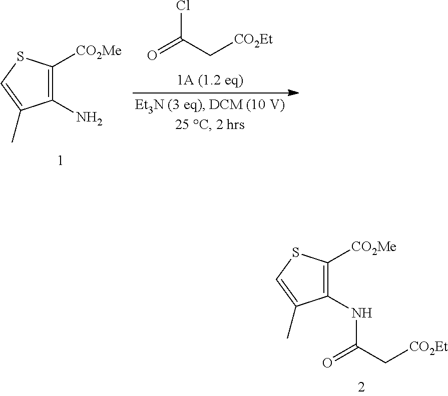 [SMILES: COC(=O)c1scc(C)c1N ; CCOC(=O)CC(=O)Cl ; CCOC(=O)CC(=O)Nc1c(C)csc1C(=O)OC]

Step 1. To a solution of compound 1 (15 g, 87.61 mmol, 1 eq), Et₃N (26.60 g, 262.83 mmol, 36.58 mL, 3 eq) in CH₂Cl₂ (150 mL) was added compound 1A (15.83 g, 105.13 mmol, 13.23 mL, 1.2 eq) and the mixture was stirred at 25 °C for 2 hrs. LCMS showed the starting material was consumed completely and desired product was observed. To the mixture was added water (200 mL) and the mixture was extracted with CH₂Cl₂ (200 mL×3). The combined organic layers were dried over anhydrous Na₂SO₄ and concentrated under reduced pressure. The residue was purified by column chromatography on silica gel (Ethyl acetate/Petroleum ether=0 to 25%) to afford compound 2 (10.73 g, 42.93% yield) as a yellow solid. LCMS (ESI+): m/z=307.9 (M+23), RT: 0.331 min. 5-95AB_0.8 MIN: LC/MS (The column used for chromatography was a Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

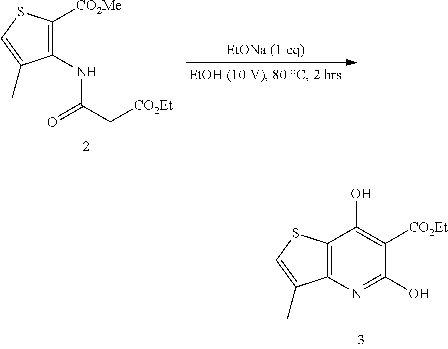 [SMILES: CCOC(=O)CC(=O)Nc1c(C)csc1C(=O)OC ; CCOC(=O)c1c(O)nc2c(C)csc2c1O]

Step 2. To a solution of compound 2 (10.73 g, 37.61 mmol, 1 eq) in EtOH (100 mL) was added NaOEt (2.56 g, 37.61 mmol, 1 eq) and the mixture was stirred at 80 °C for 2 hrs. LCMS showed desired product was observed. The mixture was cooled to 25 °C and filtered. The filter cake was dried under reduced pressure to give compound 3 (11.76 g, crude) as a white solid. LCMS (ESI+): m/z=254.0 (M+1), RT: 0.353 min.

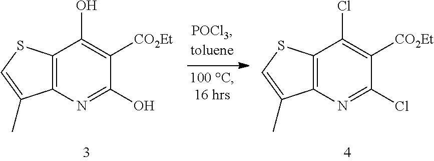 [SMILES: CCOC(=O)c1c(O)nc2c(C)csc2c1O ; CCOC(=O)c1c(Cl)nc2c(C)csc2c1Cl]

Step 3. To a solution of compound 3 (5 g, 19.74 mmol, 1 eq) in toluene (19 mL) was added POCl₃ (19 mL) and the mixture was stirred at 100 °C for 16 hrs. LCMS showed the starting material was consumed completely and desired product was observed. The mixture was poured into saturated aqueous NaHCO₃ (500 mL) and extracted with EtOAc (500 mL×3). The combined organic layers were dried over anhydrous Na₂SO₄ and concentrated under reduced pressure. The residue was combined with a parallel batch (5 g compound 3 was used) and purified by column chromatography on silica gel (Ethyl acetate/Petroleum ether=0 to 5%) to afford compound 4 (3.7 g, 32.30% yield) as a white solid. LCMS (ESI+): m/z=290.0 (M+1), RT: 0.503 min.

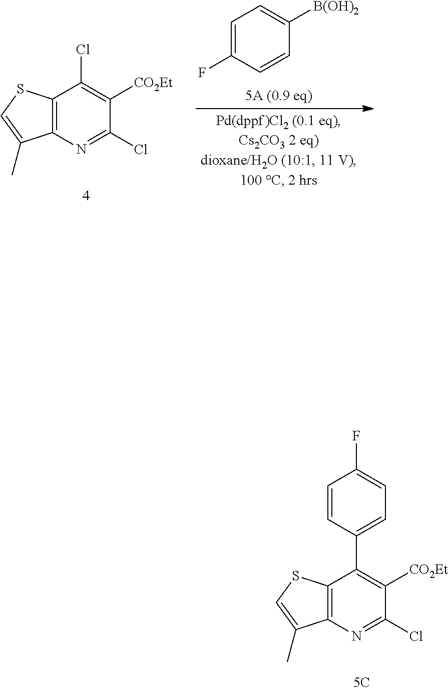 [SMILES: CCOC(=O)c1c(Cl)nc2c(C)csc2c1Cl ; OB(O)c1ccc(F)cc1 ; CCOC(=O)c1c(Cl)nc2c(C)csc2c1-c1ccc(F)cc1]

Step 4. A solution of compound 4 (2.17 g, 7.48 mmol, 1 eq), compound 5A (941.75 mg, 6.73 mmol, 0.9 eq), Cs₂CO₃ (4.87 g, 14.96 mmol, 2 eq), Pd(dppf)Cl₂·CH₂Cl₂ (610.72 mg, 747.85 μmol, 0.1 eq) in dioxane (20 mL) and H₂O (2 mL) was degassed and purged with N₂ for 3 times and the mixture was stirred at 100 °C under N₂ for 2 hrs. LCMS showed the starting material was consumed completely and desired MS was observed. The reaction mixture was concentrated under reduced pressure to give a crude product. The crude product was purified by column chromatography on silica gel (Ethyl acetate/Petroleum ether=0 to 5%) to afford compound 5C (1.38 g, 52.75% yield) as colorless oil. LCMS (ESI+): m/z=350.0 (M+1), RT: 0.526 min.

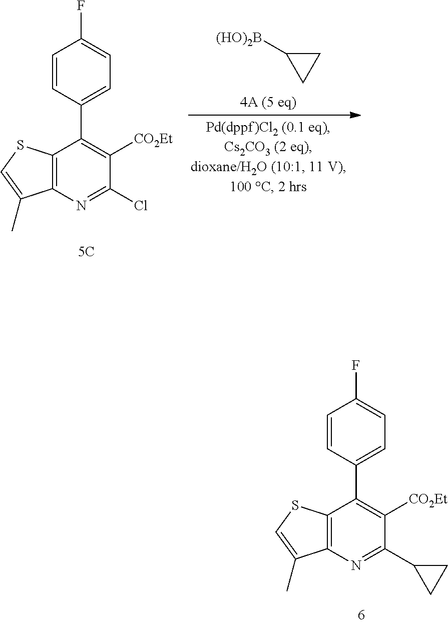 [SMILES: CCOC(=O)c1c(Cl)nc2c(C)csc2c1-c1ccc(F)cc1 ; OB(O)C1CC1 ; CCOC(=O)c1c(C2CC2)nc2c(C)csc2c1-c1ccc(F)cc1]

Step 5. A solution of compound 5C (480.00 mg, 1.37 mmol, 1 eq), compound 4A (471.47 mg, 5.49 mmol, 4 eq), Cs₂CO₃ (894.17 mg, 2.74 mmol, 2 eq), Pd(dppf)Cl₂·CH₂Cl₂ (112.06 mg, 137.22 μmol, 0.1 eq) in dioxane (5 mL) and H₂O (0.5 mL) was degassed and purged with N₂ for 3 times and the mixture was stirred at 100 °C under N₂ for 2 hrs. LCMS showed the starting material was consumed completely and desired product was observed. The reaction mixture was concentrated under reduced pressure to give a crude product. The crude product was purified by column chromatography on silica gel (Ethyl acetate/Petroleum ether=0 to 5%) to afford compound 6 (135 mg, 27.68% yield) as colorless oil. ¹H NMR: (400 MHz, CDCl₃) δ 1.10-0.90 (m, 5H), 1.31 (br dd, J=4.4, 2.8 Hz, 2H), 2.20-2.35 (m, 1H), 2.47 (s, 3H), 4.14 (q, J=7.2 Hz, 2H), 7.18 (t, J=8.8 Hz, 2H), 7.40 (s, 1H), 7.50 (dd, J=8.4, 5.2 Hz, 2H). LCMS (ESI+): m/z=356.1 (M+1), RT: 0.567 min.

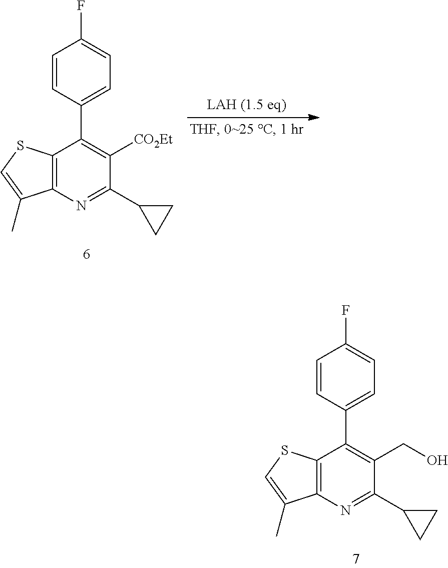 [SMILES: CCOC(=O)c1c(C2CC2)nc2c(C)csc2c1-c1ccc(F)cc1 ; Cc1csc2c(-c3ccc(F)cc3)c(CO)c(C3CC3)nc12]

Step 6. To a solution of compound 6 (890 mg, 2.50 mmol, 1 eq) in THF (10 mL) was added LiAlH₄ (2.5 M, 1.50 mL, 1.5 eq) at 0 °C and the mixture was stirred at 25 °C under N₂ for 1 hr. TLC showed the starting material was consumed completely and a new spot with larger polarity was observed. The reaction was quenched by adding water (50 mL) and extracted with EtOAc (50 mL×3). The combined organic layers were dried over anhydrous Na₂SO₄ and concentrated under reduced pressure to give compound 7 (714 mg, 90.99% yield) as a white solid which was used directly to next step.

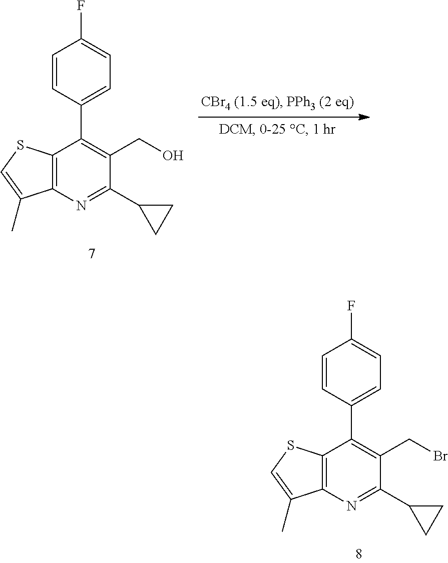 [SMILES: Cc1csc2c(-c3ccc(F)cc3)c(CO)c(C3CC3)nc12 ; Cc1csc2c(-c3ccc(F)cc3)c(CBr)c(C3CC3)nc12]

Step 7. A solution of compound 7 (714 mg, 2.28 mmol, 1 eq), PPh₃ (1.20 g, 4.56 mmol, 2 eq) in DCM (10 mL) was cooled to 0 °C under N₂. CBr₄ (1.13 g, 3.42 mmol, 1.5 eq) was added at 0 °C and the mixture was stirred at 25 °C for 1 hr. LCMS showed the starting material was consumed completely and desired product was observed. The mixture was concentrated under reduced pressure to give a crude product. The crude product was purified by column chromatography on silica gel (Ethyl acetate/Petroleum ether=0 to 5%) to afford compound 8 (646 mg, 75.35% yield) as a white solid. LCMS (ESI+): m/z=376.0 (M+1), RT: 0.593 min.

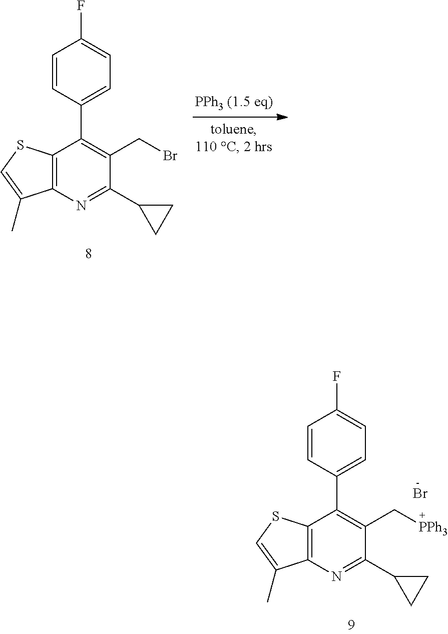

Step 8. To a solution of compound 8 (646 mg, 1.72 mmol, 1 eq) in toluene (5 mL) was added PPh₃ (675.43 mg, 2.58 mmol, 1.5 eq) and the mixture was stirred at 110 °C for 2 hrs. LCMS showed the starting material was consumed completely and desired MS was observed. The mixture was concentrated under reduced pressure to give compound 9 (1.09 g, 99.43% yield) as a white solid, which was used directly in the next step. LCMS (ESI+): m/z=558.2 (M-Br), RT: 0.450 min.

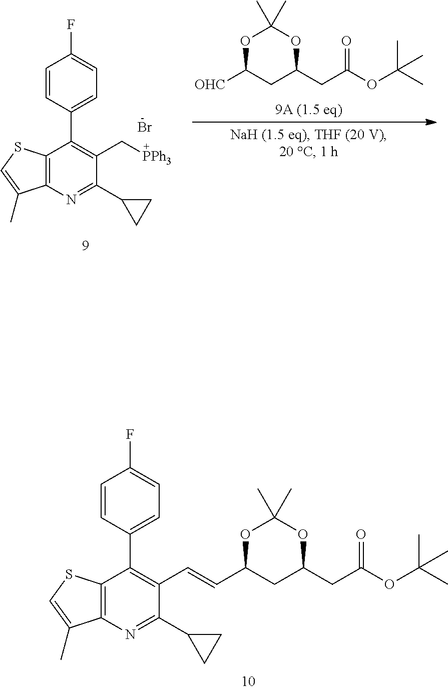

Step 9. To a solution of compound 9 (1.09 g, 1.71 mmol, 1 eq) in THF (20 mL) was added NaH (102.41 mg, 2.56 mmol, 60% purity, 1.5 eq) and compound 9A (661.38 mg, 2.56 mmol, 1.5 eq). The mixture was stirred at 20 °C for 1 hr. LCMS showed the starting material was consumed completely and desired product was observed. The mixture was quenched by adding water (3 mL) and concentrated under reduced pressure to give a crude product. The crude product was purified by column chromatography on silica gel (Ethyl acetate/Petroleum ether=0 to 10%) to afford compound 10 (595 mg, 32.41% yield) as a white solid. LCMS (ESI+): m/z=538.2 (M+1), RT: 0.623 min.

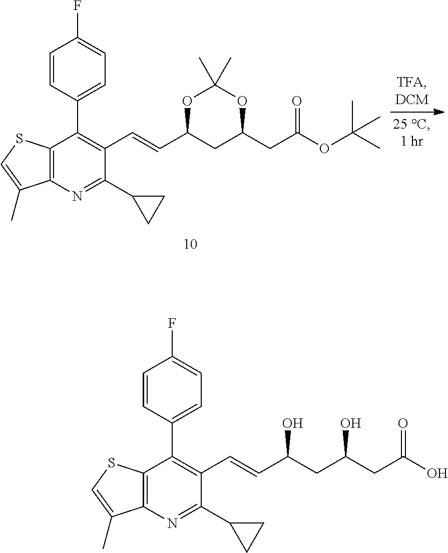 [SMILES: Cc1csc2c(-c3ccc(F)cc3)c(/C=C/[C@@H]3C[C@H](CC(=O)OC(C)(C)C)OC(C)(C)O3)c(C3CC3)nc12 ; Cc1csc2c(-c3ccc(F)cc3)c(/C=C/[C@@H](O)C[C@@H](O)CC(=O)O)c(C3CC3)nc12]

Step 10. To a solution of compound 10 (595 mg, 1.11 mmol, 1 eq) in DCM (6 mL) was added TFA (2 mL) and the mixture was stirred at 25 °C for 1 hr. LCMS showed the starting material was consumed completely and desired product was observed. The mixture was concentrated under reduced pressure to give a residue. The residue was purified by prep-HPLC: column: Phenomenex luna C18 150*25 mm*10 μm; mobile phase: [water (FA)-ACN]; gradient: 55%-85% B over 10 min to give a crude product, which was further purified by column chromatography on silica gel (Ethyl acetate/Petroleum ether=0 to 50%) to afford (3R,5S,E)-7-(5-cyclopropyl-7-(4-fluorophenyl)-3-methylthieno[3,2-b]pyridin-6-yl)-3,5-dihydroxyhept-6-enoic acid (87.3 mg, 17.87% yield) as a white solid. ¹H NMR: (400 MHz, CDCl₃) δ 1.02 (dd, J=8.0, 2.4 Hz, 2H), 1.26-1.34 (m, 2H), 1.60-1.67 (m, 1H), 1.75-1.89 (m, 2H), 2.28-2.40 (m, 1H), 2.46 (s, 3H), 2.56-2.66 (m, 1H), 2.67-2.79 (m, 1H), 4.18-4.35 (m, 1H), 5.12-5.28 (m, 1H), 5.61 (dd, J=16.0, 6.4 Hz, 1H), 6.80 (dd, J=16.0, 1.2 Hz, 1 H), 7.17 (t, J=8.8 Hz, 2H), 7.29 (d, J=1.2 Hz, 1H), 7.36 (dd, J=8.8, 5.6 Hz, 2H). LCMS (ESI+): m/z=424.0 (M−17), RT: 2.232 min.

### Example 2. (3R,5S,E)-7-(5-cyclopropyl-7-(4-fluorophenyl)thieno[3,2-b]pyridin-6-yl)-3,5-dihydroxyhept-6-enoic acid

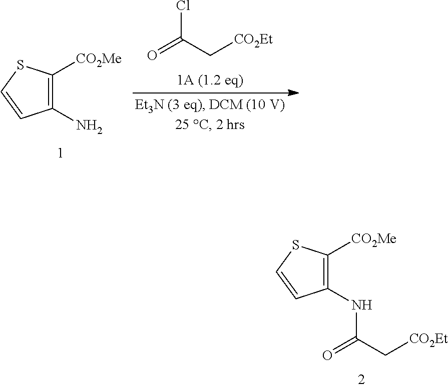 [SMILES: CCOC(=O)CC(=O)Cl ; COC(=O)c1sccc1N ; CCOC(=O)CC(=O)Nc1ccsc1C(=O)OC]

Step 1. To a solution of compound 1 (15 g, 95.43 mmol, 1 eq) in DCM (150 mL) was added Et₃N (28.97 g, 286.28 mmol, 39.85 mL, 3 eq) and compound 1A (17.24 g, 114.51 mmol, 14.42 mL, 1.2 eq). The mixture was stirred at 25 °C for 2 hrs. TLC showed the starting material was consumed completely and a major new spot was detected. To the reaction mixture was added water (200 mL). The mixture was extracted with DCM (200 mL×3). The combined organic layers were washed with brine (200 mL), dried over anhydrous sodium sulfate, filtered and concentrated under reduced pressure. The residue was purified by column chromatography on silica gel (Ethyl acetate/Petroleum ether=0 to 9%) to give compound 2 (14.22 g, 54.93% yield) as yellow oil. LCMS (ESI+): m/z=272.0 (M+1), RT: 0.375 min.

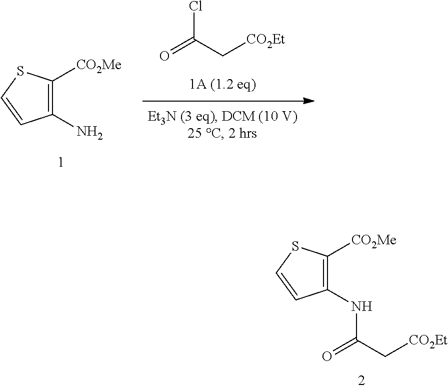 [SMILES: CCOC(=O)CC(=O)Cl ; COC(=O)c1sccc1N ; CCOC(=O)CC(=O)Nc1ccsc1C(=O)OC]

Step 2. To a solution of compound 2 (14.22 g, 52.42 mmol, 1 eq) in EtOH (143 mL) was added EtONa (5.35 g, 78.62 mmol, 1.5 eq). The mixture was stirred at 80 °C for 16 hrs. LCMS showed the starting material was consumed and desired MS was detected. The reaction mixture was filtered and the solid was dried under reduced pressure to give compound 3 (12.51 g, 99.76% yield) as a yellow solid. LCMS (ESI+): m/z=240.1 (M+1), RT: 0.328 min.

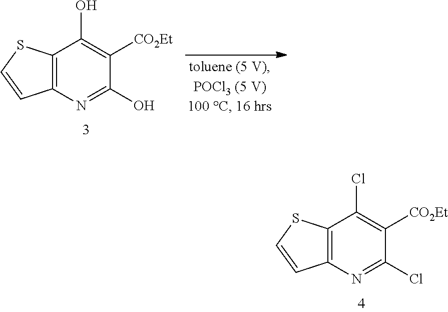 [SMILES: CCOC(=O)c1c(O)nc2ccsc2c1O ; CCOC(=O)c1c(Cl)nc2ccsc2c1Cl]

Step 3. To a solution of compound 3 (4 g, 16.72 mmol, 1 eq) in toluene (19 mL) was added POCl₃ (19 mL) The mixture was stirred at 100 °C for 16 hrs. TLC showed the starting material was consumed completely and a major new spot was detected. After cooling to room temperature, the reaction mixture was added to saturated aqueous sodium bicarbonate solution (500 mL) at 25 °C, and then extracted with ethyl acetate (500 mL×3). The combined organic layers were washed with brine (300 mL), dried over anhydrous sodium sulfate, filtered and concentrated under reduced pressure. The residue was purified by column chromatography on silica gel (Ethyl acetate/Petroleum ether=0 to 2%) to give compound 4 (1.51 g, 32.71% yield) as a white solid. LCMS (ESI+): m/z=275.9 (M+1), RT: 0.455 min.

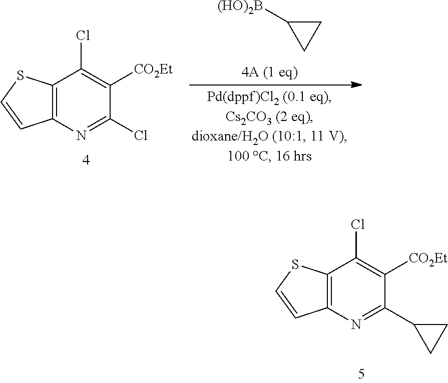 [SMILES: CCOC(=O)c1c(Cl)nc2ccsc2c1Cl ; OB(O)C1CC1 ; CCOC(=O)c1c(C2CC2)nc2ccsc2c1Cl]

Step 4. To a solution of compound 4 (520 mg, 1.88 mmol, 1 eq) and compound 4A (129.40 mg, 1.51 mmol, 0.8 eq) in dioxane (5 mL) and H₂O (0.5 mL) was added Pd(dppf)Cl₂·CH₂Cl₂ (153.78 mg, 188.31 μmol, 0.1 eq) and Cs₂CO₃ (1.23 g, 3.77 mmol, 2 eq). The mixture was stirred at 100 °C for 16 hrs under N₂. TLC showed a part of compound 4 was remained, and two major new spots were detected. The reaction mixture was concentrated under reduced pressure to give a residue. The residue was purified by column chromatography on silica gel (Ethyl acetate/Petroleum ether=0 to 2%) to give compound 5 (100 mg, 17.72% yield) as colorless oil. ¹H NMR: (400 MHz, CDCl₃) δ 1.04 (dd, J=8.0, 2.8 Hz, 2H) 1.24 (dd, J=4.4, 2.8 Hz, 2H) 1.46 (t, J=7.2 Hz, 3H) 2.10-2.17 (m, 1H) 4.52 (q, J=7.2 Hz, 2H) 7.45 (d, J=5.6 Hz, 1H) 7.79 (d, J=5.6 Hz, 1H).

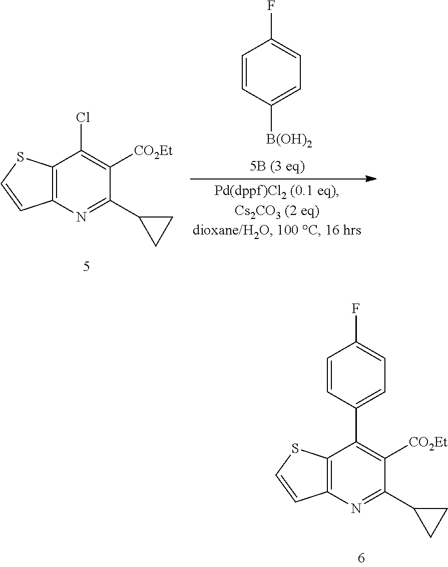 [SMILES: CCOC(=O)c1c(C2CC2)nc2ccsc2c1Cl ; OB(O)c1ccc(F)cc1 ; CCOC(=O)c1c(C2CC2)nc2ccsc2c1-c1ccc(F)cc1]

Step 5. To a solution of compound 5 (300 mg, 1.06 mmol, 1 eq) and compound 5B (446.94 mg, 3.19 mmol, 3 eq) in dioxane (2 mL) and H₂O (0.2 mL) was added Pd(dppf)Cl₂·CH₂Cl₂ (86.95 mg, 106.47 μmol, 0.1 eq) and Cs₂CO₃ (693.83 mg, 2.13 mmol, 2 eq). The mixture was stirred at 100 °C for 16 hrs under N₂. TLC showed the starting material was consumed completely and a major new spot was detected. The reaction mixture was concentrated under reduced pressure. The residue was purified by column chromatography on silica gel (Ethyl acetate/Petroleum ether=0 to 5%) to give compound 6 (262 mg, 68.47% yield) as brown oil. LCMS (ESI+): m/z=342.1 (M+1), RT: 0.500 min.

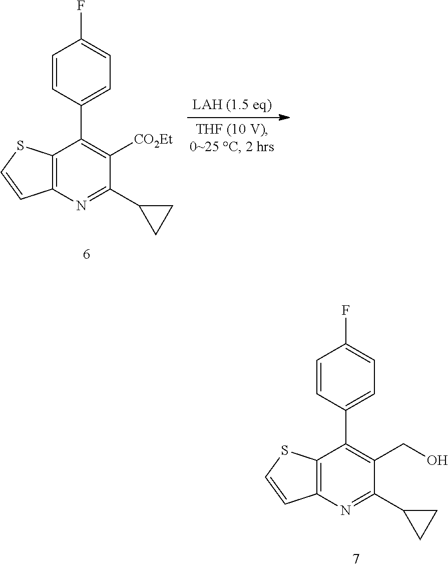 [SMILES: CCOC(=O)c1c(C2CC2)nc2ccsc2c1-c1ccc(F)cc1 ; OCc1c(C2CC2)nc2ccsc2c1-c1ccc(F)cc1]

Step 6. To a solution of compound 6 (466 mg, 1.36 mmol, 1 eq) in THF (5 mL) was added LAH (2.5 M, 818.98 μL, 1.5 eq) at 0 °C. The mixture was stirred at 25 °C for 2 hrs under N₂. LCMS showed the starting material was consumed completely and the product was detected. The reaction was cooled to −10~0 °C, water (0.1 mL) was slowly added dropwise to the reaction, followed by 15% aq·NaOH (0.1 mL) and water (0.3 mL). The mixture was warmed to room temperature and stirred for 15 minutes. Then, anhydrous Na₂SO₄ was added, and the mixture was filtered. The filter cake was washed with ethyl acetate (10 mL×3), the filtrate was concentrated under reduced pressure to give compound 7 (414 mg, 1.31 mmol, 96.25% yield) as yellow oil. LCMS (ESI+): m/z=300 (M+1), RT: 0.313 min.

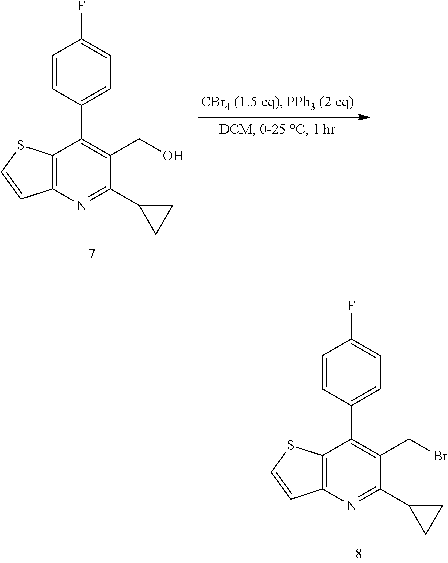 [SMILES: OCc1c(C2CC2)nc2ccsc2c1-c1ccc(F)cc1 ; Fc1ccc(-c2c(CBr)c(C3CC3)nc3ccsc23)cc1]

Step 7. To a solution of compound 7 (200 mg, 668.09 μmol, 1 eq) in DCM (3 mL) was added PPh₃ (350.46 mg, 1.34 mmol, 2 eq) and CBr₄ (332.33 mg, 1.00 mmol, 1.5 eq) at 0 °C. The mixture was stirred at 25 °C for 1 hr under N₂. LCMS showed the starting material was consumed completely and the product was detected. The reaction mixture was concentrated under reduced pressure to give a residue. The residue was purified by column chromatography on silica gel (Ethyl acetate/Petroleum ether=0 to 5%) to give compound 8 (111 mg, 45.86% yield) as yellow oil. LCMS (ESI+): m/z=362.0 (M+1), RT: 0.500 min.

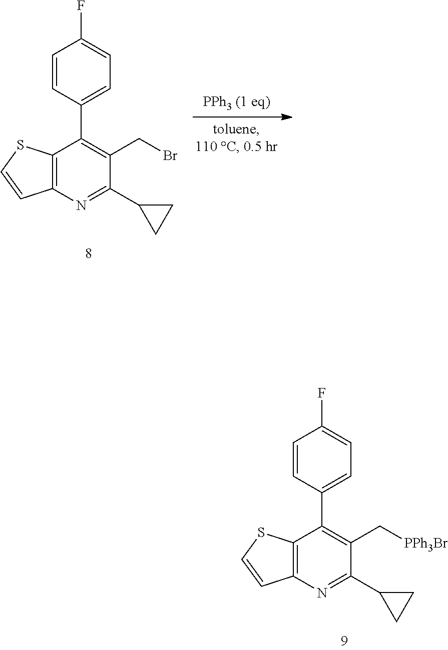

Step 8. To a solution of compound 8 (170 mg, 469.28 μmol, 1 eq) in toluene (2 mL) was added PPh₃ (123.08 mg, 469.28 μmol, 1 eq). The mixture was stirred at 110 °C for 0.5 hr under N₂. LCMS showed the starting material was consumed completely and the product was detected. The reaction mixture was concentrated under reduced pressure to give compound 9 (300 mg, crude) as a white solid. LCMS (ESI+): m/z=544.1 (M-Br), RT: 0.427 min.

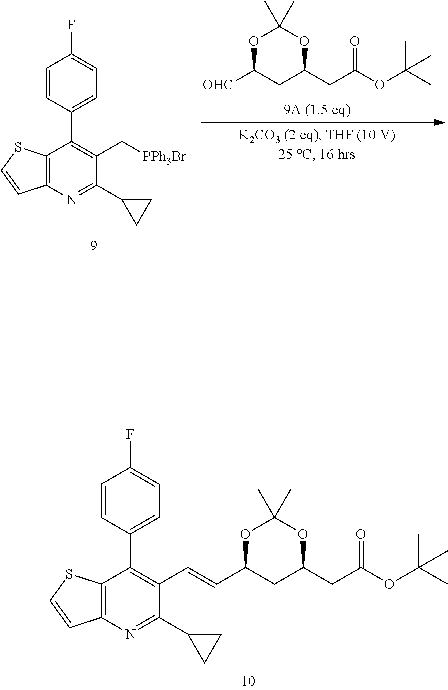

Step 9. To a solution of compound 9 (293 mg, 469.14 μmol, 1 eq) and compound 9A (181.78 mg, 703.71 μmol, 1.5 eq) in THF (3 mL) was added K₂CO₃ (129.68 mg, 938.29 μmol, 2 eq). The mixture was stirred at 25 °C for 16 hrs under N₂. LCMS showed the starting material was consumed completely and the product was detected. The reaction mixture was concentrated under reduced pressure to give a residue. The residue was purified by column chromatography on silica gel (Ethyl acetate/Petroleum ether=0 to 7%) to give compound 10 (107 mg, 43.12% yield) as a white solid. ¹H NMR: (400 MHz, CDCl₃) δ 1.01-1.04 (m, 2H) 1.18-1.22 (m, 1H) 1.24-1.31 (m, 2H) 1.38 (s, 3H) 1.47 (s, 9H) 1.59 (s, 3H) 2.24-2.31 (m, 1H) 2.33-2.48 (m, 3H) 4.22-4.30 (m, 1H) 4.35-4.41 (m, 1H) 5.56 (dd, J=16.0, 6.0 Hz, 1H) 6.64 (dd, J=16.4, 1.2 Hz, 1H) 7.16 (t, J=8.8 Hz, 2H) 7.37 (dd, J=8.4, 5.6 Hz, 2H) 7.45 (d, J=5.6 Hz, 1H) 7.62 (d, J=5.6 Hz, 1H).

In some embodiments, compound 10 is the sodium salt of compound 10.

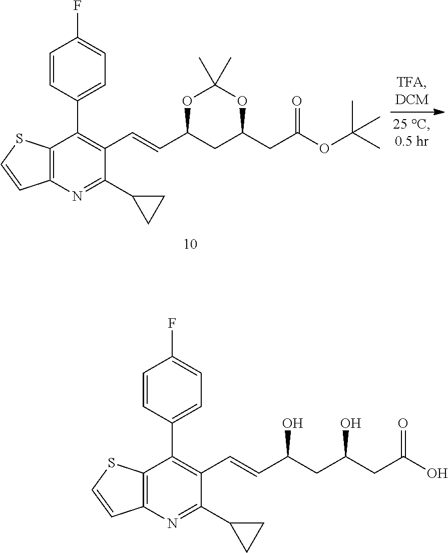 [SMILES: CC(C)(C)OC(=O)C[C@H]1C[C@@H](/C=C/c2c(C3CC3)nc3ccsc3c2-c2ccc(F)cc2)OC(C)(C)O1 ; O=C(O)C[C@H](O)C[C@H](O)/C=C/c1c(C2CC2)nc2ccsc2c1-c1ccc(F)cc1]

Step 10. To a solution of compound 10 (107 mg, 204.33 μmol, 1 eq) in DCM (1.5 mL) was added TFA (0.5 mL). The mixture was stirred at 25 °C for 0.5 hr under N₂. LCMS showed the starting material was consumed completely and the product was detected. The reaction mixture was concentrated under reduced pressure. The residue was purified by prep-HPLC (column: Phenomenex luna C18 150*25 mm*10 μm; mobile phase: [water (FA)-ACN]; gradient: 36%-66% B over 10 min) to give a crude product, which was dissolved in THF/H₂O (1:1, 1 mL) and adjusted to pH=9 and then was furtherly purified by prep-HPLC (column: Waters Xbridge 150*25 mm*5 μm; mobile phase: [water (NH₃·H₂O)-ACN]; gradient: 8%-28% B over 10 min) to afford (3R,5S,E)-7-(5-cyclopropyl-7-(4-fluorophenyl)thieno[3,2-b]pyridin-6-yl)-3,5-dihydroxyhept-6-enoic acid (20.10 mg, 17.83% yield) as a white solid. ¹H NMR: (400 MHz, DMSO-d₆) δ 0.98 (dd, J=8.0, 3.6 Hz, 2H), 1.06-1.12 (m, 2H), 1.16 (ddd, J=13.6, 6.4, 4.4 Hz, 1H), 1.38-1.46 (m, 1H), 1.93 (dd, J=15.2, 8.4 Hz, 1H), 2.05-2.13 (m, 1H), 2.40-2.47 (m, 1H), 3.61-3.64 (m, 1H), 4.11-4.17 (m, 1H), 5.57 (dd, J=16.0, 6.0 Hz, 1H), 6.54 (dd, J=16.0, 1.0 Hz, 1H), 7.30-7.37 (m, 2H), 7.42-7.48 (m, 3H), 7.98 (d, J=5.6 Hz, 1H). LCMS (ESI+): m/z=428.2 (M−17), RT: 1.573 min. LC/MS: HALO C18 90A 2.7 μm 3.0×30 mm. Detection methods are diode array detector (DAD). MS mode was positive electrospray ionization. MS range was 50-1050. Mobile phase: Ramp from 5% ACN (0.018% TFA) in water (0.037% TFA) to 95% ACN in 3.0 min, Flow rate is set at 1.0 mL/min; then hold at 95% ACN for 0.60 minutes Flow rate is set from 1.0 mL/min to 1.5 mL/min; return back to 5% ACN in water and hold for 0.40 min. Flow rate is set at 1.5 mL/min.

### Example 3. Synthesis of (3R,5S,E)-7-(5-cyclopropyl-7-(4-fluorophenyl)thiazolo[5,4-b]pyridin-6-yl)-3,5-dihydroxyhept-6-enoic acid

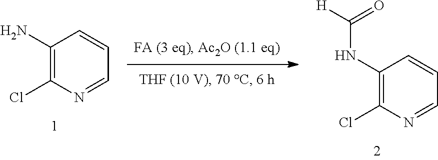 [SMILES: Nc1cccnc1Cl ; O=CNc1cccnc1Cl]

Step 1. A solution of formic acid (23.63 g, 513.38 mmol, 3 eq) and Ac₂O (19.22 g, 188.24 mmol, 1.1 eq) in THF (100 mL) was stirred at 70 °C for 3 hours. Compound 1 (22 g, 171.13 mmol, 1 eq) was added at 25 °C. The mixture was stirred at 70 °C for 3 hours under N₂. LCMS showed 96% of desired compound was detected. The reaction mixture was concentrated under reduced pressure. The residue was triturated with PE/EtOAc=3:1 (20 mL) at 25 °C for 30 minutes and filtered. The filter cake was dried under reduced pressure to afford compound 2 (21.5 g, 80.24% yield) as a yellow solid. ¹H NMR: (400 MHz, CDCl₃) δ 7.29 (dd, J=8.4, 4.8 Hz, 1H), 7.78 (s, 1H), 8.16 (dd, J=4.8, 1.2 Hz, 1H), 8.56 (s, 1H), 8.77 (dd, J=8.0, 0.8 Hz, 1H).

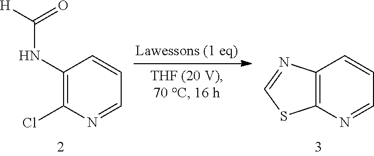 [SMILES: O=CNc1cccnc1Cl ; c1cnc2scnc2c1]

Step 2. To a solution of compound 2 (10.0 g, 63.87 mmol, 1 eq) in THF (400 mL) was added 2,4-bis(4-methoxyphenyl)-2,4-dithioxo-1,3,2,4dithiadiphosphetane (25.83 g, 63.87 mmol, 1 eq). The mixture was stirred at 70 °C for 16 hours under N₂. LCMS showed 32% of desired compound was detected. The reaction mixture was added to aq·NaHCO₃ (100 mL), the mixture was extracted with EtOAc (200 mL×3). The combined organic layers were washed with brine (100 mL), dried over anhydrous Na₂SO₄, filtered and concentrated under reduced pressure. The crude product was combined with five batches (10 g×4 and 5 g compound 2 was used) and purified by flash column chromatography on silica gel (0-9% EtOAc in PE) to afford compound 3 (34.4 g, 79.10% yield) as a yellow solid. LCMS (ESI+): m/z=137.1 (M+1). RT: 0.204 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate was set at 2.0 mL/min; then hold at 95% ACN for 0.18 minutes Flow rate was set at 2.0 mL/min; return back to 5% ACN in water and hold for 0.02 min. Flow rate was set at 2.0 mL/min.

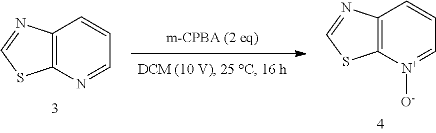 [SMILES: c1cnc2scnc2c1 ; [O-][n+]1cccc2ncsc21]

Step 3. To a solution of compound 3 (10.0 g, 73.44 mmol, 1 eq) in DCM (190 mL) was added m-CPBA (29.82 g, 146.87 mmol, 85% purity, 2 eq) The mixture was stirred at 25 °C. for 16 hours under N₂. LCMS showed 52% of desired compound was detected. The reaction was added to aq·Na₂SO₃ (200 mL). Wet starch potassium iodide test paper was used to test the quenching liquid. The test paper was still white in the acid system. The reaction mixture was adjusted to pH=8 with aq·NaHCO₃ and extracted with DCM (300 mL×4). The combined organic layers were washed with brine (300 mL), dried over anhydrous Na₂SO₄, filtered and concentrated under reduced pressure. The residue was combined with a parallel batch (10 g compound 3 was used) and triturated with PE/EtOAc=3:1 (10 mL) at 25 °C for 30 minutes and filtered. The filter cake was concentrated under reduced pressure to afford compound 4 (2.80 g, 12.52% yield) as a brown solid. ¹H NMR: (400 MHz, CDCl₃) δ 7.48 (dd, J=8.0, 6.4 Hz, 1H) 8.06 (d, J=8.4 Hz, 1H) 8.40 (d, J=6.4 Hz, 1H) 9.12 (s, 1H).

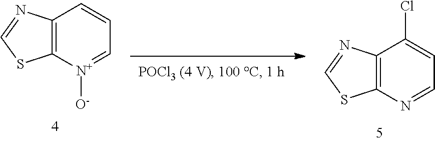 [SMILES: [O-][n+]1cccc2ncsc21 ; Clc1ccnc2scnc12]

Step 4. A solution of compound 4 (2.80 g, 18.40 mmol, 1 eq) in POCl₃ (10 mL) was stirred at 100 °C for 1 hour. LCMS showed 31% of desired compound was detected. The reaction mixture was added to saturated sodium bicarbonate solution (500 mL) at 25 °C, and then extracted with EtOAc (500 mL×3). The combined organic layers were washed with brine (200 mL), dried over anhydrous Na₂SO₄, filtered and concentrated under reduced pressure. The residue was purified by reversed-phase HPLC (column: Daisogel C18 250*70 mm*10 μm; mobile phase: [water (ammonia hydroxide v/v)-ACN]; gradient: 16%-46% B over 23 min) to afford compound 5 (1.20 g, 38.22% yield) as a white solid. ¹H NMR: (400 MHz, CDCl₃) δ 7.54 (d, J=5.2 Hz, 1H), 8.56 (d, J=5.2 Hz, 1H), 9.19 (s, 1H).

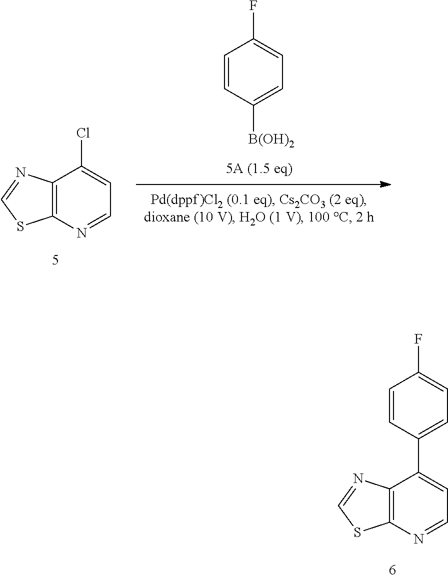 [SMILES: Clc1ccnc2scnc12 ; OB(O)c1ccc(F)cc1 ; Fc1ccc(-c2ccnc3scnc23)cc1]

Step 5. To a solution of compound 5 (1.20 g, 7.03 mmol, 1 eq) and compound 5A (1.48 g, 10.55 mmol, 1.5 eq) in dioxane (10 mL) and H₂O (1 mL) was added Cs₂CO₃ (4.58 g, 14.07 mmol, 2 eq) and Pd(dppf)Cl₂·CH₂Cl₂ (574.36 mg, 703.32 μmol, 0.1 eq). The mixture was stirred at 100 °C for 2 hours under N₂. LCMS showed 48% of desired compound was detected. The reaction mixture was concentrated under reduced pressure. The residue was purified by flash column chromatography on silica gel (0-10% EtOAc in PE) to afford compound 6 (1.50 g, 77.80% yield) as a yellow solid. LCMS (ESI+): m/z=231.0 (M+1), RT: 0.397 min. 5-95AB_0.8 MIN: LC/MS (The column used for chromatography was a Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate was set at 2.0 mL/min; then hold at 95% ACN for 0.18 minutes Flow rate was set at 2.0 mL/min; return back to 5% ACN in water and hold for 0.02 min. Flow rate was set at 2.0 mL/min.

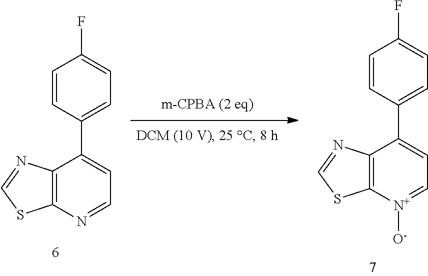 [SMILES: Fc1ccc(-c2ccnc3scnc23)cc1 ; [O-][n+]1ccc(-c2ccc(F)cc2)c2ncsc21]

Step 6. To a solution of compound 6 (1.4 g, 6.08 mmol, 1 eq) in DCM (14 mL) was added m-CPBA (2.47 g, 12.16 mmol, 85% purity, 2 eq). The mixture was stirred at 25 °C for 8 hours under N₂. LCMS showed 91% of desired compound was detected. The reaction was added to aq·Na₂SO₃ (150 mL). Wet starch potassium iodide test paper was used to test the quenching liquid. The test paper was still white in the acid system. The reaction mixture was adjusted to pH=8 with aq·NaHCO₃ and extracted with DCM (200 mL×3). The combined organic layers were washed with brine (200 mL), dried over anhydrous Na₂SO₄, filtered and concentrated under reduced pressure. The residue was purified by flash column chromatography on silica gel (0-6% MeOH in DCM) to afford compound 7 (1.31 g, 43.75% yield) as a light yellow solid. LCMS (ESI+): m/z=246.9 (M+1), RT: 0.445 min. LC/MS: Gemini@5 umNX-C18 110A 2*30 mm, 5 μm. Detection methods are photo-diode array (PDA). MS mode was positive electrospray ionization. MS range was 50-1050. Mobile phase: Ramp from 5% ACN in water (0.025% NH₃·H₂O) to 95% ACN in water in 0.70 min, Flow rate was set at 1.7 mL/min, then hold at 95% ACN for 0.20 min Flow rate was set at 1.7 mL/min; return back to 5% ACN in water and hold for 0.10 min. Flow rate was set at 1.7 mL/min.

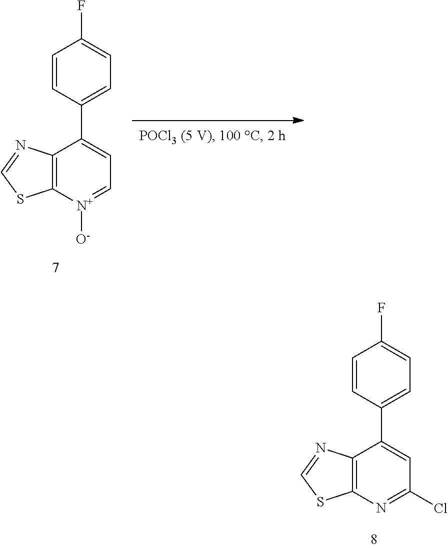 [SMILES: [O-][n+]1ccc(-c2ccc(F)cc2)c2ncsc21 ; Fc1ccc(-c2cc(Cl)nc3scnc23)cc1]

Step 7. A solution of compound 7 (1.31 g, 5.32 mmol, 1 eq) in POCl₃ (6 mL) was stirred at 100 °C for 2 hrs. LCMS showed 94% of desired compound was detected. The reaction mixture was added to saturated sodium bicarbonate solution (500 mL) at 25 °C, and then extracted with EtOAc (500 mL×3). The combined organic layers were washed with brine (500 mL), dried over anhydrous Na₂SO₄, filtered and concentrated under reduced pressure to afford compound 8 (1.28, 90.90% yield) as a white solid. LCMS (ESI+): m/z=265.0 (M+1), RT: 0.464 min. 5-95AB_0.8 MIN: LC/MS (The column used for chromatography was a Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate was set at 2.0 mL/min; then hold at 95% ACN for 0.18 minutes Flow rate was set at 2.0 mL/min; return back to 5% ACN in water and hold for 0.02 min. Flow rate was set at 2.0 mL/min.

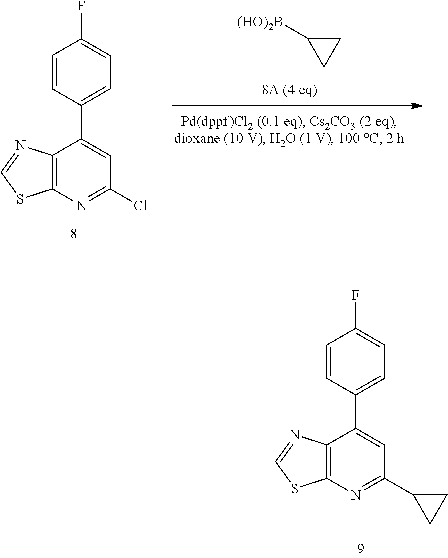 [SMILES: Fc1ccc(-c2cc(Cl)nc3scnc23)cc1 ; OB(O)C1CC1 ; Fc1ccc(-c2cc(C3CC3)nc3scnc23)cc1]

Step 8. To a solution of compound 8 (1.28 g, 4.84 mmol, 1 eq) and compound 8A (1.66 g, 19.34 mmol, 4 eq) in dioxane (12 mL) and H₂O (1.2 mL) was added Cs₂CO₃ (3.15 g, 9.67 mmol, 2 eq) and Pd(dppf)Cl₂ (353.82 mg, 483.56 μmol, 0.1 eq). The mixture was stirred at 100 °C for 2 hours under N₂. LCMS showed 82% of desired compound was detected. The reaction mixture was concentrated under reduced pressure. The residue was purified by flash column chromatography on silica gel (0-5% EtOAc in PE) to afford compound 9 (1.45 g, 99.83% yield) as a white solid. LCMS (ESI+): m/z=271.0 (M+1), RT: 0.483 min. 5-95AB 0.8 MIN: LC/MS (The column used for chromatography was a Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate was set at 2.0 mL/min; then hold at 95% ACN for 0.18 minutes Flow rate was set at 2.0 mL/min; return back to 5% ACN in water and hold for 0.02 min. Flow rate was set at 2.0 mL/min.

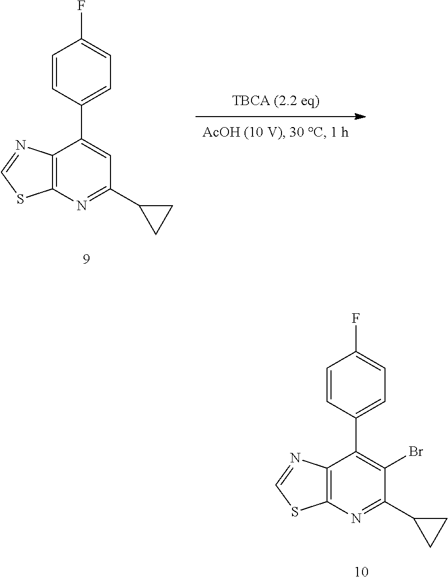 [SMILES: Fc1ccc(-c2cc(C3CC3)nc3scnc23)cc1 ; Fc1ccc(-c2c(Br)c(C3CC3)nc3scnc23)cc1]

Step 9. To a solution of compound 9 (750 mg, 2.77 mmol, 1 eq) in AcOH (10 mL) was added TBCA (2.83 g, 6.10 mmol, 2.2 eq). The mixture was stirred at 30 °C for 1 hour under N₂. LCMS showed 83% of desired compound was detected. The reaction was diluted with H₂O (20 mL) and extracted with EtOAc (20 mL×3). The combined organic layers were washed with brine (20 mL), dried over anhydrous Na₂SO₄, filtered and concentrated under reduced pressure. The residue was purified by flash column chromatography on silica gel (0-5% EtOAc in PE) to afford compound 10 (600 mg, 61.93% yield) as yellow oil. ¹H NMR: (400 MHz, CDCl₃) δ 1.12-1.17 (m, 2H), 1.22-1.27 (m, 2H), 2.75-2.85 (m, 1H), 7.20-7.26 (m, 2H), 7.46 (dd, J=8.8, 5.6 Hz, 2H), 8.93 (s, 1H).

In some embodiments, compound 10 is the sodium salt of compound 10.

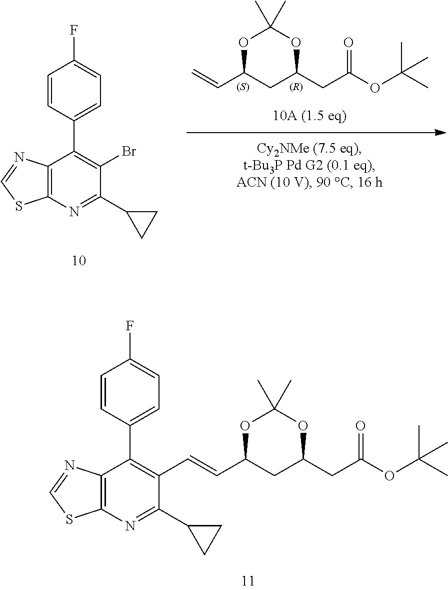

Step 10. To a solution of compound 10 (600 mg, 1.72 mmol, 1 eq) and compound 10A (660.62 mg, 2.58 mmol, 1.5 eq) in ACN (3 mL) was added tBu₃P Pd G2 (88.04 mg, 171.81 μmol, 0.1 eq) and N-cyclohexyl-N-methyl-cyclohexanamine (2.52 g, 12.89 mmol, 7.5 eq). The mixture was stirred at 90 °C for 16 hours under N₂. LCMS showed 58% of desired compound was detected. The reaction mixture was concentrated under reduced pressure. The residue was purified by flash column chromatography on silica gel (0-10% EtOAc in PE) to afford a crude product. The crude product was purified by prep-TLC (PE/DCM=1:3) to afford compound 11 (100 mg, 11.09% yield) as a white solid. ¹H NMR: (400 MHz, CDCl₃) δ 1.03-1.10 (m, 3H), 1.19-1.24 (m, 1H), 1.39 (s, 3H), 1.46-1.49 (m, 12H), 2.24-2.33 (m, 1H), 2.36-2.50 (m, 2H), 4.22-4.32 (m, 1H), 4.35-4.48 (m, 1H), 5.61 (dd, J=16.0, 5.6 Hz, 1H), 6.61 (dd, J=16.0, 1.2 Hz, 1H), 7.16 (t, J=8.8 Hz, 2H), 7.38 (dd, J=8.8, 5.6 Hz, 2H), 8.91 (s, 1H).

In some embodiments, compound 11 is the sodium salt of compound 11.

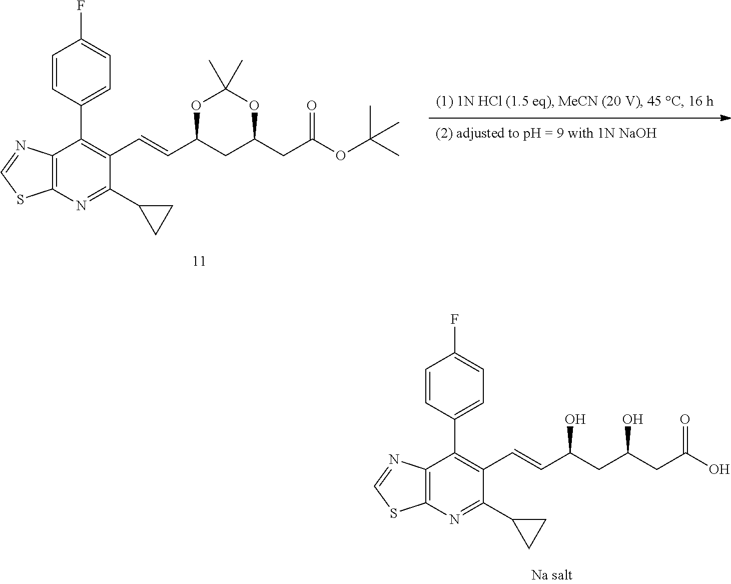 [SMILES: CC(C)(C)OC(=O)C[C@H]1C[C@@H](/C=C/c2c(C3CC3)nc3scnc3c2-c2ccc(F)cc2)OC(C)(C)O1 ; O=C(O)C[C@H](O)C[C@H](O)/C=C/c1c(C2CC2)nc2scnc2c1-c1ccc(F)cc1]

Step 11. To a solution of compound 11 (100 mg, 190.60 μmol, 1 eq) in MeCN (1 mL) was added HCl (1 M, 1.5 eq). The mixture was stirred at 45 °C for 16 hrs. LCMS showed 88% of desired compound was detected. The reaction mixture was adjusted to pH=9 with IN aq. NaOH and concentrated under reduced pressure. The residue was purified by reversed-phase HPLC (column: CD02-Waters Xbridge BEH C18 150*25*10 μm; mobile phase: [water (NH₃·H₂O)-ACN]; gradient: 0%-29% B over 12 min) to afford (3R,5S,E)-7-(5-cyclopropyl-7-(4-fluorophenyl)thiazolo[5,4-b]pyridin-6-yl)-3,5-dihydroxyhept-6-enoic acid (12.34 mg, 14.34% yield) as a white solid. ¹H NMR: (400 MHz, DMSO-d₆) δ 1.03 (dd, J=7.6, 3.6 Hz, 2H), 1.10 (d, J=4.4 Hz, 2H), 1.17-1.27 (m, 1H), 1.41-1.50 (m, 1H), 1.95-2.04 (m, 1H), 2.10-2.18 (m, 1H), 2.45-2.49 (m, 1H), 3.68 (td, J=8.0, 4.0 Hz, 1H), 4.17 (q, J=6.0 Hz, 1H), 5.62 (dd, J=16.0, 5.6 Hz, 1H), 6.53 (d, J=16.0 Hz, 1H), 7.27 (t, J=8.8 Hz, 2H), 7.43 (dd, J=8.8, 5.6 Hz, 2H), 9.28 (s, 1H). LCMS (ESI+): m/z=429.2 (M+1), RT: 1.635 min. LC/MS: Kinetex® EVO C18 3.0×50 mm 2.6 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in 3.40 min, Flow rate was set at 0.9 mL/min; then hold at 95% ACN for 0.30 minutes, Flow rate was set at, 0.9 mL/min; return back to 5% ACN in water and hold for 0.30 min. Flow rate was set at 1.2 mL/min.

### Example 4. Synthesis of (3R,5S,E)-7-(5-cyclopropyl-7-(4-fluorophenyl)-2-methylthiazolo[5,4-b]pyridin-6-yl)-3,5-dihydroxyhept-6-enoic acid

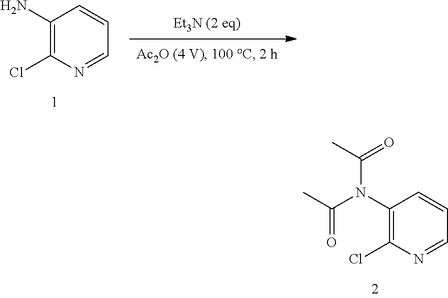

Step 1. To a solution of compound 1 (31.23 g, 242.92 mmol, 1 eq) in Ac₂O (125 mL) was added Et₃N (49.16 g, 485.85 mmol, 67.62 mL, 2 eq). The mixture was stirred at 100 °C for 2 hours. LCMS showed 93% of desired compound was detected. The reaction mixture was added water (300 mL), and then extracted with EtOAc (300 mL×3). The combined organic layers were washed with brine (300 mL), dried over anhydrous Na₂SO₄, filtered and concentrated under reduced pressure to give compound 2 (55.3 g, crude) as yellow oil. LCMS (ESI+): m/z=213.1 (M+1), RT: 0.241 min. 5-95AB_0.8 MIN: LC/MS (The column used for chromatography was a Kinetex® EVO C18 2.1×30 mm 5 μm. Detection methods are Photo-Diode Array (PDA). MS mode was positive electrospray ionization. MS range was 50-1050. Mobile phase A was 0.04% TFA in water, and mobile phase B was 0.02% TFA in HPLC grade acetonitrile. The gradient was 5-95% B in 0.60 min, flow rate was 2.0 mL/min; then hold at 95% ACN for 0.18 minutes, flow rate was 2.0 mL/min; return back to 5% ACN in water and hold for 0.02 min, flow rate was 2.0 mL/min.

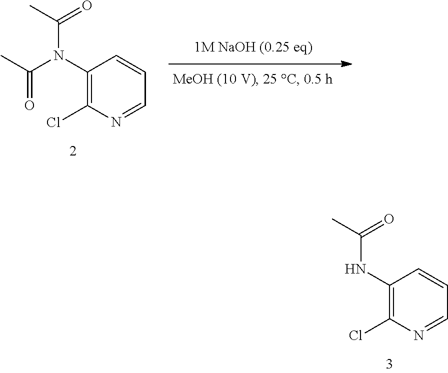

Step 2. To a solution of compound 2 (55 g, 258.66 mmol, 1 eq) in MeOH (500 mL) was added NaOH (1 M, 0.25 eq). The mixture was stirred at 25 °C for 0.5 hour. LCMS showed 100% of desired compound was detected. The reaction was concentrated under reduced pressure. The residue was diluted with water (400 mL), adjusted to pH=8 with 1 N HCl and extracted with EtOAc (500 ml×3). The combined organic layers were washed with brine (500 mL), dried over anhydrous Na₂SO₄, filtered and concentrated under reduced pressure to give compound 3 (46.46 g, crude) as a brown solid. LCMS (ESI+): m/z=171.1 (M+1), RT: 0.169 min. 5-95AB_0.8 MIN: LC/MS (The column used for chromatography was a Kinetex® EVO C18 2.1×30 mm 5 μm. Detection methods are Photo-Diode Array (PDA). MS mode was positive electrospray ionization. MS range was 50-1050. Mobile phase A was 0.04% TFA in water, and mobile phase B was 0.02% TFA in HPLC grade acetonitrile. The gradient was 5-95% B in 0.60 min, flow rate was 2.0 mL/min; then hold at 95% ACN for 0.18 minutes, flow rate was 2.0 mL/min; return back to 5% ACN in water and hold for 0.02 min, flow rate was 2.0 mL/min.

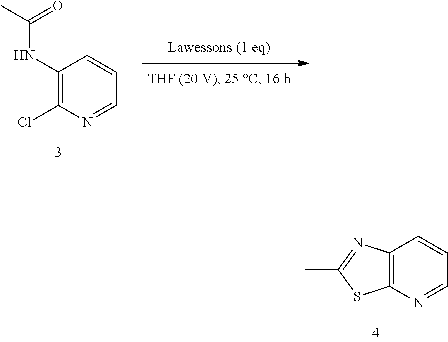

Step 3. To a solution of compound 3 (51.1 g, 299.54 mmol, 1 eq) in THF (500 mL) was added 2,4-bis(4-methoxyphenyl)-2,4-dithioxo-1,3,2,4dithiadiphosphetane (96.92 g, 239.63 mmol, 0.8 eq). The mixture was stirred at 25 °C for 16 hours under N₂. LCMS showed 45% of desired compound was detected. The reaction was added to aq·NaHCO₃ (500 mL), the mixture was extracted with EtOAc (500 mL×3). The combined organic layers were washed with brine (500 mL), dried over anhydrous Na₂SO₄, filtered and concentrated under reduced pressure. The residue was purified by flash column chromatography on silica gel (0-16% EtOAc in PE) to give compound 4 (42.47 g, 57.58% yield) as yellow oil. LCMS (ESI+): m/z=151.1 (M+1), RT: 0.249 min. 5-95AB_0.8 MIN: LC/MS (The column used for chromatography was a Kinetex® EVO C18 2.1×30 mm 5 μm. Detection methods are Photo-Diode Array (PDA). MS mode was positive electrospray ionization. MS range was 50-1050. Mobile phase A was 0.04% TFA in water, and mobile phase B was 0.02% TFA in HPLC grade acetonitrile. The gradient was 5-95% B in 0.60 min, flow rate was 2.0 mL/min; then hold at 95% ACN for 0.18 minutes, flow rate was 2.0 mL/min; return back to 5% ACN in water and hold for 0.02 min, flow rate was 2.0 mL/min.

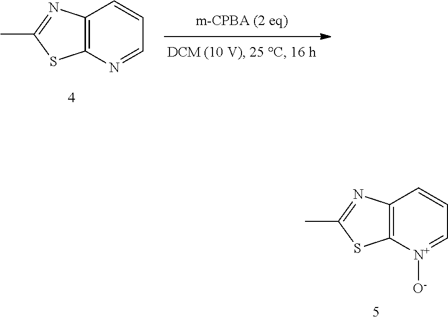

Step 4. To a solution of compound 4 (13.3 g, 88.55 mmol, 1 eq) in DCM (140 mL) was added m-CPBA (35.95 g, 177.10 mmol, 85% purity, 2 eq). The mixture was stirred at 25 °C for 16 hours under N₂. LCMS showed 86% of desired compound was detected. The reaction was added to aq·Na₂SO₃ (100 mL). Wet starch potassium iodide test paper was used to test the quenching liquid. The test paper was still white in the acid system. The reaction mixture was adjusted to pH=8 with aq·NaHCO₃ and extracted with DCM (200 mL×3). The combined organic layers were washed with brine (100 mL), dried over anhydrous Na₂SO₄, filtered and concentrated under reduced pressure. The residue was purified by flash column chromatography on silica gel (0-6% MeOH in DCM) to give compound 5 (5.99 mg, 40.70% yield) as yellow oil. ¹H NMR: (400 MHz, CDCl₃) δ 2.89 (s, 3H), 7.39 (t, J=7.2 Hz, 1H), 7.86 (d, J=8.4 Hz, 1H), 8.30 (d, J=6.4 Hz, 1H).

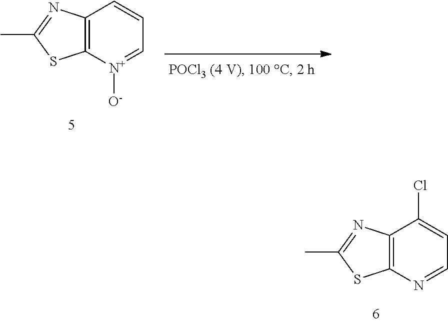

Step 5. A solution of compound 5 (6.48 g, 38.99 mmol, 1 eq) in POCl₃ (19 mL) was stirred at 100 °C for 2 hours. LCMS showed 65% of product was detected. The reaction mixture was added to saturated sodium bicarbonate solution (500 mL) at 25 °C and extracted with EtOAc (500 mL×3). The combined organic layers were washed with brine (500 mL), dried over Na₂SO₄, filtered and concentrated under reduced pressure. The residue was purified by reversed-phase HPLC (column: Daisogel C18 250*70 mm*10 μm; mobile phase: [water (NH₃·H₂O)-ACN]; gradient: 10%-40% B over 25 min) to give compound 6 (7.28 g, 50.56% yield) as a white solid. ¹H NMR: (400 MHz, CDCl₃) δ 2.91 (s, 3H), 7.44 (d, J=5.2 Hz, 1H), 8.43 (d, J=5.2 Hz, 1H). LCMS (ESI+): m/z=184.9 (M+1), RT: 0.292 min. 5-95AB_0.8 MIN: LC/MS (The column used for chromatography was a Kinetex® EVO C18 2.1×30 mm 5 μm. Detection methods are Photo-Diode Array (PDA). MS mode was positive electrospray ionization. MS range was 50-1050. Mobile phase A was 0.04% TFA in water, and mobile phase B was 0.02% TFA in HPLC grade acetonitrile. The gradient was 5-95% B in 0.60 min, flow rate was 2.0 mL/min; then hold at 95% ACN for 0.18 minutes, flow rate was 2.0 mL/min; return back to 5% ACN in water and hold for 0.02 min, flow rate was 2.0 mL/min.

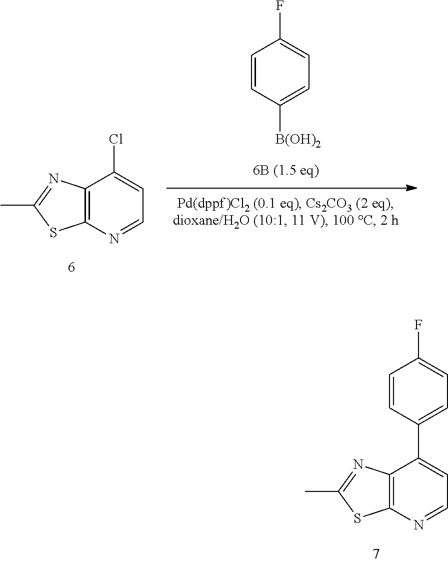 [SMILES: Cc1nc2c(Cl)ccnc2s1 ; OB(O)c1ccc(F)cc1 ; Cc1nc2c(-c3ccc(F)cc3)ccnc2s1]

Step 6. To a solution of compound 6 (7.28 g, 39.43 mmol, 1 eq) and compound 6B (8.27 g, 59.14 mmol, 1.5 eq) in dioxane (70 mL) and H₂O (7 mL) was added Cs₂CO₃ (25.69 g, 78.85 mmol, 2 eq) and Pd(dppf)Cl₂·CH₂Cl₂ (3.22 g, 3.94 mmol, 0.1 eq). The mixture was stirred at 100 °C for 2 hours under N₂. LCMS showed 66% of desired compound was detected. The reaction was concentrated under reduced pressure. The residue was purified by flash column chromatography on silica gel (0-5% EtOAc in PE) to give compound 7 (10.7 g, 99.98% yield) as a white solid. LCMS (ESI+): m/z=245.1 (M+1), RT: 0.421 min. 5-95AB_0.8 MIN: LC/MS (The column used for chromatography was a Kinetex® EVO C18 2.1×30 mm 5 μm. Detection methods are Photo-Diode Array (PDA). MS mode was positive electrospray ionization. MS range was 50-1050. Mobile phase A was 0.04% TFA in water, and mobile phase B was 0.02% TFA in HPLC grade acetonitrile. The gradient was 5-95% B in 0.60 min, flow rate was 2.0 mL/min; then hold at 95% ACN for 0.18 minutes, flow rate was 2.0 mL/min; return back to 5% ACN in water and hold for 0.02 min, flow rate was 2.0 mL/min.

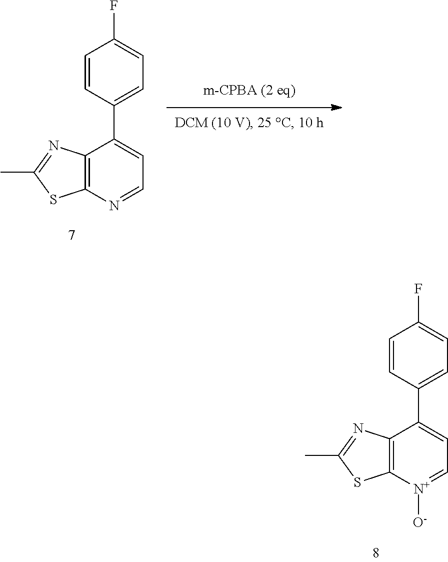 [SMILES: Cc1nc2c(-c3ccc(F)cc3)ccnc2s1 ; Cc1nc2c(-c3ccc(F)cc3)cc[n+]([O-])c2s1]

Step 7. To a solution of compound 7 (10.7 g, 43.80 mmol, 1 eq) in DCM (100 mL) was added m-CPBA (15.12 g, 87.60 mmol, 2 eq). The mixture was stirred at 25 °C for 10 hours under N₂. LCMS showed 95% of desired compound was detected. The reaction was added to aq·Na₂SO₃ (150 mL). Wet starch potassium iodide test paper was used to test the quenching liquid. The test paper was still white in the acid system. The reaction mixture was adjusted to pH=8 with aq·NaHCO₃ and extracted with DCM (200 mL×3). The combined organic layers were washed with brine (200 mL), dried over anhydrous Na₂SO₄, filtered and concentrated under reduced pressure. The residue was purified by flash column chromatography on silica gel (0-6% MeOH in DCM) to give compound 8 (9.54 g, 83.68% yield) as a light yellow solid. LCMS (ESI+): m/z=260.9 (M+1), RT: 0.635 min. LC/MS: Gemini@5 umNX-C18 110A 2*30 mm, 5 μm. Detection methods are photo-diode array (PDA). MS mode was positive electrospray ionization. MS range was 50-1050. Mobile phase: Ramp from 0% ACN in water (0.025% NH₃·H₂O) to 60% ACN in water in 0.70 min, Flow rate was set at 1.7 mL/min, then hold at 60% ACN for 0.20 min Flow rate was set at 1.7 mL/min; return back to 0% ACN in water and hold for 0.10 min. Flow rate was set at 1.7 mL/min.

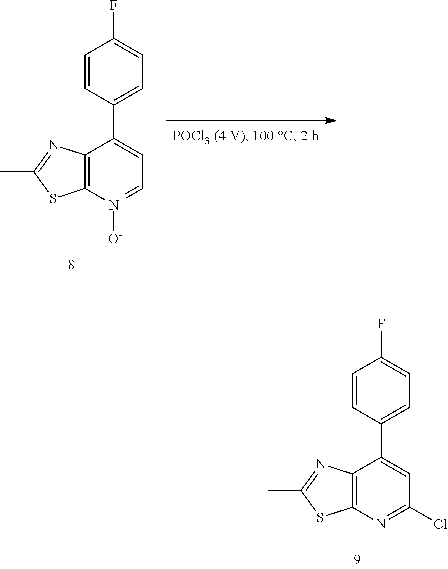 [SMILES: Cc1nc2c(-c3ccc(F)cc3)cc[n+]([O-])c2s1 ; Cc1nc2c(-c3ccc(F)cc3)cc(Cl)nc2s1]

Step 8. A solution of compound 8 (4.77 g, 18.33 mmol, 1 eq) in POCl₃ (19 mL) was stirred at 100 °C for 2 hours. LCMS showed 96% of desired compound was detected. The reaction mixture was added to saturated sodium bicarbonate solution (1500 mL) at 25 °C and extracted with EtOAc (1500 mL×3). The combined organic layers were washed with brine (1000 mL), dried over anhydrous Na₂SO₄, filtered and concentrated under reduced pressure to give compound 9 (9.67 g, 94.65% yield) as a white solid. LCMS (ESI+): m/z=279.0 (M+1), RT: 0.488 min. 5-95AB_0.8 MIN: LC/MS (The column used for chromatography was a Kinetex® EVO C18 2.1×30 mm 5 μm. Detection methods are Photo-Diode Array (PDA). MS mode was positive electrospray ionization. MS range was 50-1050. Mobile phase A was 0.04% TFA in water, and mobile phase B was 0.02% TFA in HPLC grade acetonitrile. The gradient was 5-95% B in 0.60 min, flow rate was 2.0 mL/min; then hold at 95% ACN for 0.18 minutes, flow rate was 2.0 mL/min; return back to 5% ACN in water and hold for 0.02 min, flow rate was 2.0 mL/min.

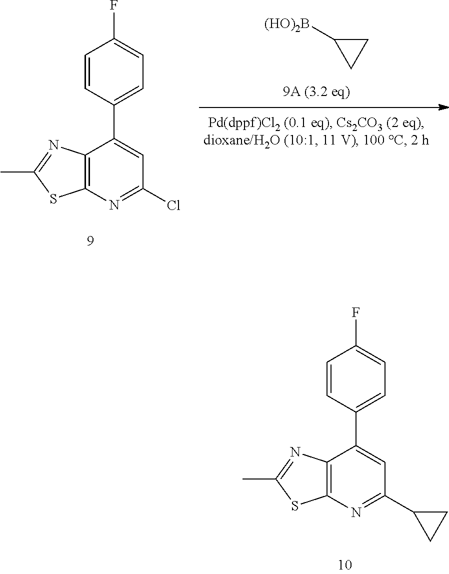 [SMILES: Cc1nc2c(-c3ccc(F)cc3)cc(Cl)nc2s1 ; OB(O)C1CC1 ; Cc1nc2c(-c3ccc(F)cc3)cc(C3CC3)nc2s1]

Step 9. To a solution of compound 9 (9.67 g, 34.69 mmol, 1 eq) and compound 9A (9.54 g, 111.02 mmol, 3.2 eq) in dioxane (90 mL) and H₂O (9 mL) was added Cs₂CO₃ (22.61 g, 69.39 mmol, 2 eq) and Pd(dppf)Cl₂ (2.54 g, 3.47 mmol, 0.1 eq). The mixture was stirred at 100 °C for 2 hours under N₂. LCMS showed 80% of desired compound was detected. The reaction was concentrated under reduced pressure. The residue was purified by flash column chromatography on silica gel (0-5% EtOAc in PE) to give compound 10 (8.8 g, 89.20% yield) as a white solid. LCMS (ESI+): m/z=285.1 (M+1), RT: 0.503 min. 5-95AB_0.8 MIN: LC/MS (The column used for chromatography was a Kinetex® EVO C18 2.1×30 mm 5 μm. Detection methods are Photo-Diode Array (PDA). MS mode was positive electrospray ionization. MS range was 50-1050. Mobile phase A was 0.04% TFA in water, and mobile phase B was 0.02% TFA in HPLC grade acetonitrile. The gradient was 5-95% B in 0.60 min, flow rate was 2.0 mL/min; then hold at 95% ACN for 0.18 minutes, flow rate was 2.0 mL/min; return back to 5% ACN in water and hold for 0.02 min, flow rate was 2.0 mL/min.

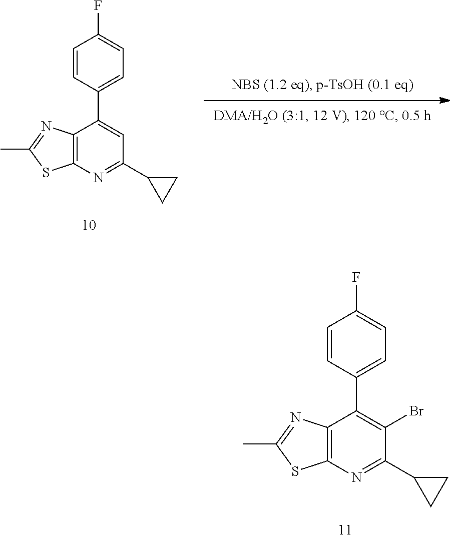 [SMILES: Cc1nc2c(-c3ccc(F)cc3)cc(C3CC3)nc2s1 ; Cc1nc2c(-c3ccc(F)cc3)c(Br)c(C3CC3)nc2s1]

Step 10. To a solution of compound 10 (2.80 g, 9.85 mmol, 1 eq) in DMA (30 mL) and H₂O (20 mL) was added NBS (2.10 g, 11.82 mmol, 1.2 eq) and TsOH·H₂O (187.31 mg, 984.70 μmol, 0.1 eq). The mixture was stirred at 120 °C for 0.5 hours under N₂. LCMS showed 44% of compound 10 and ~15% of desired compound was detected. The reaction mixture was concentrated under reduced pressure. The residue was combined with two batches (3 g×2 compound 10 was used) and purified by reversed-phase HPLC (column: Phenomenex luna C18 (250*70 mm, 10 μm); mobile phase: [water (FA)-ACN]; gradient: 60%-90% B over 5 min) to give compound 11 (1.52 g, 13.57% yield) was obtained as black oil. LCMS (ESI+): m/z=363.0 (M+1), RT: 0.549 min. 5-95AB_0.8 MIN: LC/MS (The column used for chromatography was a Kinetex® EVO C18 2.1×30 mm 5 μm. Detection methods are Photo-Diode Array (PDA). MS mode was positive electrospray ionization. MS range was 50-1050. Mobile phase A was 0.04% TFA in water, and mobile phase B was 0.02% TFA in HPLC grade acetonitrile. The gradient was 5-95% B in 0.60 min, flow rate was 2.0 mL/min; then hold at 95% ACN for 0.18 minutes, flow rate was 2.0 mL/min; return back to 5% ACN in water and hold for 0.02 min, flow rate was 2.0 mL/min.

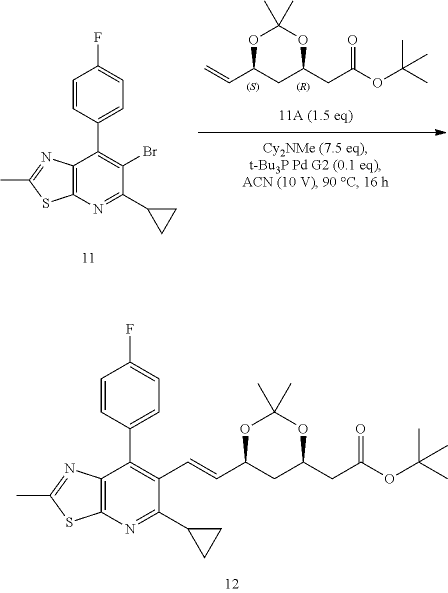

Step 11. To a solution of compound 11 (400 mg, 1.10 mmol, 1 eq) and compound 11A (423.41 mg, 1.65 mmol, 1.5 eq) in ACN (8 mL) was added tBu₃P Pd G2 (56.42 mg, 110.12 μmol, 0.1 eq) and N-cyclohexyl-N-methyl-cyclohexanamine (1.61 g, 8.26 mmol, 7.5 eq). The mixture was stirred at 90 °C for 16 hours under N₂. LCMS showed 54% of desired compound was detected. The reaction mixture was concentrated under reduced pressure. The residue was combined with a paralleled batch (200 mg compound 11 was used) and purified by flash silica gel chromatography (100% EtOAc) to give a crude product. The crude product was purified by reversed-phase HPLC (column: Welch Ultimate C18 150*25 mm*5 μm; mobile phase: [water (TFA)-ACN]; gradient: 80%-100% B over 10 min) and prep-TLC (PE/EtOAc=5:1) to give compound 12 (100 mg, 16.86% yield) as a white solid. ¹H NMR: (400 MHz, CDCl₃) δ 1.02 (br dd, J=8.4, 3.2 Hz, 2H), 1.15-1.31 (m, 3H), 1.38 (s, 3H), 1.43 (s, 1H), 1.47 (s, 9H), 1.56 (s, 3H), 2.25-2.32 (m, 1H), 2.33-2.40 (m, 1H), 2.40-2.47 (m, 1H), 2.74 (s, 3H), 4.22-4.30 (m, 1H), 4.39 (d, J=10.4, 7.6 Hz, 1H), 5.57 (dd, J=16.0, 6.0 Hz, 1H), 6.56 (d, J=16.4 Hz, 1H), 7.07-7.19 (m, 2H), 7.36 (dd, J=8.0, 6.0 Hz, 2H).

In some embodiments, compound 12 is the sodium salt of compound 12.

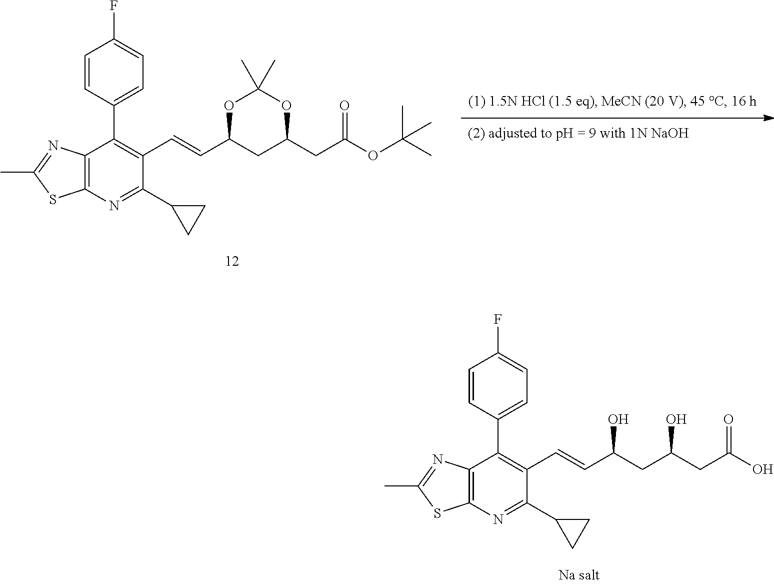 [SMILES: Cc1nc2c(-c3ccc(F)cc3)c(/C=C/[C@@H]3C[C@H](CC(=O)OC(C)(C)C)OC(C)(C)O3)c(C3CC3)nc2s1 ; Cc1nc2c(-c3ccc(F)cc3)c(/C=C/[C@@H](O)C[C@@H](O)CC(=O)O)c(C3CC3)nc2s1]

Step 12. To a solution of compound 12 (90 mg, 167.08 μmol, 1 eq) in ACN (1 mL) was added HCl (1.5 M, 1.5 eq). The mixture was stirred at 45 °C for 16 hours. LCMS showed 79% of desired compound was detected. The reaction mixture was adjusted with IN aq·NaOH to pH=9, filtered and the filtrate was concentrated under reduced pressure. The residue was purified by reversed-phase HPLC (column: Waters Xbridge 150*25 mm*5 μm; mobile phase: [water (NH₃·H₂O)-ACN]; gradient: 4%-34% B over 10 min) to give (3R,5S,E)-7-(5-cyclopropyl-7-(4-fluorophenyl)-2-methylthiazolo[5,4-b]pyridin-6-yl)-3,5-dihydroxyhept-6-enoic acid sodium salt (35.3 mg, 47.75% yield) as a white solid. ¹H NMR: (400 MHz, DMSO-d₆) δ 0.94-1.03 (m, 2H), 1.05-1.13 (m, 2H), 1.26-1.55 (m, 2H), 1.96-2.08 (m, 1H), 2.11-2.19 (m, 1H), 2.41-2.48 (m, 1H), 2.69 (s, 3H), 3.77-3.90 (m, 1H), 4.17-4.24 (m, 1H), 5.65 (ddd, J=16.0, 5.6, 2.0 Hz, 1H), 6.50 (dd, J=16.0, 1.2 Hz, 1H), 7.19-7.28 (m, 2H), 7.31-7.46 (m, 2H). LCMS (ESI+): m/z=443.2 (M+1), RT: 1.610 min. LC/MS: Kinetex® XBridge C18 3.0*50 mm, 5 μm. Detection methods are photo-diode array (PDA). MS mode was positive electrospray ionization. MS range was 50-1050. Mobile phase: Ramp from 0% ACN in water (0.025% NH₃·H₂O) to 60% ACN in 3.00 min, Flow rate was set at 0.9 mL/min; then hold at 60% ACN for 0.70 minutes Flow rate was set at 0.9 mL/min; return back to 0% ACN in water and hold for 0.30 min. Flow rate was set at 1.2 mL/min.

### Example 5. Synthesis of (3R,5S,E)-7-(6-cyclopropyl-4-(4-fluorophenyl)-2-methylthieno[2,3-b]pyridin-5-yl)-3,5-dihydroxyhept-6-enoic acid

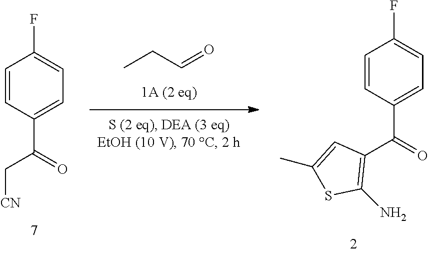

Step 1: To a solution of compound 1 (10 g, 61.29 mmol, 1 eq) in EtOH (100 mL) was added compound 1A (7.12 g, 122.59 mmol, 8.92 mL, 2 eq), S (3.93 g, 122.59 mmol, 2 eq) and diethylamine (13.45 g, 183.88 mmol, 18.94 mL, 3 eq). The mixture was degassed and purged with N₂ for 3 times and stirred at 70 °C for 2 hours under N₂ atmosphere. LCMS showed reactant was consumed completely and one peak with 58% desired mass was detected. The reaction mixture was concentrated under reduced pressure and the residue was diluted with water (20 mL), extracted with ethyl acetate (30 mL×3) and washed with brine (30 mL×3). The combined organic layers were dried over anhydrous Na₂SO₄, filtered and the filtrate was concentrated under reduced pressure to obtain a residue. The residue was purified by column chromatography on silica gel (PE/EtOAc=1/0 to 10/1) to give compound 2 (5.3 g, 26.75% yield) as yellow solid.

LCMS (ESI+): m/z=236.0 (M+1), RT: 0.423 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes, Flow rate is set at 2.0 mL/min; return back to 5% ACN in water and hold for 0.02 min. Flow rate is set at 2.0 mL/min.

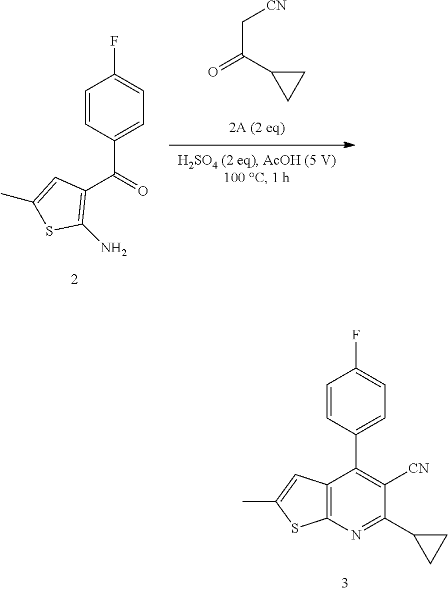 [SMILES: Cc1cc(C(=O)c2ccc(F)cc2)c(N)s1 ; N#CCC(=O)C1CC1 ; Cc1cc2c(-c3ccc(F)cc3)c(C#N)c(C3CC3)nc2s1]

Step 2: To a solution of compound 2 (4.3 g, 13.30 mmol, 1 eq) in AcOH (20 mL) was added 3-cyclopropyl-3-oxo-propanenitrile (2.90 g, 26.61 mmol, 2 eq) and H₂SO₄ (2.61 g, 26.61 mmol, 1.42 mL, 2 eq). The mixture was stirred at 100 °C for 1 hour. LCMS showed reactant was consumed completely and one peak with 56% desired mass was detected. The reaction quenched by adding water 50 mL at 25 °C. The mixture was extracted with ethyl acetate (50 mL×3) and washed with brine (50 mL×3), then the organic layer was evaporated, dried with anhydrous Na₂SO₄, filtered and concentrated in vacuum to give a residue. The residue was purified by prep-HPLC (column: Phenomenex luna C18 (250*70 mm, 10 μm); mobile phase: [water (FA)-ACN]; gradient: 60%-90% B over 22 min) and concentrated in vacuum to give compound 3 (4 g, 75.09% yield) as yellow solid. ¹H NMR: (400 MHz, CDCl₃) δ ppm 1.05-1.14 (m, 2H), 1.19-1.26 (m, 2H), 2.47 (s, 3H), 2.53-2.58 (m, 1H), 6.65 (s, 1H), 7.15-7.20 (m, 2H), 7.46 (dd, J=8.0, 5.6 Hz, 2H). LCMS (ESI+): m/z=309.0 (M+1), RT: 0.466 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes, Flow rate is set at 2.0 mL/min; return back to 5% ACN in water and hold for 0.02 min. Flow rate is set at 2.0 mL/min.

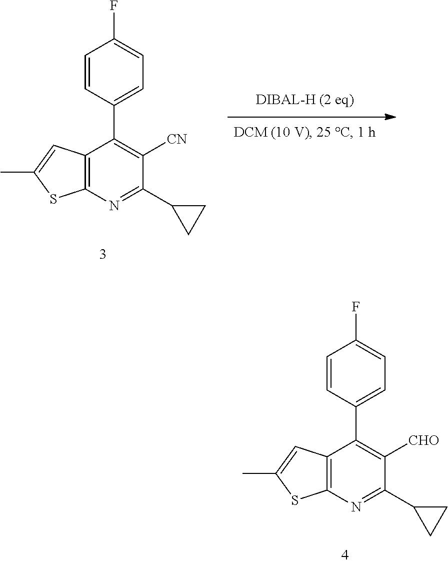 [SMILES: Cc1cc2c(-c3ccc(F)cc3)c(C#N)c(C3CC3)nc2s1 ; Cc1cc2c(-c3ccc(F)cc3)c(C=O)c(C3CC3)nc2s1]

Step 3: A mixture of compound 3 (2 g, 6.24 mmol, 1 eq) in DCM (20 mL) was degassed and purged with N₂ for 3 times, then DIBAL-H (1 M, 12.49 mL, 2 eq) was added slowly to the solution at 0 °C. The mixture was stirred at 25 °C for 1 hr under N₂ atmosphere. LCMS showed reactant was consumed completely and one peak with 36% desired mass was detected. The reaction was quenched by adding slowly MeOH 10 mL and 15% NaOH solution 5 mL at 0 °C. The mixture was stirred at 25 °C for 15 min under N₂ atmosphere, dried with anhydrous Na₂SO₄, filtered and concentrated under reduced pressure. The residue was purified by column chromatography on silica gel (PE/EtOAc=1/0 to 10/1) to give compound 4 (0.8 g, 19.21% yield) as yellow solid. LCMS (ESI+): m/z=311.9 (M+1), RT: 0.618 min. LC/MS: HALO C18 90A 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes, Flow rate is set at 2.0 mL/min; return back to 5% ACN in water and hold for 0.02 min. Flow rate is set at 2.0 mL/min.

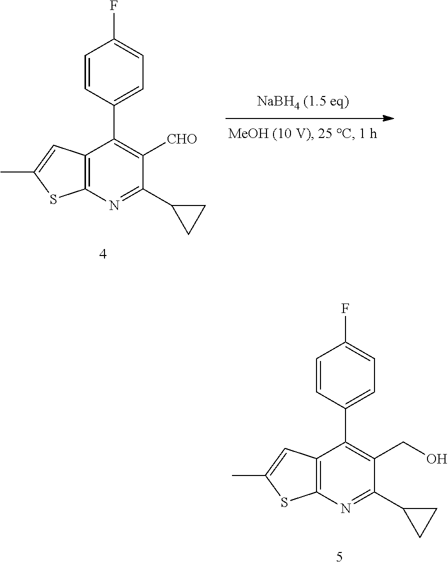 [SMILES: Cc1cc2c(-c3ccc(F)cc3)c(C=O)c(C3CC3)nc2s1 ; Cc1cc2c(-c3ccc(F)cc3)c(CO)c(C3CC3)nc2s1]

Step 4: To a solution of compound 4 (0.8 g, 2.40 mmol, 1 eq) in MeOH (8 mL) was added NaBH₄ (136.12 mg, 3.60 mmol, 1.5 eq) at 0 °C under N₂ atmosphere. The mixture was degassed and purged with N₂ for 3 times and stirred at 25 °C for 1 hour under N₂ atmosphere. LCMS showed reactant was consumed completely and one peak with 89% desired mass was detected. The reaction was quenched by adding slowly H₂O 20 mL at 0 °C. The mixture was extracted with ethyl acetate (10 mL×3) and washed with brine (10 mL×3), then the organic layer was dried with anhydrous Na₂SO₄, filtered and concentrated under reduced pressure. The residue was purified by column chromatography on silica gel (PE/EtOAc=1/0 to 10/1) to give compound 5 (0.6 g, 78.55% yield) as white solid. ¹H NMR: (400 MHz, CDCl₃) δ ppm 1.00-1.11 (m, 2H), 1.24-1.31 (m, 2H), 1.66 (br s, 1H), 2.50 (s, 3H), 4.74 (s, 2H), 6.47 (s, 1H), 7.20 (t, J=8.4 Hz, 2H), 7.38 (dd, J=8.4, 5.6 Hz, 2H). LCMS (ESI+): m/z=314.0 (M+1), RT: 0.465 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes, Flow rate is set at 2.0 mL/min; return back to 5% ACN in water and hold for 0.02 min. Flow rate is set at 2.0 mL/min.

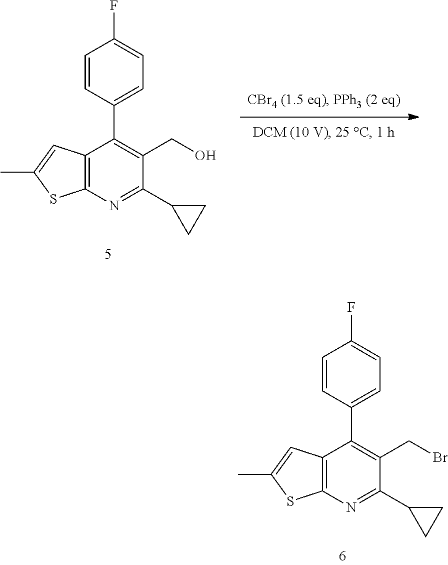 [SMILES: Cc1cc2c(-c3ccc(F)cc3)c(CO)c(C3CC3)nc2s1 ; Cc1cc2c(-c3ccc(F)cc3)c(CBr)c(C3CC3)nc2s1]

Step 5: To a solution of compound 5 (0.3 g, 942.06 μmol, 1 eq) in DCM (3 mL) was added PPh₃ (494.18 mg, 1.88 mmol, 2 eq) and CBr₄ (468.62 mg, 1.41 mmol, 1.5 eq) at 0 °C. The mixture was stirred at 25 °C for 1 hour. LCMS showed reactant was consumed completely and one peak with 24% desired mass was detected. The mixture was concentrated under reduced pressure. The residue was purified by column chromatography on silica gel (PE/EtOAc=1/0 to 10/1) to give compound 6 (0.65 g, 90.23% yield) as white solid. LCMS (ESI+): m/z=375.9 (M+1), RT: 0.588 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes, Flow rate is set at 2.0 mL/min; return back to 5% ACN in water and hold for 0.02 min. Flow rate is set at 2.0 mL/min.

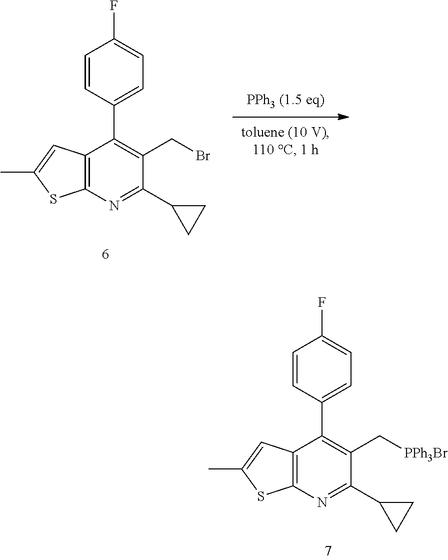

Step 6: A mixture of 5 compound 6 (0.65 g, 1.70 mmol, 1 eq), PPh₃ (668.88 mg, 2.55 mmol, 1.5 eq) in Toluene (6 mL) was degassed and purged with N₂ for 3 times, and then the mixture was stirred at 110 °C for 1 hour under N₂ atmosphere. LCMS showed reactant was consumed completely and one peak with 11% desired mass was detected. The mixture was concentrated under reduced pressure to give compound 7 (1.09 g, 100.00% yield) as white solid.

LCMS (ESI+): m/z=558.1 (M−79), RT: 0.458 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes, Flow rate is set at 2.0 mL/min; return back to 5% ACN in water and hold for 0.02 min. Flow rate is set at 2.0 mL/min.

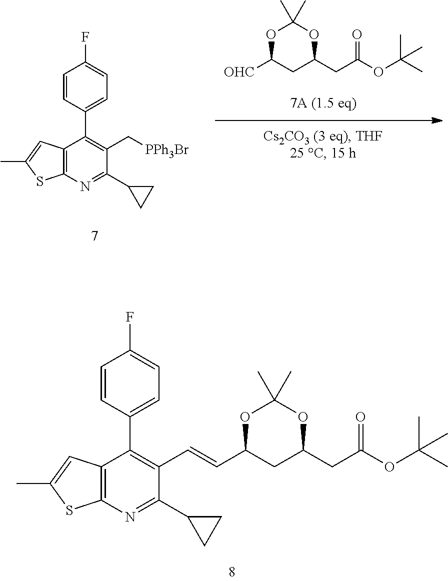

Step 7: LCMS showed reactant was consumed completely and one peak with 18% desired mass was detected. The mixture was concentrated under reduced pressure. The residue was purified by column chromatography on silica gel (PE/EtOAc=1/0 to 10/1) to give a crude product. The crude product was purified by prep-TLC (PE/EtOAc=10/1) to give compound 8 (0.2 g, 21.53% yield) as colorless oil. ¹H NMR: (400 MHz, CDCl₃) δ ppm 0.97-1.01 (m, 2H), 1.14-1.24 (m, 2H), 1.37 (s, 3H), 1.43-1.49 (m, 12H), 1.60-1.61 (m, 2H), 2.22-2.30 (m, 1H), 2.31-2.38 (m, 1H), 2.39-2.46 (m, 1H), 2.49 (s, 3H), 4.19-4.31 (m, 1H), 4.32-4.41 (m, 1H), 5.53 (dd, J=16.0, 6.0 Hz, 1H), 6.48 (s, 1H), 6.54 (d, J=16.0 Hz, 1H), 7.08-7.17 (m, 2H), 7.18-7.27 (m, 2H). LCMS (ESI+): m/z=538.2 (M+1), RT: 2.402 min. LC/MS: Kinetex® EVO C18 3.0×50 mm 2.6 μm. Detection methods are PDA. MS mode was positive electrospray ionization. MS range was 50-1050. Mobile phase: Ramp from 5% ACN (0.01875% TFA) in water (0.0375% TFA) to 95% ACN in 2.40 min, Flow rate is set at 1.2 mL/min; then hold at 95% ACN for 0.30 minutes, Flow rate is set at 1.2 mL/min; return back to 5% ACN in water and hold for 0.30 min. Flow rate is set at 1.2 mL/min.

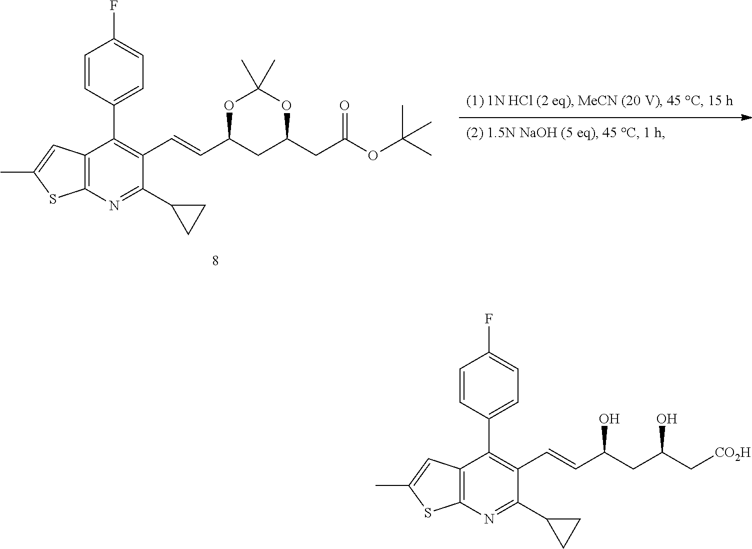 [SMILES: Cc1cc2c(-c3ccc(F)cc3)c(/C=C/[C@@H]3C[C@H](CC(=O)OC(C)(C)C)OC(C)(C)O3)c(C3CC3)nc2s1 ; Cc1cc2c(-c3ccc(F)cc3)c(/C=C/[C@@H](O)C[C@@H](O)CC(=O)O)c(C3CC3)nc2s1]

Step 8: To a solution of compound 8 (200 mg, 367.43 μmol, 1 eq) in MeCN (2 mL) was added HCl (1 M, 734.86 μL, 2 eq). The mixture was stirred at 45 °C for 12 hours. Then NaOH (1.5 M, 1.22 mL, 5 eq) was added to the solution. The mixture was stirred at 45 °C for 1 hour. LCMS showed reactant was consumed completely and one peak with 100% desired mass was detected. The mixture was concentrated under reduced pressure to give a residue. The residue was purified by prep-HPLC (column: CD02-Waters Xbidge BEH C18 150*25*10 μm; mobile phase: [water (NH₃H₂O)-ACN]; gradient: 15%-35% B over 10 min) and lyophilized directly to afford (3R,5S,E)-7-(6-cyclopropyl-4-(4-fluorophenyl)-2-methylthieno[2,3-b]pyridin-5-yl)-3,5-dihydroxyhept-6-enoic acid (68.22 mg, 37.44% yield) as white solid. ¹H NMR: (400 MHz, CDCl₃) δ ppm 0.70-0.87 (m, 2H), 0.98-1.14 (m, 2H), 1.21-1.45 (m, 2H), 2.14-2.26 (m, 2H), 2.27-2.36 (m, 1H), 2.37-2.47 (m, 3H), 3.98-4.37 (m, 2H), 5.38 (dd, J=16.0, 5.2 Hz, 1H), 6.34-6.49 (m, 2H), 6.89-7.01 (m, 2H), 7.01-7.12 (m, 2H). LCMS (ESI+): m/z=442.0 (M+1), RT: 1.403 min. LC/MS: Kinetex® XBridge C18 3.0*50 mm, 5 μm. Detection methods are PDA. MS mode was positive electrospray ionization. MS range was 50-1050. Mobile phase: Ramp from 5% ACN in water (0.025% NH₃·H₂O) to 95% ACN in 3.00 min, Flow rate is set at 0.9 mL/min; then hold at 95% ACN for 0.70 minutes, Flow rate is set at 0.9 mL/min; return back to 5% ACN in water and hold for 0.30 min. Flow rate is set at 1.2 mL/min.

### Example 6. Synthesis of (3R,5S,E)-7-(6-cyclopropyl-4-(4-fluorophenyl)-3-methylthieno[2,3-b]pyridin-5-yl)-3,5-dihydroxyhept-6-enoic acid sodium salt

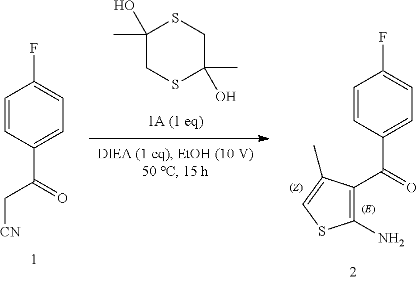

Step 1: A mixture of compound 1 (10.0 g, 61.29 mmol, 1 eq), compound 1A (11.05 g, 61.29 mmol, 1 eq) and DIEA (7.92 g, 61.29 mmol, 10.68 mL, 1 eq) in EtOH (100 mL) was degassed and purged with N₂ for 3 times, and then the mixture was stirred at 50 °C for 15 hours under N₂ atmosphere. LCMS showed 63% of desired compound was detected. The mixture was concentrated under reduced pressure. The residue was diluted with H₂O (50 mL) and extracted with DCM (100 mL×3). The combined organic layers were washed with brine (100 mL×2), dried over Na₂SO₄, filtered and concentrated under reduced pressure. The crude product was triturated with PE/EtOAc=10:1 (50 mL) and filtered. The cake was dried under reduced pressure to give compound 2 (6.8 g, 44.80% yield) as a yellow solid. LCMS (ESI+): m/z=235.9.0 (M+23), RT: 0.446 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 min, Flow rate is set at 2.0 mL/min; return back to 5% ACN in water and hold for 0.02 min, Flow rate is set at 2.0 mL/min.

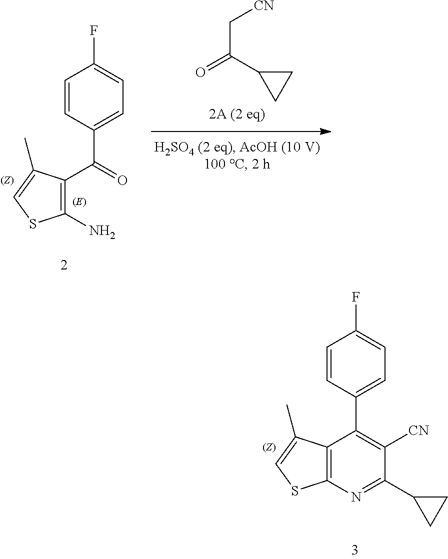

Step 2: To a solution of compound 2 (1.6 g, 6.80 mmol, 1 eq) and compound 2A (1.48 g, 13.60 mmol, 2 eq) in AcOH (15 mL) was added concentrated H₂SO₄ (1.33 g, 13.60 mmol, 724.98 μL, 2 eq) and the mixture was stirred at 100 °C for 2 hours. LCMS showed the reactant was consumed completely and 58% of desired product was observed. The mixture was poured into saturated aqueous Na₂CO₃ (200 mL) and extracted with EtOAc (200 mL×3). The combined organic layers were dried over anhydrous Na₂SO₄ and concentrated under reduced pressure. The crude product was purified by flash column chromatography on silica gel (0-5% EtOAc in PE) to give compound 3 (1.25 g, 59.61% yield) as a yellow solid. LCMS (ESI+): m/z=309.1 (M+1), RT: 0.529 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 min, Flow rate is set at 2.0 mL/min; return back to 5% ACN in water and hold for 0.02 min, Flow rate is set at 2.0 mL/min.

In some embodiments, compound 2 is the sodium salt of compound 2.

In some embodiments, compound 3 is the sodium salt of compound 3.

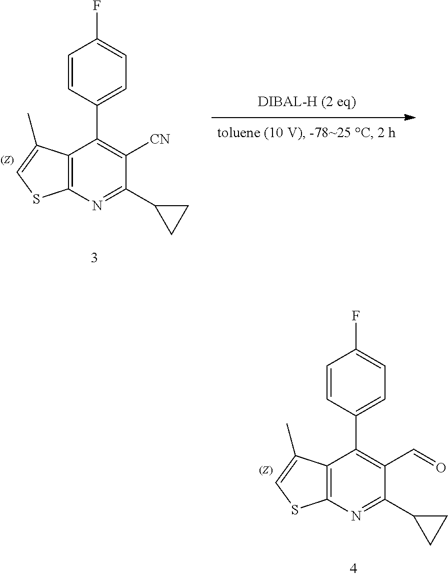 [SMILES: Cc1csc2nc(C3CC3)c(C#N)c(-c3ccc(F)cc3)c12 ; Cc1csc2nc(C3CC3)c(C=O)c(-c3ccc(F)cc3)c12]

Step 3: To a solution of compound 3 (1.25 g, 4.05 mmol, 1 eq) in toluene (10 mL) was added DIBAL-H (1 M, 8.11 mL, 2 eq) at −78 °C and the mixture was stirred at −78 °C under N₂ for 0.5 hour. The mixture was allowed to warm up to 25 °C and stirred for 2 hours under N₂. LCMS showed the reactant was consumed completely and 58% of desired product was observed. The reaction was quenched with MeOH (10 mL) at 0 °C and the resulting mixture was filtered. The filtrate was concentrated under reduced pressure. The crude product was purified by flash column chromatography on silica gel (0-5% EtOAc in PE) to give compound 4 (400 mg, 31.69% yield) as a yellow solid. LCMS (ESI+): m/z=312.1 (M+1), RT: 0.531 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 min, Flow rate is set at 2.0 mL/min; return back to 5% ACN in water and hold for 0.02 min, Flow rate is set at 2.0 mL/min.

In some embodiments, compound 4 is the sodium salt of compound 4.

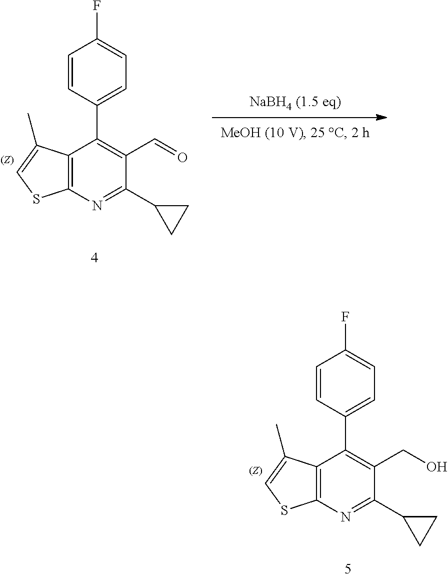 [SMILES: Cc1csc2nc(C3CC3)c(C=O)c(-c3ccc(F)cc3)c12 ; Cc1csc2nc(C3CC3)c(CO)c(-c3ccc(F)cc3)c12]

Step 4: To a solution of compound 4 (400 mg, 1.28 mmol, 1 eq) in MeOH (4 mL) was added NaBH₄ (72.90 mg, 1.93 mmol, 1.5 eq) and the mixture was stirred at 25 °C under N₂ for 2 hours. LCMS showed the reactant was consumed completely and 92% of desired product was observed. The reaction was quenched with saturated aqueous NH₄Cl (20 mL) and the mixture was extracted with EtOAc (30 mL×3). The combined organic layers were concentrated under reduced pressure. The crude product was purified by flash column chromatography on silica gel (0-10% EtOAc in PE) to give compound 5 (355 mg, 88.18% yield) as yellow oil. LCMS (ESI+): m/z=314.1 (M+1), RT: 0.453 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 min, Flow rate is set at 2.0 mL/min; return back to 5% ACN in water and hold for 0.02 min, Flow rate is set at 2.0 mL/min.

In some embodiments, compound 5 is the sodium salt of compound 5.

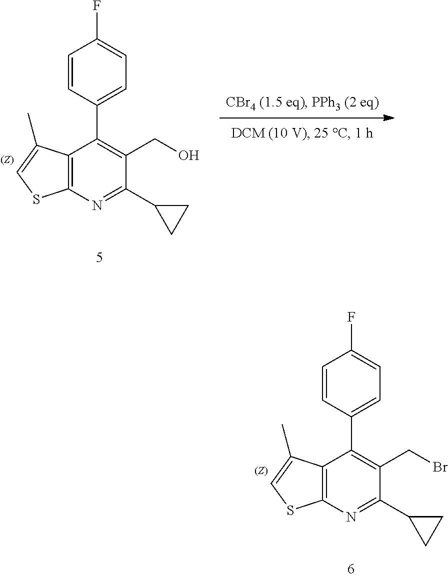 [SMILES: Cc1csc2nc(C3CC3)c(CO)c(-c3ccc(F)cc3)c12 ; Cc1csc2nc(C3CC3)c(CBr)c(-c3ccc(F)cc3)c12]

Step 5: To a solution of compound 5 (355 mg, 1.13 mmol, 1 eq) in DCM (5 mL) was added PPh₃ (594.22 mg, 2.27 mmol, 2 eq) and CBr₄ (563.49 mg, 1.70 mmol, 1.5 eq) at 0 °C and the mixture was stirred at 25 °C for 1 hour. LCMS showed the reactant was consumed completely and 80% of desired product was observed. The mixture was concentrated under reduced pressure. The crude product was purified by flash column chromatography on silica gel (0-5% EtOAc in PE) to give compound 6 (256 mg, 60.06% yield) as a white solid. LCMS (ESI+): m/z=376.0 (M+1), RT: 0.570 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 min, Flow rate is set at 2.0 mL/min; return back to 5% ACN in water and hold for 0.02 min, Flow rate is set at 2.0 mL/min.

In some embodiments, compound 6 is the sodium salt of compound 6.

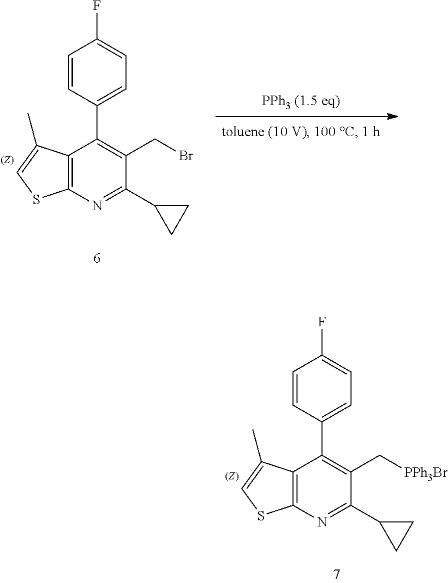

Step 6: To a solution of compound 6 (256 mg, 680.34 μmol, 1 eq) in toluene (3 mL) was added PPh₃ (267.66 mg, 1.02 mmol, 1.5 eq) and the mixture was stirred at 100 °C for 1 hour. LCMS showed the reactant was consumed completely and 66% of desired product was observed. The mixture was concentrated under reduced pressure to give compound 7 (434 mg, crude) as a white solid. LCMS (ESI+): m/z=558.2 (M−79), RT: 0.455 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 min, Flow rate is set at 2.0 mL/min; return back to 5% ACN in water and hold for 0.02 min, Flow rate is set at 2.0 mL/min.

In some embodiments, compound 7 is the sodium salt of compound 7.

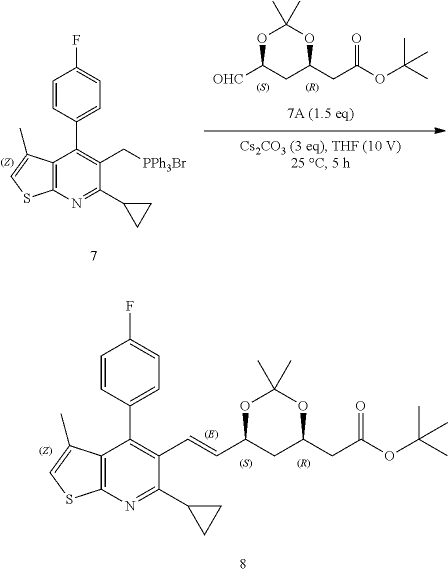

Step 7: To a solution of compound 7 (434 mg, 679.64 μmol, 1 eq), compound 7A (263.34 mg, 1.02 mmol, 1.5 eq) in THF (3 mL) was added Cs₂CO₃ (664.32 mg, 2.04 mmol, 3 eq) and the mixture was stirred at 25 °C for 5 hours. LCMS showed the reactant was consumed completely and 55% of desired product was observed. The reaction was quenched with water (20 mL) and extracted with EtOAc (20 mL×3). The combined organic layers were dried over anhydrous Na₂SO₄ and concentrated under reduced pressure. The crude product was purified by flash column chromatography on silica gel (0-5% EtOAc in PE) to give compound 8 (177 mg, 48.44% yield) as colorless oil. LCMS (ESI+): m/z=538.3 (M+1), RT: 0.623 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 min, Flow rate is set at 2.0 mL/min; return back to 5% ACN in water and hold for 0.02 min, Flow rate is set at 2.0 mL/min. 1H NMR: (400 MHz, CDCl₃) δ 0.83-0.90 (m, 2H), 0.95-1.02 (m, 3H), 1.22 (dd, J=4.8, 3.2 Hz, 1H), 1.36 (s, 3H), 1.45 (s, 3H), 1.46 (s, 9H), 1.65 (d, J=0.8 Hz, 3H), 2.22-2.30 (m, 1H), 2.32-2.46 (m, 2H), 4.18-4.27 (m, 1H), 4.27-4.37 (m, 1H), 5.57 (dd, J=16.4, 6.0 Hz, 1H), 6.33-6.40 (m, 1H), 6.92 (d, J=0.8 Hz, 1H), 7.06-7.19 (m, 4H).

In some embodiments, compound 8 is the sodium salt of compound 8.

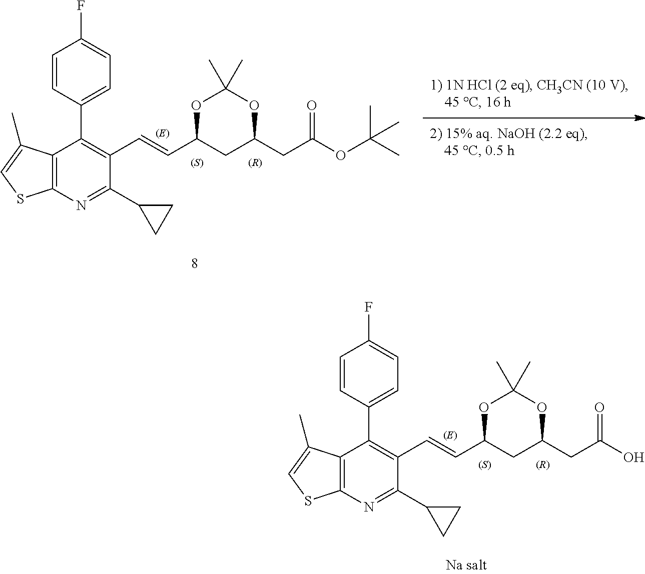

Step 8: To a solution of compound 8 (177 mg, 329.19 μmol, 1 eq) in ACN (2 mL) was added HCl (1 M, 658.38 μL, 2 eq) and the mixture was stirred at 45 °C for 16 hours. Aqueous NaOH (193.11 mg, 724.22 μmol, 15% purity, 2.2 eq) was added and the mixture was stirred at 45 °C for 0.5 hour. LCMS showed the reactant was consumed completely and 99% of desired product was observed. The mixture was purified by prep-HPLC (column: CD02-Waters Xbidge BEH C18 150*25*10 μm; mobile phase: [water (NH₃·H₂O)-ACN]; gradient: 11%-41% B over 10 min) to afford (3R,5S,E)-7-(6-cyclopropyl-4-(4-fluorophenyl)-3-methylthieno[2,3-b]pyridin-5-yl)-3,5-dihydroxyhept-6-enoic acid sodium salt (104.80 mg, 72.11% yield) as a white solid. ¹H NMR: (400 MHz, CDCl₃) δ 0.85 (s, 2H), 1.13 (s, 2H), 1.23 (d, J=0.8 Hz, 1H), 1.30-1.44 (m, 1H), 1.46-1.54 (m, 3H), 2.09-2.17 (m, 1H), 2.18-2.28 (m, 2H), 3.95-4.08 (m, 1H), 4.10-4.36 (m, 1H), 5.39-5.52 (m, 1H), 6.23-6.38 (m, 1H), 6.72-6.83 (m, 1H), 6.84-7.01 (m, 4H). LCMS (ESI+): m/z=442.1 (M+1), RT: 1.365 min. LC/MS: Kinetex® EVO C18 3.0×50 mm 2.6 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in 3.40 min, Flow rate is set at 0.9 mL/min; then hold at 95% ACN for 0.30 min, Flow rate is set at 0.9 mL/min; return back to 5% ACN in water and hold for 0.30 min, Flow rate is set at 1.2 mL/min.

### Example 7. Synthesis of (3R,5S,E)-7-(5-cyclopropyl-7-(4-fluorophenyl)-2-methylthieno[3,2-b]pyridin-6-yl)-3,5-dihydroxyhept-6-enoic acid sodium salt

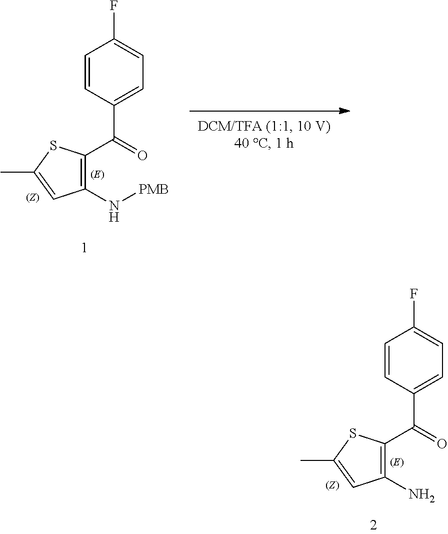

Step 1: A mixture of compound 1 (28 g, 79.98 mmol, 1 eq) in DCM (140 mL) and TFA (140 mL) was degassed and purged with N₂ for 3 times, and the mixture was stirred at 40 °C for 1 hour under N₂ atmosphere. LCMS showed the starting material was consumed completely and desired MS was observed. After cooling to room temperature, the reaction mixture was concentrated under reduced pressure. The residue was purified by flash column chromatography on silica gel (0 to 20% EtOAc in PE) to afford compound 2 (12 g, 64.74% yield) as a yellow solid.

LCMS (ESI+): m/z=236.0 (M+1), RT: 0.417 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

In some embodiments, compound 1 is the sodium salt of compound 1.

In some embodiments, compound 2 is the sodium salt of compound 2.

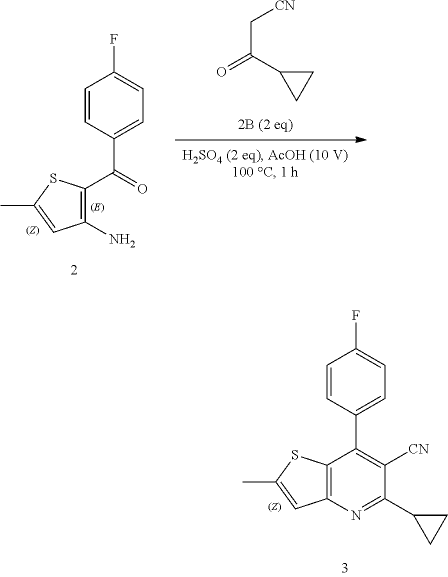 [SMILES: Cc1cc(N)c(C(=O)c2ccc(F)cc2)s1 ; N#CCC(=O)C1CC1 ; Cc1cc2nc(C3CC3)c(C#N)c(-c3ccc(F)cc3)c2s1]

Step 2: A mixture of compound 2 (6.6 g, 28.06 mmol, 1 eq), compound 2B (6.12 g, 56.10 mmol, 2 eq) and H₂SO₄ (5.50 g, 56.10 mmol, 2 eq) in AcOH (80 mL) was degassed and purged with N₂ for 3 times, then the mixture was stirred at 100 °C for 1 hour under N₂ atmosphere. LCMS showed desired MS was observed. After cooling to room temperature, the reaction mixture was diluted with H₂O (100 mL) and extracted with EtOAc (100 mL×3). The combined organic layers were washed with brine (100 mL×3), dried over Na₂SO₄, filtered and concentrated under reduced pressure. The residue was purified by flash column chromatography on silica gel (0 to 10% EtOAc in PE) to afford compound 3 (4.4 g, 50.86% yield) as a yellow solid. LCMS (ESI+): m/z=309.1 (M+1), RT: 0.517 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

In some embodiments, compound 3 is the sodium salt of compound 3.

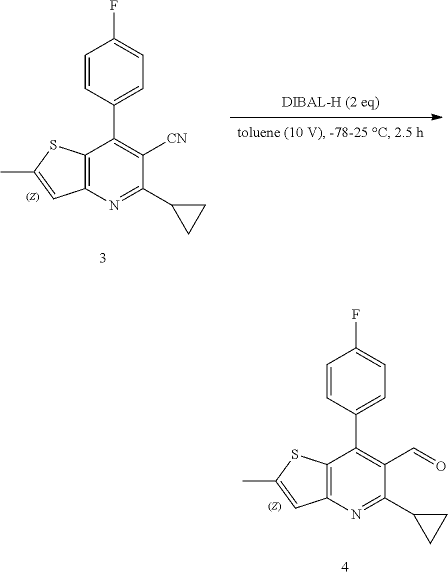 [SMILES: Cc1cc2nc(C3CC3)c(C#N)c(-c3ccc(F)cc3)c2s1 ; Cc1cc2nc(C3CC3)c(C=O)c(-c3ccc(F)cc3)c2s1]

Step 3: To a solution of compound 3 (1.8 g, 5.84 mmol, 1 eq) in toluene (20 mL) was added DIBAL-H (1 M, 11.67 mL, 2 eq) at −78 °C and the mixture was stirred at −78 °C under N₂ for 0.5 hour. The mixture was allowed to warm to 25 °C and stirred for 2 hours under N₂. LCMS showed the desired MS was observed. The reaction was quenched by adding CH₃OH (20 mL) at 0 °C and the resulting mixture was filtered. The filtrate was concentrated under reduced pressure. The residue was purified by flash column chromatography on silica gel (0 to 6% EtOAc in PE) to afford compound 4 (760 mg, 41.82% yield) as a yellow solid. LCMS (ESI+): m/z=312.0 (M+1), RT: 0.487 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

In some embodiments, compound 4 is the sodium salt of compound 4.

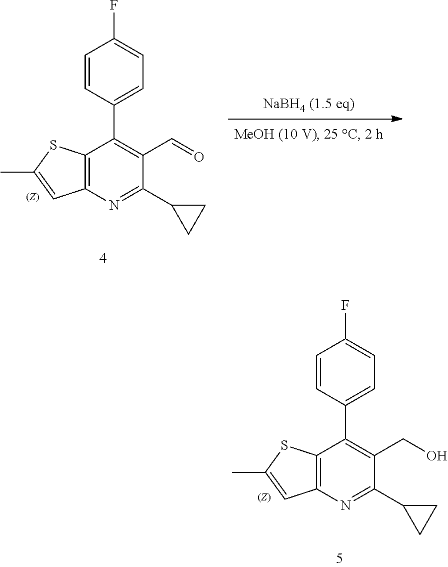 [SMILES: Cc1cc2nc(C3CC3)c(C=O)c(-c3ccc(F)cc3)c2s1 ; Cc1cc2nc(C3CC3)c(CO)c(-c3ccc(F)cc3)c2s1]

Step 4: To a solution of compound 4 (760 mg, 2.44 mmol, 1 eq) in MeOH (8 mL) was added NaBH₄ (138.51 mg, 3.66 mmol, 1.5 eq) and the mixture was stirred at 25 °C under N₂ for 2 hour. LCMS showed the starting material was consumed completely and desired MS was observed. The reaction was quenched by adding saturated NH₄Cl (20 mL) and the mixture was extracted with EtOAc (20 mL×3). The combined organic layers were concentrated under reduced pressure. The residue was purified by flash column chromatography on silica gel (0 to 20% EtOAc in PE) to afford compound 5 (610 mg, 79.75% yield) as a white solid. LCMS (ESI+): m/z=314.5 (M+1), RT: 0.322 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

In some embodiments, compound 5 is the sodium salt of compound 5.

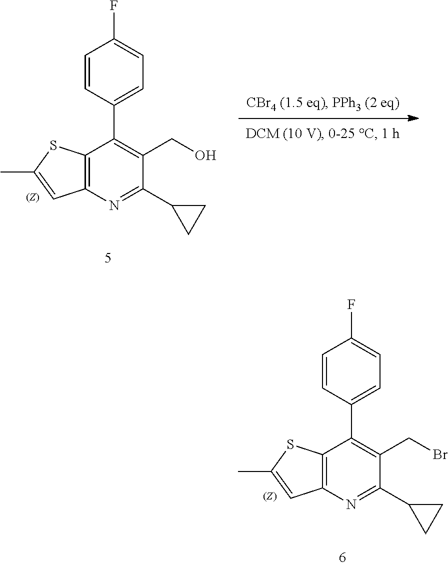 [SMILES: Cc1cc2nc(C3CC3)c(CO)c(-c3ccc(F)cc3)c2s1 ; Cc1cc2nc(C3CC3)c(CBr)c(-c3ccc(F)cc3)c2s1]

Step 5: To a solution of compound 5 (0.61 g, 1.95 mmol, 1 eq) in DCM (8 mL) was added CBr₄ (968.25 mg, 2.92 mmol, 1.5 eq) and PPh₃ (1.02 g, 3.89 mmol, 2 eq) at 0 °C. The resulting mixture was stirred at 25 °C for 1 hour under N₂ atmosphere. LCMS showed the desired MS was observed. The reaction was concentrated under reduced pressure. The residue was purified by flash column chromatography on silica gel (0 to 5% EtOAc in PE) to afford compound 6 (770 mg, 50% purity 52.57% yield) as a white solid. LCMS (ESI+): m/z=376.0 (M+1), RT: 0.483 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

In some embodiments, compound 6 is the sodium salt of compound 6.

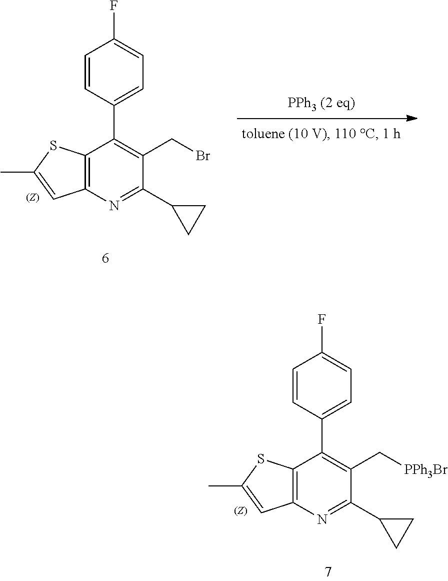

Step 6: To a solution of compound 6 (0.77 g, 1.02 mmol, 1 eq) in toluene (10 mL) was added PPh₃ (268.36 mg, 1.02 mmol, 1 eq), then the mixture was stirred at 110 °C for 1 hour under N₂ atmosphere. LCMS showed the starting material was consumed completely and desired product was observed. After cooling to room temperature, the reaction mixture was concentrated under reduced pressure to give compound 7 (655 mg, crude) as colorless liquid. LCMS (ESI+): m/z=558.2 (M-Br), RT: 0.433 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

In some embodiments, compound 7 is the sodium salt of compound 7.

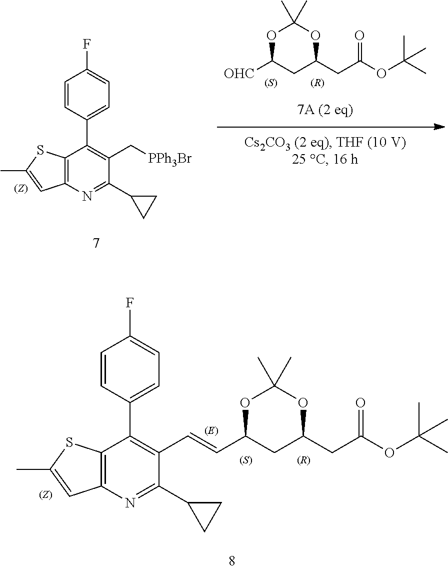

Step 7: To a solution of compound 7 (655 mg, 1.03 mmol, 1 eq) and compound 7A (397.43 mg, 1.54 mmol, 1.5 eq) in THF (10 mL) was added Cs₂CO₃ (668.40 mg, 2.05 mmol, 2 eq), then the mixture was stirred at 25 °C for 16 hours under N₂ atmosphere. LCMS showed the starting material was consumed completely and desired MS was observed. The reaction was concentrated under reduced pressure. The residue was purified by flash column chromatography on silica gel (0 to 6% EtOAc in PE) to afford compound 8 (210 mg, 37.70% yield) as a white solid. ¹H NMR: (400 MHz, CDCl₃) δ 0.90-1.00 (m, 3H), 1.07-1.28 (m, 3H), 1.32 (s, 3H), 1.41 (s, 12H), 2.17-2.43 (m, 3H), 2.50 (s, 3H), 4.15-4.25 (m, 1H), 4.32 (td, J=5.6, 4.4 Hz, 1H), 5.48 (dd, J=16.0, 6.0 Hz, 1H), 6.56 (d, J=16.0 Hz, 1H), 7.03-7.12 (m, 3H), 7.22 (s, 1H), 7.26-7.30 (m, 1H). LCMS (ESI+): m/z=538.4 (M+1), RT: 0.506 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

In some embodiments, compound 8 is the sodium salt of compound 8.

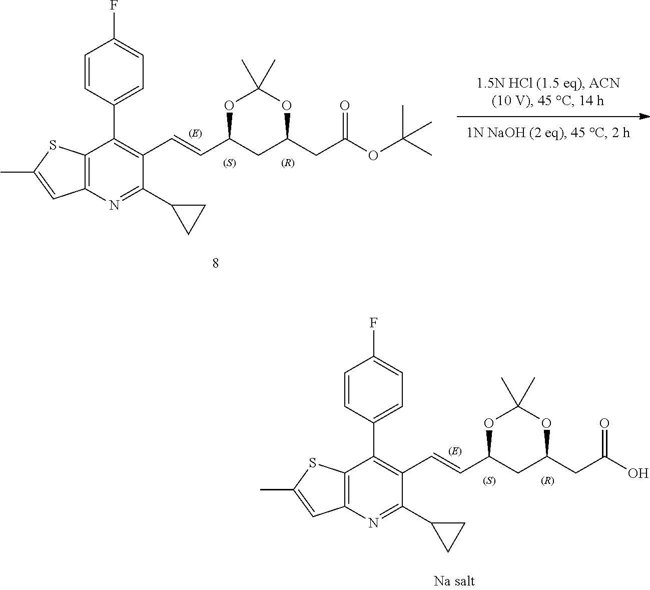

Step 8: To a solution compound 8 (130 mg, 241.78 μmol, 1 eq) in ACN (2 mL) was added HCl (1.5 M, 1.5 eq), then the mixture was stirred at 45 °C for 14 hours under N₂ atmosphere. NaOH (1 M, 2 eq) was added and stirred at 45 °C for 2 hours under N₂ atmosphere. LCMS showed the starting material was consumed completely and desired MS was observed. The reaction mixture was purified by prep-HPLC (column: CD02-Waters Xbidge BEH C18 150*25*10 μm; mobile phase: [water (NH₃H₂O)-ACN]; gradient: 12%-32% B over 10 min) to afford (3R,5S,E)-7-(5-cyclopropyl-7-(4-fluorophenyl)-2-methylthieno[3,2-b]pyridin-6-yl)-3,5-dihydroxyhept-6-enoic acid sodium salt (50.30 mg, 46.65% yield) as a white solid. 1H NMR: (400 MHz, DMSO-d₆) δ 0.96 (dd, J=7.6, 3.6 Hz, 2H), 1.05 (d, J=4.4 Hz, 2H), 1.09-1.18 (m, 1H), 1.35-1.46 (m, 1H), 1.87 (dd, J=14.8, 8.4 Hz, 1H), 2.04 (dd, J=14.8, 4.0 Hz, 1H), 2.37-2.45 (m, 1H), 2.52 (s, 3H), 3.59 (tt, J=8.4, 4.0 Hz, 1H), 4.13 (q, J=6.0 Hz, 1H), 5.54 (dd, J=16.0, 5.6 Hz, 1H), 6.51 (d, J=16.0 Hz, 1H), 7.15 (d, J=0.8 Hz, 1H), 7.27-7.36 (m, 2H), 7.38-7.46 (m, 2H). LCMS (ESI+): m/z=442.2 (M+1), RT: 1.358 min. LC/MS: Kinetex® EVO C18 3.0×50 mm 2.6 μm Detection methods are photo-diode array (PDA&ELSD). MS mode was positive electrospray ionization. MS range was 50-1050. Mobile phase: Ramp from 5% ACN (0.01875% TFA) in water (0.0375% TFA) to 95% ACN in water in 3.4 min, Flow rate is set at 0.9 mL/min; then hold at 95% ACN for 0.3 minutes Flow rate is set 0.9 mL/min; return back to 5% ACN in water and hold for 0.3 min. Flow rate is set at 1.2 mL/min.

### Example 8. Synthesis of (3R,5S,E)-7-(6-cyclopropyl-4-(4-fluorophenyl)-2-methylbenzo[d]thiazol-5-yl)-3,5-dihydroxyhept-6-enoic acid sodium salt

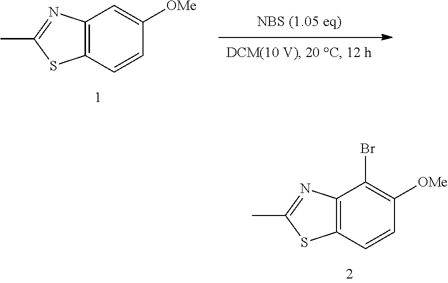

Step 1: To a solution of compound 1 (10 g, 55.79 mmol, 1 eq) in DCM (100 mL) was added NBS (10.43 g, 58.58 mmol, 1.05 eq). The mixture was stirred at 20 °C for 12 hours. The reaction mixture was combined with a paralleled batch (10 g compound 1 was used), quenched by adding sat. Na₂SO₃ (100 mL) and extracted with DCM (200 mL×2). The combined organic layers were dried over Na₂SO₄, filtered and concentrated under reduced pressure. The residue was purified by column chromatography on silica gel (PE/EtOAc=40/1 to 1/1) to give compound 2 (17.5 g, 60.15% yield) as a white solid. LCMS (ESI+): m/z=258.0 (M+1), RT: 0.415 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

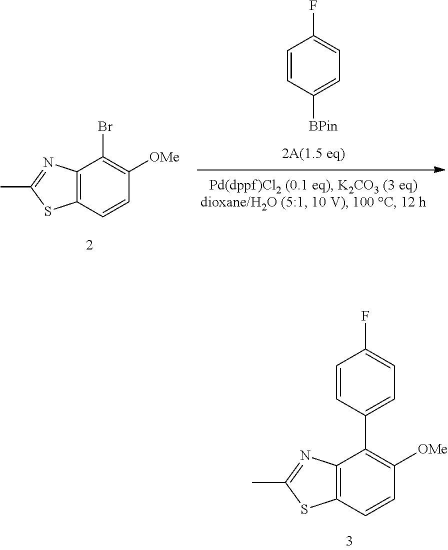

Step 2: To a solution of compound 2 (5 g, 19.37 mmol, 1 eq) in dioxane (50 mL) and H₂O (10 mL) was added K₂CO₃ (8.03 g, 58.11 mmol, 3 eq) and compound 2A (6.45 g, 29.05 mmol, 1.5 eq) and Pd(dppf)Cl₂ (1.42 g, 1.94 mmol, 0.1 eq). The mixture was stirred at 100 °C for 12 hours. The reaction mixture was combined with a paralleled batch (5 g compound 2 was used), diluted with H₂O (300 mL) and extracted with EtOAc (150 mL×2). The combined organic layers were dried over Na₂SO₄, filtered and concentrated under reduced pressure. The residue was purified by column chromatography on silica gel (PE/EtOAc=1/0 to 4/1) to give compound 3 (9 g, 85.00% yield, 100% purity) as a white solid. LCMS (ESI+): m/z=274.0 (M+1), RT: 0.486 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

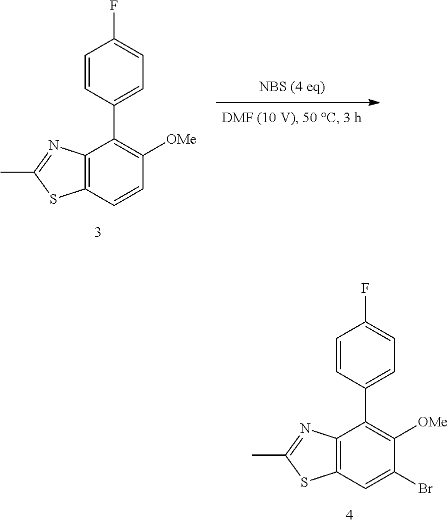 [SMILES: COc1ccc2sc(C)nc2c1-c1ccc(F)cc1 ; COc1c(Br)cc2sc(C)nc2c1-c1ccc(F)cc1]

Step 3: To a solution of compound 3 (3 g, 10.98 mmol, 1 eq) in DMF (30 mL) was added NBS (7.81 g, 43.90 mmol, 4 eq) and the mixture was stirred at 50 °C for 3 hours. The mixture was diluted with H₂O (100 mL) and extracted with EtOAc (130 mL×2). The combined organic layers were washed with brine (200 mL×2), dried over Na₂SO₄, filtered and concentrated under reduced pressure. The residue was purified by column chromatography on silica gel (PE/EtOAc=1/0 to 50/1) to give compound 4 (3.5 g, 89.63% yield) as a white solid. LCMS (ESI+): m/z=351.9 (M+1), RT: 0.532 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

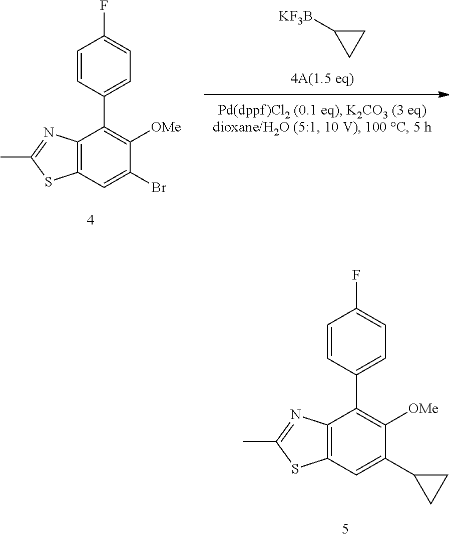

Step 4: To a solution of compound 4 (3.5 g, 9.94 mmol, 1 eq) in dioxane (35 mL) and H₂O (7 mL) was added K₂CO₃ (4.12 g, 29.81 mmol, 3 eq) and compound 4A (2.21 g, 14.91 mmol, 1.5 eq) and Pd(dppf)Cl₂ (727.10 mg, 993.69 μmol, 0.1 eq). The mixture was stirred at 100 °C for 5 hours. The mixture was diluted with H₂O (200 mL) and extracted with EtOAc (150 mL×2). The combined organic layers were dried over Na₂SO₄, filtered and concentrated under reduced pressure. The residue was purified by column chromatography on silica gel (PE/EtOAc=1/0 to 50/1) to give compound 5 (2.4 g, 77% yield) as a white solid. LCMS (ESI+): m/z=314.0 (M+1), RT: 0.533 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

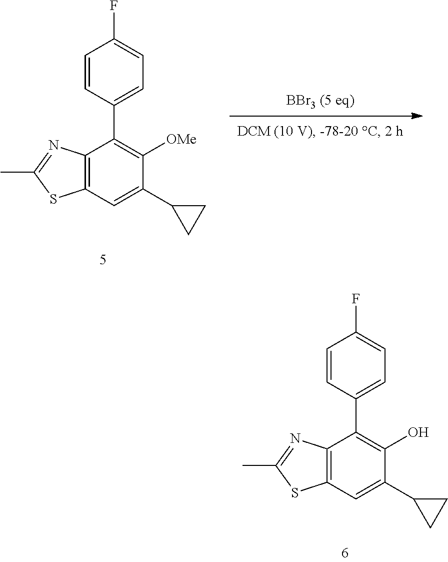 [SMILES: COc1c(C2CC2)cc2sc(C)nc2c1-c1ccc(F)cc1 ; Cc1nc2c(-c3ccc(F)cc3)c(O)c(C3CC3)cc2s1]

Step 5: To a solution of compound 5 (3.5 g, 11.17 mmol, 1 eq) in DCM (35 mL) was added BBr₃ (13.99 g, 55.84 mmol, 5.38 mL, 5 eq) at −78 °C under N₂. The mixture was stirred at −78 °C for 0.25 hour. Then the reaction mixture was allowed to warm to 20 °C and stirred for 1.75 hours. The reaction was combined with a paralleled batch (1.5 g compound 5 was used), quenched by adding ice-water (100 mL). The mixture was diluted with sat. NaHCO₃ (100 mL) and extracted with DCM (100 mL×2). The combined organic layers were dried over Na₂SO₄, filtered and concentrated under reduced pressure. The crude product was purified by column chromatography on silica gel (PE/EtOAc=1/0 to 35/1) to give compound 6 (2.3 g, 48% yield) as a yellow solid. LCMS (ESI+): m/z=300.0 (M+1), RT: 0.478 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

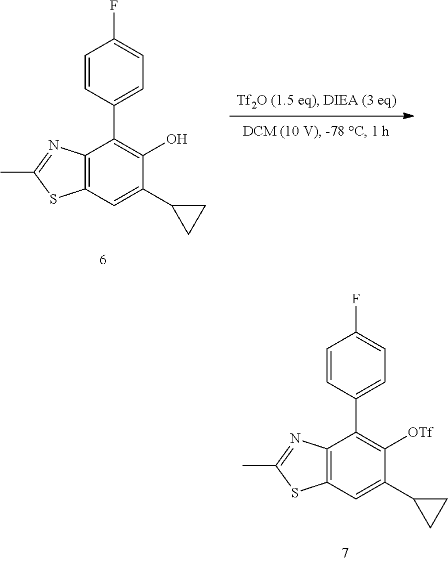 [SMILES: Cc1nc2c(-c3ccc(F)cc3)c(O)c(C3CC3)cc2s1 ; Cc1nc2c(-c3ccc(F)cc3)c(OS(=O)(=O)C(F)(F)F)c(C3CC3)cc2s1]

Step 6: To a solution of compound 6 (2.03 g, 6.78 mmol, 1 eq) in DCM (20 mL) was added DIEA (2.63 g, 20.34 mmol, 3.54 mL, 3 eq) and Tf₂O (2.87 g, 10.17 mmol, 1.68 mL, 1.5 eq) and the mixture was stirred at −78 °C for 1 hour. The mixture was diluted with H₂O (150 mL) and extracted with EtOAc (200 mL×2). The combined organic layers were dried over Na₂SO₄, filtered and concentrated under reduced pressure. The residue was purified by column chromatography on silica gel (PE/EtOAc=1/0 to 80/1) to give compound 7 (2.7 g, 94.74% yield) as a yellow solid. LCMS (ESI+): m/z=431.9 (M+1), RT: 0.568 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

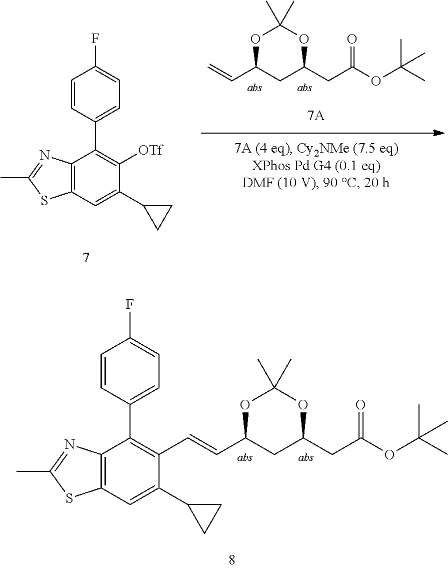 [SMILES: Cc1nc2c(-c3ccc(F)cc3)c(OS(=O)(=O)C(F)(F)F)c(C3CC3)cc2s1 ; C=C[C@@H]1C[C@H](CC(=O)OC(C)(C)C)OC(C)(C)O1 ; Cc1nc2c(-c3ccc(F)cc3)c(/C=C/[C@@H]3C[C@H](CC(=O)OC(C)(C)C)OC(C)(C)O3)c(C3CC3)cc2s1]

Step 7: To a solution of compound 7 (0.5 g, 1.16 mmol, 1 eq) and compound 7A (1.19 g, 4.64 mmol, 4 eq) in DMF (5 mL) was added Xphos Pd G4 (99.72 mg, 115.90 μmol, 0.1 eq) and N-cyclohexyl-N-methyl-cyclohexanamine (1.70 g, 8.69 mmol, 1.84 mL, 7.5 eq). The mixture was stirred at 90 °C for 20 hours. The mixture was combined with a paralleled batch (0.5 g compound 7 was used), diluted with H₂O (60 mL) and extracted with EtOAc (60 mL×2). The combined organic layers were dried over Na₂SO₄, filtered and concentrated under reduced pressure. The crude product was purified by flash silica gel chromatography on silica gel (0~10% EtOAc in PE) and furtherly purified by prep-HPLC (column: Waters Xbridge BEH C18 150*25 mm*5 μm; mobile phase: [water (NH₄HCO₃)-ACN]; gradient: 70%-100% B over 10 min) to give compound 8 (260 mg, 20.24% yield) as a white solid. ¹H NMR: (400 MHz, CDCl₃) δ 0.70-0.80 (m, 2H), 0.93-1.01 (m, 2H), 1.37-1.43 (m, 5H), 1.47 (s, 12H), 2.07-2.15 (m, 1H), 2.24-2.31 (m, 1H), 2.39-2.46 (m, 1H), 2.73 (s, 3H), 4.20-4.29 (m, 1H), 4.30-4.38 (m, 1H), 5.44 (dd, J=16.0, 6.4 Hz, 1H), 6.61 (d, J=16.0 Hz, 1H), 7.10 (t, J=8.8 Hz, 2H), 7.32 (J=8.4, 5.75 Hz, 2H), 7.47 (s, 1H). LCMS (ESI+): m/z=538.2 (M+1), RT: 1.941 min. LC/MS: Kinetex® EVO C18 3.0×50 mm 2.6 μm. Detection methods are photo-diode array (PDA). MS mode was positive electrospray ionization. MS range was 50-1050. Mobile phase: Ramp from 30% ACN (0.01875% TFA) in water (0.0375% TFA) to 90% ACN in 2.00 min, Flow rate is set at 0.9 mL/min; then ramp from 90% ACN in water to 100% ACN in 1.70 min, Flow rate is set at 0.9 mL/min; return back to 30% ACN in water and hold for 0.30 min. Flow rate is set at 1.2 mL/min.

In some embodiments, compound 8 is the sodium salt of compound 8.

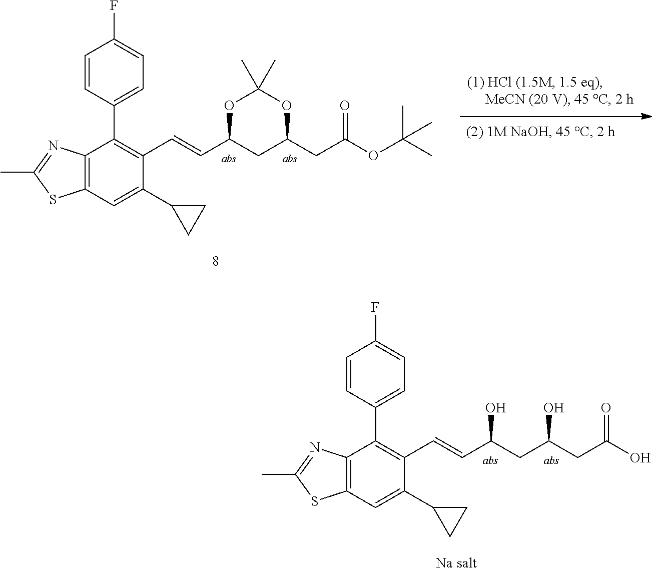

Step 8: To a solution of compound 8 (50 mg, 92.99 μmol, 1 eq) in MeCN (1.2 mL) was added HCl (1.5 M, 92.99 μL, 1.5 eq). The mixture was stirred at 45 °C for 2 hours. The mixture was adjusted with 1M NaOH to pH=9 and stirred at 45 °C for 2 hours. The reaction mixture was concentrated under reduced pressure. The crude product was purified by prep-HPLC (column: Waters Xbridge 150*25 mm*5 μm; mobile phase: [water (NH₃·H₂O)-ACN]; gradient: 3%-33% B over 10 min) to give (3R,5S,E)-7-(6-cyclopropyl-4-(4-fluorophenyl)-2-methylbenzo[d]thiazol-5-yl)-3,5-dihydroxyhept-6-enoic acid sodium salt (17 mg, 41.41% yield) as a pink solid. ¹H NMR: (400 MHz, DMSO-d₆) δ ppm 0.74-0.87 (m, 2H), 0.95-1.07 (m, 2H), 1.11-1.46 (m, 2H), 1.94-2.08 (m, 1H), 2.09-2.19 (m, 2H), 3.66-3.70 (m, 1H), 4.08-4.15 (m, 1H), 5.43-5.52 (m, 1H), 6.59 (d, J=16.0 Hz, 1H), 7.25-7.33 (m, 2H), 7.35-7.45 (m, 2H), 7.68 (s, 1H), 9.26 (s, 1H). LCMS (ESI+): m/z=442.1 (M+1), RT: 1.251 min. LC/MS: XBridge C18 3.0*50 mm, 5 μm. Detection methods are photo-diode array (PDA). MS mode was positive electrospray ionization. MS range was 50-1050. Mobile phase: Ramp from 5% ACN in water (0.025% NH₃·H₂O) to 95% ACN in 3.00 min, Flow rate is set at 0.9 mL/min; then hold at 95% ACN for 0.70 minutes Flow rate is set at 0.9 mL/min; return back to 5% ACN in water and hold for 0.30 min. Flow rate is set at 1.2 mL/min.

### Example 9. Synthesis of (3R,5S,E)-7-(5-cyclopropyl-7-(4-fluorophenyl)-2-methylbenzo[d]thiazol-6-yl)-3,5-dihydroxyhept-6-enoic acid sodium salt

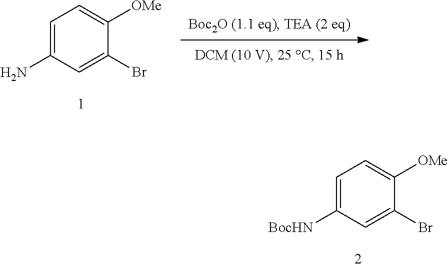 [SMILES: COc1ccc(N)cc1Br ; COc1ccc(NC(=O)OC(C)(C)C)cc1Br]

Step 1: A mixture of compound 1 (40 g, 197.97 mmol, 1 eq), Boc₂O (47.53 g, 217.77 mmol, 50.03 mL, 1.1 eq), Et₃N (40.07 g, 395.95 mmol, 55.11 mL, 2 eq) in DCM (400 mL) was degassed and purged with N₂ for 3 times, and then the mixture was stirred at 25 °C for 15 hours under N₂ atmosphere. TLC showed the starting material was consumed completely and a new spot with lower polarity was observed. The mixture was diluted with DCM (200 mL) and washed with H₂O (200 mL×3), dried over Na₂SO₄, filtered and concentrated under reduced pressure to give compound 2 (60 g, 66.20% yield) as a black solid. ¹H NMR: (400 MHz, DMSO-d₆) δ 1.46 (s, 9H), 3.78 (s, 3H), 7.02 (d, J=9.2 Hz, 1H), 7.32-7.39 (m, 1H), 7.73 (s, 1H), 9.30 (s, 1H).

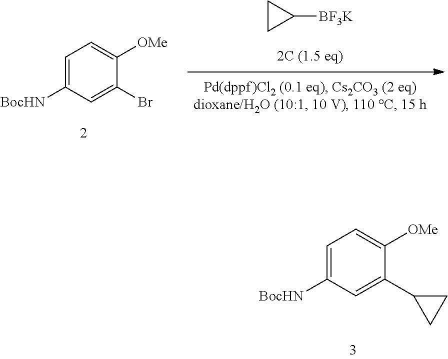

Step 2: A mixture of compound 2 (60 g, 131.05 mmol, 1 eq), compound 2C (29.09 g, 196.58 mmol, 1.5 eq), Pd(dppf)Cl₂ (9.59 g, 13.11 mmol, 0.1 eq) and Cs₂CO₃ (85.40 g, 262.11 mmol, 2 eq) in dioxane (600 mL) and H₂O (60 mL) was degassed and purged with N₂ for 3 times, and then the mixture was stirred at 110 °C for 15 hours under N₂ atmosphere. LCMS showed the starting material was consumed completely and desired product was observed. The reaction mixture was concentrated under reduced pressure. The residue was purified by flash column chromatography on silica gel (0~15% EtOAc in PE) to give compound 3 (34.1 g, 93.87% yield) as a white solid. LCMS (ESI+): m/z=207.9 (M−56+1), RT: 0.532 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

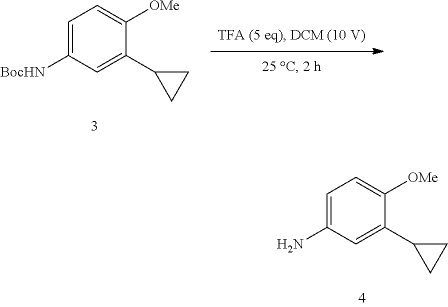 [SMILES: COc1ccc(NC(=O)OC(C)(C)C)cc1C1CC1 ; COc1ccc(N)cc1C1CC1]

Step 3: To a solution of compound 3 (34.1 g, 129.49 mmol, 1 eq) in DCM (300 mL) was added TFA (73.83 g, 647.47 mmol, 48.10 mL, 5 eq). The mixture was stirred at 25 °C for 2 hours. TLC showed the starting material was consumed completely and a new spot with larger polarity was observed. The reaction mixture was concentrated under reduced pressure to give compound 4 (30.2 g, 84.12% yield, TFA salt) as a black solid, which was used directly in the next step.

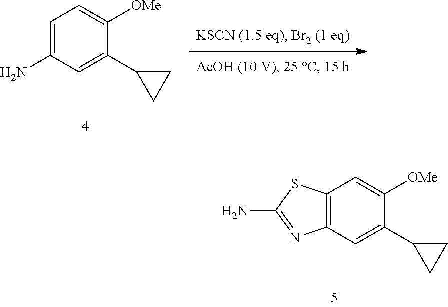 [SMILES: COc1ccc(N)cc1C1CC1 ; COc1cc2sc(N)nc2cc1C1CC1]

Step 4: To a solution of compound 4 (30.2 g, 108.94 mmol, 1 eq, TFA salt) in AcOH (150 mL) was added KSCN (18.88 g, 16.34 mmol, 15.84 mL, 1.5 eq). The mixture was stirred at 25 °C for 2 hours. This was followed by the addition of Br₂ (17.40 g, 108.94 mmol, 5.62 mL, 1 eq) in AcOH (300 mL) dropwise with stirring. The resulting mixture was stirred at 25 °C for 13 hours. TLC showed the starting material was consumed completely and a new main spot was observed. The reaction mixture was concentrated under reduced pressure. The resulting solution was diluted with 200 mL of H₂O. The pH value of the solution was adjusted to 10 with ammonia solution. The mixture was filtered, and the cake was washed with H₂O (100 mL). The cake was dried under reduced pressure to give (18.5 g, 72.47% yield) as a yellow solid. LCMS (ESI+): m/z=220.9 (M+1), RT: 0.313 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

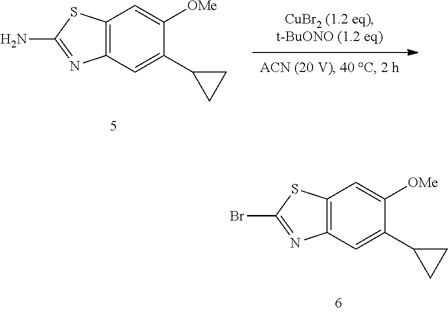 [SMILES: COc1cc2sc(N)nc2cc1C1CC1 ; COc1cc2sc(Br)nc2cc1C1CC1]

Step 5: A solution of CuBr₂ (12.17 g, 54.47 mmol, 2.55 mL, 1.2 eq) in CH₃CN (100 mL) was warmed to 40 °C, t-BuONO (5.62 g, 54.47 mmol, 6.48 mL, 1.2 eq) was slowly added, and the mixture was stirred for 1 hour. Compound 5 (10 g, 45.39 mmol, 1 eq) in CH₃CN (100 mL) was slowly added dropwise and the mixture was stirred at 40 °C for 2 hours. LCMS showed the starting material was consumed completely and desired product was observed. The mixture was quenched with HCl aqueous (0.5 M, 300 mL), extracted with EtOAc (3×100 mL). The combined organic layer was washed with saturated NaHCO₃ aqueous (400 mL) and brine (400 mL), dried with anhydrous Na₂SO₄, evaporated under reduced pressure to give compound 6 (8.9 g, 62.09% yield) as a black solid. LCMS (ESI+): m/z=285.7 (M+1), RT: 0.503 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

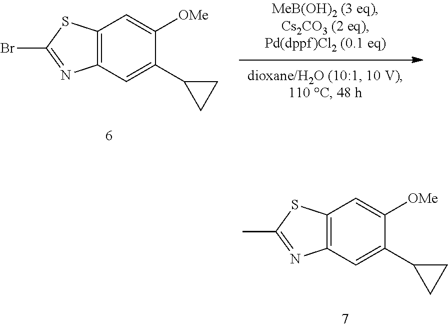

Step 6: A mixture of compound 6 (8.9 g, 28.19 mmol, 1 eq), Pd(dppf)Cl₂·CH₂Cl₂ (2.30 g, 2.82 mmol, 0.1 eq), MeB(OH)₂ (3.37 g, 56.37 mmol, 2 eq) and Cs₂CO₃ (27.55 g, 84.56 mmol, 3 eq) in dioxane (89 mL) and H₂O (8.9 mL) was degassed and purged with N₂ for 3 times, and then the mixture was stirred at 110 °C for 48 hours under N₂ atmosphere. TLC showed the starting material was consumed completely and a new spot with larger polarity was observed. The reaction was quenched by adding water (50 mL) and extracted with EtOAc (50 mL×3). The combined organic layers were dried over anhydrous Na₂SO₄ and concentrated under reduced pressure. The residue was purified by flash column chromatography on silica gel (0~15% EtOAc in PE) to afford compound 7 (3.9 g, 58.67% yield) as green oil. LCMS (ESI+): m/z=219.8 (M+1), RT: 0.467 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

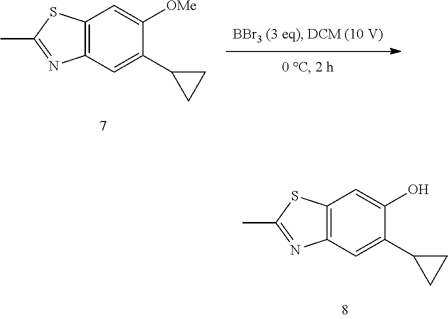

Step 7: To a solution of compound 7 (2.5 g, 11.40 mmol, 1 eq) in DCM (25 mL) was added BBr₃ (1 M, 34.20 mL, 3 eq) at 0 °C under N₂ atmosphere. The mixture was stirred at 0 °C for 2 hours under N₂ atmosphere. LCMS showed the starting material was consumed completely and desired product was observed. The reaction mixture was quenched with H₂O (10 mL) and extracted with DCM (20 mL×3). The combined organic layers were washed with brine (20 mL×2), dried over Na₂SO₄, filtered and concentrated under reduced pressure to give compound 8 (1.3 g, 55.55% yield) as a yellow solid. LCMS (ESI+): m/z=205.9 (M+1), RT: 0364 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

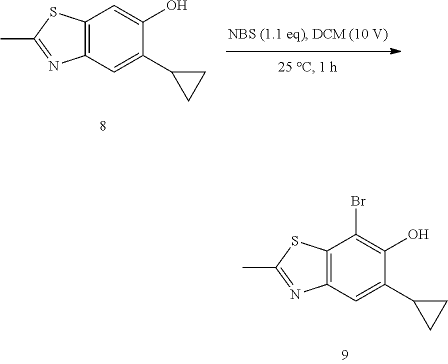

Step 8: To a solution of compound 8 (1.3 g, 6.33 mmol, 1 eq) in DCM (20 mL) was added NBS (1.35 g, 7.60 mmol, 1.2 eq). The mixture was stirred at 25 °C for 1 hour. LCMS showed the starting material was consumed completely and desired MS was observed. The mixture was diluted with H₂O (20 mL) and extracted with EtOAc (30 mL×3). The combined organic layers were washed with brine (20 mL×2), dried over Na₂SO₄, filtered and concentrated under reduced pressure to give a residue. The residue was purified by flash column chromatography on silica gel (0~10% EtOAc in PE) to give compound 9 (1.1 g, 61.12% yield) as a white solid. LCMS (ESI+): m/z=285.7 (M+1), RT: 0.456 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

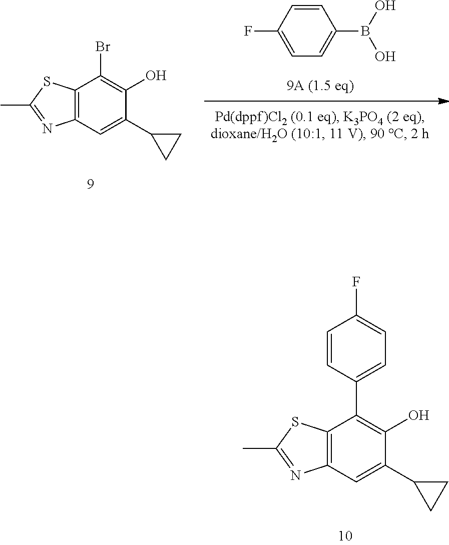 [SMILES: Cc1nc2cc(C3CC3)c(O)c(Br)c2s1 ; OB(O)c1ccc(F)cc1 ; Cc1nc2cc(C3CC3)c(O)c(-c3ccc(F)cc3)c2s1]

Step 9: A mixture of compound 9 (1.1 g, 3.87 mmol, 1 eq), compound 9A (812.42 mg, 5.81 mmol, 1.5 eq), K₃PO₄ (1.64 g, 7.74 mmol, 2 eq) and Pd(dppf)Cl₂ (283.24 mg, 387.09 μmol, 0.1 eq) in dioxane (10 mL) and H₂O (1 mL) was degassed and purged with N₂ for 3 times, and then the mixture was stirred at 90 °C for 2 hours under N₂ atmosphere. The mixture was concentrated under reduced pressure. The residue was purified by flash column chromatography on silica gel (0~8% EtOAc in PE) to give compound 10 (1.0 g, 81.98% yield) as a white solid. ¹H NMR: (400 MHz, DMSO-d₆) δ ppm 0.64-0.73 (m, 2H), 0.94-1.02 (m, 2H), 2.12-2.22 (m, 1H), 2.65 (s, 3H), 7.26-7.37 (m, 3H), 7.51-7.58 (m, 2H), 8.62 (s, 1H).

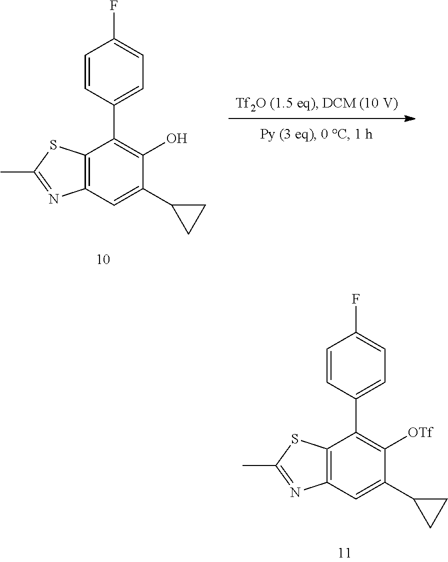 [SMILES: Cc1nc2cc(C3CC3)c(O)c(-c3ccc(F)cc3)c2s1 ; Cc1nc2cc(C3CC3)c(OS(=O)(=O)C(F)(F)F)c(-c3ccc(F)cc3)c2s1]

Step 10: To a solution of compound 10 (1.0 g, 3.17 mmol, 1 eq) and Py (753.05 mg, 9.52 mmol, 768.42 μL, 3 eq) in DCM (10 mL) was added Tf₂O (1.34 g, 4.76 mmol, 785.39 μL, 1.5 eq) at 0 °C under N₂ atmosphere. The mixture was stirred at 0 °C for 1 hour under N₂ atmosphere. The reaction was quenched with H₂O (1 mL), then the mixture was concentrated under reduced pressure. The residue was purified by flash column chromatography on silica gel (0~10% EtOAc in PE) to give compound 11 (1.2 g, 83.27% yield) as a white solid. 1H NMR: (400 MHz, DMSO-d₆) δ ppm 0.91-1.00 (m, 2H), 1.10-1.15 (m, 2H), 2.10-2.19 (m, 1H), 2.78 (s, 3H), 7.38-7.44 (m, 2H), 7.61-7.66 (m, 2H), 7.73 (s, 1H).

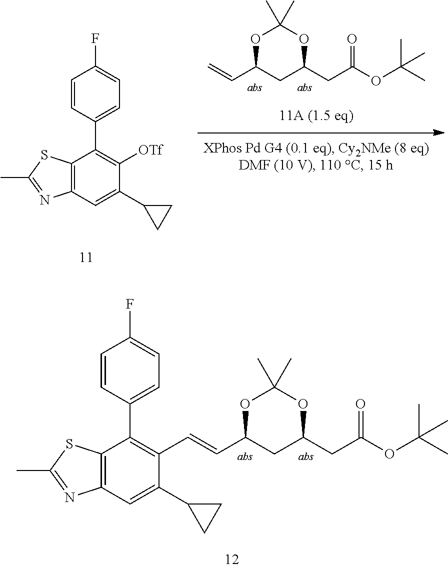 [SMILES: Cc1nc2cc(C3CC3)c(OS(=O)(=O)C(F)(F)F)c(-c3ccc(F)cc3)c2s1 ; C=C[C@@H]1C[C@H](CC(=O)OC(C)(C)C)OC(C)(C)O1 ; Cc1nc2cc(C3CC3)c(/C=C/[C@@H]3C[C@H](CC(=O)OC(C)(C)C)OC(C)(C)O3)c(-c3ccc(F)cc3)c2s1]

Step 11: A mixture of compound 11 (400 mg, 927.16 μmol, 1 eq), compound 11A (713.00 mg, 2.78 mmol, 3 eq), Xphos Pd G4 (79.78 mg, 92.72 μmol, 0.1 eq) and N-cyclohexyl-N-methyl-cyclohexanamine (1.45 g, 7.42 mmol, 1.57 mL, 8 eq) in DMF (8 mL) was degassed and purged with N₂ for 3 times, and then the mixture was stirred at 90 °C for 15 hours under N₂ atmosphere.

LCMS showed the starting material was consumed and desired product was observed. The mixture was diluted with EtOAc (20 mL) and washed with brine (10 mL×5), dried over Na₂SO₄, filtered and concentrated under reduced pressure. The residue was purified by flash column chromatography on silica gel (0~8% EtOAc in PE) to give a residue. The residue was purified by prep-HPLC (column: Welch Ultimate C18 150*25 mm*5 μm; mobile phase: [water (TFA)-ACN]; gradient: 80%-100% B over 10 min) to give compound 12 (100 mg, 21.67% yield) as white oil. LCMS (ESI+): m/z=538.2 (M+1), RT: 0.685 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

In some embodiments, compound 12 is the sodium salt of compound 12.

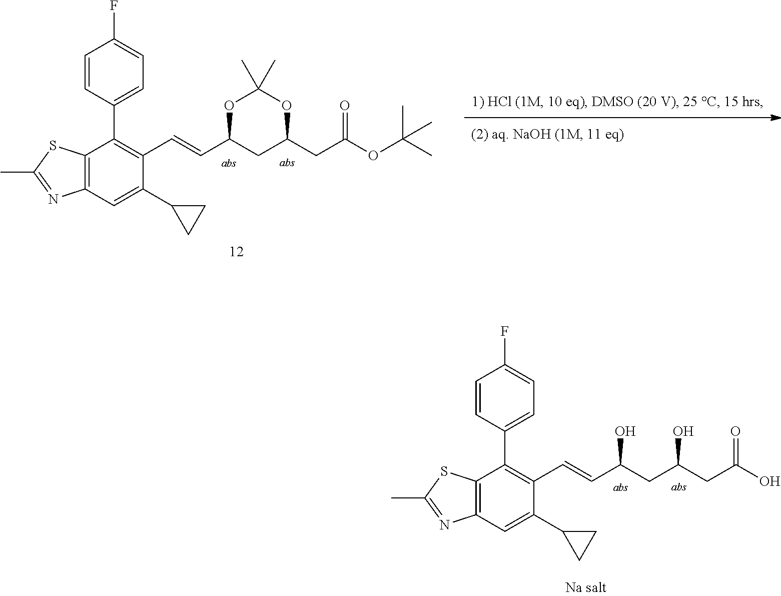 [SMILES: Cc1nc2cc(C3CC3)c(/C=C/[C@@H]3C[C@H](CC(=O)OC(C)(C)C)OC(C)(C)O3)c(-c3ccc(F)cc3)c2s1 ; Cc1nc2cc(C3CC3)c(/C=C/[C@@H](O)C[C@@H](O)CC(=O)O)c(-c3ccc(F)cc3)c2s1]

Step 12: To a solution of compound 12 (100 mg, 200.96 μmol, 1 eq) in DMSO (1 mL) was added HCl (1 M, 2.01 mL, 10 eq). The mixture was stirred at 25 °C for 15 hours. LCMS showed the starting material was consumed completely and desired product was observed. Then NaOH (1 M) was added to the mixture to make pH=10 at 25 °C. The mixture was stirred at 25 °C for 2 hours. The residue was purified by prep-HPLC (column: Waters Xbridge 150*25 mm*5 μm; mobile phase: [water (NH₃·H₂O)-ACN]; gradient: 8%-28% B over 10 min) to give (3R,5S,E)-7-(5-cyclopropyl-7-(4-fluorophenyl)-2-methylbenzo[d]thiazol-6-yl)-3,5-dihydroxyhept-6-enoic acid sodium salt (14.5 mg, 14.99% yield) as a white solid. ¹H NMR: (400 MHz, DMSO-d₆) δ ppm 0.69-0.82 (m, 2H), 0.95-1.03 (m, 2H), 1.06-1.17 (m, 1H), 1.31-1.45 (m, 1H), 1.86-1.96 (m, 1H), 2.02-2.16 (m, 2H), 2.70 (s, 3H), 3.61 (br s, 1H), 4.09 (br d, J=6.8 Hz, 1H), 5.44 (dd, J=16.0, 6.0 Hz, 1H), 6.55 (d, J=16.0 Hz, 1H), 7.24-7.31 (m, 2H), 7.34-7.40 (m, 2H), 7.50 (s, 1H). LCMS (ESI+): m/z=442.1 (M+1), RT: 1.250 min. Kinetex® XBridge C18 3.0*50 mm, 5 μm. Mobile phase: Ramp from 5% ACN in water (0.025% NH₃·H₂O) to 95% ACN in 3.00 min, Flow rate is set at 0.9 mL/min; then hold at 95% ACN for 0.70 minutes Flow rate is set at 0.9 mL/min; return back to 5% ACN in water and hold for 0.30 min. Flow rate is set at 1.2 mL/min. Column temperature at 40 °C and detector wavelength from 210 nm to 265 nm.

### Example 10. Synthesis of (3R,5S,E)-7-(6-cyclopropyl-4-(4-fluorophenyl)benzo[d]thiazol-5-yl)-3,5-dihydroxyhept-6-enoic acid sodium salt

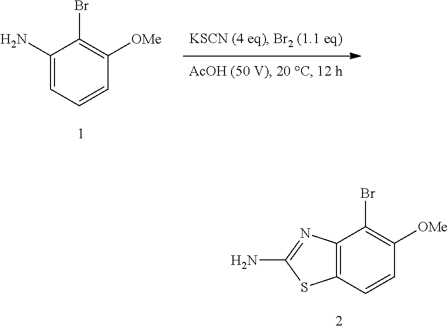 [SMILES: COc1cccc(N)c1Br ; COc1ccc2sc(N)nc2c1Br]

Step 1: To a solution of KSCN (19.24 g, 197.97 mmol, 19.18 mL, 4 eq) in AcOH (200 mL) was added compound 1 (10 g, 49.49 mmol, 1 eq) in AcOH (200 mL) dropwise. Br₂ (8.70 g, 54.44 mmol, 2.80 mL, 1.1 eq) in AcOH (100 mL) was then added dropwise and the mixture was stirred at 20 °C for 12 hours. The reaction mixture was combined with a paralleled batch (10 g compound 1 was used) and concentrated under reduced pressure. The residue was adjusted with saturated aq·NaHCO₃ to pH=7 and filtered. The filter cake was washed with DCM (120 mL) and Petroleum ether (80 mL), dried in vacuum to give compound 2 (14 g, 49.12% yield) as a yellow solid. ¹H NMR: (400 MHz, DMSO-d₆) δ 3.83 (s, 3H), 6.80 (d, J=8.4 Hz, 1H), 7.56 (d, J=8.4 Hz, 1H), 7.82 (s, 2H). LCMS (ESI+): m/z=258.9 (M−56+1), RT: 0.274 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

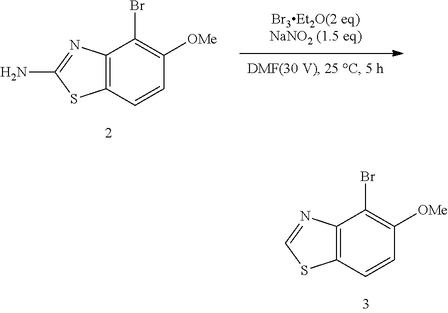 [SMILES: COc1ccc2sc(N)nc2c1Br ; COc1ccc2scnc2c1Br]

Step 2: To a solution of compound 2 (4 g, 15.44 mmol, 1 eq) and NaNO₂ (1.60 g, 23.16 mmol, 1.5 eq) in DMF (120 mL) was added dropwise BF₃·Et₂O (4.38 g, 30.87 mmol, 3.80 mL, 2 eq). The mixture was stirred at 25 °C for 5 hours. The reaction mixture was combined with a paralleled batch (4 g compound 2 was used), quenched by adding H₂O (100 mL) and extracted with EtOAc 300 mL (150 mL×2). The combined organic layers were dried over Na₂SO₄, filtered and concentrated under reduced pressure. The residue was purified by column chromatography on silica gel (PE/EtOAc=1/0 to 4/1) to give compound 3 (4.2 g, 62.03% yield) as a yellow solid. LCMS (ESI+): m/z=243.9 (M−56+1), RT: 0.376 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

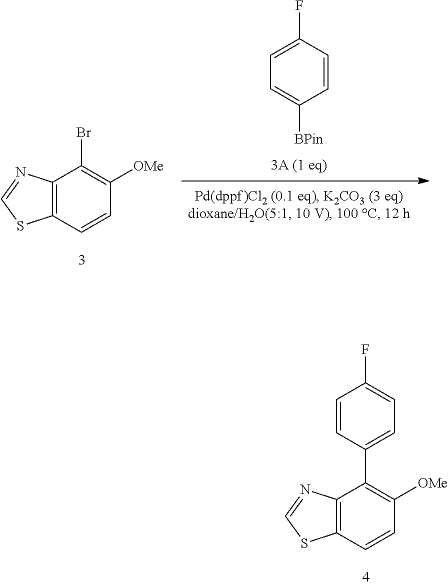

Step 3: To a solution of compound 3 (5.6 g, 22.94 mmol, 1 eq) and compound 3A (5.09 g, 22.94 mmol, 1 eq) in dioxane (60 mL) and H₂O (12 mL) was added K₂CO₃ (9.51 g, 68.82 mmol, 3 eq) and Pd(dppf)Cl₂ (1.68 g, 2.29 mmol, 0.1 eq). The mixture was stirred at 100 °C under N₂ for 12 hours. The reaction mixture was filtered and the filter cake was washed with EtOAc (100 mL). The filtrate was diluted with H₂O (200 mL) and extracted with EtOAc (150 mL×2). The combined organic layers were dried over Na₂SO₄, filtered and concentrated under reduced pressure. The residue was purified by column chromatography on silica gel (PE/EtOAc=1/0 to 5/1) to give compound 4 (4.6 g, 76.56% yield) as a white solid. LCMS (ESI+): m/z=260.0 (M−56+1), RT: 0.457 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

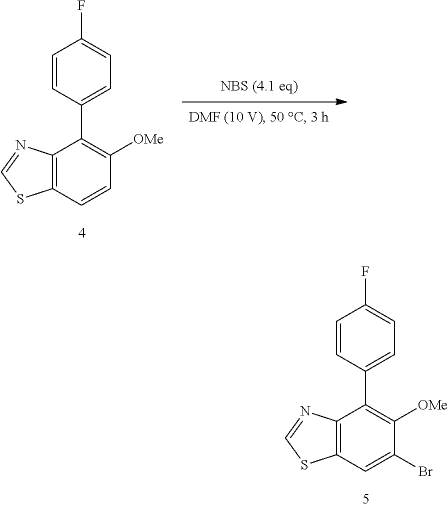 [SMILES: COc1ccc2scnc2c1-c1ccc(F)cc1 ; COc1c(Br)cc2scnc2c1-c1ccc(F)cc1]

Step 4: To a solution of compound 4 (1 g, 3.86 mmol, 1 eq) in DMF (10 mL) was added NBS (2.81 g, 15.81 mmol, 4.1 eq). The mixture was stirred at 50 °C for 3 hours. The residue was diluted with H₂O (100 mL) and extracted with EtOAc (120 mL×2). The combined organic layers were washed with brine (200 mL×2), dried over Na₂SO₄, filtered and concentrated under reduced pressure. The residue was purified by column chromatography on silica gel (PE/EtOAc=1/0 to 30/1) to give compound 5 (1.1 g, 80.96% yield) as a white solid. LCMS (ESI+): m/z=337.9 (M+1), RT: 0.513 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

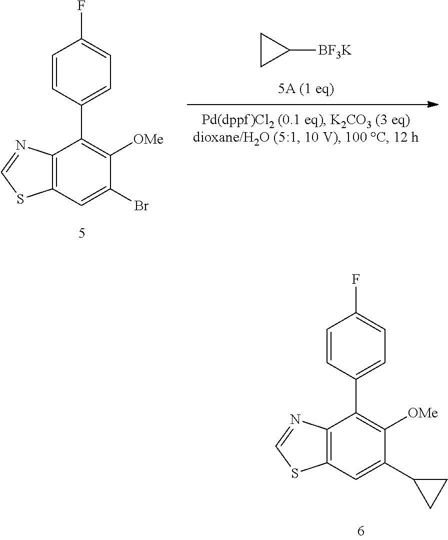

Step 5: To a solution of compound 5 (3.9 g, 11.53 mmol, 1 eq) in dioxane (40 mL) and H₂O (8 mL) was added K₂CO₃ (4.78 g, 34.60 mmol, 3 eq) and compound 5A (2.56 g, 17.30 mmol, 1.5 eq) and Pd(dppf)Cl₂ (843.80 mg, 1.15 mmol, 0.1 eq). The mixture was stirred at 100 °C under N₂ for 12 hours. The reaction mixture was filtered and the filter cake was washed with EtOAc. The filtrate was diluted with H₂O (300 mL) and extracted with EtOAc (150 mL×2). The combined organic layers were dried over Na₂SO₄, and concentrated under reduced pressure. The residue was purified by column chromatography on silica gel (PE/EtOAc=I/O to 40/1) to give compound 6 (2.95 g, 66% yield) as a white solid. LCMS (ESI+): m/z=300.0 (M+1), RT: 0.510 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

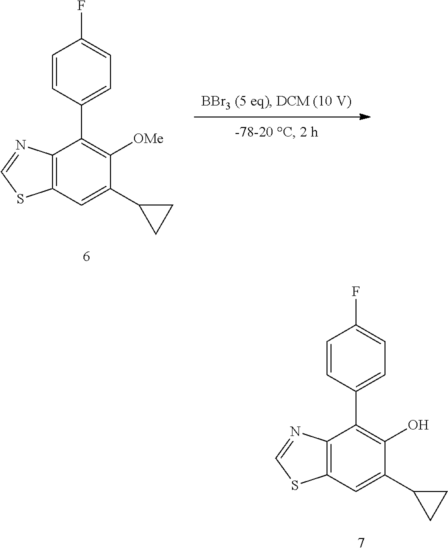 [SMILES: COc1c(C2CC2)cc2scnc2c1-c1ccc(F)cc1 ; Oc1c(C2CC2)cc2scnc2c1-c1ccc(F)cc1]

Step 6: To a solution of compound 6 (2.9 g, 9.69 mmol, 1 eq) in DCM (30 mL) was added dropwise BBr₃ (12.13 g, 48.44 mmol, 4.67 mL, 5 eq) at −78 °C under N₂. The mixture was stirred at −78 °C for 0.25 hour then 20 °C for 1.75 hour. The reaction mixture was quenched by adding ice-water (30 mL). The mixture was adjusted with saturated aqueous NaHCO₃ to pH=8 and extracted with DCM (50 mL×2). The combined organic layers were dried over Na₂SO₄, filtered and concentrated under reduced pressure. The residue was purified by column chromatography on silica gel (PE/EtOAc=1/0 to 30/1) to give compound 7 (900 mg, 32.23% yield) as a white solid. LCMS (ESI+): m/z=286.0 (M+1), RT: 0.458 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

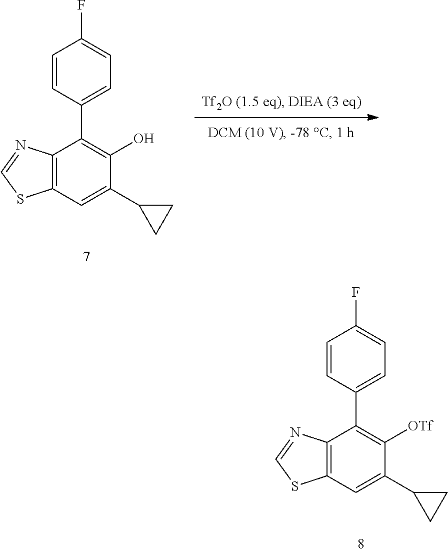 [SMILES: Oc1c(C2CC2)cc2scnc2c1-c1ccc(F)cc1 ; O=S(=O)(Oc1c(C2CC2)cc2scnc2c1-c1ccc(F)cc1)C(F)(F)F]

Step 7: To a solution of compound 7 (620 mg, 2.17 mmol, 1 eq) in DCM (6.5 mL) was added DIEA (842.47 mg, 6.52 mmol, 1.14 mL, 3 eq) and Tf₂O (919.58 mg, 3.26 mmol, 537.77 μL, 1.5 eq) at −78 °C under N₂. The mixture was stirred at −78 °C for 1 hour. The mixture was diluted with H₂O (40 mL) and extracted with DCM (50 mL×2). The combined organic layers were dried over Na₂SO₄, filtered and concentrated under reduced pressure. The residue was purified by column chromatography on silica gel (PE/EtOAc=1/0 to 85/1) to give compound 8 (800 mg, 87.85% yield) as a yellow solid. LCMS (ESI+): m/z=417.9 (M+1), RT: 0.551 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

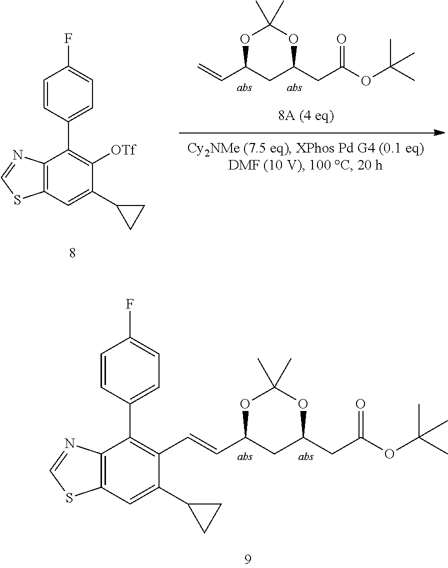 [SMILES: O=S(=O)(Oc1c(C2CC2)cc2scnc2c1-c1ccc(F)cc1)C(F)(F)F ; C=C[C@@H]1C[C@H](CC(=O)OC(C)(C)C)OC(C)(C)O1 ; CC(C)(C)OC(=O)C[C@H]1C[C@@H](/C=C/c2c(C3CC3)cc3scnc3c2-c2ccc(F)cc2)OC(C)(C)O1]

Step 8: To a solution of compound 8 (400 mg, 958.32 μmol, 1 eq) and compound 8A (982.61 mg, 3.83 mmol, 4 eq) in DMF (5 mL) was added Xphos Pd G4 (82.46 mg, 95.83 μmol, 0.1 eq) and N-cyclohexyl-N-methyl-cyclohexanamine (1.40 g, 7.19 mmol, 1.52 mL, 7.5 eq). The mixture was stirred at 100 °C for 20 hours. The reaction mixture was filtered. The filtrate was diluted with H₂O (80 mL) and extracted with EtOAc (100 mL×2). The combined organic layers were dried over Na₂SO₄, filtered and concentrated under reduced pressure. The crude product was purified by prep-HPLC (column: Waters Xbridge BEH C18 150*25 mm*5 μm; mobile phase: [water (NH₄HCO₃)-ACN]; gradient: 70%-100% B over 10 min) and prep-TLC (PE/DCM=1:5) to give compound 9 (45 mg, 8.82% yield) as a yellow solid. ¹H NMR: (400 MHz, CDCl₃) δ 0.73-0.84 (m, 2H), 0.98-1.05 (m, 2H), 1.24-1.28 (m, 2H), 1.37 (s, 3H), 1.47 (s, 12H), 2.10-2.20 (m, 1H), 2.23-2.33 (m, 1H), 2.37-2.48 (m, 1H), 4.19-4.30 (m, 1H), 4.30-4.45 (m, 1H), 5.49 (dd, J=16.0, 6.0 Hz, 1H), 6.67 (d, J=16.0 Hz, 1H), 7.12 (t, J=8.4 Hz, 2H), 7.34 (dd, J=8.0, 6.0 Hz, 2H), 7.61 (s, 1H), 8.86 (s, 1H). LCMS (ESI+): m/z=524.1 (M+1), RT: 1.848 min. LC/MS: Kinetex® EVO C18 3.0×50 mm 2.6 μm. Detection methods are photo-diode array (PDA). MS mode was positive electrospray ionization. MS range was 50-1050. Mobile phase: Ramp from 30% ACN (0.01875% TFA) in water (0.0375% TFA) to 90% ACN in 2.00 min, Flow rate is set at 0.9 mL/min; then ramp from 90% ACN in water to 100% ACN in 1.70 min, Flow rate is set at 0.9 mL/min; return back to 30% ACN in water and hold for 0.30 min. Flow rate is set at 1.2 mL/min.

In some embodiments, compound 9 is the sodium salt of compound 9.

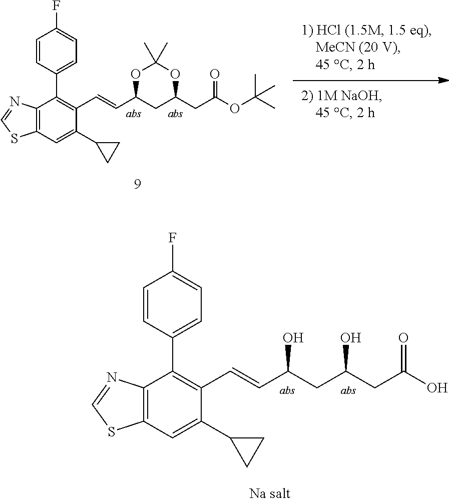 [SMILES: CC(C)(C)OC(=O)C[C@H]1C[C@@H](/C=C/c2c(C3CC3)cc3scnc3c2-c2ccc(F)cc2)OC(C)(C)O1 ; O=C(O)C[C@H](O)C[C@H](O)/C=C/c1c(C2CC2)cc2scnc2c1-c1ccc(F)cc1]

Step 9: To a solution of compound 9 (45 mg, 85.93 μmol, 1 eq) in MeCN (1 mL) was added HCl (1.5 M, 85.93 μL, 1.5 eq). The mixture was stirred at 45 °C for 2 hours. The mixture was based by 1M aq·NaOH to pH=9 and stirred at 45 °C for 2 hours. The reaction mixture was concentrated under reduced pressure. The residue was purified by prep-HPLC (column: Waters Xbridge 150*25 mm*5 μm; mobile phase: [water (NH₃·H₂O)-ACN]; gradient: 0%-29% B over 10 min) to afford (3R,5S,E)-7-(6-cyclopropyl-4-(4-fluorophenyl)benzo[d]thiazol-5-yl)-3,5-dihydroxyhept-6-enoic acid sodium salt (9 mg, 23.25% yield) as a yellow solid. 1H NMR: (400 MHz, DMSO-d₆) δ 0.78 (d, J=5.2 Hz, 2H), 0.99 (d, J=8.0 Hz, 2H), 1.10-1.21 (m, 1H), 1.34-1.49 (m, 1H), 1.93-2.02 (m, 1H), 2.08-2.19 (m, 2H), 3.67 (d, J=2.8 Hz, 1H), 4.05-4.17 (m, 1H), 5.48 (dd, J=16.0, 6.0 Hz, 1H), 6.55 (d, J=16.0 Hz, 1H), 7.21 (J=8.8 Hz, 2H), 7.27-7.37 (m, 2H), 7.79 (s, 1H), 9.15 (s, 1H). LCMS (ESI+): m/z=428.0 (M+1), RT: 1.215 min. LC/MS: XBridge C18 3.0*50 mm, 5 μm. Detection methods are photo-diode array (PDA). MS mode was positive electrospray ionization. MS range was 50-1050. Mobile phase: Ramp from 5% ACN in water (0.025% NH₃·H₂O) to 95% ACN in 3.00 min, Flow rate is set at 0.9 mL/min; then hold at 95% ACN for 0.70 minutes Flow rate is set at 0.9 mL/min; return back to 5% ACN in water and hold for 0.30 min. Flow rate is set at 1.2 mL/min.

### Example 11. Synthesis of (3R,5S,E)-7-(6-(bicyclo[1.1.1]pentan-1-yl)-4-(2-chloro-4-fluorophenyl)-2-cyclopropyl-5-(methoxymethyl)pyridin-3-yl)-3,5-dihydroxyhept-6-enoic acid

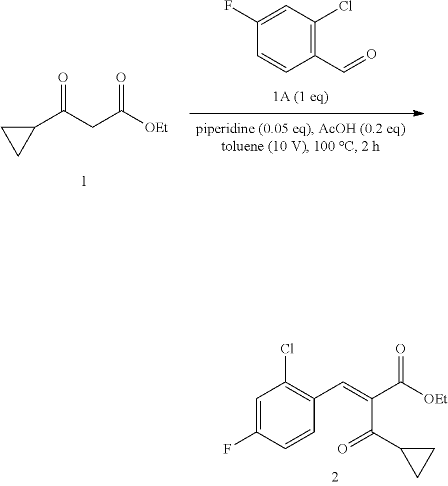 [SMILES: O=Cc1ccc(F)cc1Cl ; CCOC(=O)CC(=O)C1CC1 ; CCOC(=O)/C(=C/c1ccc(F)cc1Cl)C(=O)C1CC1]

Step 1: To a solution of compound 1 (10.0 g, 64.03 mmol, 1 eq), compound 1A (10.15 g, 64.03 mmol, 1 eq) in toluene (100 mL) was added piperidine (272.60 mg, 3.20 mmol, 316.16 μL, 0.05 eq) and HOAc (769.02 mg, 12.81 mmol, 733.10 μL, 0.2 eq) and the mixture was stirred at 100 °C for 2 hours. LCMS showed the reactant was consumed completely and 72% of desired product was observed. The mixture was diluted with water (200 mL) and extracted with EtOAc (200 mL×3). The combined organic layers were dried over anhydrous Na₂SO₄ and concentrated under reduced pressure. The crude product was purified by flash column chromatography on silica gel (0-10% EtOAc in PE) to give compound 2 (15.33 g, 80.69% yield) as colorless oil. LCMS (ESI+): m/z=297.1 (M+1), RT: 0.473 min. LC/MS: Kinetex® EVO C18 2.1×30 mm 5 μm. Detection methods are Photo-Diode Array (PDA). MS mode was positive electrospray ionization. MS range was 50-1050. Mobile phase A was 0.04% TFA in water, and mobile phase B was 0.02% TFA in HPLC grade acetonitrile. The gradient was 5-95% B in 0.60 min, Flow rate was 2.0 mL/min; then hold at 95% ACN for 0.18 minutes, Flow rate was 2.0 mL/min; return back to 5% ACN in water and hold for 0.02 min, Flow rate was 2.0 mL/min.

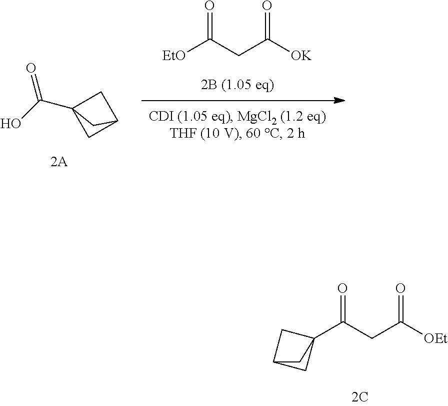

Step 2: A mixture of compound 2A (2.0 g, 17.84 mmol, 1 eq) and CDI (3.04 g, 18.73 mmol, 1.05 eq) in THF (40 mL) was degassed and purged with N₂ for 3 times and then the mixture was stirred at 60 °C for 1 hour under N₂ atmosphere. Compound 2B (3.19 g, 18.73 mmol, 1.05 eq) and MgCl₂ (2.04 g, 21.40 mmol, 878.42 μL, 1.2 eq) was added. The mixture was stirred at 60 °C for another 1 hour under N₂ atmosphere. TLC (PE/EtOAc=20:1) indicated one major new spot was detected. The reaction mixture was filtered and the filtrate was concentrated under reduced pressure. The residue was purified by flash column chromatography on silica gel (0-10% EtOAc in PE) to afford compound 2C (4.4 g, 67.69% yield) as yellow oil. LCMS (ESI−): m/z=180.9 (M−1), RT: 0.525 min. LC/MS: Gemini@5 umNX-C18 110A 2*30 mm, 5 μm. Detection methods are photo-diode array (PDA). MS mode was positive electrospray ionization. MS range was 50-1050. Mobile phase: Ramp from 5% ACN in water (0.025% NH₃·H₂O) to 95% ACN in water in 0.70 min, Flow rate is set at 1.7 mL/min; then hold at 95% ACN for 0.20 min, Flow rate is set at 1.7 mL/min; return back to 5% ACN in water and hold for 0.10 min, Flow rate is set at 1.7 mL/min.

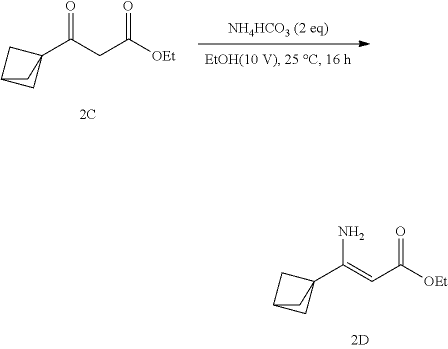 [SMILES: CCOC(=O)CC(=O)C12CC(C1)C2 ; "NH4HCO3 (2 eq)" ; CCOC(=O)/C=C(\N)C12CC(C1)C2]

Step 3: To a solution of compound 2C (4.4 g, 24.15 mmol, 1 eq) in EtOH (44 mL) was added NH₄HCO₃ (3.82 g, 48.29 mmol, 3.98 mL, 2 eq). The mixture was stirred at 25 °C for 16 hours under N₂ atmosphere. LCMS showed 98% of desired compound was detected. The reaction mixture was filtered and the filtrate was concentrated under reduced pressure. The residue was purified by flash column chromatography on silica gel (0-5% EtOAc in PE) to afford compound 2D (3.73 g, 85.23% yield) as yellow oil. ¹H NMR: (400 MHz, CDCl₃) δ 1.26 (t, J=7.2 Hz, 3H), 1.97 (s, 6H), 2.50 (s, 1H), 4.07-4.14 (m, 2H), 4.52 (s, 2H), 7.32-8.13 (m, 1H).

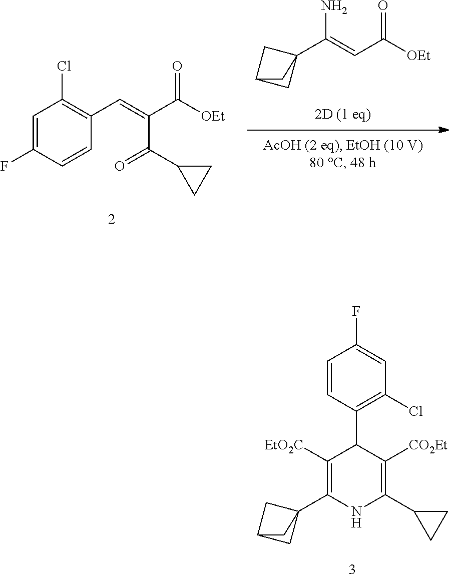 [SMILES: CCOC(=O)/C(=C/c1ccc(F)cc1Cl)C(=O)C1CC1 ; CCOC(=O)/C=C(\N)C12CC(C1)C2 ; CCOC(=O)C1=C(C2CC2)NC(C23CC(C2)C3)=C(C(=O)OCC)C1c1ccc(F)cc1Cl]

Step 4: A solution of compound 2 (2.9 g, 9.77 mmol, 1 eq), compound 2D (1.77 g, 9.77 mmol, 1 eq), AcOH (1.17 g, 19.55 mmol, 1.12 mL, 2 eq) in EtOH (20 mL) was stirred at 80 °C for 48 hours. LCMS showed 20% of compound 2 remained and 50% of desired product was observed. The mixture was concentrated under reduced pressure. The crude product was purified by flash column chromatography on silica gel (0-5% EtOAc in PE) to give compound 3 (2.61 g, 58.06% yield) as a yellow solid. LCMS (ESI+): m/z=460.1 (M+1), RT: 0.554 min. LC/MS: Kinetex® EVO C18 2.1×30 mm 5 μm. Detection methods are Photo-Diode Array (PDA). MS mode was positive electrospray ionization. MS range was 50-1050. Mobile phase A was 0.04% TFA in water, and mobile phase B was 0.02% TFA in HPLC grade acetonitrile. The gradient was 5-95% B in 0.60 min, Flow rate was 2.0 mL/min; then hold at 95% ACN for 0.18 min, Flow rate was 2.0 mL/min; return back to 5% ACN in water and hold for 0.02 min, Flow rate was 2.0 mL/min.

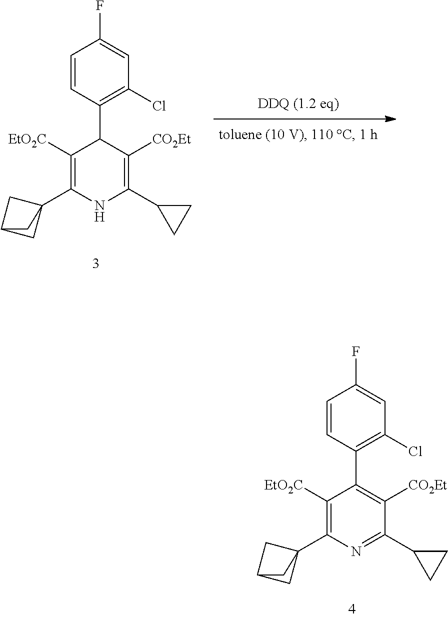 [SMILES: CCOC(=O)C1=C(C2CC2)NC(C23CC(C2)C3)=C(C(=O)OCC)C1c1ccc(F)cc1Cl ; CCOC(=O)c1c(C2CC2)nc(C23CC(C2)C3)c(C(=O)OCC)c1-c1ccc(F)cc1Cl]

Step 5: To a solution of compound 3 (2.3 g, 5.00 mmol, 1 eq) in toluene (20 mL) was added DDQ (1.36 g, 6.00 mmol, 1.2 eq) and the mixture was stirred at 110 °C for 1 hour. LCMS showed the reactant was consumed completely and 26% of desired product was observed. The mixture was concentrated under reduced pressure. The crude product was purified by flash column chromatography on silica gel (0-10% EtOAc in PE) to give compound 4 (2.1 g, 91.71% yield) as a white solid. LCMS (ESI+): m/z=458.2 (M+1), RT: 0.580 min. LC/MS: Kinetex® EVO C18 2.1×30 mm 5 μm. Detection methods are Photo-Diode Array (PDA). MS mode was positive electrospray ionization. MS range was 50-1050. Mobile phase A was 0.04% TFA in water, and mobile phase B was 0.02% TFA in HPLC grade acetonitrile. The gradient was 5-95% B in 0.60 min, Flow rate was 2.0 mL/min; then hold at 95% ACN for 0.18 min, Flow rate was 2.0 mL/min; return back to 5% ACN in water and hold for 0.02 min, Flow rate was 2.0 mL/min.

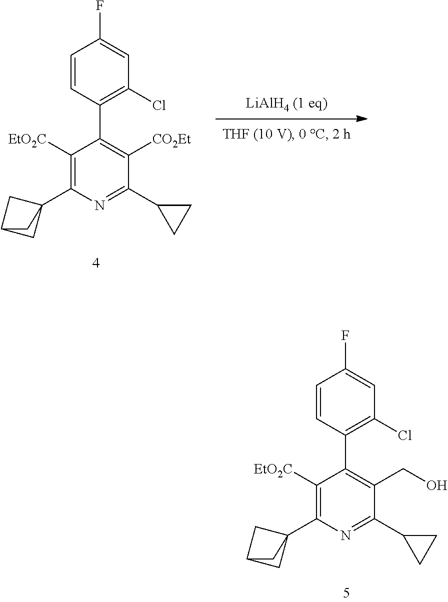 [SMILES: CCOC(=O)c1c(C2CC2)nc(C23CC(C2)C3)c(C(=O)OCC)c1-c1ccc(F)cc1Cl ; CCOC(=O)c1c(C23CC(C2)C3)nc(C2CC2)c(CO)c1-c1ccc(F)cc1Cl]

Step 6: To a solution of compound 4 (2.1 g, 4.59 mmol, 1 eq) in THF (20 mL) was added LiAlH₄ (1 M, 4.59 mL, 1 eq) and the mixture was stirred at 0 °C for 2 hours. LCMS showed the reactant was consumed completely and 86% of desired product was observed. The reaction was quenched by adding EtOAc (10 mL), H₂O (1 mL) and 15% aqueous NaOH (1 mL). The mixture was dried over Na₂SO₄, filtered and the filtrate was concentrated under reduced pressure to give compound 5 (2.15 g, crude) as a yellow solid. ¹H NMR: (400 MHz, CDCl₃) δ 0.97-1.05 (m, 5H), 1.12-1.20 (m, 1H), 1.28-1.36 (m, 1H), 1.57 (s, 1H), 2.13 (s, 6H), 2.37-2.46 (m, 1H), 2.47 (s, 1H), 3.90-4.04 (m, 2H), 4.41-4.58 (m, 2H), 7.04 (td, J=8.4, 2.4 Hz, 1H), 7.18 (dd, J=8.4, 6.0 Hz, 1H), 7.23 (dd, J=8.4, 2.4 Hz, 1H). LCMS (ESI+): m/z=416.1 (M+1), RT: 0.517 min. LC/MS: Kinetex® EVO C18 2.1×30 mm 5 μm. Detection methods are Photo-Diode Array (PDA). MS mode was positive electrospray ionization. MS range was 50-1050. Mobile phase A was 0.04% TFA in water, and mobile phase B was 0.02% TFA in HPLC grade acetonitrile. The gradient was 5-95% B in 0.60 min, Flow rate was 2.0 mL/min; then hold at 95% ACN for 0.18 min, Flow rate was 2.0 mL/min; return back to 5% ACN in water and hold for 0.02 min, Flow rate was 2.0 mL/min.

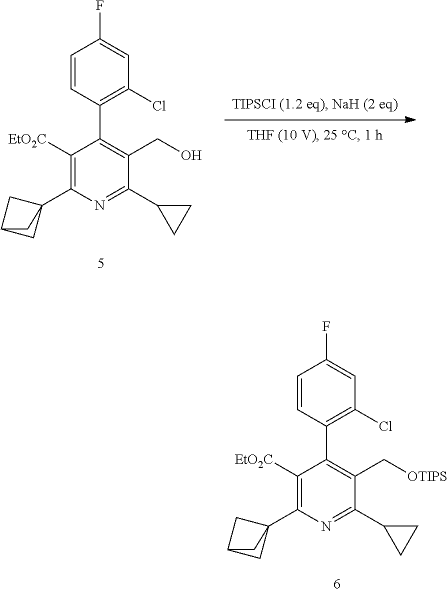

Step 7: To a solution of compound 5 (1.2 g, 2.89 mmol, 1 eq) in THF (12 mL) was added NaH (230.83 mg, 5.77 mmol, 60% purity, 2 eq) at 0 °C under N₂ and the mixture was stirred at 0 °C for 0.5 hour. Chloro(triisopropyl) silane (667.57 mg, 3.46 mmol, 740.92 μL, 1.2 eq) was added and the mixture was stirred at 25 °C for 1 hour. LCMS showed the reactant was consumed completely and 90% of desired product was observed. The reaction was quenched by adding water (100 mL) and extracted with EtOAc (100 mL×3). The combined organic layers were dried over anhydrous Na₂SO₄ and concentrated under reduced pressure. The crude product was purified by flash column chromatography on silica gel (0-5% EtOAc in PE) to give compound 6 (1.45 g, 87.82% yield) as colorless oil. LCMS (ESI+): m/z=572.3 (M+1), RT: 0.738 min. LC/MS: Kinetex® EVO C18 2.1×30 mm 5 μm. Detection methods are Photo-Diode Array (PDA). MS mode was positive electrospray ionization. MS range was 50-1050. Mobile phase A was 0.04% TFA in water, and mobile phase B was 0.02% TFA in HPLC grade acetonitrile. The gradient was 5-95% B in 0.60 min, Flow rate was 2.0 mL/min; then hold at 95% ACN for 0.18 min, Flow rate was 2.0 mL/min; return back to 5% ACN in water and hold for 0.02 min, Flow rate was 2.0 mL/min.

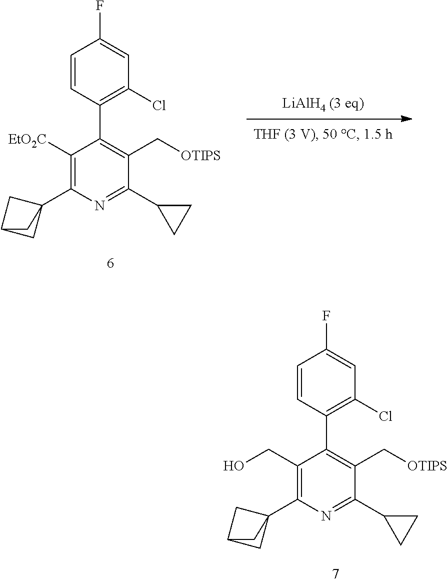

Step 8: To a solution of compound 6 (1.45 g, 2.53 mmol, 1 eq) in THF (5 mL) was added LiAlH₄ (288.49 mg, 7.60 mmol, 3 eq) at 0 °C under N₂ and the mixture was stirred at 50 °C for 1.5 hours. LCMS showed 62% of compound 6 remained and 31% of desired product was observed. The reaction was quenched by adding EtOAc (10 mL), water (0.29 mL) and 15% aqueous NaOH (0.29 mL). The resulting suspension was filtered. The filtrate was dried over anhydrous Na₂SO₄ and concentrated under reduced pressure. The crude product was purified by flash column chromatography on silica gel (0-5% EtOAc in PE) to give compound 7 (472 mg, 35.13% yield) as colorless oil. LCMS (ESI+): m/z=530.6 (M+1), RT: 0.661 min. LC/MS: Kinetex® EVO C18 2.1×30 mm 5 μm. Detection methods are Photo-Diode Array (PDA). MS mode was positive electrospray ionization. MS range was 50-1050. Mobile phase A was 0.04% TFA in water, and mobile phase B was 0.02% TFA in HPLC grade acetonitrile. The gradient was 5-95% B in 0.60 min, Flow rate was 2.0 mL/min; then hold at 95% ACN for 0.18 min, Flow rate was 2.0 mL/min; return back to 5% ACN in water and hold for 0.02 min, Flow rate was 2.0 mL/min.

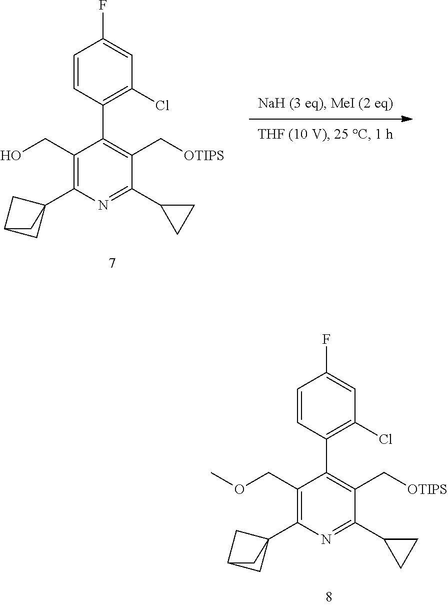

Step 9: To a solution of compound 7 (820 mg, 1.55 mmol, 1 eq) in THF (10 mL) was added NaH (185.58 mg, 4.64 mmol, 60% purity, 3 eq) at 0 °C under N₂ and the mixture was stirred at 0 °C for 15 minutes. CH₃I (439.05 mg, 3.09 mmol, 192.57 μL, 2 eq) was added and the mixture was stirred at 25 °C for 1 hour. LCMS showed the reactant was consumed completely and 90% of desired product was observed. The reaction was quenched by adding water (20 mL) and extracted with EtOAc (20 mL×3). The combined organic layers were dried over anhydrous Na₂SO₄ and concentrated under reduced pressure to give compound 8 (803 mg, 95.40% yield) as colorless oil. LCMS (ESI+): m/z=544.3 (M+1), RT: 0.733 min. LC/MS: Kinetex® EVO C18 2.1×30 mm 5 μm. Detection methods are Photo-Diode Array (PDA). MS mode was positive electrospray ionization. MS range was 50-1050. Mobile phase A was 0.04% TFA in water, and mobile phase B was 0.02% TFA in HPLC grade acetonitrile. The gradient was 5-95% B in 0.60 min, Flow rate was 2.0 mL/min; then hold at 95% ACN for 0.18 min, Flow rate was 2.0 mL/min; return back to 5% ACN in water and hold for 0.02 min, Flow rate was 2.0 mL/min.

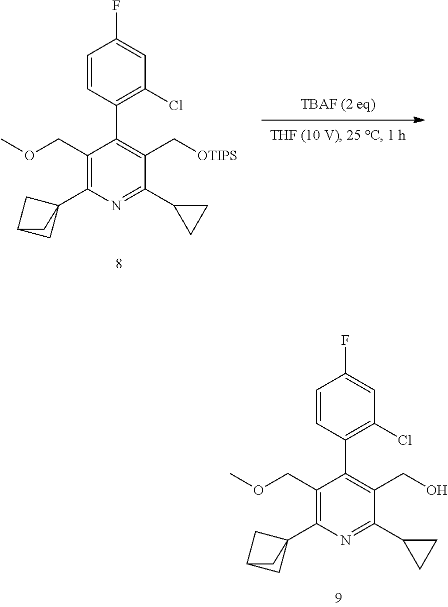

Step 10: To a solution of compound 8 (803 mg, 1.48 mmol, 1 eq) in THF (8 mL) was added TBAF (1 M, 2.95 mL, 2 eq) and the mixture was stirred at 25 °C for 1 hour. LCMS showed the reactant was consumed completely and 88% of desired product was observed. The reaction was quenched by adding water (20 mL) and extracted with EtOAc (20 mL×3). The combined organic layers were dried over anhydrous Na₂SO₄ and concentrated under reduced pressure. The crude product was purified by flash column chromatography on silica gel (0-10% EtOAc in PE) to give compound 9 (500 mg, 87.36% yield) as a white solid. LCMS (ESI+): m/z=388.2 (M+1), RT: 0.467 min. LC/MS: Kinetex® EVO C18 2.1×30 mm 5 μm. Detection methods are Photo-Diode Array (PDA). MS mode was positive electrospray ionization. MS range was 50-1050. Mobile phase A was 0.04% TFA in water, and mobile phase B was 0.02% TFA in HPLC grade acetonitrile. The gradient was 5-95% B in 0.60 min, Flow rate was 2.0 mL/min; then hold at 95% ACN for 0.18 min, Flow rate was 2.0 mL/min; return back to 5% ACN in water and hold for 0.02 min, Flow rate was 2.0 mL/min.

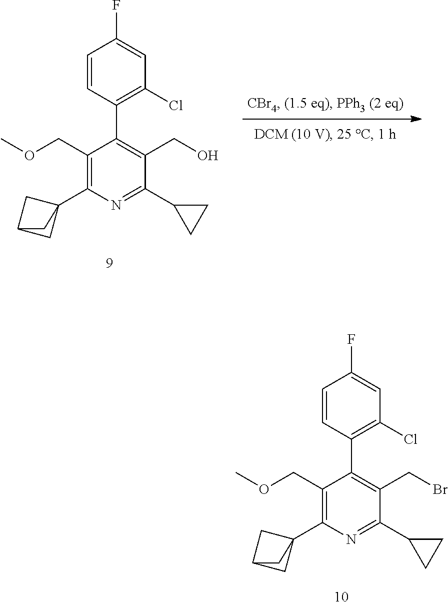 [SMILES: COCc1c(C23CC(C2)C3)nc(C2CC2)c(CO)c1-c1ccc(F)cc1Cl ; COCc1c(C23CC(C2)C3)nc(C2CC2)c(CBr)c1-c1ccc(F)cc1Cl]

Step 11: To a solution of compound 9 (500 mg, 1.29 mmol, 1 eq) in DCM (6 mL) was added PPh₃ (676.21 mg, 2.58 mmol, 2 eq) and CBr₄ (641.24 mg, 1.93 mmol, 1.5 eq) at 0 °C under N₂. The mixture was stirred at 25 °C for 1 hour. TLC showed the reactant was consumed completely and a new spot with lower polarity was observed. The mixture was concentrated under reduced pressure. The crude product was purified by flash column chromatography on silica gel (0-4% EtOAc in PE) to give compound 10 (450 mg, 77.44% yield) as a white solid. LCMS (ESI+): m/z=450.0 (M+1), RT: 0.608 min. LC/MS: Kinetex® EVO C18 2.1×30 mm 5 um. Detection methods are Photo-Diode Array (PDA). MS mode was positive electrospray ionization. MS range was 50-1050. Mobile phase A was 0.04% TFA in water, and mobile phase B was 0.02% TFA in HPLC grade acetonitrile. The gradient was 5-95% B in 0.60 min, Flow rate was 2.0 mL/min; then hold at 95% ACN for 0.18 min, Flow rate was 2.0 mL/min; return back to 5% ACN in water and hold for 0.02 min, Flow rate was 2.0 mL/min.

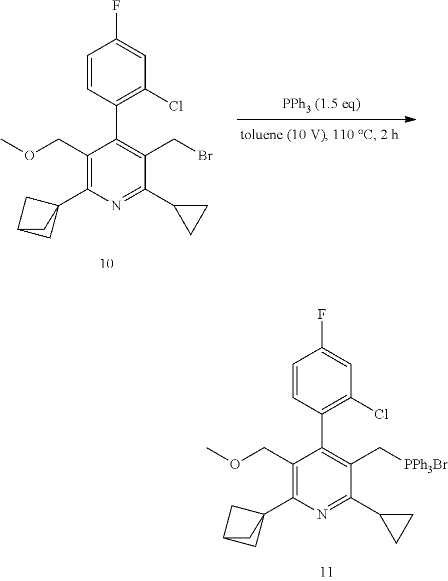

Step 12: To a solution of compound 10 (450 mg, 998.29 μmol, 1 eq) in toluene (5 mL) was added PPh₃ (392.76 mg, 1.50 mmol, 1.5 eq) and the mixture was stirred at 110 °C for 2 hours. LCMS showed the reactant was consumed completely and 51% of desired product was observed. The mixture was concentrated under reduced pressure to give compound 11 (711 mg, crude) as a white solid. LCMS (ESI+): m/z=632.2 (M−79), RT: 0.485 min. LC/MS: Kinetex® EVO C18 2.1×30 mm 5 μm. Detection methods are Photo-Diode Array (PDA). MS mode was positive electrospray ionization. MS range was 50-1050. Mobile phase A was 0.04% TFA in water, and mobile phase B was 0.02% TFA in HPLC grade acetonitrile. The gradient was 5-95% B in 0.60 min, Flow rate was 2.0 mL/min; then hold at 95% ACN for 0.18 min, Flow rate was 2.0 mL/min; return back to 5% ACN in water and hold for 0.02 min, Flow rate was 2.0 mL/min.

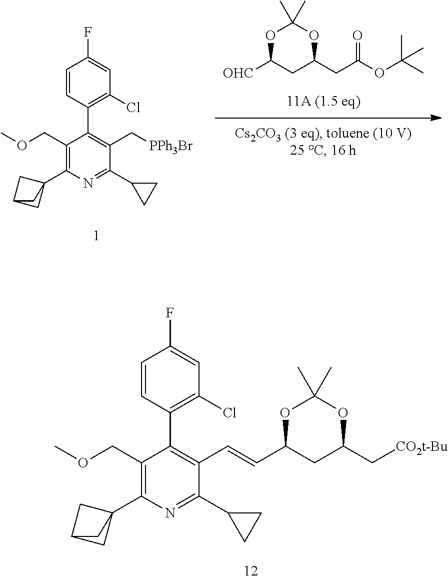

Step 13: To a solution of compound 11 (711 mg, 997.12 μmol, 1 eq), compound 11A (386.35 mg, 1.50 mmol, 1.5 eq) in toluene (5 mL) was added Cs₂CO₃ (974.64 mg, 2.99 mmol, 3 eq) and the mixture was stirred at 25 °C for 16 hours. LCMS showed the reactant was consumed completely and 37% of desired product was observed. The mixture was concentrated under reduced pressure. The crude product was purified by flash column chromatography on silica gel (0-5% EtOAc in PE) and prep-TLC (DCM/PE=3:1) to give compound 12 (105 mg, 17.20% yield) as colorless oil. LCMS (ESI+): m/z=612.7 (M+1), RT: 0.631 min. LC/MS: Kinetex® EVO C18 2.1×30 mm 5 μm. Detection methods are Photo-Diode Array (PDA). MS mode was positive electrospray ionization. MS range was 50-1050. Mobile phase A was 0.04% TFA in water, and mobile phase B was 0.02% TFA in HPLC grade acetonitrile. The gradient was 5-95% B in 0.60 min, Flow rate was 2.0 mL/min; then hold at 95% ACN for 0.18 min, Flow rate was 2.0 mL/min; return back to 5% ACN in water and hold for 0.02 min, Flow rate was 2.0 mL/min. ¹H NMR: (400 MHz, CDCl₃) δ 0.90 (dd, J=8.0, 3.2 Hz, 2H), 1.34 (d, J=7.2 Hz, 4H), 1.40-1.50 (m, 13H), 1.57 (s, 2H), 2.24 (q, J=9.6 Hz, 8H), 2.34-2.44 (m, 1H), 2.51 (s, 1H), 3.10 (d, J=6.0 Hz, 3H), 3.74 (dd, J=10.0, 2.8 Hz, 1H), 4.16-4.32 (m, 2H), 4.38 (dd, J=10.0, 3.6 Hz, 1H), 5.33-5.58 (m, 1H), 6.17-6.37 (m, 1H), 6.96-7.07 (m, 1H), 7.10-7.23 (m, 2H).

In some embodiments, compound 12 is the sodium salt of compound 12.

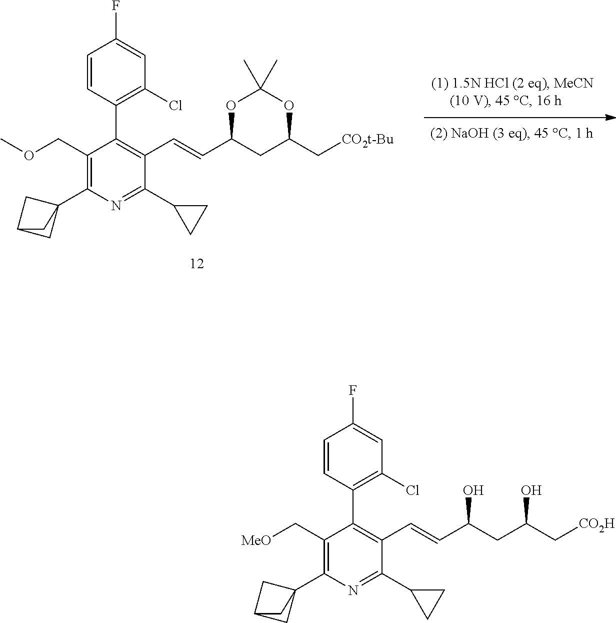 [SMILES: COCc1c(C23CC(C2)C3)nc(C2CC2)c(/C=C/[C@@H]2C[C@H](CC(=O)OC(C)(C)C)OC(C)(C)O2)c1-c1ccc(F)cc1Cl ; COCc1c(C23CC(C2)C3)nc(C2CC2)c(/C=C/[C@@H](O)C[C@@H](O)CC(=O)O)c1-c1ccc(F)cc1Cl]

Step 14: To a solution of compound 12 (105 mg, 171.52 μmol, 1 eq) in CH₃CN (1 mL) was added HCl (1.5 M, 228.69 μL, 2 eq) and the mixture was stirred at 45 °C for 16 hours. NaOH (20.58 mg, 514.56 μmol, 3 eq) was added and the mixture was stirred at 45 °C for 1 hour. LCMS showed the reactant was consumed completely and 91% of desired product was observed. The mixture was purified by prep-HPLC (column: Phenomenex luna C18 150*25 mm*10 μm; mobile phase: [water (FA)-ACN]; gradient: 51%-81% B over 10 min) to give (3R,5S,E)-7-(6-(bicyclo[1.1.1]pentan-1-yl)-4-(2-chloro-4-fluorophenyl)-2-cyclopropyl-5-(methoxymethyl)pyridin-3-yl)-3,5-dihydroxyhept-6-enoic acid sodium salt (45.63 mg, 51.56% yield) as a brown solid. ¹H NMR: (400 MHz, CDCl₃) δ 0.84-0.95 (m, 2H), 1.06-1.16 (m, 1H), 1.20-1.28 (m, 1H), 1.30-1.39 (m, 1H), 1.41-1.58 (m, 1H), 2.14-2.29 (m, 8H), 2.47-2.53 (m, 3H), 3.11 (d, J=1.2 Hz, 3H), 3.73 (d, J=10.0 Hz, 1H), 4.07-4.22 (m, 1H), 4.29-4.46 (m, 2H), 5.53 (td, J=15.6, 6.4 Hz, 1H), 6.29-6.40 (m, 1H), 6.98-7.07 (m, 1H), 7.14-7.23 (m, 2H).

LCMS (ESI+): m/z=516.2 (M+1), RT: 1.982 min. LC/MS: Kinetex® EVO C18 3.0×50 mm 2.6 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in 3.40 min, Flow rate is set at 0.9 mL/min; then hold at 95% ACN for 0.30 min, Flow rate is set at 0.9 mL/min; return back to 5% ACN in water and hold for 0.30 min, Flow rate is set at 1.2 mL/min.

### Example 12. Synthesis of (3R,5S,E)-7-(6-(bicyclo[1.1.1]pentan-1-yl)-2-cyclopropyl-4-(4-fluorophenyl)-5-(methoxymethyl)pyridin-3-yl)-3,5-dihydroxyhept-6-enoic acid

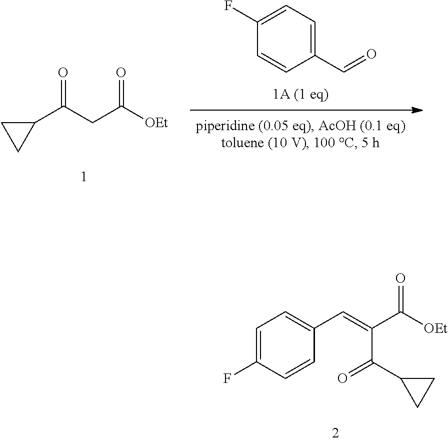 [SMILES: O=Cc1ccc(F)cc1 ; CCOC(=O)CC(=O)C1CC1 ; CCOC(=O)/C(=C/c1ccc(F)cc1)C(=O)C1CC1]

Step 1: A mixture of compound 1 (10 g, 32.01 mmol, 1 eq), compound 1A (7.94 g, 32.01 mmol, 1 eq), piperidine (272.6 mg, 1.60 mmol, 0.05 eq) and AcOH (382.5 mg, 3.20 mmol, 0.1 eq) in toluene (100 mL) was degassed and purged with N₂ for 3 times, then the mixture was stirred at 100 °C for 5 hours under N₂ atmosphere. LCMS showed the starting material was consumed completely and desired product was observed. After cooling to room temperature, the reaction mixture was concentrated under reduced pressure. The residue was purified by flash column chromatography on silica gel (0 to 10% EtOAc in PE) to afford compound 2 (12 g, 90.00% yield) as yellow oil. LCMS (ESI+): m/z=285.0 (M+23), RT: 0.434 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

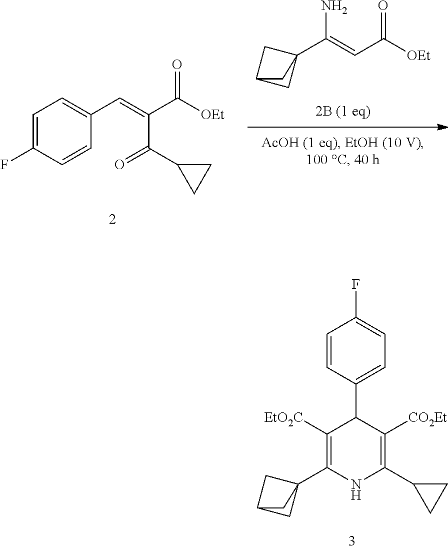 [SMILES: CCOC(=O)/C(=C/c1ccc(F)cc1)C(=O)C1CC1 ; CCOC(=O)/C=C(\N)C12CC(C1)C2 ; CCOC(=O)C1=C(C2CC2)NC(C23CC(C2)C3)=C(C(=O)OCC)C1c1ccc(F)cc1]

Step 2: A mixture of compound 2 (2.50 g, 9.53 mmol, 1 eq), compound 2B (1.73 g, 9.53 mmol, 1 eq) and AcOH (572.41 mg, 9.53 mmol, 1 eq) in EtOH (25 mL) was degassed and purged with N₂ for 3 times, then the mixture was stirred at 100 °C for 40 hours under N₂ atmosphere. LCMS showed the starting material was remained and desired product was observed. The reaction mixture was concentrated under reduced pressure. The residue was purified by flash column chromatography on silica gel (0 to 10% EtOAc in PE) to afford compound 3 (2.6 g, 74% purity, 47.44% yield) as yellow oil. LCMS (ESI+): m/z=426.2 (M+1), RT: 0.540 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

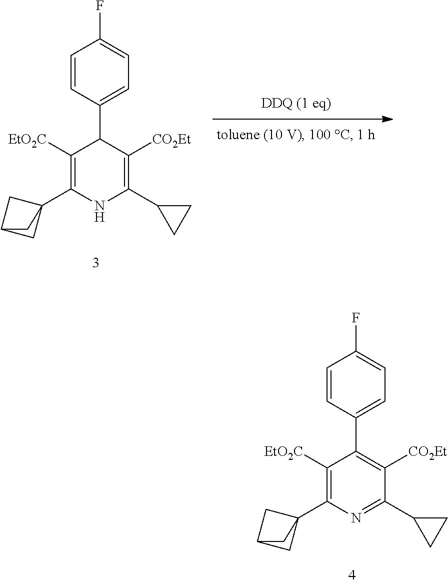 [SMILES: CCOC(=O)C1=C(C2CC2)NC(C23CC(C2)C3)=C(C(=O)OCC)C1c1ccc(F)cc1 ; CCOC(=O)c1c(C2CC2)nc(C23CC(C2)C3)c(C(=O)OCC)c1-c1ccc(F)cc1]

Step 3: A mixture of compound 3 (2.5 g, 4.35 mmol, 1 eq) and DDQ (986.99 mg, 4.35 mmol, 1 eq) in toluene. (25 mL) was degassed and purged with N₂ for 3 times, and then the mixture was stirred at 100 °C for 1 hour under N₂ atmosphere. LCMS showed the starting material was consumed completely and desired product was observed. The reaction mixture was concentrated under reduced pressure. The residue was purified by flash column chromatography on silica gel (0 to 10% EtOAc in PE) to afford compound 4 (1.6 g, 74% purity, 86.90% yield) as a yellow solid. LCMS (ESI+): m/z=424.3 (M+1), RT: 0.587 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

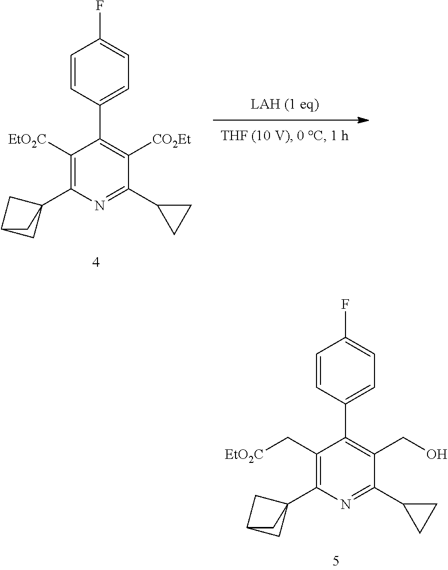 [SMILES: CCOC(=O)c1c(C2CC2)nc(C23CC(C2)C3)c(C(=O)OCC)c1-c1ccc(F)cc1 ; CCOC(=O)Cc1c(C23CC(C2)C3)nc(C2CC2)c(CO)c1-c1ccc(F)cc1]

Step 4: To a solution of compound 4 (1.5 g, 3.54 mmol, 1 eq) in THF (15 mL) was added LAH (1 M, 3.54 mL, 1 eq) at 0 °C. The mixture was stirred at 0 °C for 1 hour under N₂ atmosphere. LCMS showed desired product was observed. The reaction was quenched with (H₂O: 15% NaOH:H₂O=1 mL: 1 mL: 3 mL) at 0 °C and stirred for 5 minutes. Na₂SO₄ was added. The mixture was stirred for 15 minutes and filtered. The filtrate was concentrated under reduced pressure. The residue was purified by flash column chromatography on silica gel (0 to 10% EtOAc in PE) to afford compound 5 (1.1 g, 81.42% yield) as a white solid. ¹H NMR: (400 MHz, CDCl₃) δ 0.95-1.04 (m, 5H), 1.21-1.27 (m, 2H), 1.45 (t, J=5.6 Hz, 1H), 2.13 (s, 6H), 2.33-2.42 (m, 1H), 2.47 (s, 1H), 3.97 (q, J=7.2 Hz, 2H), 4.56 (d, J=5.6 Hz, 2H), 7.07-7.14 (m, 2H), 7.22-7.27 (m, 2H). LCMS (ESI+): m/z=382.4 (M+1), RT: 0.508 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

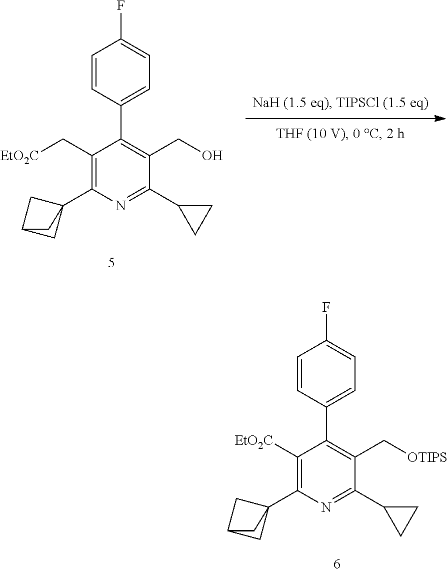

Step 5: To a solution of compound 5 (960 mg, 2.52 mmol, 1 eq) in THF (10 mL) was added NaH (201.32 mg, 5.03 mmol, 60% purity, 2 eq) at 0 °C under N₂. After addition, the mixture was stirred at this temperature for 0.5 hour, then TIPSCl (727.86 mg, 3.78 mmol, 1.5 eq) was added dropwise at 0 °C. The resulting mixture was stirred at 25 °C for 1.5 hour under N₂ atmosphere. LCMS showed the starting material was consumed completely and desired product was observed. The reaction was quenched with water 10 mL and extracted with EtOAc (10 mL×2). The combined organic layers were washed with brine (10 mL×2), dried over Na₂SO₄, filtered and concentrated under reduced pressure. The residue was purified by flash column chromatography on silica gel (0 to 10% EtOAc in PE) to afford compound 6 (1.3 g, 96.05% yield) as yellow oil. LCMS (ESI+): m/z=538.9 (M+1), RT: 0.724 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

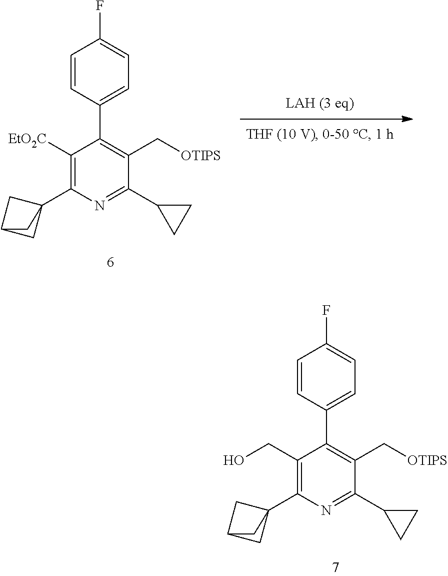

Step 6: To a solution of compound 6 (1.3 g, 2.42 mmol, 1 eq) in THF (15 mL) was added LAH (275.22 mg, 7.25 mmol, 3 eq) at 0 °C. The mixture was stirred at 50 °C for 1 hour under N₂ atmosphere. LCMS showed the starting material was remained and desired product was observed. The reaction was quenched with (H₂O: 15% NaOH:H₂O=0.3 mL: 0.3 mL: 0.9 mL) at 0 °C and stirred for 5 minutes. Na₂SO₄ was added. The mixture was stirred for 15 minutes and filtered. The filtrate was concentrated under reduced pressure. The residue was purified by flash column chromatography on silica gel (0 to 10% EtOAc in PE) to afford compound 7 (680 mg, 81.42% yield) as yellow oil. ¹H NMR: (400 MHz, CDCl₃) δ 0.92-0.98 (m, 22H), 1.14-1.24 (m, 3H), 2.27 (s, 6H), 2.36-2.49 (m, 1H), 2.54 (s, 1H), 4.40 (d, J=5.6 Hz, 2H), 4.57 (s, 2H), 7.08-7.15 (m, 2H), 7.18-7.25 (m, 2H). LCMS (ESI+): m/z=496.3 (M+1), RT: 0.618 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

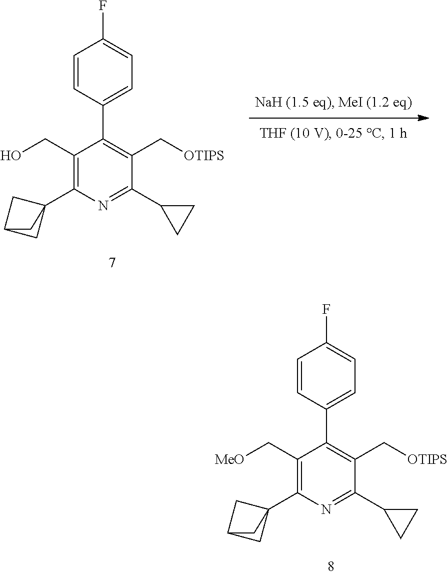

Step 7: To a solution of compound 7 (860 mg, 1.73 mmol, 1 eq) in THF (10 mL) was added NaH (138.78 mg, 3.47 mmol, 60% purity, 2 eq) at 0 °C. After addition, the mixture was stirred at this temperature for 0.5 hour, then CH₃I (369.35 mg, 2.60 mmol, 1.5 eq) was added dropwise at 0 °C. The resulting mixture was stirred at 25 °C for 0.5 hour under N₂ atmosphere. LCMS showed the starting material was remained and desired product was observed. The reaction was quenched with water (10 mL) and extracted with EtOAc (10 mL×2). The combined organic layers were washed with brine (10 mL×2), dried over Na₂SO₄, filtered and concentrated under reduced pressure to give a residue. The residue was purified by flash column chromatography on silica gel (0 to 10% EtOAc in PE) to afford compound 8 (710 mg, 80.29% yield) as colorless oil. LCMS (ESI+): m/z=510.9 (M+1), RT: 0.691 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

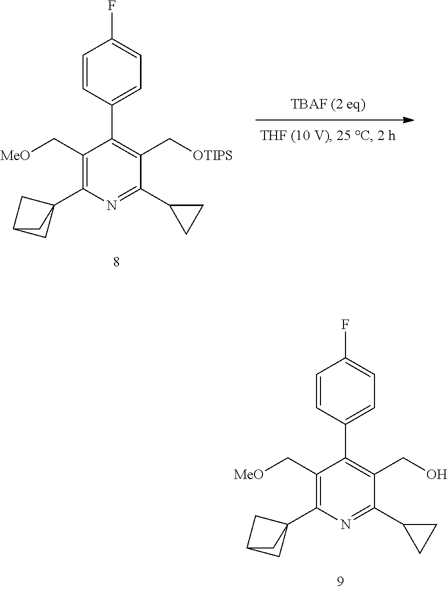

Step 8: To a solution of compound 8 (710 mg, 1.39 mmol, 1 eq) in THF (8 mL) was added TBAF (1 M, 2.79 mL, 2 eq). The mixture was stirred at 25 °C for 2 hours under N₂ atmosphere. LCMS showed the starting material was consumed completely and desired product was observed. The reaction was diluted with water (10 mL) and extracted with EtOAc (10 mL×2). The combined organic layers were washed with brine (10 mL×2), dried over Na₂SO₄, filtered and concentrated under reduced pressure. The residue was purified by flash column chromatography on silica gel (0 to 10% EtOAc in PE) to afford compound 9 (430 mg, 87.35% yield) as yellow oil. LCMS (ESI+): m/z=354.4 (M+1), RT: 0.401 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

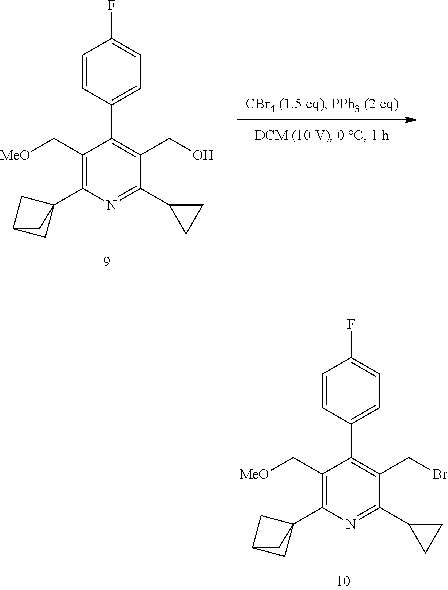 [SMILES: COCc1c(C23CC(C2)C3)nc(C2CC2)c(CO)c1-c1ccc(F)cc1 ; COCc1c(C23CC(C2)C3)nc(C2CC2)c(CBr)c1-c1ccc(F)cc1]

Step 9: To a solution of compound 9 (300 mg, 848.83 μmol, 1 eq) in DCM (4 mL) was added CBr₄ (422.24 mg, 1.27 mmol, 1.5 eq) and PPh₃ (445.28 mg, 1.70 mmol, 2 eq) at 0 °C under N₂. The resulting mixture was stirred at 0 °C for 1 hour. LCMS showed the starting material was consumed completely and desired product was observed. The reaction was concentrated under reduced pressure. The residue was purified by flash column chromatography on silica gel (0 to 10% EtOAc in PE) to afford compound 10 (440 mg, 80.00% yield) as a white solid. LCMS (ESI+): m/z=417.7 (M+1), RT: 0.594 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

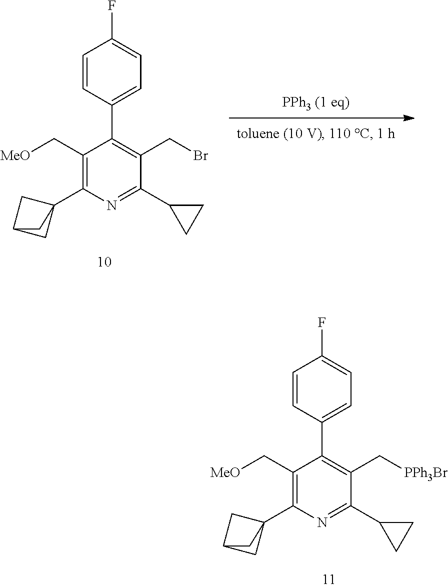

Step 10: A mixture of compound 10 (440.00 mg, 845.49 μmol, 1 eq), PPh₃ (221.76 mg, 845.49 μmol, 1 eq) in toluene (5 mL) was degassed and purged with N₂ for 3 times, then the mixture was stirred at 110 °C for 1 hour under N₂ atmosphere. LCMS showed the starting material was consumed completely and desired product was observed. After cooling to room temperature, the reaction mixture was concentrated under reduced pressure to give compound 11 (580 mg, crude) as colorless liquid. LCMS (ESI+): m/z=598.7 (M-Br), RT: 0.468 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

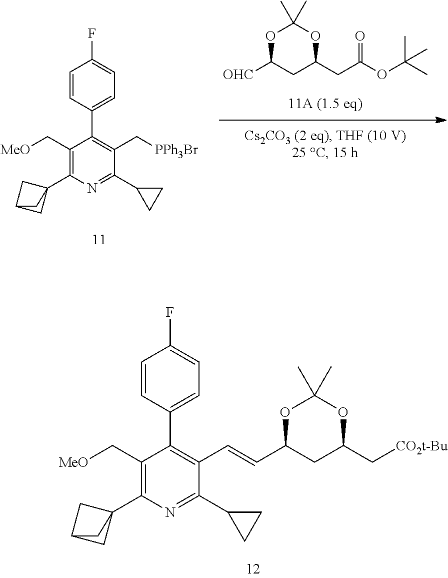

Step 11: A mixture of compound 11 (580 mg, 854.69 μmol, 1 eq), compound 11A (331.16 mg, 1.28 mmol, 1.5 eq) and Cs₂CO₃ (556.95 mg, 1.71 mmol, 2 eq) in THF (8 mL) was degassed and purged with N₂ for 3 times, then the mixture was stirred at 25 °C for 15 hours under N₂ atmosphere. LCMS showed the starting material was consumed completely and desired MS was observed. The reaction mixture was concentrated under reduced pressure. The residue was purified by flash column chromatography on silica gel (0 to 10% EtOAc in PE) to afford compound 12 (230 mg, 40.52% yield, 87% purity) as a white solid. ¹H NMR: (400 MHz, CDCl₃) δ 0.88 (dd, J=8.0, 2.4 Hz, 2H), 1.12-1.22 (m, 2H), 1.24-1.32 (m, 2H), 1.35 (s, 3H), 1.43 (s, 3H), 1.44-1.47 (m, 9H), 2.24 (s, 6H), 2.25-2.29 (m, 2H), 2.40 (dd, J=15.2, 6.8 Hz, 1H), 2.51 (s, 1H), 3.16 (s, 3H), 4.02-4.11 (m, 2H), 4.17-4.32 (m, 2H), 5.50 (dd, J=16.4, 6.0 Hz, 1H), 6.23 (d, J=16.4 Hz, 1H), 7.03-7.16 (m, 4H). LCMS (ESI+): m/z=578.7 (M+1), RT: 0.594 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

In some embodiments, compound 12 is the sodium salt of compound 12.

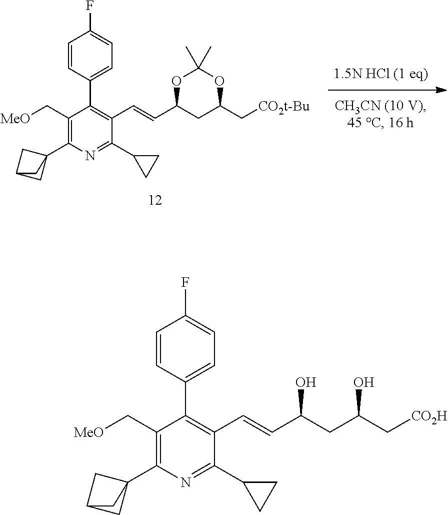 [SMILES: COCc1c(C23CC(C2)C3)nc(C2CC2)c(/C=C/[C@@H]2C[C@H](CC(=O)OC(C)(C)C)OC(C)(C)O2)c1-c1ccc(F)cc1 ; COCc1c(C23CC(C2)C3)nc(C2CC2)c(/C=C/[C@@H](O)C[C@@H](O)CC(=O)O)c1-c1ccc(F)cc1]

Step 12: To a solution compound 12 (100 mg, 150.59 μmol, 1 eq) in CH₃CN (1 mL) was added HCl (1.5 M, 1.5 eq) and the mixture was stirred at 45 °C for 16 hours under N₂ atmosphere. LCMS showed desired product was observed. The mixture was purified by prep-HPLC (column: Phenomenex luna C18 150*25 mm*10 μm; mobile phase: [water (FA)-ACN]; gradient: 45%-75% B over 10 min) to afford (3R,5S,E)-7-(6-(bicyclo[1.1.1]pentan-1-yl)-2-cyclopropyl-4-(4-fluorophenyl)-5-(methoxymethyl)pyridin-3-yl)-3,5-dihydroxyhept-6-enoic acid (18.90 mg, 23.98% yield) as a white solid. ¹H NMR: (400 MHz, CDCl₃) δ 0.80-0.96 (m, 2H), 1.16 (s, 2H), 1.24-1.57 (m, 2H), 2.16-2.21 (m, 1H), 2.24 (s, 6H), 2.42-2.54 (m, 3H), 3.16 (s, 3H), 4.07 (s, 2H), 4.10-4.36 (m, 2H), 5.50 (dd, J=16.8, 5.6 Hz, 1H), 6.31 (d, J=16.0 Hz, 1H), 7.02-7.17 (m, 4H). LCMS (ESI+): m/z=482.3 (M+1), RT: 1.690 min. LC/MS: Kinetex® EVO C18 3.0×50 mm 2.6 μm Detection methods are photo-diode array (PDA&ELSD). MS mode was positive electrospray ionization. MS range was 50-1050. Mobile phase: Ramp from 5% ACN (0.01875% TFA) in water (0.0375% TFA) to 95% ACN in water in 3.4 min, Flow rate is set at 0.9 mL/min; then hold at 95% ACN for 0.3 minutes Flow rate is set 0.9 mL/min; return back to 5% ACN in water and hold for 0.3 min. Flow rate is set at 1.2 mL/min.

### Example 13. Synthesis of (3R,5S,E)-7-(4-(2-chloro-4-fluorophenyl)-2,6-dicyclobutyl-5-(methoxymethyl)pyridin-3-yl)-3,5-dihydroxyhept-6-enoic acid

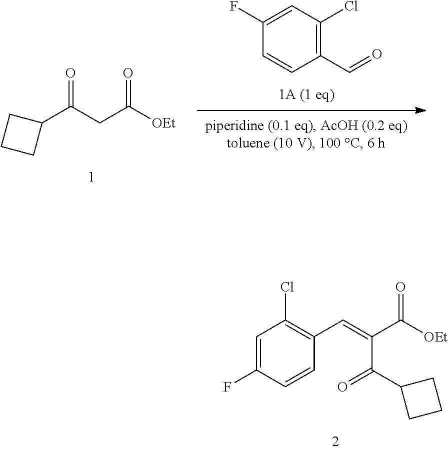 [SMILES: O=Cc1ccc(F)cc1Cl ; CCOC(=O)CC(=O)C1CCC1 ; CCOC(=O)/C(=C/c1ccc(F)cc1Cl)C(=O)C1CCC1]

Step 1: To a solution of compound 1 (4 g, 23.50 mmol, 1 eq) and compound 1A (3.73 g, 23.50 mmol, 1 eq) in toluene (40 mL) was added piperidine (200.10 mg, 2.35 mmol, 0.1 eq) and AcOH (282.26 mg, 4.70 mmol, 0.2 eq). The mixture was stirred at 100 °C for 6 hours. LCMS showed the desired MS was observed. The reaction mixture was concentrated under reduced pressure. The residue was diluted with H₂O (150 mL) and extracted with EtOAc (200 mL×2). The combined organic layers were dried over Na₂SO₄, filtered and concentrated under reduced pressure. The residue was purified by flash column chromatography on silica gel (0 to 10% EtOAc in PE) to afford compound 2 (2.3 g, 31.19% yield) as a luminous yellow oil. LCMS (ESI+): m/z=332.9 (M+23), RT: 0.518 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

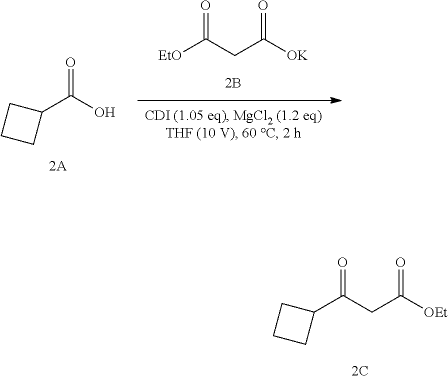

Step 2: To a solution of compound 2A (25 g, 249.72 mmol, 1 eq) in THF (300 mL) was added CDI (42.56 g, 262.20 mmol, 1.05 eq) and the mixture was stirred at 60 °C for 1 hour. MgCl₂ (28.54 g, 299.66 mmol, 1.2 eq) and compound 2B (44.62 g, 262.20 mmol, 1.05 eq) was added and the mixture was stirred at 60 °C for 1 hour. The reaction mixture was filtered and concentrated under reduced pressure. The residue was purified by flash column chromatography on silica gel (0 to 5% EtOAc in PE) to afford compound 2C (21 g, 70% purity 34.59% yield) as colorless oil. ¹H NMR: (400 MHz, DMSO-d₆) δ 1.18 (t, J=7.2 Hz, 3H), 1.66-1.80 (m, 1H), 1.85-1.95 (m, 1H), 2.06-2.13 (m, 4H), 3.39 (q, J=8.8 Hz, 1H), 3.50 (s, 2H), 4.08 (q, J=7.2 Hz, 2H).

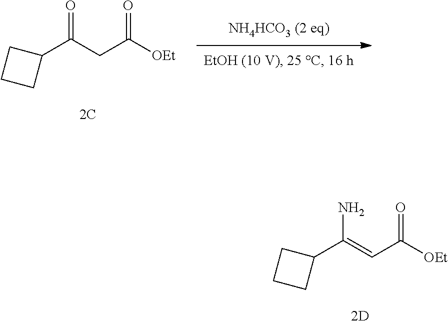 [SMILES: CCOC(=O)CC(=O)C1CCC1 ; "NH4HCO3 (2 eq)" ; CCOC(=O)/C=C(\N)C1CCC1]

Step 3: A mixture of compound 2C (21 g, 86.36 mmol, 1 eq) and NH₄HCO₃ (13.66 g, 172.74 mmol, 2 eq) in EtOH (210 mL) was degassed and purged with N₂ for 3 times and the mixture was stirred at 25 °C for 16 hours under N₂ atmosphere. The reaction mixture was filtered and concentrated under reduced pressure. The residue was purified by flash column chromatography on silica gel (0 to 5% EtOAc in PE) to afford compound 2D (11 g, 90% purity 67.74% yield) as yellow oil. ¹H NMR: (400 MHz, DMSO-d₆) δ 1.14 (t, J=7.2, 3H), 1.63-1.76 (m, 1H), 1.79-1.94 (m, 1H), 2.00-2.16 (m, 4H), 2.93-3.08 (m, 1H), 3.96 (q, J=7.2 Hz, 2H), 4.32 (s, 1H), 6.75 (s, 1H), 7.68 (s, 1H).

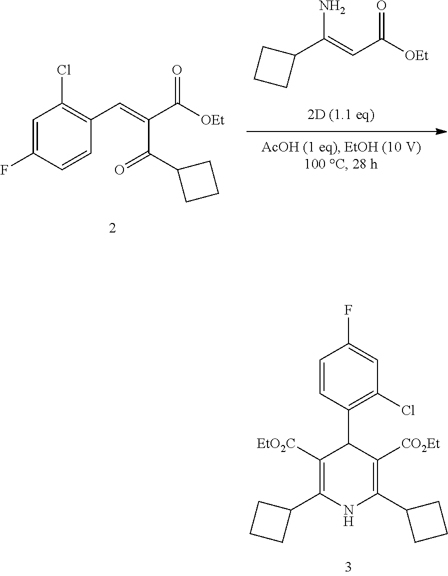 [SMILES: CCOC(=O)/C(=C/c1ccc(F)cc1Cl)C(=O)C1CCC1 ; CCOC(=O)/C=C(\N)C1CCC1 ; CCOC(=O)C1=C(C2CCC2)NC(C2CCC2)=C(C(=O)OCC)C1c1ccc(F)cc1Cl]

Step 4: To a solution of compound 2 (2.22 g, 7.14 mmol, 1 eq) and compound 2D (1.45 g, 8.57 mmol, 1.2 eq) in EtOH (23 mL) was added AcOH (858.03 mg, 14.29 mmol, 2 eq). The mixture was stirred at 100 °C for 28 hours. LCMS showed the desired MS was observed. The reaction mixture was concentrated under reduced pressure. The residue was purified by flash column chromatography on silica gel (0 to 10% EtOAc in PE) to afford compound 3 (1.9 g, 57.58% yield) as a white solid. LCMS (ESI+): m/z=462.1 (M+1), RT: 0.587 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

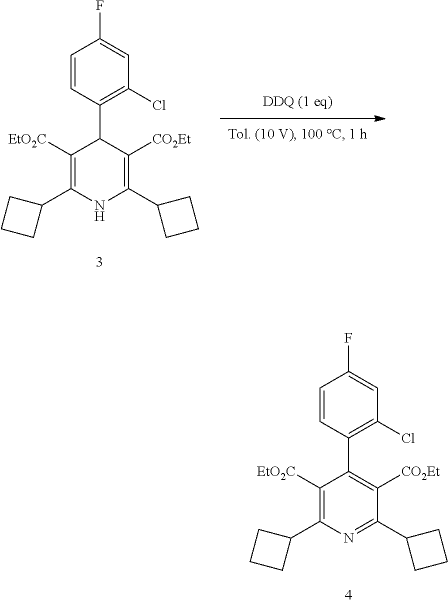 [SMILES: CCOC(=O)C1=C(C2CCC2)NC(C2CCC2)=C(C(=O)OCC)C1c1ccc(F)cc1Cl ; CCOC(=O)c1c(C2CCC2)nc(C2CCC2)c(C(=O)OCC)c1-c1ccc(F)cc1Cl]

Step 5: To a solution of compound 3 (1.9 g, 4.11 mmol, 1 eq) in Toluene (20 mL) was added DDQ (933.66 mg, 4.11 mmol, 1 eq). The mixture was stirred at 100 °C for 1 hour. LCMS showed the starting material was consumed completely and desired MS was observed. The reaction mixture was concentrated under reduced pressure. The residue was purified by flash column chromatography on silica gel (0 to 6% EtOAc in PE) to afford compound 4 (1.75 g, 91.67% yield) as a white solid. LCMS (ESI+): m/z=460.4 (M+1), RT: 0.615 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

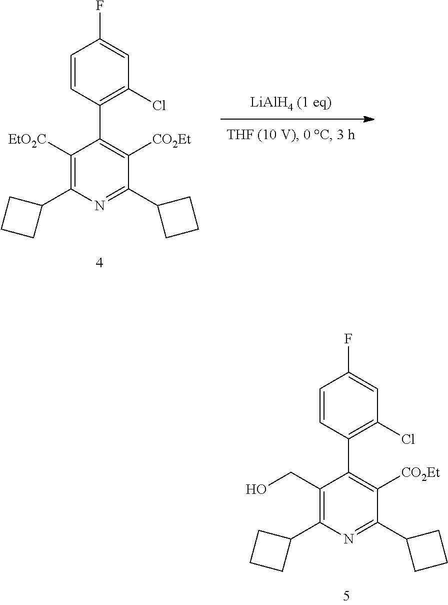 [SMILES: CCOC(=O)c1c(C2CCC2)nc(C2CCC2)c(C(=O)OCC)c1-c1ccc(F)cc1Cl ; CCOC(=O)c1c(C2CCC2)nc(C2CCC2)c(CO)c1-c1ccc(F)cc1Cl]

Step 6: To a solution of compound 4 (1.4 g, 3.04 mmol, 1 eq) in THF (15 mL) was added LiAlH₄ (1 M, 3.04 mL, 1 eq) at 0 °C under N₂. The mixture was stirred at 0 °C for 3 hours under N₂ atmosphere. LCMS showed the desired MS was observed. The reaction was quenched with Na₂SO₄·10 H₂O (150 mg) at 0 °C under N₂. The reaction mixture was filtered and the filter cake was washed with THF (20 mL) and the filtrate was concentrated under reduced pressure. The residue was purified by flash column chromatography on silica gel (0 to 20% EtOAc in PE) to afford compound 5 (1 g, 78.61% yield) as a luminous yellow oil. LCMS (ESI+): m/z=418.2 (M+1), RT: 0.546 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

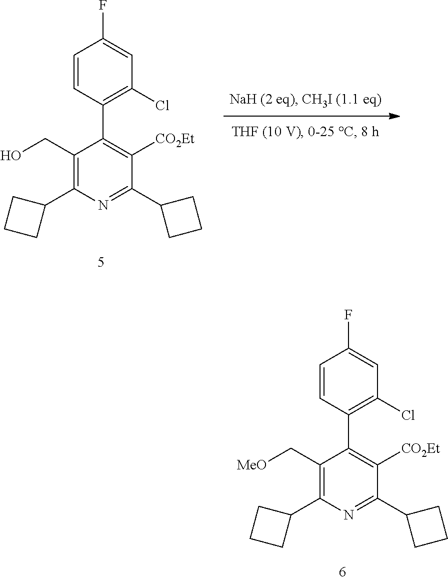 [SMILES: CCOC(=O)c1c(C2CCC2)nc(C2CCC2)c(CO)c1-c1ccc(F)cc1Cl ; CCOC(=O)c1c(C2CCC2)nc(C2CCC2)c(COC)c1-c1ccc(F)cc1Cl]

Step 7: To a solution compound 5 (0.53 g, 1.27 mmol, 1 eq) in THF (15 mL) was added NaH (60.87 mg, 1.52 mmol, 60% purity, 1.2 eq) at 0 °C. After addition, the mixture was stirred at this temperature for 0.5 hour, then CH₃I (270.02 mg, 1.90 mmol, 1.5 eq) was added dropwise at 0 °C. The resulting mixture was stirred at 25 °C for 7.5 hours under N₂ atmosphere. LCMS showed the starting material was remained and desired product was observed. The reaction was quenched with water (10 mL) and extracted with EtOAc (10 mL×2). The combined organic layers were washed with brine (10 mL×2), dried over Na₂SO₄, filtered and concentrated under reduced pressure. The residue was purified by flash column chromatography on silica gel (0 to 10% EtOAc in PE) to afford compound 6 (1 g, 66.67% yield) as yellow oil. LCMS (ESI+): m/z=433.3 (M+1), RT: 0.606 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

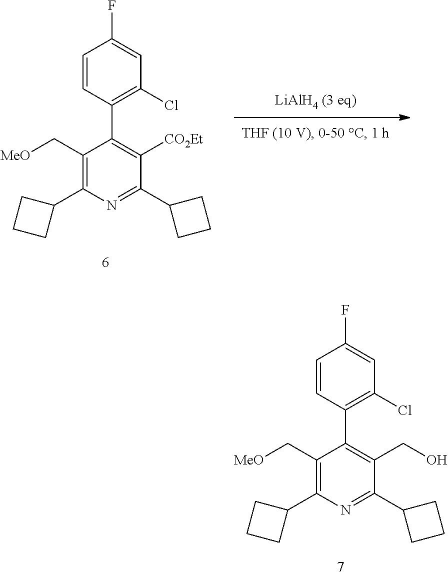 [SMILES: CCOC(=O)c1c(C2CCC2)nc(C2CCC2)c(COC)c1-c1ccc(F)cc1Cl ; COCc1c(C2CCC2)nc(C2CCC2)c(CO)c1-c1ccc(F)cc1Cl]

Step 8: To a solution of compound 6 (900 mg, 2.08 mmol, 1 eq) in THF (10 mL) was added LAH (316.34 mg, 8.33 mmol, 4 eq) at 0 °C. The mixture was stirred at 50 °C for 1 hour under N₂ atmosphere. LCMS showed the starting material was remained and desired product was observed. After cooling to room temperature, the reaction was quenched with (H₂O: 15% NaOH:H₂O=0.3 mL: 0.3 mL: 0.9 mL) at 0 °C, and stirred for 5 minutes. Na₂SO₄ was added. The mixture was stirred for 15 minutes and filtered. The filtrate was concentrated under reduced pressure. The residue was purified by flash column chromatography on silica gel (0 to 10% EtOAc in PE) to afford compound 7 (620 mg, 67.16% yield) as yellow oil. LCMS (ESI+): m/z=390.1 (M+1), RT: 0.464 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

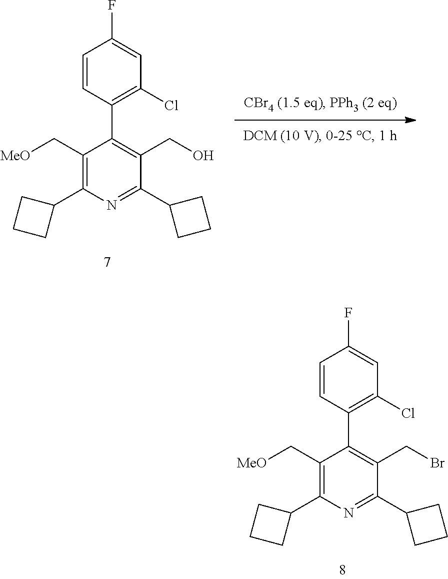 [SMILES: COCc1c(C2CCC2)nc(C2CCC2)c(CO)c1-c1ccc(F)cc1Cl ; COCc1c(C2CCC2)nc(C2CCC2)c(CBr)c1-c1ccc(F)cc1Cl]

Step 9: To a solution of compound 7 (620 mg, 1.40 mmol, 1 eq) in DCM (10 mL) was added CBr₄ (696.10 mg, 2.10 mmol, 1.5 eq) and PPh₃ (734.07 mg, 2.80 mmol, 2 eq) at 0 °C. The resulting mixture was stirred at 25 °C for 1 hour under N₂ atmosphere. LCMS showed the starting material was consumed completely and desired product was observed. The reaction was concentrated under reduced pressure. The residue was purified by flash column chromatography on silica gel (0 to 10% EtOAc in PE) to afford compound 8 (620 mg, 92% purity, 80.00% yield) as a white solid. LCMS (ESI+): m/z=454.1 (M+1), RT: 0.632 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

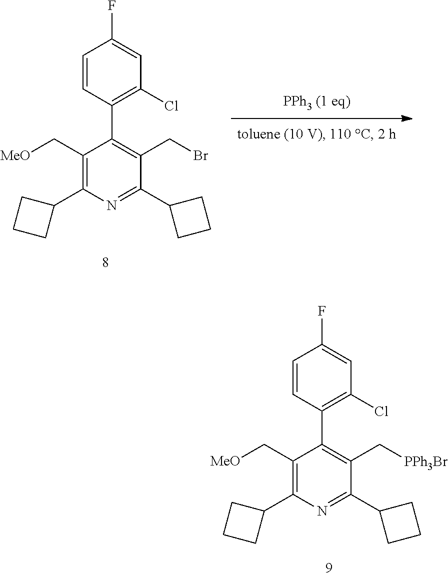

Step 10: A mixture of compound 8 (620 mg, 1.26 mmol, 1 eq), PPh₃ (330.42 mg, 1.26 mmol, 1 eq) in toluene (8 mL) was degassed and purged with N₂ for 3 times, and the mixture was stirred at 100 °C for 2 hours under N₂ atmosphere. LCMS showed the starting material was consumed completely and desired product was observed. After cooling to room temperature, the reaction mixture was concentrated under reduced pressure to give compound 9 (1.1 g, crude) as a white solid. LCMS (ESI+): m/z=634.5 (M-Br), RT: 0.487 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

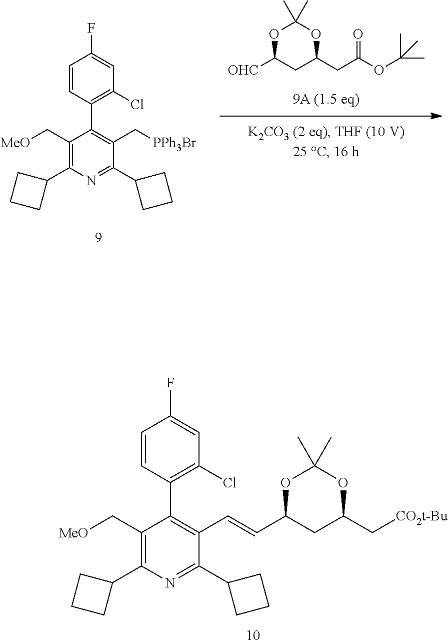

Step 11: A mixture of compound 9 (1.1 g, 1.54 mmol, 1 eq), compound 9A (596.04 mg, 2.31 mmol, 1.5 eq) and K₂CO₃ (425.21 mg, 3.08 mmol, 2 eq) in THF (15 mL) was degassed and purged with N₂ for 3 times, and the mixture was stirred at 25 °C for 16 hours under N₂ atmosphere. LCMS showed the starting material was consumed completely and desired MS was observed. The reaction mixture was concentrated under reduced pressure. The residue was purified by flash column chromatography on silica gel (0 to 10% EtOAc in PE). The crude product was purified by prep-TLC (DCM:PE=1:1) to afford compound 10 (190 mg, 53% purity, 20.11% yield) as colorless oil. ¹H NMR: (400 MHz, CDCl₃) δ 1.42-1.48 (m, 15H), 2.00 (dd, J=18.4, 9.2 Hz, 4H), 2.12-2.96 (m, 14H), 3.12 (d, J=5.2 Hz, 3H), 3.78-4.05 (m, 4H), 5.09-5.33 (m, 1H), 6.09-6.26 (m, 1H), 7.01-7.17 (m, 3H). LCMS (ESI+): m/z=614.5 (M+1), RT: 0.620 min. LC/MS: HALO C18 90A 2.7 μm 3.0×30 mm Detection methods are photo-diode array (DAD&ELSD). MS mode was positive electrospray ionization. MS range was 50-1050. Mobile phase: Ramp from 5% ACN (0.018% TFA) in water (0.037% TFA) to 95% ACN in water in 3.0 min, Flow rate is set at 1.0 mL/min; then hold at 95% ACN for 0.6 minutes Flow rate is set from 1.0 mL/min to 1.5 mL/min; return back to 5% ACN in water and hold for 0.4 min. Flow rate is set at 1.5 mL/min.

In some embodiments, compound 10 is the sodium salt of compound 10.

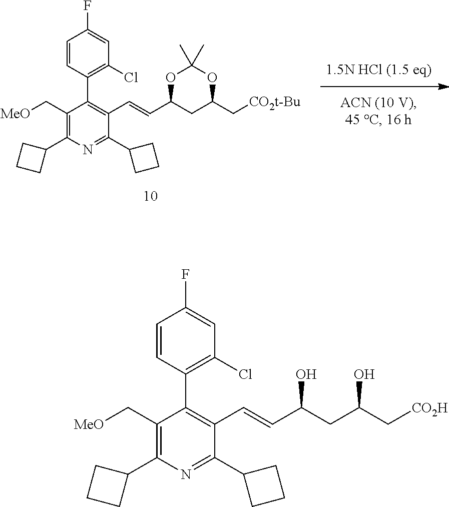 [SMILES: COCc1c(C2CCC2)nc(C2CCC2)c(/C=C/[C@@H]2C[C@H](CC(=O)OC(C)(C)C)OC(C)(C)O2)c1-c1ccc(F)cc1Cl ; COCc1c(C2CCC2)nc(C2CCC2)c(/C=C/[C@@H](O)C[C@@H](O)CC(=O)O)c1-c1ccc(F)cc1Cl]

Step 12: To a solution compound 10 (190 mg, 163.96 μmol, 1 eq) in ACN (2 mL) was added HCl (1.5 M, 1.5 eq) and the mixture was stirred at 45 °C for 16 hours under N₂ atmosphere. LCMS showed desired product was observed. After cooling to room temperature, the mixture was purified by prep-HPLC (column: Waters Xbridge 150*25 mm*5 μm; mobile phase: [water (NH₃·H₂O)-ACN]; gradient: 24%-44% B over 12 min) to afford (3R,5S,E)-7-(4-(2-chloro-4-fluorophenyl)-2,6-dicyclobutyl-5-(methoxymethyl)pyridin-3-yl)-3,5-dihydroxyhept-6-enoic acid (23.00 mg, 25.46% yield) as a brown solid. ¹H NMR: (400 MHz, CDCl₃) δ 1.23-1.78 (m, 2H), 1.91-2.11 (m, 4H), 2.19-2.34 (m, 4H), 2.43-2.72 (m, 6H), 3.12 (s, 3H), 3.79-3.86 (m, 2H), 3.92-4.02 (m, 2H), 4.19-4.33 (m, 2H), 5.22-5.41 (m, 1H), 6.20-6.38 (m, 1H), 6.98-7.07 (m, 1H), 7.10-7.22 (m, 2H). LCMS (ESI+): m/z=518.3 (M+1), RT: 1.898 min. LC/MS: Kinetex® EVO C18 3.0×50 mm 2.6 μm Detection methods are photo-diode array (PDA&ELSD). MS mode was positive electrospray ionization. MS range was 50-1050. Mobile phase: Ramp from 5% ACN (0.01875% TFA) in water (0.0375% TFA) to 95% ACN in water in 3.4 min, Flow rate is set at 0.9 mL/min; then hold at 95% ACN for 0.3 minutes Flow rate is set 0.9 mL/min; return back to 5% ACN in water and hold for 0.3 min. Flow rate is set at 1.2 mL/min.

### Example 14. Synthesis of (3R,5S,E)-7-(4-(2-chloro-4-fluorophenyl)-6-cyclobutyl-2-cyclopropyl-5-(methoxymethyl)pyridin-3-yl)-3,5-dihydroxyhept-6-enoic acid sodium salt

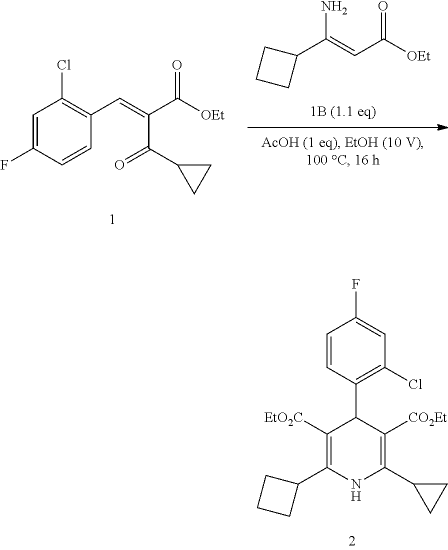 [SMILES: CCOC(=O)/C(=C/c1ccc(F)cc1Cl)C(=O)C1CC1 ; CCOC(=O)/C=C(\N)C1CCC1 ; CCOC(=O)C1=C(C2CCC2)NC(C2CC2)=C(C(=O)OCC)C1c1ccc(F)cc1Cl]

Step 1: A mixture of compound 1 (8 g, 26.96 mmol, 1 eq), AcOH (1.62 g, 26.96 mmol, 1 eq) and compound 1B (5.02 g, 29.66 mmol, 1.1 eq) in EtOH (100 mL) was degassed and purged with N₂ for 3 times, and then the mixture was stirred at 100 °C for 16 hours under N₂ atmosphere. LCMS showed the starting material was consumed completely and desired product was observed. The reaction mixture was concentrated under reduced pressure. The residue was purified by column chromatography on silica gel (PE/EtOAc=1:0 to 10:1) to afford compound 2 (9.1 g, 37.67% yield) as yellow oil. LCMS (ESI+): m/z=448.0 (M+1), RT: 0.550 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes, Flow rate is set at 2.0 mL/min; return back to 5% ACN in water and hold for 0.02 min. Flow rate is set at 2.0 mL/min.

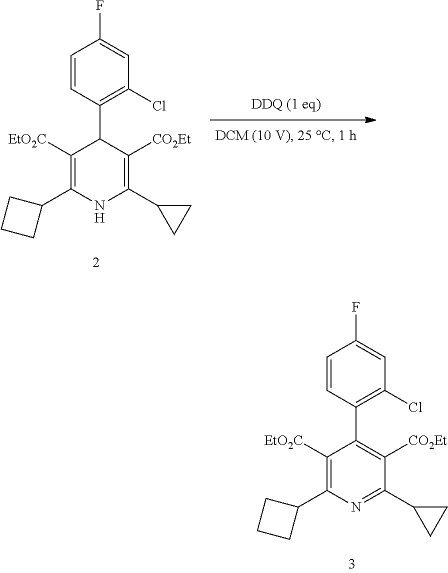 [SMILES: CCOC(=O)C1=C(C2CCC2)NC(C2CC2)=C(C(=O)OCC)C1c1ccc(F)cc1Cl ; CCOC(=O)c1c(C2CCC2)nc(C2CC2)c(C(=O)OCC)c1-c1ccc(F)cc1Cl]

Step 2: A mixture of compound 2 (9.1 g, 20.32 mmol, 1 eq), DDQ (4.61 g, 20.32 mmol, 1 eq) in toluene (100 mL) was degassed and purged with N₂ for 3 times, and then the mixture was stirred at 100 °C for 1 hour under N₂ atmosphere. LCMS showed the starting material was consumed completely and desired product was observed. The reaction mixture was concentrated under reduced pressure. The residue was purified by column chromatography on silica gel (PE/EtOAc=1:0 to 10:1) to afford compound 3 (7.6 g, 74% purity, 83.89% yield) as yellow oil. LCMS (ESI+): m/z=446.3 (M+1), RT: 0.586 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes, Flow rate is set at 2.0 mL/min; return back to 5% ACN in water and hold for 0.02 min. Flow rate is set at 2.0 mL/min.

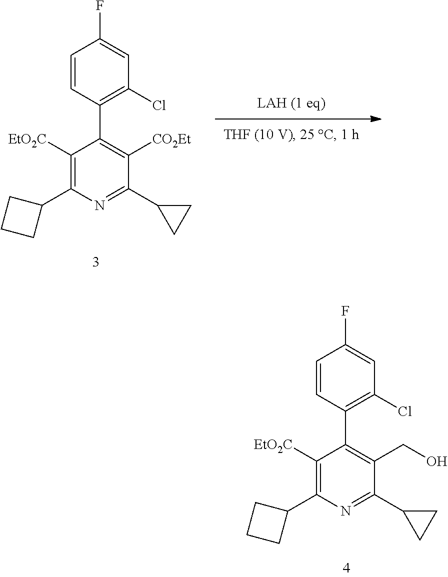 [SMILES: CCOC(=O)c1c(C2CCC2)nc(C2CC2)c(C(=O)OCC)c1-c1ccc(F)cc1Cl ; CCOC(=O)c1c(C2CCC2)nc(C2CC2)c(CO)c1-c1ccc(F)cc1Cl]

Step 3: A mixture of compound 3 (7.6 g, 17.04 mmol, 1 eq) in THF (70 mL) was degassed and purged with N₂ for 3 times, then LiAlH₄ (1 M, 17.04 mL, 1 eq) was added dropwise to the solution at 0 °C. The mixture was stirred at 25 °C for 1 hour under N₂ atmosphere. LCMS showed reactant was consumed completely and one main peak with 72% desired mass was detected. The reaction mixture was quenched by adding slowly 0.6 mL H₂O at 0 °C, then 0.6 mL 15% aq·NaOH solution and 1.8 mL H₂O were added. The mixture was dried with anhydrous Na₂SO₄, filtered and concentrated under reduced pressure. The residue was purified by flash column chromatography on silica gel (PE/EtOAc=1:0 to 5:1) to afford compound 4 (5.6 g, 74.68% yield) as yellow oil. ¹H NMR: (400 MHz, CDCl₃) δ 0.85-0.95 (m, 3H), 0.99 (br s, 2H), 1.17-1.22 (m, 2H), 1.75-1.92 (m, 2H), 2.07-2.22 (m, 2H), 2.27-2.52 (m, 3H), 3.55-3.72 (m, 1H), 3.83-3.99 (m, 2H), 4.17-4.54 (m, 2H), 6.97 (d, J=7.6 Hz, 1H), 7.08 (d, J=2.4 Hz, 1H), 7.12-7.20 (m, 1H). LCMS (ESI+): m/z=404.2 (M+1), RT: 0.443 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

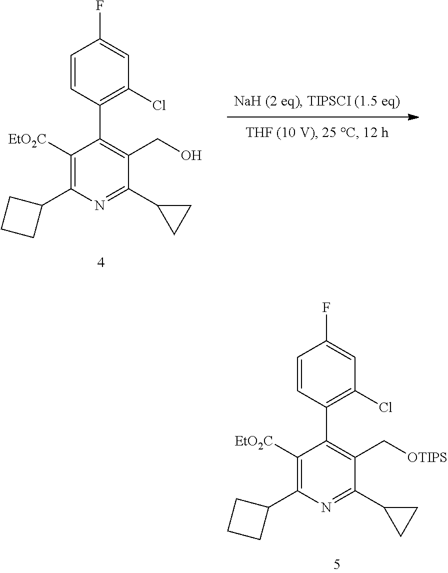

Step 4: A mixture of compound 4 (5.6 g, 12.73 mmol, 1 eq) in THF (50 mL) was degassed and purged with N₂ for 3 times, and NaH (1.02 g, 25.46 mmol, 231.10 μL, 60% purity, 2 eq) was added to the solution at 0 °C. The mixture was stirred at 0 °C for 30 min under N₂ atmosphere. Then TIPSCl (3.68 g, 19.09 mmol, 4.09 mL, 1.5 eq) was added dropwise to the solution. The mixture was stirred at 25 °C for 12 hours under N₂ atmosphere. LCMS showed ~37% of reactant remained. Two new peaks were shown on LCMS and ~41% of desired compound was detected. The reaction mixture was quenched by adding slowly 20 mL NH₄Cl solution at 0 °C. The mixture was extracted with ethyl acetate (30 mL×3) and washed with brine (20 mL×3), then the organic layer was dried with anhydrous Na₂SO₄, filtered and concentrated under reduced pressure to give a residue. The residue was purified by column chromatography on silica gel (PE/EtOAc=1:0) to afford compound 5 (5.4 g, 65.38% yield) as yellow solid. ¹H NMR: (400 MHz, CDCl₃) δ ppm 0.87-0.96 (m, 21H), 0.97-1.00 (m, 3H), 1.00-1.03 (m, 2H), 1.18-1.23 (m, 2H), 1.85-2.01 (m, 2H), 2.12-2.30 (m, 2H), 2.36-2.46 (m, 1H), 2.46-2.59 (m, 2H), 3.72-3.80 (m, 1H), 3.91-4.03 (m, 2H), 4.22-4.46 (m, 1H), 4.57-4.89 (m, 1H), 6.99 (td, J=8.4, 2.4 Hz, 1H), 7.14-7.24 (m, 2H). LCMS (ESI+): m/z=560.3 (M+1), RT: 0.743 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes, Flow rate is set at 2.0 mL/min; return back to 5% ACN in water and hold for 0.02 min. Flow rate is set at 2.0 mL/min.

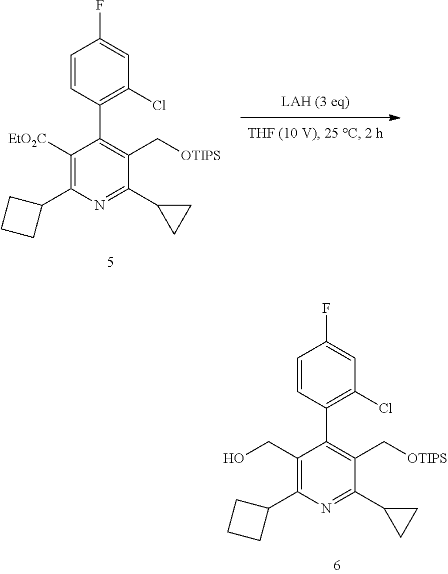

Step 5: A mixture of compound 5 (4.4 g, 6.78 mmol, 1 eq) in THF (40 mL) was degassed and purged with N₂ for 3 times, then LiAlH₄ (772.05 mg, 20.34 mmol, 3 eq) was added slowly to the solution at 0 °C. The mixture was stirred at 25 °C for 2 hours under N₂ atmosphere. LCMS showed the reactant was consumed completely and one main peak with 80% desired mass was detected. The reaction mixture was quenched by adding slowly 0.8 mL of H₂O, 0.8 mL of 15% aqueous NaOH solution and 2.4 mL of H₂O at 0 °C. The mixture was dried with anhydrous Na₂SO₄, filtered and concentrated under reduced pressure to give compound 6 (5 g, 97.45% yield) as yellow oil. ¹H NMR: (400 MHz, CDCl₃) δ ppm 0.92-0.98 (m, 21H), 0.99-1.01 (m, 2H), 1.30-1.35 (m, 2H), 1.87-2.03 (m, 2H), 2.20-2.31 (m, 2H), 2.42-2.65 (m, 3H), 3.73-3.82 (m, 1H), 3.92-4.19 (m, 2H), 4.32-4.37 (m, 1H), 4.72-4.81 (m, 1H), 7.02-7.10 (m, 1H), 7.17-7.26 (m, 2H). LCMS (ESI+): m/z=518.6 (M+1), RT: 0.548 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes, Flow rate is set at 2.0 mL/min; return back to 5% ACN in water and hold for 0.02 min. Flow rate is set at 2.0 mL/min.

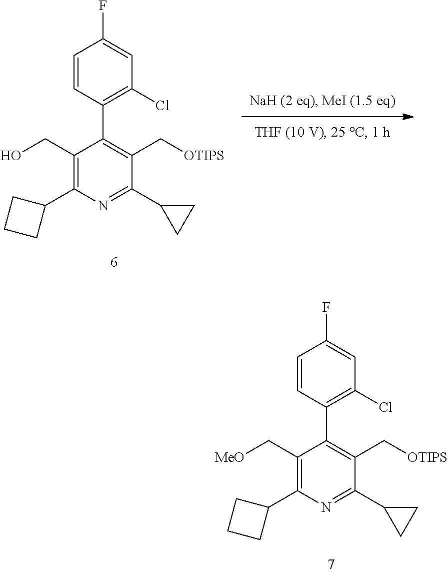

Step 6: A mixture of compound 6 (4 g, 6.34 mmol, 1 eq) in THF (40 mL) was degassed and purged with N₂ for 3 times, then NaH (507.39 mg, 12.69 mmol, 60% purity, 2 eq) was added slowly to the solution at 0 °C. The mixture was stirred at 0 °C for 30 min under N₂ atmosphere. Then Mel (1.35 g, 9.51 mmol, 592.31 μL, 1.5 eq) was added dropwise to the solution. The mixture was stirred at 25 °C for 1 hour under N₂ atmosphere. LCMS showed reactant was consumed completely and one main peak with 78% desired mass was detected. The reaction mixture was quenched by adding slowly NH₄Cl solution 20 mL at 0 °C. The mixture was extracted with ethyl acetate (20 mL×3) and washed with brine (20 mL×3), then the organic layer was dried with anhydrous Na₂SO₄, filtered and concentrated under reduced pressure to give compound 7 (3.7 g, 98.40% yield) as yellow oil. ¹H NMR: (400 MHz, CDCl₃) δ ppm 0.89-0.99 (m, 21H), 1.24-1.28 (m, 2H), 1.28-1.33 (m, 2H), 1.88-2.05 (m, 2H), 2.15-2.27 (m, 2H), 2.38-2.61 (m, 3H), 3.42-3.57 (m, 3H), 3.68-3.79 (m, 1H), 3.83-3.96 (m, 1H), 4.10-4.23 (m, 1H), 4.30-4.84 (m, 2H), 6.98-7.07 (m, 1H), 7.16-7.27 (m, 2H). LCMS (ESI+): m/z=533.2 (M+1), RT: 0.606 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes, Flow rate is set at 2.0 mL/min; return back to 5% ACN in water and hold for 0.02 min. Flow rate is set at 2.0 mL/min.

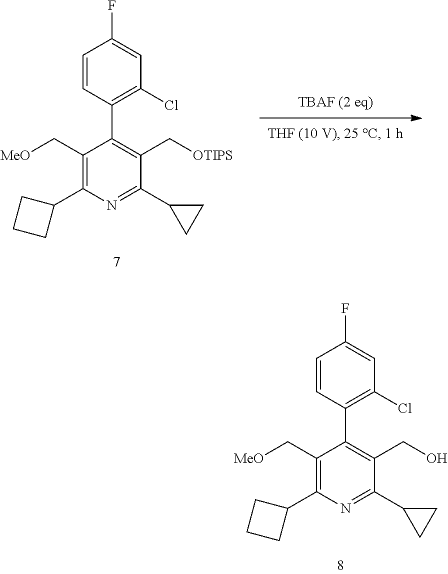

Step 7: To a solution of compound 7 (1.8 g, 3.04 mmol, 1 eq) in THF (20 mL) was added TBAF (1 M, 6.07 mL, 2 eq). The mixture was stirred at 25 °C for 1 hour. LCMS showed reactant was consumed completely and one main peak with 80% desired mass was detected. The mixture was concentrated under reduced pressure. The crude product was purified by column chromatography on silica gel (PE/EtOAc=1/0 to 5/1) to afford compound 8 (2 g, 77.00% yield) as colorless oil. ¹H NMR: (400 MHz, CDCl₃) δ 0.99-1.03 (m, 2H), 1.29-1.46 (m, 2H), 1.84-2.04 (m, 2H), 2.16-2.31 (m, 2H), 2.36-2.45 (m, 1H), 2.45-2.61 (m, 2H), 3.07-3.19 (m, 3H), 3.72-3.79 (m, 1H), 3.86-3.97 (m, 1H), 4.13-4.16 (m, 1H), 4.36-4.56 (m, 2H), 7.04-7.12 (m, 1H), 7.17-7.27 (m, 2H). LCMS (ESI+): m/z=376.5 (M+1), RT: 0.371 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes, Flow rate is set at 2.0 mL/min; return back to 5% ACN in water and hold for 0.02 min. Flow rate is set at 2.0 mL/min.

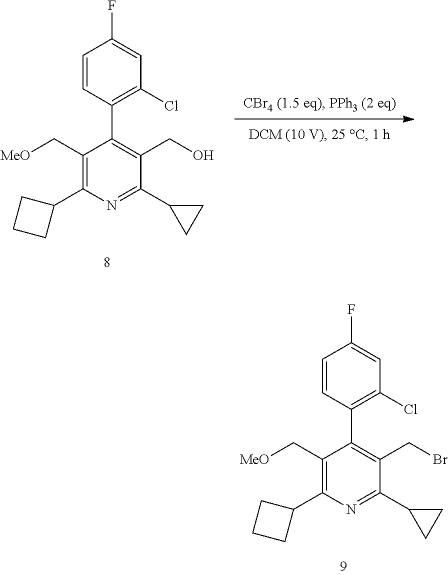 [SMILES: COCc1c(C2CCC2)nc(C2CC2)c(CO)c1-c1ccc(F)cc1Cl ; COCc1c(C2CCC2)nc(C2CC2)c(CBr)c1-c1ccc(F)cc1Cl]

Step 8: To a solution of compound 8 (0.5 g, 1.17 mmol, 1 eq) in DCM (5 mL) was added PPh₃ (613.25 mg, 2.34 mmol, 2 eq) and CBr₄ (581.53 mg, 1.75 mmol, 1.5 eq) at 0 °C. The mixture was stirred at 25 °C for 1 hour. LCMS showed the reactant was consumed completely and one peak with 35% desired mass was detected. The mixture was concentrated under reduced pressure to give a residue. The residue was purified by column chromatography on silica gel (PE/EtOAc=1/0 to 10/1) to give compound 9 (0.5 g, 97.24% yield) as colorless oil. ¹H NMR: (400 MHz, CDCl₃) δ 0.94-1.01 (m, 2H), 1.25-1.37 (m, 2H), 1.79-1.99 (m, 2H), 2.11-2.33 (m, 3H), 2.36-2.56 (m, 2H), 3.00-3.17 (m, 3H), 3.64-3.75 (m, 1H), 3.78-4.03 (m, 2H), 4.09-4.57 (m, 2H), 7.03-7.12 (m, 1H), 7.18-7.26 (m, 2H). LCMS (ESI+): m/7.=438.0 (M+1), RT: 0.605 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes, Flow rate is set at 2.0 mL/min; return back to 5% ACN in water and hold for 0.02 min. Flow rate is set at 2.0 mL/min.

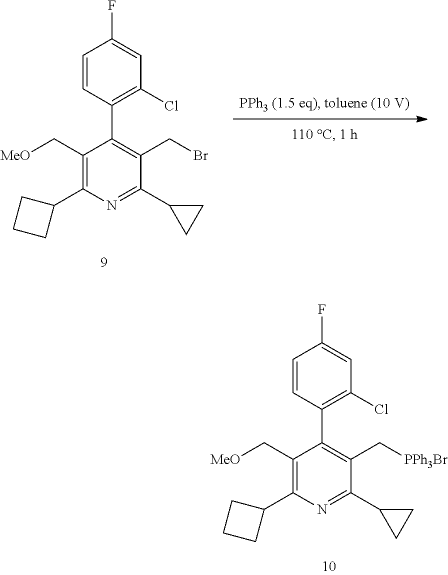

Step 9: A mixture of compound 9 (0.5 g, 1.14 mmol, 1 eq), PPh₃ (447.22 mg, 1.71 mmol, 1.5 eq) in toluene (5 mL) was degassed and purged with N₂ for 3 times, and then the mixture was stirred at 110 °C for 1 hour under N₂ atmosphere. LCMS showed the reactant was consumed completely and one peak with 61% desired mass was detected. The mixture was concentrated under reduced pressure to give compound 10 (79 mg, 100.00% yield) as a white solid. LCMS (ESI+): m/z=620.2 (M−79), RT: 0.475 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes, Flow rate is set at 2.0 mL/min; return back to 5% ACN in water and hold for 0.02 min. Flow rate is set at 2.0 mL/min.

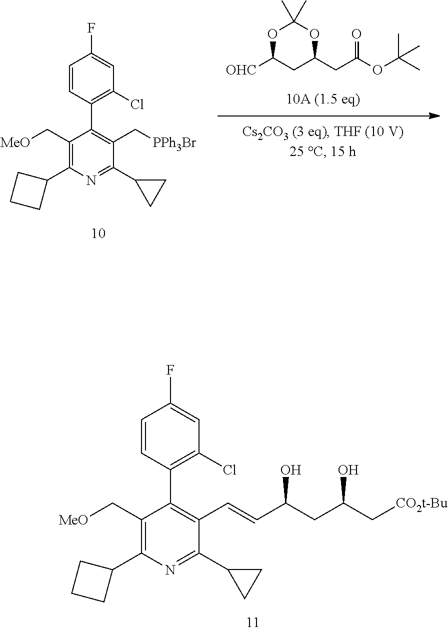

Step 10: A mixture of compound 10 (1.59 g, 2.27 mmol, 1 eq), compound 10A (878.79 mg, 3.40 mmol, 1.5 eq), Cs₂CO₃ (2.22 g, 6.80 mmol, 3 eq) in THF (10 mL) was degassed and purged with N₂ for 3 times, and then the mixture was stirred at 25 °C for 15 hours under N₂ atmosphere. LCMS showed reactant was consumed completely and one peak with 38% desired mass was detected. The mixture was concentrated under reduced pressure. The residue was purified by column chromatography on silica gel (PE/EtOAc=1/0 to 10/1) to give a crude product. The crude product was purified by prep-HPLC (column: Phenomenex luna C18 150*25 mm*10 μm; mobile phase: [water (TFA)-ACN]; gradient: 70%-100% B over 10 min) to give compound 11 (0.3 g, 23.23% yield) as a white solid. ¹H NMR: (400 MHz, CDCl₃) δ ppm 0.98-1.04 (m, 2H), 1.14-1.21 (m, 1H), 1.29-1.33 (m, 1H), 1.47 (s, 9H), 1.87-1.97 (m, 1H), 1.98-2.05 (m, 1H), 2.06-2.11 (m, 1H), 2.16-2.42 (m, 6H), 2.51-2.62 (m, 2H), 3.11 (s, 3H), 3.78-3.84 (m, 1H), 3.91-4.01 (m, 1H), 4.05-4.11 (m, 1H), 4.18-4.22 (m, 1H), 4.27-4.32 (m, 1H), 5.47-5.66 (m, 1H), 6.35 (dd, J=16.0, 7.6 Hz, 1H), 7.01-7.09 (m, 1H), 7.11-7.16 (m, 1H), 7.17-7.23 (m, 1H). LCMS (ESI+): m/z=560.2 (M+1), RT: 2.727 min. LC/MS: Kinetex® EVO C18 3.0×50 mm 2.6 μm. Detection methods are PDA&ELSD. MS mode was positive electrospray ionization. MS range was 50-1050. Mobile phase: Ramp from 5% ACN (0.01875% TFA) in water (0.0375% TFA) to 95% ACN in 3.40 min, Flow rate is set at 0.9 mL/min; then hold at 95% ACN for 0.30 minutes, Flow rate is set at 0.9 mL/min; return back to 5% ACN in water and hold for 0.30 min. Flow rate is set at 1.2 mL/min.

In some embodiments, compound 11 is the sodium salt of compound 11.

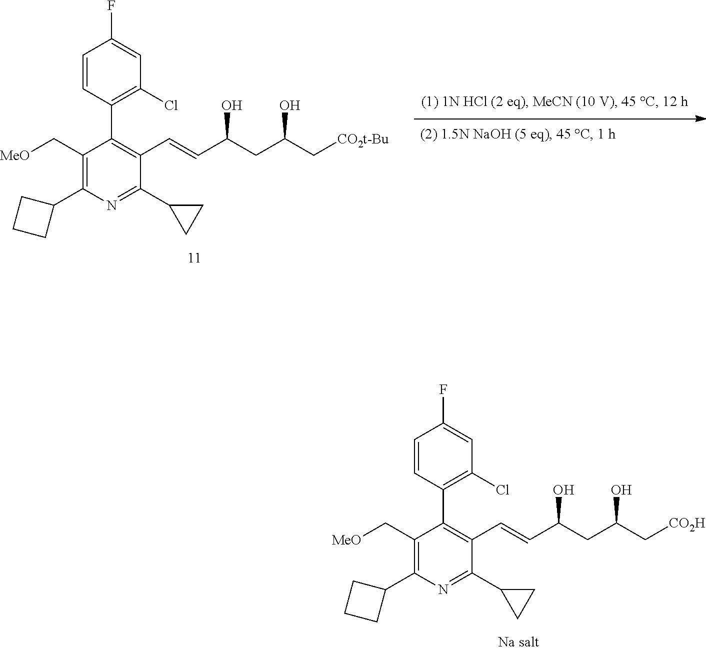 [SMILES: COCc1c(C2CCC2)nc(C2CC2)c(/C=C/[C@@H](O)C[C@@H](O)CC(=O)OC(C)(C)C)c1-c1ccc(F)cc1Cl ; COCc1c(C2CCC2)nc(C2CC2)c(/C=C/[C@@H](O)C[C@@H](O)CC(=O)O)c1-c1ccc(F)cc1Cl]

Step 11: To a solution of compound 11 (100 mg, 178.54 μmol, 1 eq) in MeCN (1 mL) was added HCl (1 M, 357.08 μL, 2 eq). The mixture was stirred at 45 °C for 12 hours. Then NaOH (1.5 M, 595.12 μL, 5 eq) was added to the solution. The mixture was stirred at 45 °C for 1 hour. LCMS showed reactant was consumed completely and one peak with 75% desired mass was detected. The mixture was concentrated under reduced pressure. The residue was purified by prep-HPLC (column: Waters Xbridge 150*25 mm*5 μm; mobile phase: [water (NH₃·H₂O)-ACN]; gradient: 15%-45% B over 12 min) and lyophilized directly to afford (3R,5S,E)-7-(4-(2-chloro-4-fluorophenyl)-6-cyclobutyl-2-cyclopropyl-5-(methoxymethyl)pyridin-3-yl)-3,5-dihydroxyhept-6-enoic acid sodium salt (42.26 mg, 35.81% yield) as yellow solid. ¹H NMR: (400 MHz, CDCl₃) δ 0.70-0.88 (m, 2H), 1.05-1.27 (m, 3H), 1.29-1.47 (m, 1H), 1.78-1.91 (m, 1H), 1.93-2.05 (m, 1H), 2.06-2.34 (m, 5H), 2.39-2.55 (m, 2H), 3.06 (s, 3H), 3.74-3.79 (m, 1H), 3.94-4.28 (m, 4H), 5.34-5.53 (m, 1H), 6.29 (dd, J=16.0, 8.0 Hz, 1H), 6.86-6.96 (m, 1H), 7.01-7.09 (m, 1H), 7.09-7.16 (m, 1H). LCMS (ESI+): m/z=504.1 (M+1), RT: 2.104 min. LC/MS: Kinetex® EVO C18 3.0×50 mm 2.6 μm. Detection methods are PDA&ELSD. MS mode was positive electrospray ionization. MS range was 50-1050. Mobile phase: Ramp from 5% ACN (0.01875% TFA) in water (0.0375% TFA) to 95% ACN in 3.40 min, Flow rate is set at 0.9 mL/min; then hold at 95% ACN for 0.30 minutes, Flow rate is set at 0.9 mL/min; return back to 5% ACN in water and hold for 0.30 min. Flow rate is set at 1.2 mL/min.

### Example 15. Synthesis of (3R,5S,E)-7-(6-cyclobutyl-2-cyclopropyl-4-(4-fluorophenyl)-5-(methoxymethyl)pyridin-3-yl)-3,5-dihydroxyhept-6-enoic acid sodium salt

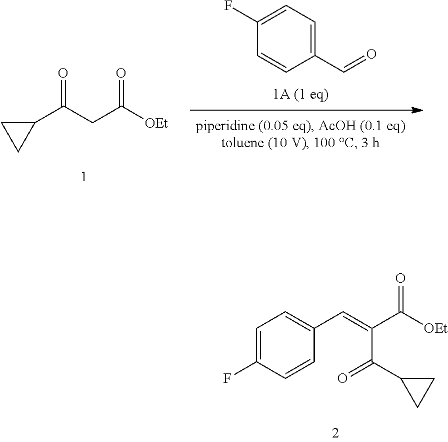 [SMILES: O=Cc1ccc(F)cc1 ; CCOC(=O)CC(=O)C1CC1 ; CCOC(=O)/C(=C/c1ccc(F)cc1)C(=O)C1CC1]

Step 1: A mixture of compound 1 (5 g, 32.01 mmol, 1 eq), compound 1A (3.97 g, 32.01 mmol, 3.38 mL, 1 eq), piperidine (136.30 mg, 1.60 mmol, 158.08 μL, 0.05 eq) and AcOH (192.25 mg, 3.20 mmol, 183.27 μL, 0.1 eq) in Toluene (50 mL) was degassed and purged with N₂ for 3 times, and then the mixture was stirred at 100 °C for 3 hours under N₂ atmosphere. LCMS showed desired compound was observed. The reaction mixture was concentrated under reduced pressure. The residue was purified by column chromatography on silica gel (PE/EtOAc=1:0 to 10:1) to afford compound 2 (12 g, 64.31% yield) as yellow oil. LCMS (ESI+): m/z=285.0 (M+23), RT: 0.440 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes, Flow rate is set at 2.0 mL/min; return back to 5% ACN in water and hold for 0.02 min. Flow rate is set at 2.0 mL/min.

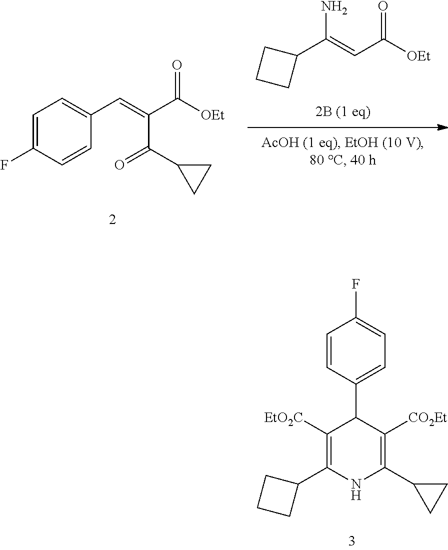 [SMILES: CCOC(=O)/C(=C/c1ccc(F)cc1)C(=O)C1CC1 ; CCOC(=O)/C=C(\N)C1CCC1 ; CCOC(=O)C1=C(C2CCC2)NC(C2CC2)=C(C(=O)OCC)C1c1ccc(F)cc1]

Step 2: To a solution of compound 2 (5.16 g, 19.67 mmol, 1 eq) and compound 2B (3.33 g, 19.67 mmol, 1 eq) in EtOH (50 mL) was added AcOH (1.18 g, 19.67 mmol, 1.13 mL, 1 eq). The mixture was stirred at 80 °C for 40 hours under N₂ atmosphere. LCMS showed 53% of desired compound was detected. The reaction mixture was concentrated under reduced pressure. The residue was purified by column chromatography on silica gel (PE/EtOAc=1:0 to 20:1) to afford compound 3 (6.45 g, 39.64% yield) as white solid. LCMS (ESI+): m/z=414.2 (M+1), RT: 0.534 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes, Flow rate is set at 2.0 mL/min; return back to 5% ACN in water and hold for 0.02 min. Flow rate is set at 2.0 mL/min.

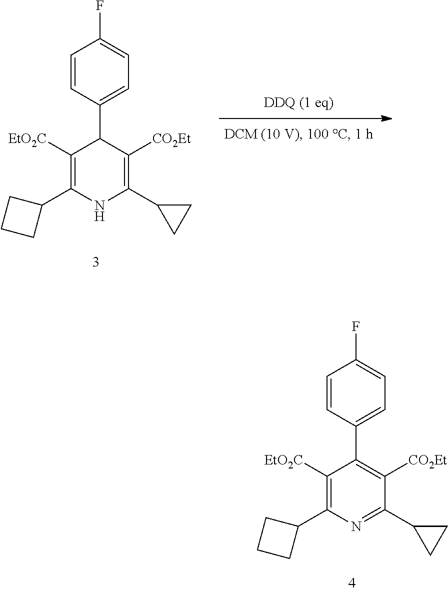 [SMILES: CCOC(=O)C1=C(C2CCC2)NC(C2CC2)=C(C(=O)OCC)C1c1ccc(F)cc1 ; CCOC(=O)c1c(C2CCC2)nc(C2CC2)c(C(=O)OCC)c1-c1ccc(F)cc1]

Step 3: To a solution of compound 3 (6.45 g, 15.60 mmol, 1 eq) in Toluene (60 mL) was added DDQ (3.54 g, 15.60 mmol, 1 eq). The mixture was stirred at 100 °C for 1 hour under N₂ atmosphere. LCMS showed 40% of desired compound was detected. The reaction mixture was concentrated under reduced pressure to give a residue. The residue was purified by column chromatography on silica gel (PE/EtOAc=1:0 to 20:1) to afford compound 4 (6.42 g, 74.78% yield) as a white solid. LCMS (ESI+): m/z=412.2 (M+1), RT: 0.578 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes, Flow rate is set at 2.0 mL/min; return back to 5% ACN in water and hold for 0.02 min. Flow rate is set at 2.0 mL/min.

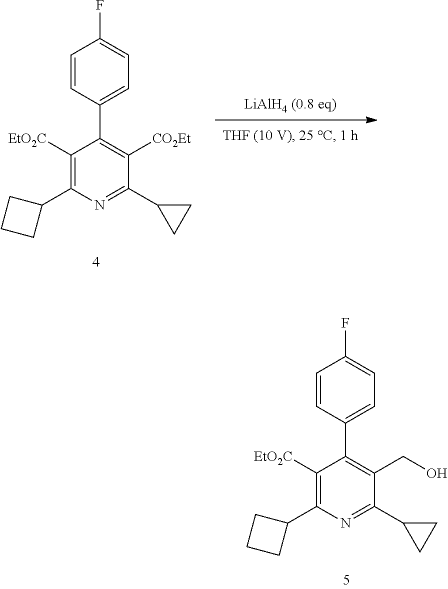 [SMILES: CCOC(=O)c1c(C2CCC2)nc(C2CC2)c(C(=O)OCC)c1-c1ccc(F)cc1 ; CCOC(=O)c1c(C2CCC2)nc(C2CC2)c(CO)c1-c1ccc(F)cc1]

Step 4: A mixture of compound 4 (4.6 g, 11.18 mmol, 1 eq) in THF (40 mL) was degassed and purged with N₂ for 3 times, then LiAlH₄ (1 M, 8.94 mL, 0.8 eq) was added dropwise to the solution at 0 °C. The mixture was stirred at 25 °C for 1 hour under N₂ atmosphere. LCMS showed reactant was consumed completely and one main peak with 84% desired mass was detected. The reaction mixture was quenched by adding slowly H₂O 0.3 mL, 15% NaOH solution 0.3 mL and H₂O 0.9 mL at 0 °C then dried with anhydrous Na₂SO₄, filtered and concentrated under reduced pressure. The residue was purified by column chromatography on silica gel (PE/EtOAc=1/0 to 5/1) to give compound 5 (3.3 g, 78.40% yield) as yellow oil. ¹H NMR: (400 MHz, CDCl₃) δ ppm 0.92-0.98 (m, 3H), 0.99-1.08 (m, 2H), 1.29-1.38 (m, 2H), 1.81-2.02 (m, 2H), 2.12-2.31 (m, 2H), 2.34-2.57 (m, 3H), 3.60-3.73 (m, 1H), 3.89-4.05 (m, 2H), 4.34-4.60 (m, 2H), 7.05-7.14 (m, 2H), 7.21-7.26 (m, 2H). LCMS (ESI+): m/z=370.5 (M+1), RT: 0.436 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes, Flow rate is set at 2.0 mL/min; return back to 5% ACN in water and hold for 0.02 min. Flow rate is set at 2.0 mL/min.

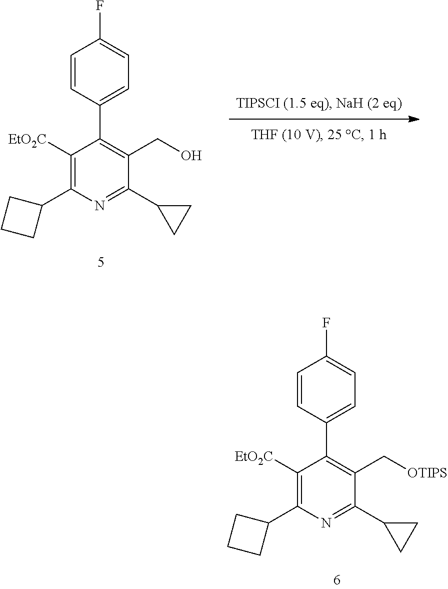

Step 5: A mixture of compound 5 (3.3 g, 8.76 mmol, 1 eq) in THF (30 mL) was degassed and purged with N₂ for 3 times, and NaH (701.18 mg, 17.53 mmol, 231.10 μL, 60% purity, 2 eq) was added to the solution at 0 °C. The mixture was stirred at 0 °C for 30 min under N₂ atmosphere. Then TIPSCl (2.53 g, 13.15 mmol, 2.81 mL, 1.5 eq) was added dropwise to the solution. The mixture was stirred at 25 °C for 1 hour under N₂ atmosphere. LCMS showed reactant was consumed completely. Two new peaks were shown on LCMS and ~65% of desired mass was detected. The reaction mixture was quenched by adding slowly NH₄Cl solution 20 mL at 0 °C. The mixture was extracted with ethyl acetate (20 mL×3) and washed with brine (20 mL×3), then the organic layer was dried with anhydrous Na₂SO₄, filtered and concentrated under reduced pressure. The residue was purified by column chromatography on silica gel (PE/EtOAc=1/0) to give compound 6 (4.1 g, 86.39% yield) as yellow solid. 1H NMR: (400 MHz, CDCl₃) δ 0.85-0.91 (m, 21H), 0.98-1.00 (m, 3H), 1.03-1.05 (m, 2H), 1.17-1.20 (m, 2H), 1.73-1.97 (m, 2H), 2.07-2.24 (m, 2H), 2.25-2.41 (m, 2H), 3.50-3.64 (m, 1H), 3.66-3.76 (m, 1H), 3.85-3.98 (m, 2H), 4.41-4.74 (m, 2H), 7.17-7.23 (m, 2H), 7.23-7.31 (m, 2H). LCMS (ESI+): m/z=526.4 (M+1), RT: 0.638 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes, Flow rate is set at 2.0 mL/min; return back to 5% ACN in water and hold for 0.02 min. Flow rate is set at 2.0 mL/min.

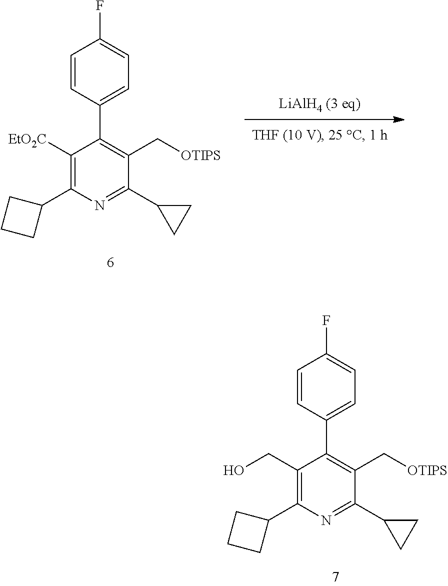

Step 6: A mixture of compound 6 (3.1 g. 5.73 mmol, 1 eq) in THF (30 mL) was degassed and purged with N₂ for 3 times, then LiAlH₄ (651.81 mg, 17.18 mmol, 3 eq) was added slowly to the solution at 0 °C. The mixture was stirred at 25 °C for 2 hours under N₂ atmosphere. LCMS showed reactant was consumed completely and one main peak with 86% desired mass was detected. The reaction mixture was quenched by adding slowly H₂O (0.7 mL), 15% NaOH solution (0.7 mL) and H₂O (2.1 mL) at 0 °C. The mixture was dried with anhydrous Na₂SO₄, filtered and concentrated under reduced pressure to give compound 7 (3.6 g, 88.78% yield) as yellow oil. ¹H NMR: (400 MHz, CDCl₃) δ ppm 0.92-1.01 (m, 21H), 1.26-1.29 (m, 2H), 1.29-1.34 (m, 2H), 1.84-2.03 (m, 2H), 2.18-2.31 (m, 2H), 2.41-2.60 (m, 3H), 3.63-3.83 (m, 1H), 3.89-4.08 (m, 1H), 4.26-4.29 (m, 1H), 4.46-4.63 (m, 2H), 7.07-7.15 (m, 2H), 7.16-7.24 (m, 2H). LCMS (ESI+): m/z=484.7 (M+1), RT: 0.523 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes, Flow rate is set at 2.0 mL/min; return back to 5% ACN in water and hold for 0.02 min. Flow rate is set at 2.0 mL/min.

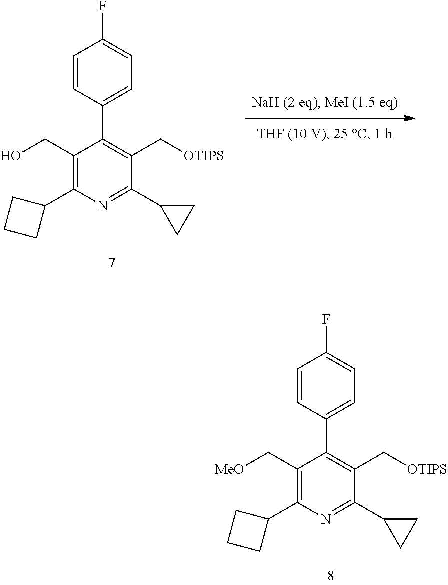

Step 7: A mixture of compound 7 (3 g, 5.51 mmol, 1 eq) in THF (30 mL) was degassed and purged with N₂ for 3 times, then NaH (440.53 mg, 11.01 mmol, 60% purity, 2 eq) was added slowly to the solution at 0 °C. The mixture was stirred at 0 °C for 30 min under N₂ atmosphere. Then Mel (1.17 g, 8.26 mmol, 514.21 μL, 1.5 eq) was added dropwise to the solution. The mixture was stirred at 25 °C for 1 hr under N₂ atmosphere. LCMS showed reactant was consumed completely and one main peak with 83% desired mass was detected. The reaction mixture was quenched by adding slowly NH₄Cl solution 20 mL at 0 °C. The mixture was extracted with ethyl acetate (20 mL×3) and washed with brine (20 mL×3), then the organic layer was dried with anhydrous Na₂SO₄, filtered and concentrated under reduced pressure to give compound 8 (2.7 g, 88.70% yield) as yellow oil. ¹H NMR: (400 MHz, CDCl₃) δ ppm 0.87-0.99 (m, 21H), 1.22-1.27 (m, 4H), 1.86-2.06 (m, 2H), 2.12-2.26 (m, 2H), 2.37-2.61 (m, 3H), 3.07-3.22 (m, 3H), 3.43-3.48 (m, 1H), 3.87-3.98 (m, 2H), 4.31-4.68 (m, 2H), 6.99-7.12 (m, 2H), 7.13-7.25 (m, 2H). LCMS (ESI+): m/z=498.7 (M+1), RT: 0.576 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes, Flow rate is set at 2.0 mL/min; return back to 5% ACN in water and hold for 0.02 min. Flow rate is set at 2.0 mL/min.

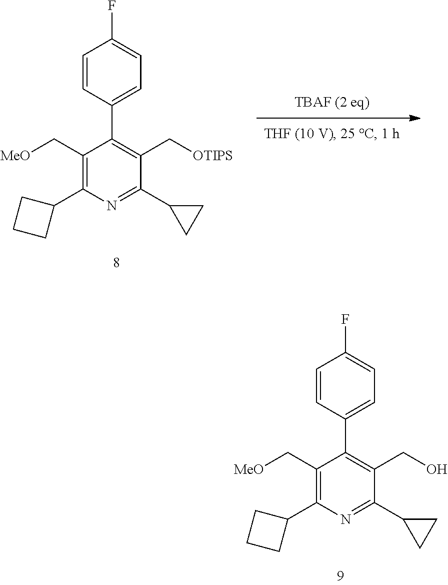

Step 8: To a solution of compound 8 (1.3 g, 2.35 mmol, 1 eq) in THF (10 mL) was added TBAF (1 M, 4.70 mL, 2 eq). The mixture was stirred at 25 °C for 1 hour. LCMS showed reactant was consumed completely and one main peak with 90% desired mass was detected. The mixture was concentrated under reduced pressure. The residue was purified by column chromatography on silica gel (PE/EtOAc=1/0 to 5/1) to give compound 9 (1.6 g, 97.41% yield) as colorless oil. ¹H NMR: (400 MHz, CDCl₃) δ 0.97-1.03 (m, 2H), 1.29-1.34 (m, 2H), 1.85-2.04 (m, 2H), 2.16-2.27 (m, 2H), 2.30-2.42 (m, 1H), 2.43-2.57 (m, 2H), 3.10-3.26 (m, 3H), 3.82-3.94 (m, 1H), 3.94-4.00 (m, 2H), 4.27-4.55 (m, 2H), 7.08-7.17 (m, 2H), 7.18-7.26 (m, 2H). LCMS (ESI+): m/z=342.5 (M+1), RT: 0.301 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes, Flow rate is set at 2.0 mL/min; return back to 5% ACN in water and hold for 0.02 min. Flow rate is set at 2.0 mL/min.

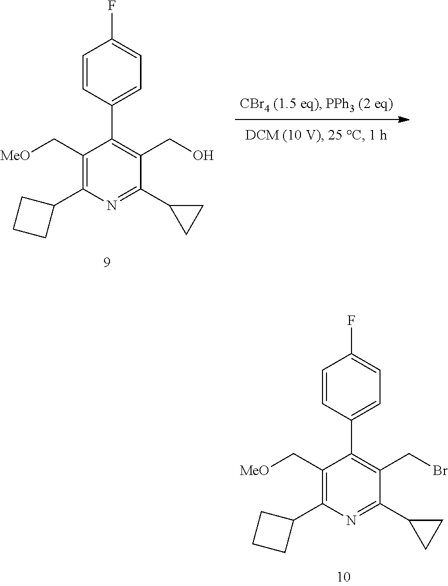 [SMILES: COCc1c(C2CCC2)nc(C2CC2)c(CO)c1-c1ccc(F)cc1 ; COCc1c(C2CCC2)nc(C2CC2)c(CBr)c1-c1ccc(F)cc1]

Step 9: To a solution of compound 9 (0.5 g, 1.43 mmol, 1 eq) in DCM (5 mL) was added PPh₃ (751.03 mg, 2.86 mmol, 2 eq) and CBr₄ (712.18 mg, 2.15 mmol, 1.5 eq) at 0 °C under N₂. The mixture was stirred at 25 °C for 1 hour. LCMS showed reactant was consumed completely and one peak with 28% desired mass was detected. The mixture was concentrated under reduced pressure. The residue was purified by column chromatography on silica gel (PE/EtOAc=1/0 to 10/1) to give compound 10 (860 mg, 72.94% yield) as colorless oil. 1H NMR: (400 MHz, CDCl₃) δ 0.98-1.02 (m, 2H), 1.27-1.33 (m, 2H), 1.81-2.01 (m, 2H), 2.14-2.33 (m, 3H), 2.39-2.54 (m, 2H), 3.07-3.19 (m, 3H), 3.79-3.89 (m, 1H), 3.89-4.08 (m, 2H), 4.13-4.36 (m, 2H), 7.07-7.17 (m, 2H), 7.20-7.27 (m, 2H). LCMS (ESI+): m/z=404.0 (M+1), RT: 0.593 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes, Flow rate is set at 2.0 mL/min; return back to 5% ACN in water and hold for 0.02 min. Flow rate is set at 2.0 mL/min.

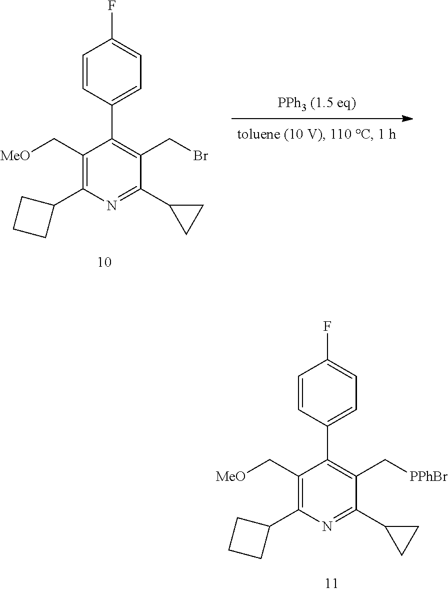

Step 10: A mixture of compound 10 (0.43 g, 1.04 mmol, 1 eq), PPh₃ (410.86 mg, 1.57 mmol, 1.5 eq) in Toluene (4 mL) was degassed and purged with N₂ for 3 times, and then the mixture was stirred at 110 °C for 1 hour under N₂ atmosphere. LCMS showed reactant was consumed completely and one peak with 46% desired mass was detected. The mixture was concentrated under reduced pressure to give compound 11 (1.39 g, 100.00% yield) as white solid. LCMS (ESI+): m/z=586.2 (M−79), RT: 0.485 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes, Flow rate is set at 2.0 mL/min; return back to 5% ACN in water and hold for 0.02 min. Flow rate is set at 2.0 mL/min.

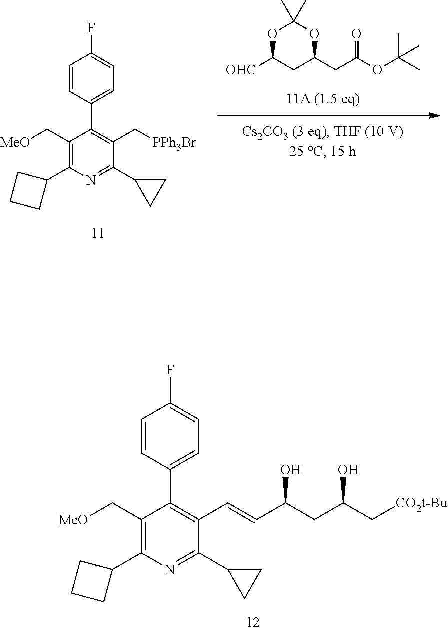

Step 11: A mixture of compound 11 (1.39 g, 2.09 mmol, 1 eq), compound 11A (807.95 mg, 3.13 mmol, 1.5 eq), Cs₂CO₃ (2.04 g, 6.26 mmol, 3 eq) in THF (10 mL) was degassed and purged with N₂ for 3 times, and then the mixture was stirred at 25 °C for 12 hours under N₂ atmosphere. LCMS showed reactant was consumed completely and one peak with 27% desired mass was detected. The mixture was concentrated under reduced pressure. The residue was purified by column chromatography on silica gel (PE/EtOAc=1/0 to 10/1) to give a crude product. The crude product was purified by prep-HPLC (column: Phenomenex luna C18 150*25 mm*10 μm; mobile phase: [water (TFA)-ACN]; gradient: 66%-96% B over 10 min) and concentrated directly under reduced pressure to give compound 12 (0.25 g, 22.33% yield) as colorless oil. ¹H NMR: (400 MHz, CDCl₃) δ ppm 0.99-1.05 (m, 2H), 1.15-1.20 (m, 2H), 1.47 (s, 9H), 1.88-1.98 (m, 1H), 1.99-2.13 (m, 2H), 2.19-2.45 (m, 6H), 2.53-2.65 (m, 2H), 3.17 (s, 3H), 3.94-3.98 (m, 1H), 4.00 (s, 2H), 4.07-4.12 (m, 1H), 4.31-4.40 (m, 1H), 5.58 (dd, J=16.0, 6.0 Hz, 1H), 6.32 (dd, J=16.0, 1.2 Hz, 1H), 7.06-7.16 (m, 4H). LCMS (ESI+): m/z=526.3 (M+1), RT: 2.454 min. LC/MS: Kinetex® EVO C18 3.0×50 mm 2.6 μm. Detection methods are PDA & ELSD. MS mode was positive electrospray ionization. MS range was 50-1050. Mobile phase: Ramp from 5% ACN (0.01875% TFA) in water (0.0375% TFA) to 95% ACN in 3.40 min, Flow rate is set at 0.9 mL/min; then hold at 95% ACN for 0.30 minutes, Flow rate is set at 0.9 mL/min; return back to 5% ACN in water and hold for 0.30 min. Flow rate is set at 1.2 mL/min.

In some embodiments, compound 12 is the sodium salt of compound 12.

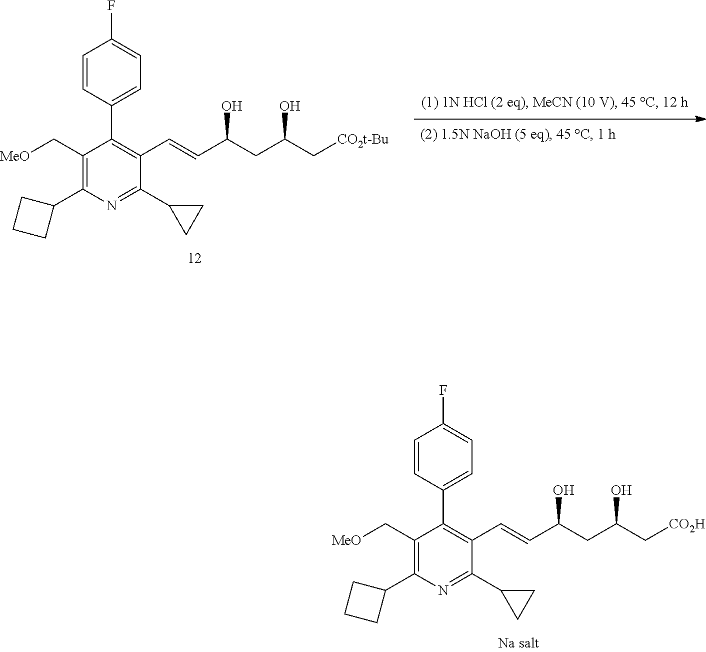 [SMILES: COCc1c(C2CCC2)nc(C2CC2)c(/C=C/[C@@H](O)C[C@@H](O)CC(=O)OC(C)(C)C)c1-c1ccc(F)cc1 ; COCc1c(C2CCC2)nc(C2CC2)c(/C=C/[C@@H](O)C[C@@H](O)CC(=O)O)c1-c1ccc(F)cc1]

Step 12: To a solution of compound 12 (0.1 g, 190.24 μmol, 1 eq) in MeCN (1 mL) was added HCl (1 M, 380.48 μL, 2 eq). The mixture was stirred at 45 °C for 12 hours. Then NaOH (1.5 M, 760.95 μL, 5 eq) was added to the solution. The mixture was stirred at 45 °C for 1 hr. LCMS showed reactant was consumed completely and one peak with 97% desired mass was detected. The mixture was concentrated under reduced pressure to give a residue. The residue was purified by prep-HPLC (column: Waters Xbridge 150*25 mm*5 μm; mobile phase: [water (NH₃·H₂O)-ACN]; gradient: 15%-45% B over 12 min) and lyophilized directly to afford (3R,5S,E)-7-(6-cyclobutyl-2-cyclopropyl-4-(4-fluorophenyl)-5-(methoxymethyl)pyridin-3-yl)-3,5-dihydroxyhept-6-enoic acid sodium salt (37.54 mg, 32.45% yield) as a yellow solid. ¹H NMR: (400 MHz, CDCl₃) δ 0.70-0.90 (m, 2H), 1.09-1.29 (m, 3H), 1.30-1.47 (m, 1H), 1.81-1.91 (m, 1H), 1.93-2.05 (m, 1H), 2.06-2.33 (m, 5H), 2.38-2.53 (m, 2H), 3.10 (s, 3H), 3.80-3.87 (m, 1H), 3.92-3.96 (m, 2H), 4.06-4.14 (m, 2H), 5.43 (dd, J=16.0, 5.6 Hz, 1H), 6.21 (d, J=16.0 Hz, 1H), 6.88-7.05 (m, 4H). LCMS (ESI+): m/z=470.2 (M+1), RT: 1.792 min. LC/MS: Kinetex® EVO C18 3.0×50 mm 2.6 μm. Detection methods are PDA & ELSD. MS mode was positive electrospray ionization. MS range was 50-1050. Mobile phase: Ramp from 5% ACN (0.01875% TFA) in water (0.0375% TFA) to 95% ACN in 3.40 min, Flow rate is set at 0.9 mL/min; then hold at 95% ACN for 0.30 minutes, Flow rate is set at 0.9 mL/min; return back to 5% ACN in water and hold for 0.30 min. Flow rate is set at 1.2 mL/min.

### Example 16. Synthesis of (3R,5S,E)-7-(2,6-dicyclopropyl-4-(4-fluoro-2-methylphenyl)-5-(methoxymethyl)pyridin-3-yl)-3,5-dihydroxyhept-6-enoate sodium salt

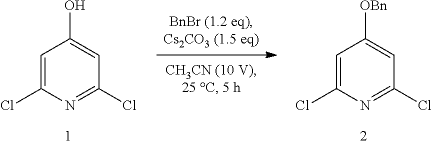 [SMILES: Oc1cc(Cl)nc(Cl)c1 ; Clc1cc(OCc2ccccc2)cc(Cl)n1]

Step 1: To a solution of compound 1 (10 g, 60.98 mmol, 1 eq) in CH₃CN (200 mL) was added BnBr (12.52 g, 73.18 mmol, 8.69 mL, 1.2 eq) and Cs₂CO₃ (29.80 g, 91.47 mmol, 1.5 eq). The mixture was stirred at 25 °C for 5 hours under N₂ atmosphere. The reaction was quenched with H₂O (150 mL) and extracted with EtOAc (150 mL×3). The combined organic layers were washed with brine (150 mL×3), dried over Na₂SO₄, filtered and concentrated under reduced pressure. The residue was purified by column chromatography on silica gel (PE/EtOAc=100:1 to 1:1) to give compound 2 (28 g, 90.35% yield) as a white solid. LCMS (ESI+): m/z=254.0 (M+23), RT: 0.506 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 min, Flow rate is set at 2.0 mL/min; return back to 5% ACN in water and hold for 0.02 min, Flow rate is set at 2.0 mL/min.

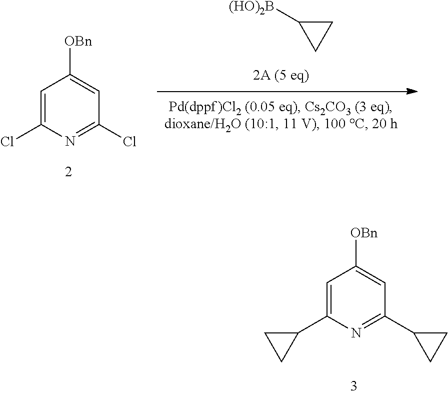 [SMILES: Clc1cc(OCc2ccccc2)cc(Cl)n1 ; OB(O)C1CC1 ; c1ccc(COc2cc(C3CC3)nc(C3CC3)c2)cc1]

Step 2: A mixture of compound 2 (5 g, 19.68 mmol, 1 eq), compound 2A (8.45 g, 98.38 mmol, 5 eq), Pd(dppf)Cl₂·CH₂Cl₂ (803.42 mg, 983.82 μmol, 0.05 eq), Cs₂CO₃ (19.23 g, 59.03 mmol, 3 eq) in dioxane (50 mL) and H₂O (5 mL) was degassed and purged with N₂ for 3 times, and then the mixture was stirred at 100 °C for 20 hours under N₂ atmosphere. After cooling to room temperature, the reaction mixture was combined with two paralleled batches (5 g and 8 g compound 2 were used), diluted with water (100 mL) and extracted with EtOAc (100 mL×3). The combined organic layer was washed with brine (100 mL), dried over Na₂SO₄, filtered and concentrated under reduced pressure. The residue was purified by column chromatography on silica gel (PE/EtOAc=1:0 to 10:1) to give compound 3 (14.3 g, 76.10% yield) as a white solid. LCMS (ESI+): m/z=266.0 (M+1), RT: 0.310 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 min, Flow rate is set at 2.0 mL/min; return back to 5% ACN in water and hold for 0.02 min, Flow rate is set at 2.0 mL/min.

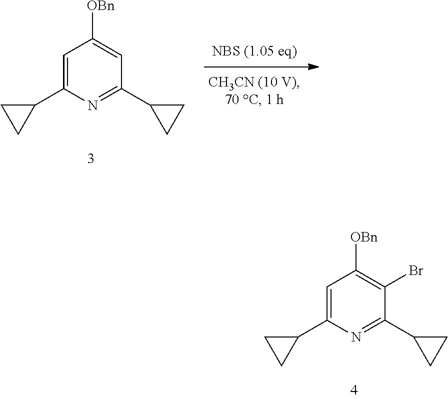 [SMILES: c1ccc(COc2cc(C3CC3)nc(C3CC3)c2)cc1 ; Brc1c(OCc2ccccc2)cc(C2CC2)nc1C1CC1]

Step 3: To a solution of compound 3 (11 g, 41.45 mmol, 1 eq) in MeCN (100 mL) was added NBS (7.75 g, 43.53 mmol, 1.05 eq). The mixture was stirred at 70 °C for 1 hour under N₂. LCMS showed 80% of desired product was detected. The reaction mixture was combined with a paralleled batch (3 g compound 3 was used), and concentrated under reduced pressure. The crude product was purified by column chromatography on silica gel (PE/EtOAc=50:1 to 10:1) to give Compound 4 (14 g, 77.09% yield) as a yellow solid. LCMS (ESI+): m/z=343.9 (M+1), RT: 0.446 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 min, Flow rate is set at 2.0 mL/min; return back to 5% ACN in water and hold for 0.02 min, Flow rate is set at 2.0 mL/min.

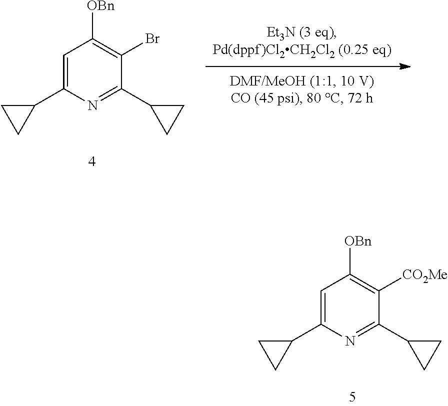 [SMILES: Brc1c(OCc2ccccc2)cc(C2CC2)nc1C1CC1 ; COC(=O)c1c(OCc2ccccc2)cc(C2CC2)nc1C1CC1]

Step 4: A solution of compound 4 (5 g, 14.52 mmol, 1 eq), Et₃N (4.41 g, 43.57 mmol, 6.06 mL, 3 eq), Pd(dppf)Cl₂·CH₂Cl₂ (2.97 g. 3.63 mmol, 0.25 eq) in DMF (50 mL) and MeOH (50 mL) was degassed and purged with CO for 3 times. The mixture was stirred under CO (45 psi) at 80 °C for 72 hours. LCMS showed 12% of compound 4 remained and 60% of desired product was observed. The mixture was combined with a paralleled batch (5 g compound 4 was used), filtered and the filtrate was concentrated under reduced pressure. The crude product was purified by flash column chromatography on silica gel (0-10% EtOAc in PE) to give compound 5 (2.57 g, 27.36% yield) as yellow oil. LCMS (ESI+): m/z=324.2 (M+1), RT: 0.375 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 min, Flow rate is set at 2.0 mL/min; return back to 5% ACN in water and hold for 0.02 min, Flow rate is set at 2.0 mL/min.

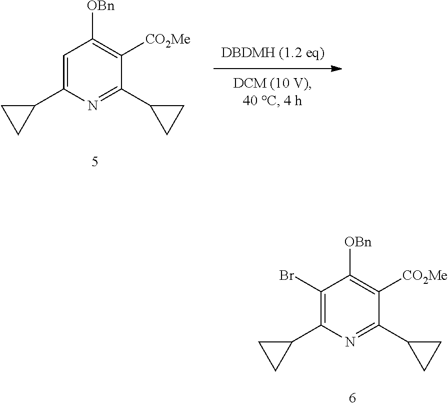 [SMILES: COC(=O)c1c(OCc2ccccc2)cc(C2CC2)nc1C1CC1 ; COC(=O)c1c(C2CC2)nc(C2CC2)c(Br)c1OCc1ccccc1]

Step 5: To a solution of compound 5 (2.57 g, 7.95 mmol, 1 eq) in DCM (20 mL) was added DBDMH (2.73 g, 9.54 mmol, 1.2 eq) and the mixture was stirred at 40 °C for 4 hours. LCMS showed the reactant was consumed completely and 84% of desired product was observed. The mixture was washed with water (20 mL) and the organic layer was concentrated under reduced pressure. The crude product was purified by flash column chromatography on silica gel (0-3% EtOAc in PE) to give compound 6 (2.84 g, 88.83% yield) as a yellow solid. LCMS (ESI+): m/z=402.0 (M+1), RT: 0.580 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 min, Flow rate is set at 2.0 mL/min; return back to 5% ACN in water and hold for 0.02 min, Flow rate is set at 2.0 mL/min.

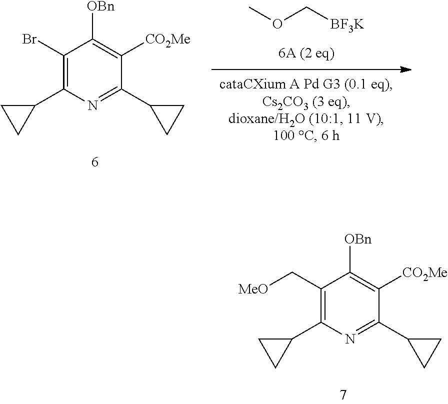

Step 6: A mixture of compound 6 (2.34 g, 4.42 mmol, 1 eq), compound 6A (1.34 g, 8.84 mmol, 2 eq), cataCXium A Pd G3 (321.95 mg, 442.08 μmol, 0.1 eq) and Cs₂CO₃ (4.32 g, 13.26 mmol, 3 eq) in dioxane (20 mL) and H₂O (2 mL) was purged and degassed with N₂ for 3 times. The mixture was stirred at 100 °C under N₂ for 6 hours. The mixture was concentrated under reduced pressure. The residue was purified by flash column chromatography on silica gel (0-10% EtOAc in PE) to afford a crude product, which was purified by prep-HPLC (column: Phenomenex luna C18 (250*70 mm, 10 μm); mobile phase: [water (FA)-ACN]; gradient: 50%-80% B over 5 min) to give compound 7 (1.6 g, 98.50% yield) as a brown solid.

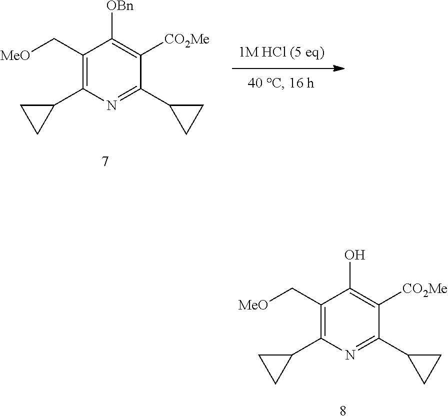 [SMILES: COCc1c(C2CC2)nc(C2CC2)c(C(=O)OC)c1OCc1ccccc1 ; COCc1c(C2CC2)nc(C2CC2)c(C(=O)OC)c1O]

Step 7: A solution of compound 7 (1.6 g, 4.35 mmol, 1 eq) in HCl (1 M, 21.77 mL, 5 eq) was stirred at 40 °C for 16 hours. LCMS showed 29% of reactant remained and 53% of desired product was observed. The reaction mixture was basified with saturated aqueous Na₂CO₃ to pH=7 and extracted with EtOAc (20 mL×3). The combined organic layers were dried over anhydrous Na₂SO₄ and concentrated under reduced pressure. The crude product was purified by flash column chromatography on silica gel (0-20% EtOAc in PE) to give compound 8 (680 mg, 56.31% yield) as a yellow solid. LCMS (ESI+): m/z=277.9 (M+1), RT: 0.274 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 min, Flow rate is set at 2.0 mL/min; return back to 5% ACN in water and hold for 0.02 min, Flow rate is set at 2.0 mL/min.

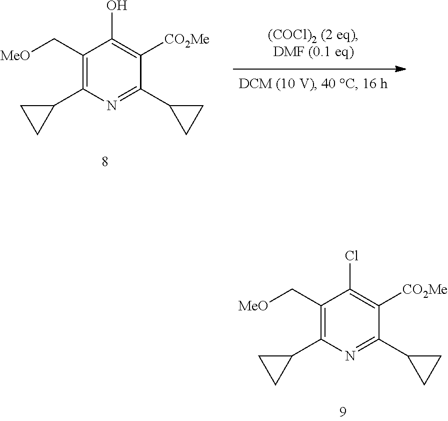 [SMILES: COCc1c(C2CC2)nc(C2CC2)c(C(=O)OC)c1O ; COCc1c(C2CC2)nc(C2CC2)c(C(=O)OC)c1Cl]

Step 8: To a solution of compound 8 (580 mg, 2.09 mmol, 1 eq) in 1,2-dichloroethane (5 mL) was added oxalyl chloride (530.93 mg, 4.18 mmol, 366.16 μL, 2 eq) and DMF (15.29 mg, 209.15 μmol, 16.09 μL, 0.1 eq) and the mixture was stirred at 40 °C for 16 hours. LCMS showed the reactant was consumed completely and 85% of desired product was observed. The mixture was combined with a paralleled batch (330 mg compound 8 was used) and concentrated under reduced pressure. The crude product was purified by flash column chromatography on silica gel (0-5% EtOAc in PE) to afford compound 9 (791 mg, 81.63% yield) as colorless oil. LCMS (ESI+): m/z=296.1 (M+1), RT: 0.483 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 min, Flow rate is set at 2.0 mL/min; return back to 5% ACN in water and hold for 0.02 min, Flow rate is set at 2.0 mL/min.

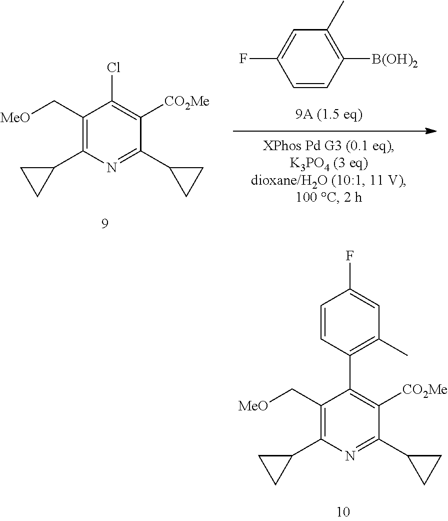 [SMILES: COCc1c(C2CC2)nc(C2CC2)c(C(=O)OC)c1Cl ; Cc1cc(F)ccc1B(O)O ; COCc1c(C2CC2)nc(C2CC2)c(C(=O)OC)c1-c1ccc(F)cc1C]

Step 9: A mixture of compound 9 (400 mg, 1.35 mmol, 1 eq), compound 9A (312.31 mg, 2.03 mmol, 1.5 eq), XPhos Pd G3 (114.48 mg, 135.24 μmol, 0.1 eq), K₃PO₄ (861.24 mg, 4.06 mmol, 3 eq) in dioxane (5 mL) and H₂O (0.5 mL) was degassed and purged with N₂ for 3 times and the mixture was stirred at 100 °C under N₂ for 2 hours. LCMS showed the reactant was consumed completely and 60% of desired product was observed. The reaction mixture was combined with a paralleled batch (390 mg compound 9 was used) and concentrated under reduced pressure. The crude product was purified by flash column chromatography on silica gel (0-5% EtOAc in PE) to give compound 10 (700 mg, 70.05% yield) as yellow oil. LCMS (ESI+): m/z=370.1 (M+1), RT: 0.543 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 min, Flow rate is set at 2.0 mL/min; return back to 5% ACN in water and hold for 0.02 min, Flow rate is set at 2.0 mL/min.

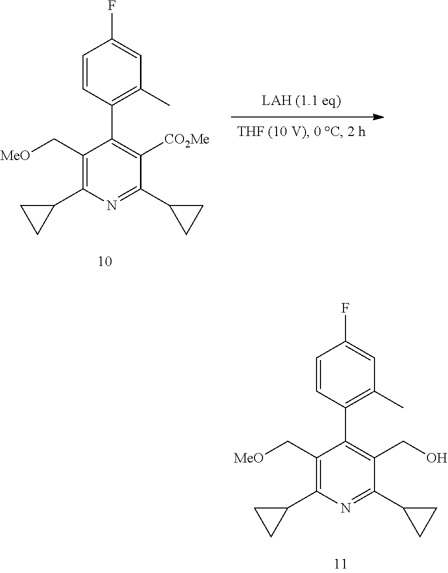 [SMILES: COCc1c(C2CC2)nc(C2CC2)c(C(=O)OC)c1-c1ccc(F)cc1C ; COCc1c(C2CC2)nc(C2CC2)c(CO)c1-c1ccc(F)cc1C]

Step 10: To a solution of compound 10 (600 mg, 1.62 mmol, 1 eq) in THF (6 mL) was added LiAlH₄ (1 M, 1.79 mL, 1.1 eq) at 0 °C and the mixture was stirred at 0 °C for 2 hours. LCMS showed the reactant was consumed completely and 81% of desired product was observed. The reaction was quenched with water (5 mL) and concentrated under reduced pressure to give a crude product compound 11 (659 mg, crude) as a brown solid. LCMS (ESI+): m/z=342.2 (M+1), RT: 0.402 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 min, Flow rate is set at 2.0 mL/min; return back to 5% ACN in water and hold for 0.02 min, Flow rate is set at 2.0 mL/min.

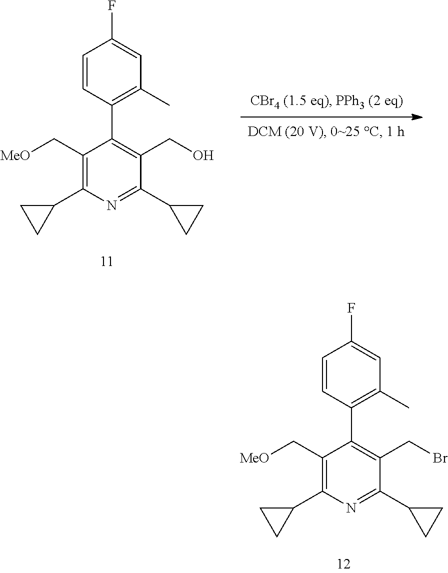 [SMILES: COCc1c(C2CC2)nc(C2CC2)c(CO)c1-c1ccc(F)cc1C ; COCc1c(C2CC2)nc(C2CC2)c(CBr)c1-c1ccc(F)cc1C]

Step 11: To a solution of compound 11 (550 mg, 1.61 mmol, 1 eq) in DCM (10 mL) was added PPh₃ (845.05 mg, 3.22 mmol, 2 eq) and CBr₄ (801.34 mg, 2.42 mmol, 1.5 eq) at 0 °C then the mixture was stirred at 25 °C under N₂ for 1 hour. LCMS showed the reactant was consumed completely and 62% of desired product was observed. The mixture was concentrated under reduced pressure. The crude product was purified by flash column chromatography on silica gel (0-5% EtOAc in PE) to afford compound 12 (498 mg, 76.46% yield) as yellow oil. LCMS (ESI+): m/z=404.0 (M+1), RT: 0.580 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 min, Flow rate is set at 2.0 mL/min; return back to 5% ACN in water and hold for 0.02 min, Flow rate is set at 2.0 mL/min.

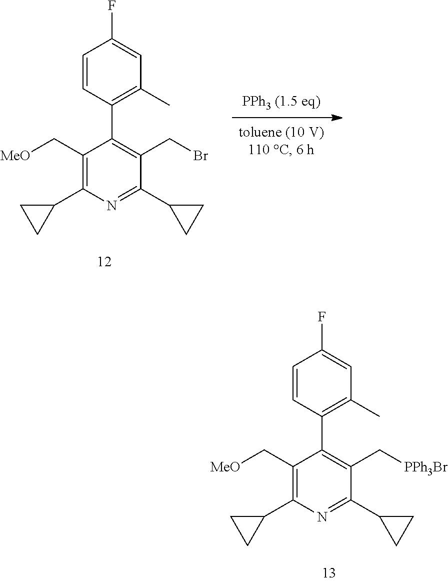

Step 12: To a solution of compound 12 (498 mg, 1.23 mmol, 1 eq) in toluene (5 mL) was added PPh₃ (484.59 mg, 1.85 mmol, 1.5 eq) and the mixture was stirred at 110 °C under N₂ for 6 hours. LCMS showed the reactant was consumed completely and 24% of desired product was observed. The mixture was concentrated under reduced pressure to give compound 13 (821 mg, 99.99% yield) as yellow oil. LCMS (ESI+): m/z=586.2 (M−79), RT: 0.470 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 min, Flow rate is set at 2.0 mL/min; return back to 5% ACN in water and hold for 0.02 min, Flow rate is set at 2.0 mL/min.

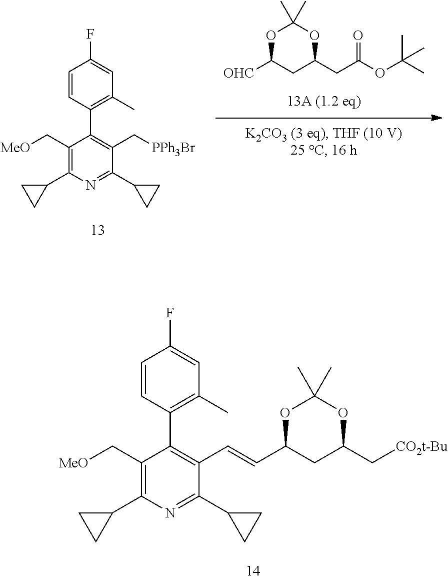

Step 13: To a solution of compound 13 (821 mg, 1.23 mmol, 1 eq) in toluene (5 mL) was added K₂CO₃ (510.65 mg, 3.69 mmol, 3 eq) and the mixture was stirred at 25 °C for 0.5 hour. Compound 13A (381.77 mg, 1.48 mmol, 1.2 eq) was added and the mixture was stirred at 25 °C under N₂ for 16 hours. LCMS showed the reactant was consumed completely and 34% of desired product was observed. The mixture was concentrated under reduced pressure. The crude product was purified by flash column chromatography on silica gel (0-5% EtOAc in PE, then 50-100% DCM in PE) to afford compound 14 (254 mg, 63.50% yield) as colorless oil. ¹H NMR: (400 MHz, CDCl₃) δ 0.84-0.93 (m, 4H), 1.04-1.14 (m, 4H), 1.34 (d, J=5.2 Hz, 2H), 1.42 (s, 3H), 1.45 (s, 9H), 1.57 (s, 3H), 1.94 (d, J=11.2 Hz, 3H), 2.19-2.28 (m, 3H), 2.33-2.45 (m, 1H), 3.16 (s, 3H), 4.02 (dd, J=10.4, 3.2 Hz, 1H), 4.16-4.29 (m, 3H), 5.48 (ddd, J=16.0, 12.0, 6.4 Hz, 1H), 6.17 (dd, J=16.0, 8.8 Hz, 1H), 6.86-7.01 (m, 3H). LCMS (ESI+): m/z=566.4 (M+1), RT: 3.636 min. LC/MS: XBridge C18 3.0*50 mm, 5 μm. Detection methods are photo-diode array (PDA). MS mode was positive electrospray ionization. MS range was 50-1050. Mobile phase: Ramp from 5% ACN in water (0.025% NH₃·H₂O) to 95% ACN in 3.00 min, Flow rate is set at 0.9 mL/min; then hold at 95% ACN for 0.70 min, Flow rate is set at 0.9 mL/min; return back to 5% ACN in water and hold for 0.30 min, Flow rate is set at 1.2 mL/min.

In some embodiments, compound 14 is the sodium salt of compound 14.

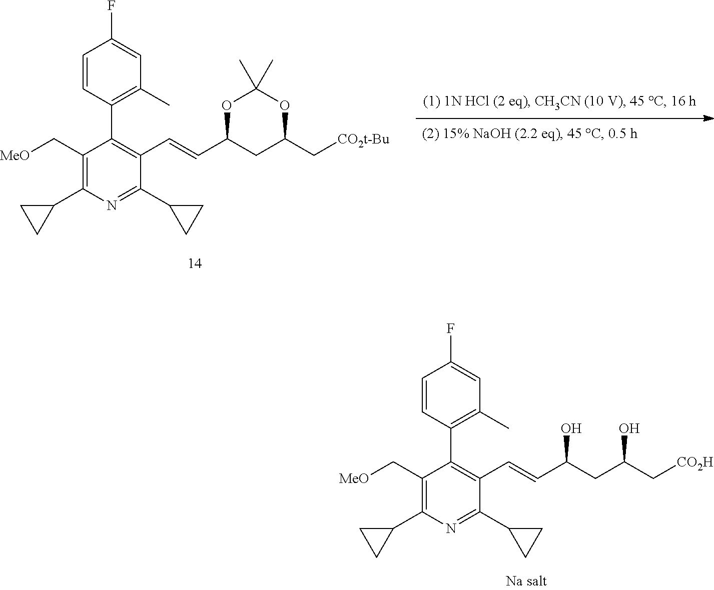 [SMILES: COCc1c(C2CC2)nc(C2CC2)c(/C=C/[C@@H]2C[C@H](CC(=O)OC(C)(C)C)OC(C)(C)O2)c1-c1ccc(F)cc1C ; COCc1c(C2CC2)nc(C2CC2)c(/C=C/[C@@H](O)C[C@@H](O)CC(=O)O)c1-c1ccc(F)cc1C]

Step 14: To a solution of compound 14 (100 mg, 176.77 μmol, 1 eq) in CH₃CN (1 mL) was added HCl (1 M, 353.54 μL, 2 eq) and the mixture was stirred at 45 °C for 16 hours. Aqueous NaOH (103.68 mg, 388.89 μmol, 15% purity, 2.2 eq) was added and the mixture was stirred at 45 °C for 0.5 hour. LCMS showed the reactant was consumed completely and 88% of desired product was observed. The mixture was purified by prep-HPLC (column: Waters Xbridge 150*25 mm*5 μm; mobile phase: [water (NH₃·H₂O)-ACN]; gradient: 10%-40% B over 12 min) to afford (3R,5S,E)-7-(2,6-dicyclopropyl-4-(4-fluoro-2-methylphenyl)-5-(methoxymethyl)pyridin-3-yl)-3,5-dihydroxyhept-6-enoate sodium salt (32 mg, 38.55% yield) as a white solid. ¹H NMR: (400 MHz, DMSO-d₆) δ 0.62-1.18 (m, 9H), 1.20-1.39 (m, 1H), 1.72-1.93 (m, 4H), 1.93-2.05 (m, 1H), 2.18-2.37 (m, 2H), 3.04 (s, 3H), 3.51-3.61 (m, 1H), 3.90-4.06 (m, 2H), 4.14 d, J=10.4 Hz, 1H), 5.46 (ddt, J=16.0, 10.8, 5.6, 5.6 Hz, 1H), 6.10 (d, J=16.0 Hz, 1H), 6.89-7.20 (m, 3H). LCMS (ESI+): m/z=470.3 (M+1), RT: 1.666 min. LC/MS: Kinetex® EVO C18 3.0×50 mm 2.6 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in 3.40 min, Flow rate is set at 0.9 mL/min; then hold at 95% ACN for 0.30 min, Flow rate is set at 0.9 mL/min; return back to 5% ACN in water and hold for 0.30 min, Flow rate is set at 1.2 mL/min.

### Example 17. Synthesis of (3R,5S,E)-7-(2,6-dicyclobutyl-4-(4-fluorophenyl)-5-(methoxymethyl)pyridin-3-yl)-3,5-dihydroxyhept-6-enoic acid sodium salt

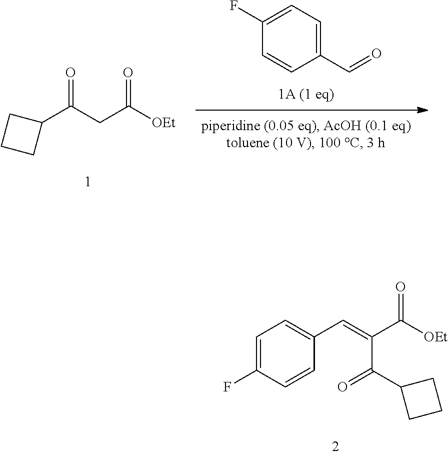 [SMILES: O=Cc1ccc(F)cc1 ; CCOC(=O)CC(=O)C1CCC1 ; CCOC(=O)/C(=C/c1ccc(F)cc1)C(=O)C1CCC1]

Step 1: To a solution of compound 1 (5.00 g, 29.38 mmol, 1 eq) and compound 1A (3.65 g, 29.38 mmol, 3.10 mL, 1 eq) in toluene (50 mL) was added piperidine (125.07 mg, 1.47 mmol, 0.05 eq) and AcOH (176.41 mg, 2.94 mmol, 0.1 eq). The mixture was stirred at 100 °C. for 3 hours under N₂. LCMS showed 28% of desired compound was detected. The reaction was concentrated under reduced pressure. The residue was purified by flash column chromatography on silica gel (0-3% EtOAc in PE) to give compound 2 (6.25 g, 77.00% yield) as colorless oil. LCMS (ESI+): m/z=277.1 (M+1), RT: 0.483 min. LC/MS: Kinetex® EVO C18 2.1×30 mm 5 μm. Detection methods are Photo-Diode Array (PDA). MS mode was positive electrospray ionization. MS range was 50-1050. Mobile phase A was 0.04% TFA in water, and mobile phase B was 0.02% TFA in HPLC grade acetonitrile. The gradient was 5-95% B in 0.60 min, flow rate was 2.0 mL/min; then hold at 95% ACN for 0.18 minutes, flow rate was 2.0 mL/min; return back to 5% ACN in water and hold for 0.02 min, flow rate was 2.0 mL/min.

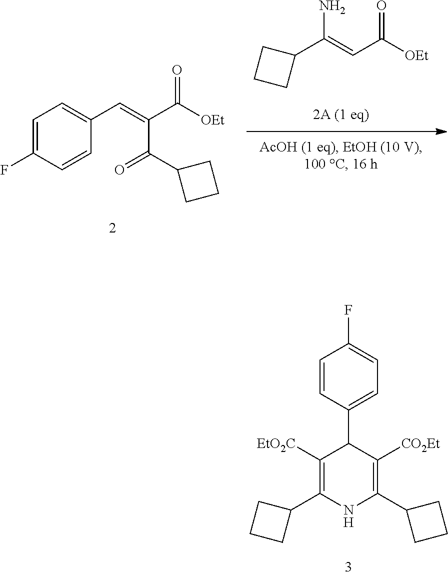 [SMILES: CCOC(=O)/C(=C/c1ccc(F)cc1)C(=O)C1CCC1 ; CCOC(=O)/C=C(\N)C1CCC1 ; CCOC(=O)C1=C(C2CCC2)NC(C2CCC2)=C(C(=O)OCC)C1c1ccc(F)cc1]

Step 2: To a solution of compound 2 (6.25 g, 22.62 mmol, 1 eq) and compound 2A (3.83 g, 22.62 mmol, 1 eq) in EtOH (60 mL) was added AcOH (1.36 g, 22.62 mmol, 1.29 mL, 1 eq). The mixture was stirred at 100 °C for 16 hours under N₂. LCMS showed compound 2 was consumed completely and 55% of desired compound was detected. The reaction was concentrated under reduced pressure. The residue was purified by flash column chromatography on silica gel (0-6% EtOAc in PE) to give compound 3 (3.70 g, 38.26% yield) as a white solid. ¹H NMR: (400 MHz, CDCl₃) δ 1.23 (t, J=7.2 Hz, 6H) 1.82-1.90 (m, 2H) 2.00-2.09 (m, 6H) 2.28-2.39 (m, 4H) 4.06-4.14 (m, 4H) 4.33-4.46 (m, 2H) 4.96 (s, 1H) 6.53 (s, 1H) 6.88 (t, J=8.8 Hz, 2H) 7.20 (dd, J=8.4, 5.6 Hz, 2H).

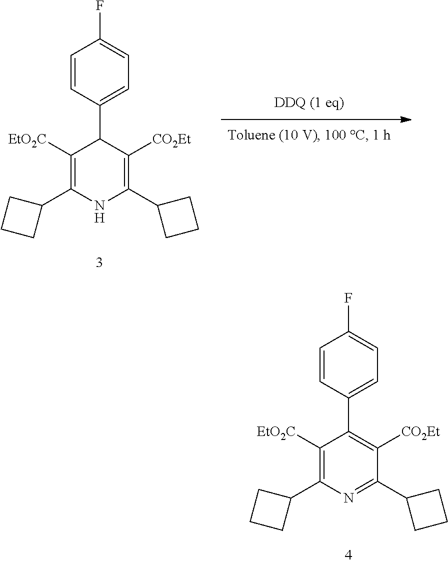 [SMILES: CCOC(=O)C1=C(C2CCC2)NC(C2CCC2)=C(C(=O)OCC)C1c1ccc(F)cc1 ; CCOC(=O)c1c(C2CCC2)nc(C2CCC2)c(C(=O)OCC)c1-c1ccc(F)cc1]

Step 3: To a solution of compound 3 (3.7 g, 8.65 mmol, 1 eq) in toluene (38 mL) was added DDQ (1.96 g, 8.65 mmol, 1 eq). The mixture was stirred at 100 °C for 1 hour under N₂. LCMS showed reactant was consumed completely and 20% of desired compound was detected. The reaction was concentrated under reduced pressure. The residue was purified by flash column chromatography on silica gel (0-6% EtOAc in PE) to give compound 4 (3.27 g, 88.80% yield) as a white solid. LCMS (ESI+): m/z=426.2 (M+1), RT: 0.610 min. LC/MS: Kinetex® EVO C18 2.1×30 mm 5 μm. Detection methods are Photo-Diode Array (PDA). MS mode was positive electrospray ionization. MS range was 50-1050. Mobile phase A was 0.04% TFA in water, and mobile phase B was 0.02% TFA in HPLC grade acetonitrile. The gradient was 5-95% B in 0.60 min, flow rate was 2.0 mL/min; then hold at 95% ACN for 0.18 minutes, flow rate was 2.0 mL/min; return back to 5% ACN in water and hold for 0.02 min, flow rate was 2.0 mL/min.

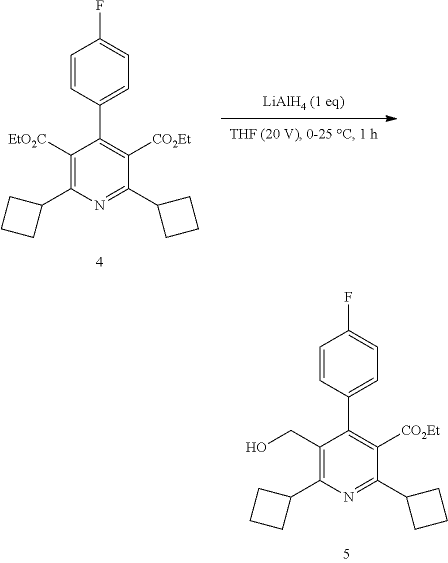 [SMILES: CCOC(=O)c1c(C2CCC2)nc(C2CCC2)c(C(=O)OCC)c1-c1ccc(F)cc1 ; CCOC(=O)c1c(C2CCC2)nc(C2CCC2)c(CO)c1-c1ccc(F)cc1]

Step 4: To a solution of compound 4 (3.17 g, 7.45 mmol, 1 eq) in THF (30 mL) was added LAH (1 M, 1 eq) at 0 °C under N₂. The mixture was stirred at 25 °C for 1 hour under N₂. LCMS showed 4% of reactant remained and 83% of desired compound was detected. The reaction was cooled to 0 °C, water (0.3 mL) was slowly added to the reaction, followed by 15% aq·NaOH (0.3 mL) and water (0.9 mL) then the mixture was warmed to room temperature and stirred for 15 minutes. Anhydrous MgSO₄ was added, and the mixture was filtered. The filter cake was washed with EtOAc (10 mL×3), the filtrate was concentrated under reduced pressure to give residue. The residue was purified by flash column chromatography on silica gel (0-20% EtOAc in PE) to give compound 5 (2.38 g, 83.31% yield) as a light yellow solid. LCMS (ESI+): m/z=384.1 (M+1), RT: 0.452 min. LC/MS: Kinetex® EVO C18 2.1×30 mm 5 μm. Detection methods are Photo-Diode Array (PDA). MS mode was positive electrospray ionization. MS range was 50-1050. Mobile phase A was 0.04% TFA in water, and mobile phase B was 0.02% TFA in HPLC grade acetonitrile. The gradient was 5-95% B in 0.60 min, flow rate was 2.0 mL/min; then hold at 95% ACN for 0.18 minutes, flow rate was 2.0 mL/min; return back to 5% ACN in water and hold for 0.02 min, flow rate was 2.0 mL/min.

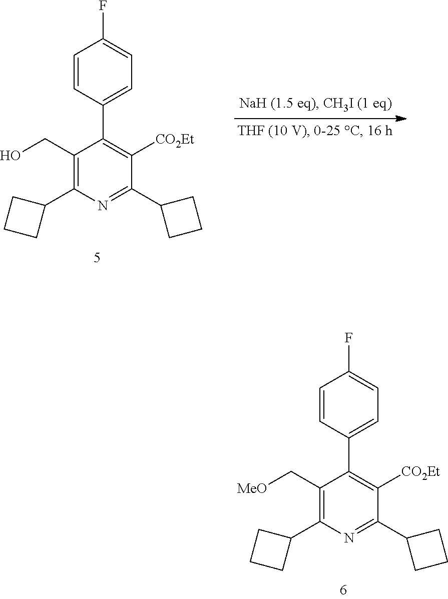 [SMILES: CCOC(=O)c1c(C2CCC2)nc(C2CCC2)c(CO)c1-c1ccc(F)cc1 ; CCOC(=O)c1c(C2CCC2)nc(C2CCC2)c(COC)c1-c1ccc(F)cc1]

Step 5: To a solution of compound 5 (2.38 g, 6.21 mmol, 1 eq) in THF (25 mL) was added NaH (372.68 mg, 9.32 mmol, 60% purity, 1.5 eq) at 0 °C under N₂, the mixture was stirred at 25 °C for 0.5 hour under N₂. CH₃I (881.71 mg, 6.21 mmol, 1 eq) was added to the reaction at 0 °C, the mixture was stirred at 25 °C for 16 hours under N₂. LCMS showed 99% of desired compound was detected. The reaction mixture was added dropwise to H₂O 20 mL at 0 °C, and then extracted with EtOAc (30 mL×3). The combined organic layers were washed with brine (30 mL), dried over anhydrous Na₂SO₄, filtered and concentrated under reduced pressure to give compound 6 (2.46 g, 99.63% yield) as a yellow oil. LCMS (ESI+): m/z=398.1 (M+1), RT: 0.503 min. LC/MS: Kinetex® EVO C18 2.1×30 mm 5 μm. Detection methods are Photo-Diode Array (PDA). MS mode was positive electrospray ionization. MS range was 50-1050. Mobile phase A was 0.04% TFA in water, and mobile phase B was 0.02% TFA in HPLC grade acetonitrile. The gradient was 5-95% B in 0.60 min, flow rate was 2.0 mL/min; then hold at 95% ACN for 0.18 minutes, flow rate was 2.0 mL/min; return back to 5% ACN in water and hold for 0.02 min, flow rate was 2.0 mL/min.

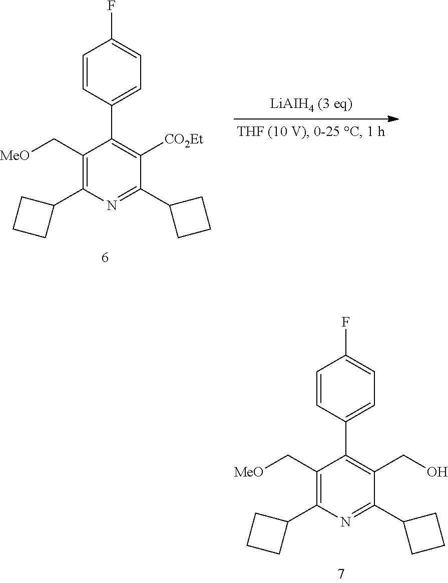 [SMILES: CCOC(=O)c1c(C2CCC2)nc(C2CCC2)c(COC)c1-c1ccc(F)cc1 ; COCc1c(C2CCC2)nc(C2CCC2)c(CO)c1-c1ccc(F)cc1]

Step 6: To a solution of compound 6 (2.36 g, 5.94 mmol, 1 eq) in THF (2 mL) was added LAH (676.05 mg, 17.81 mmol, 3 eq) at 0 °C. The mixture was stirred at 25 °C for 1 hour under N₂. LCMS showed no Reactant remained and 95% of desired compound was detected. The reaction was cooled to 0 °C, water (0.7 mL) was slowly added to the reaction, followed by 15% aq·NaOH (0.7 mL) and water (2.1 mL), then then mixture was warmed to room temperature and stirred for 15 minutes. Anhydrous MgSO₄ was added, and the mixture was filtered. The filter cake was washed with EtOAc (20 mL×3), the filtrate was concentrated under reduced pressure to give compound 7 (2.10 g, 99.51% yield) as a yellow oil. LCMS (ESI+): m/z=356.2 (M+1), RT: 0.396 min. LC/MS: Kinetex® EVO C18 2.1×30 mm 5 μm. Detection methods are Photo-Diode Array (PDA). MS mode was positive electrospray ionization. MS range was 50-1050. Mobile phase A was 0.04% TFA in water, and mobile phase B was 0.02% TFA in HPLC grade acetonitrile. The gradient was 5-95% B in 0.60 min, flow rate was 2.0 mL/min; then hold at 95% ACN for 0.18 minutes, flow rate was 2.0 mL/min; return back to 5% ACN in water and hold for 0.02 min, flow rate was 2.0 mL/min.

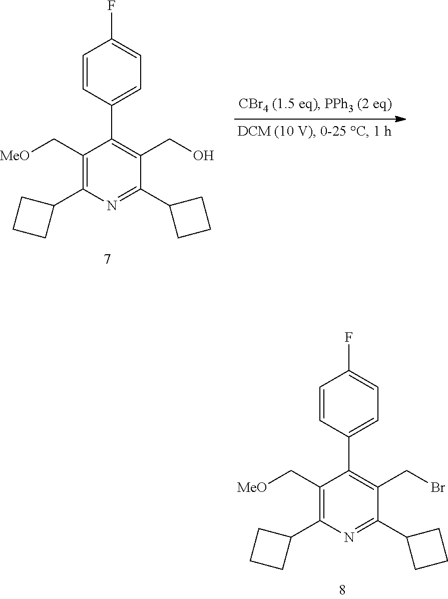 [SMILES: COCc1c(C2CCC2)nc(C2CCC2)c(CO)c1-c1ccc(F)cc1 ; COCc1c(C2CCC2)nc(C2CCC2)c(CBr)c1-c1ccc(F)cc1]

Step 7: To a solution of compound 7 (2.1 g, 5.91 mmol, 1 eq) in DCM (20 mL) was added PPh₃ (3.10 g, 11.82 mmol, 2 eq) and CBr₄ (2.94 g, 8.86 mmol, 1.5 eq) at 0 °C. The mixture was stirred at 25 °C for 1 hour under N₂. LCMS showed no Reactant remained and 22% of desired compound was detected. The reaction mixture was concentrated under reduced pressure. The residue was purified by flash column chromatography on silica gel (0-5% EtOAc in PE) to give compound 8 (2.03 g, 82.13% yield) as a yellow oil. LCMS (ESI+): m/z=419.1 (M+1), RT: 0.611 min. LC/MS: Kinetex® EVO C18 2.1×30 mm 5 μm. Detection methods are Photo-Diode Array (PDA). MS mode was positive electrospray ionization. MS range was 50-1050. Mobile phase A was 0.04% TFA in water, and mobile phase B was 0.02% TFA in HPLC grade acetonitrile. The gradient was 5-95% B in 0.60 min, flow rate was 2.0 mL/min; then hold at 95% ACN for 0.18 minutes, flow rate was 2.0 mL/min; return back to 5% ACN in water and hold for 0.02 min, flow rate was 2.0 mL/min.

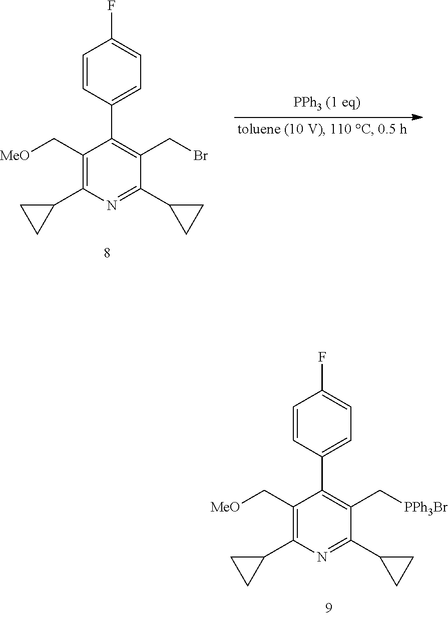

Step 8: To a solution of compound 8 (2.03 g, 4.85 mmol, 1 eq) in toluene (20 mL) was added PPh₃ (1.27 g, 4.85 mmol, 1 eq). The mixture was stirred at 110 °C for 0.5 hour under N₂. LCMS showed no Reactant remained and 63% of desired compound was detected. The reaction mixture was concentrated under reduced pressure to give compound 9 (3.3 g, crude) as a white solid. LCMS (ESI+): m/z=600.7 (M-Br), RT: 0.473 min. LC/MS: Kinetex® EVO C18 2.1×30 mm 5 μm. Detection methods are Photo-Diode Array (PDA). MS mode was positive electrospray ionization. MS range was 50-1050. Mobile phase A was 0.04% TFA in water, and mobile phase B was 0.02% TFA in HPLC grade acetonitrile. The gradient was 5-95% B in 0.60 min, flow rate was 2.0 mL/min; then hold at 95% ACN for 0.18 minutes, flow rate was 2.0 mL/min; return back to 5% ACN in water and hold for 0.02 min, flow rate was 2.0 mL/min.

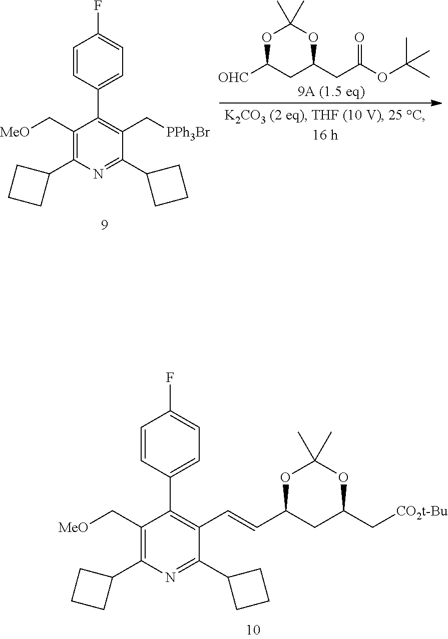

Step 9: To a solution of compound 9 (3.30 g, 4.85 mmol, 1 eq) in THF (40 mL) was added K₂CO₃ (1.34 g, 9.70 mmol, 2 eq) and compound 9A (1.88 g, 7.27 mmol, 1.5 eq). The mixture was stirred at 25 °C for 16 hours under N₂. LCMS showed 39% of compound 9 remained and 47% of desired compound was detected. The reaction mixture was concentrated under reduced pressure. The residue was purified by flash column chromatography on silica gel (0-6% EtOAc in PE) to give crude product. The crude product was purified by prep-HPLC (column: Phenomenex luna C18 150*25 mm*10 μm; mobile phase: [water (TFA)-ACN]; gradient: 61%-91% B over 10 min) to give compound 10 (800 mg, 24.19% yield) as a white solid. ¹H NMR: (400 MHz, CDCl₃) δ 0.97 (q, J=11.6 Hz, 1H), 1.27 (t, J=7.2 Hz, 2H), 1.30-1.37 (m, 4H), 1.42-1.50 (m, 12H), 1.88-2.04 (m, 3H), 2.07-2.16 (m, 1H), 2.23-2.44 (m, 6H), 2.49-2.62 (m, 2H), 2.63-2.77 (m, 2H), 3.90-4.08 (m, 4H), 4.13 (q, J=7.2 Hz, 1H), 4.17-4.34 (m, 2H), 5.20-5.34 (m, 1H), 6.09-6.27 (m, 1H), 7.01-7.19 (m, 4H). In some embodiments, compound 10 is the sodium salt of compound 10.

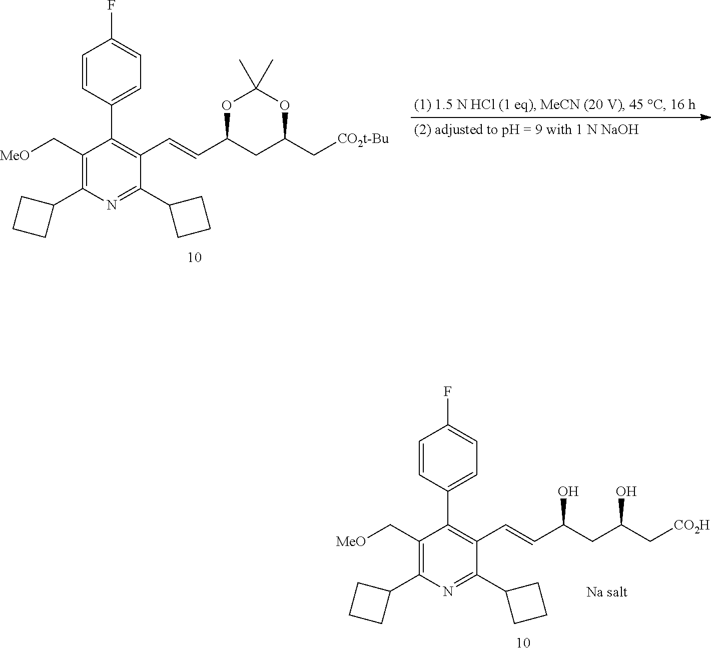 [SMILES: COCc1c(C2CCC2)nc(C2CCC2)c(/C=C/[C@@H]2C[C@H](CC(=O)OC(C)(C)C)OC(C)(C)O2)c1-c1ccc(F)cc1 ; COCc1c(C2CCC2)nc(C2CCC2)c(/C=C/[C@@H](O)C[C@@H](O)CC(=O)O)c1-c1ccc(F)cc1]

Step 10: To a solution of compound 10 (200 mg, 293.23 μmol, 1 eq) in ACN (2 mL) was added HCl (1.5 M, 1 eq). The mixture was stirred at 45 °C for 16 hours. LCMS showed 95% of desired compound was detected. The reaction mixture was adjusted to pH=9 with IN aq·NaOH, filtered and the filtrate was concentrated under reduced pressure. The residue was purified by reversed-phase HPLC (column: Waters Xbridge 150*25 mm*5 μm; mobile phase: [water (NH₃H₂O)-ACN]; gradient: 16%-46% B over 12 min) to give (3R,5S,E)-7-(2,6-dicyclobutyl-4-(4-fluorophenyl)-5-(methoxymethyl)pyridin-3-yl)-3,5-dihydroxyhept-6-enoic acid sodium salt (65.76 mg, 44.27% yield) as a white solid. ¹H NMR: (400 MHz, CDCl₃) § 1.22 (d, J=12.4 Hz, 1H), 1.34-1.49 (m, 1H), 1.76-1.89 (m, 2H), 1.89-1.97 (m, 1H), 1.98-2.08 (m, 1H), 2.10-2.36 (m, 6H), 2.44-2.56 (m, 2H), 2.57-2.68 (m, 2H), 3.12 (s, 3H), 3.79 (q, J=8.4 Hz, 1H), 3.85-3.99 (m, 3H), 4.02 (s, 1H), 4.09 (s, 1H), 5.19 (dd, J=16.0, 5.6 Hz, 1H), 6.11 (d, J=16.0 Hz, 1H), 6.86-7.04 (m, 4H). LCMS (ESI+): m/z=484.3 (M+1), RT: 1.630 min. LC/MS: Kinetex® EVO C18 3.0×50 mm 2.6 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in 3.40 min, Flow rate is set at 0.9 mL/min; then hold at 95% ACN for 0.30 minutes, Flow rate is set at 0.9 mL/min; return back to 5% ACN in water and hold for 0.30 min. Flow rate is set at 1.2 mL/min.

### Example 18. Synthesis of (3R,5S,E)-7-(5-cyclopropyl-7-(4-fluorophenyl)benzo[d]thiazol-6-yl)-3,5-dihydroxyhept-6-enoic acid sodium salt

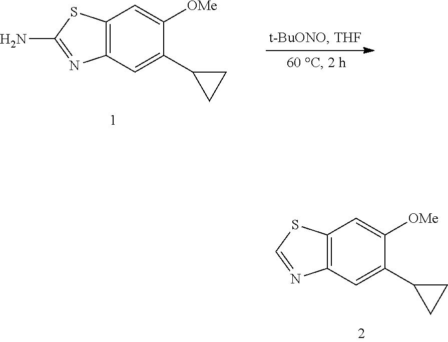 [SMILES: COc1cc2sc(N)nc2cc1C1CC1 ; COc1cc2scnc2cc1C1CC1]

Step 1: To a solution of compound 1 (12.1 g, 51.63 mmol, 1 eq) in THF (60 mL) was added t-BuONO (6.39 g, 61.96 mmol, 7.37 mL, 1.2 eq). The mixture was stirred at 60 °C for 2 hours. LCMS showed the starting material was consumed completely and desired product was observed. The mixture was concentrated under reduced pressure. The residue was purified by flash column chromatography on silica gel (0-10% EtOAc in PE) to give compound 2 (6.1 g, 51.80% yield) as yellow oil. ¹H NMR: (400 MHz, DMSO-d₆) δ 0.69-0.75 (m, 2H), 0.91-0.97 (m, 2H), 2.18 (tt, J=8.4, 5.6 Hz, 1H), 3.90 (s, 3H), 7.53 (s, 1H), 7.68 (s, 1H), 9.13 (s, 1H).

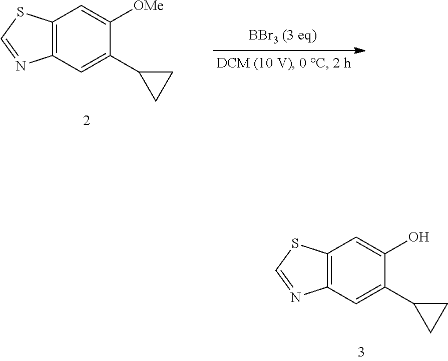 [SMILES: COc1cc2scnc2cc1C1CC1 ; Oc1cc2scnc2cc1C1CC1]

Step 2: To a solution of compound 2 (5.1 g, 24.84 mmol, 1 eq) in DCM (50 mL) was added BBr₃ (1 M, 62.11 mL, 2.5 eq) at 0 °C under N₂ atmosphere. The mixture was stirred at 0 °C for 2 hours under N₂ atmosphere. LCMS showed the starting material was consumed completely and desired product was observed. The reaction mixture was quenched with H₂O (50 mL) and extracted with DCM (50 mL×3). The combined organic layers were washed with brine (50 mL×2), dried over Na₂SO₄, filtered and concentrated under reduced pressure. The residue was purified by flash column chromatography on silica gel (0-10% MeOH in DCM) to give compound 3 (3.5 g, 71.45% yield) as a white solid. LCMS (ESI+): m/z=191.8 (M+1), RT: 0.336 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

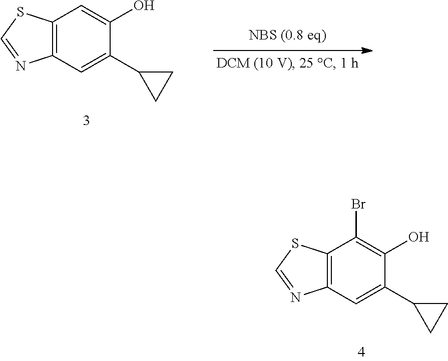 [SMILES: Oc1cc2scnc2cc1C1CC1 ; Oc1c(C2CC2)cc2ncsc2c1Br]

Step 3: To a solution of compound 3 (3.0 g, 15.69 mmol, 1 eq) in DMF (30 mL) was added NBS (2.23 g, 12.55 mmol, 0.8 eq). The mixture was stirred at 25 °C for 1 hour. LCMS showed the starting material was consumed completely and desired product was observed. The mixture was diluted with H₂O (50 mL) and extracted with EtOAc (50 mL×3). The combined organic layers were washed with brine (50 mL×5), dried over Na₂SO₄, filtered and concentrated under reduced pressure. The residue was purified by flash column chromatography on silica gel (0-10% EtOAc in PE) to give compound 4 (2.73 g, 64.42% yield) as a white solid. LCMS (ESI+): m/z=269.7 (M+1), RT: 0.415 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

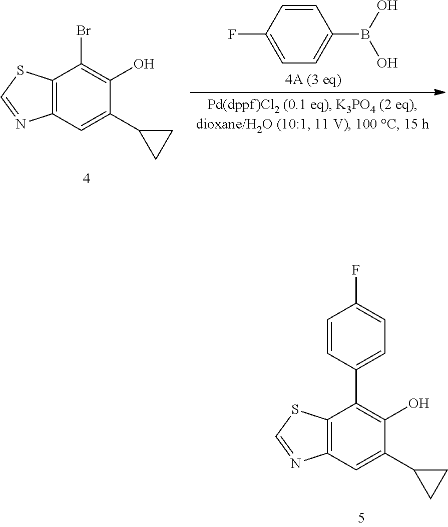 [SMILES: Oc1c(C2CC2)cc2ncsc2c1Br ; OB(O)c1ccc(F)cc1 ; Oc1c(C2CC2)cc2ncsc2c1-c1ccc(F)cc1]

Step 4: A mixture of compound 4 (3.1 g, 11.48 mmol, 1 eq), compound 4A (4.82 g, 34.43 mmol, 3 eq), K₃PO₄ (4.87 g, 22.95 mmol, 2 eq) and Pd(dppf)Cl₂ (839.65 mg, 1.15 mmol, 0.1 eq) in dioxane (30 mL) and H₂O (3 mL) was degassed and purged with N₂ for 3 times, then the mixture was stirred at 100 °C for 15 hours under N₂ atmosphere. TLC showed the starting material was consumed completely and desired product was observed. The reaction mixture was concentrated under reduced pressure. The residue was purified by flash column chromatography on silica gel (0-10% EtOAc in PE) to give crude product. The crude product was dissolved in DCM (50 mL), washed by aq·NaOH (0.5 M, 20 mL×2), brine (20 mL), dried over Na₂SO₄, filtered and concentrated under reduced pressure to give compound 5 (2.8 g, 76.96% yield) as a white solid. ¹H NMR: (400 MHz, DMSO-d₆) δ 0.70-0.78 (m, 2H), 0.95-1.02 (m, 2H), 2.21 (tt, J=8.4, 5.2 Hz, 1H), 7.14 (t, J=9.2 Hz, 1H), 7.29-7.38 (m, 2H), 7.50-7.63 (m, 3H), 7.83 (dd, J=8.4, 6.4 Hz, 1H), 8.54-8.95 (m, 1H), 9.06 (s, 1H).

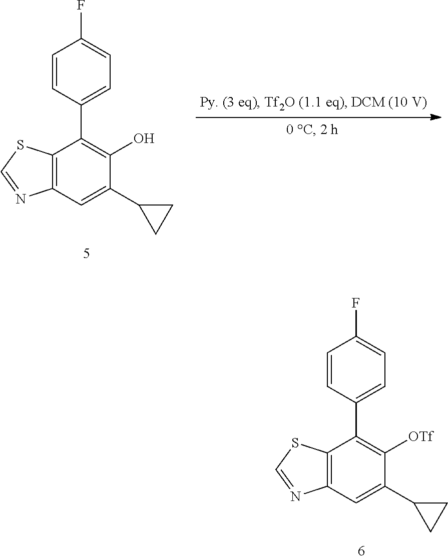 [SMILES: Oc1c(C2CC2)cc2ncsc2c1-c1ccc(F)cc1 ; O=S(=O)(Oc1c(C2CC2)cc2ncsc2c1-c1ccc(F)cc1)C(F)(F)F]

Step 5: To a solution of compound 5 (1.4 g, 4.91 mmol, 1 eq) in DCM (28 mL) at 0 °C under N₂ atmosphere was added Tf₂O (1.38 g, 4.91 mmol, 809.54 μL, 1.0 eq) and Py (1.16 g, 14.72 mmol, 1.19 mL, 3 eq). The mixture was stirred at 0 °C for 2 hours under N₂ atmosphere. LCMS showed desired product was observed. The mixture was diluted with H₂O (30 mL) and extracted with DCM (30 mL×3). The combined organic layers were washed with brine (30 mL), dried over Na₂SO₄, filtered and concentrated under reduced pressure. The residue was purified by flash column chromatography on silica gel (0~10% EtOAc in PE) to give crude product. The crude product was purified by prep-HPLC (column: Phenomenex luna C18 150*25 mm*10 μm; mobile phase: [water (TFA)-ACN]; gradient: 53%-83% B over 10 min) to give compound 6 (0.79 g, 38.19% yield) as a white solid. LCMS (ESI+): m/z=417.9 (M+1), RT: 0.603 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

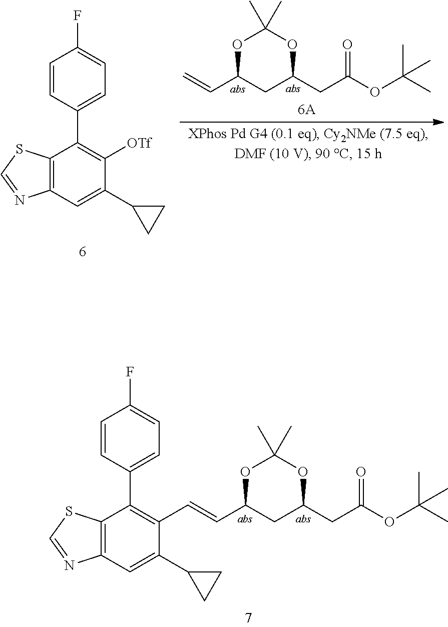 [SMILES: O=S(=O)(Oc1c(C2CC2)cc2ncsc2c1-c1ccc(F)cc1)C(F)(F)F ; C=C[C@@H]1C[C@H](CC(=O)OC(C)(C)C)OC(C)(C)O1 ; CC(C)(C)OC(=O)C[C@H]1C[C@@H](/C=C/c2c(C3CC3)cc3ncsc3c2-c2ccc(F)cc2)OC(C)(C)O1]

Step 6: A mixture of compound 6 (0.4 g, 958.32 μmol, 1 eq), compound 6A (1.23 g, 4.79 mmol, 5 eq), N-cyclohexyl-N-methyl-cyclohexanamine (1.40 g, 7.19 mmol, 1.52 mL, 7.5 eq), Xphos Pd G4 (82.46 mg, 95.83 μmol, 0.1 eq) in DMF (8 mL) was degassed and purged with N₂ for 3 times, and then the mixture was stirred at 90 °C for 15 hours under N₂ atmosphere. LCMS showed desired product was observed. The mixture was diluted with H₂O (10 mL) and extracted with EtOAc (10 mL×3). The combined organic layers were washed with brine (10 mL×5), dried over Na₂SO₄, filtered and concentrated under reduced pressure to give a residue. The residue was purified by prep-HPLC (column: Waters XBridge 150*25 mm 10 μm; mobile phase: [water (NH₄HCO₃)-ACN]; gradient: 70%-100% B over 10 min) to give compound 7 (117 mg, 23.31% yield) as a green solid. ¹H NMR: (400 MHz, DMSO-d₆) δ 0.77-0.82 (m, 2H), 0.98-1.01 (m, 2H), 1.22 (s, 3H), 1.31 (s, 2H), 1.38-1.41 (m, 12H), 2.10-2.18 (m, 1H), 2.21 (d, J=8.0 Hz, 1H), 2.28 (d, J=5.2 Hz, 1H), 4.13-4.24 (m, 1H), 4.38-4.47 (m, 1H), 5.38 (dd, J=16.4, 6.0 Hz, 1H), 6.66 (d, J=16.4 Hz, 1H), 7.26-7.32 (m, 2H), 7.37-7.43 (m, 2H), 7.70 (s, 1H), 9.29 (s, 1H).

In some embodiments, compound 7 is the sodium salt of compound 7.

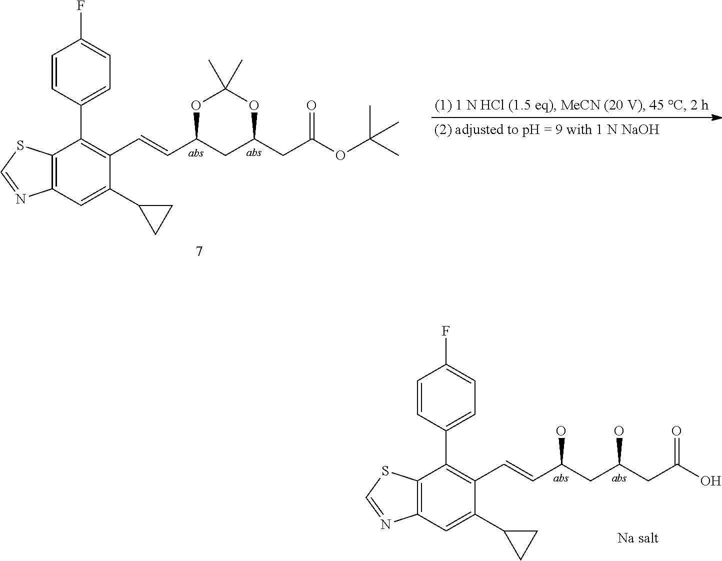 [SMILES: CC(C)(C)OC(=O)C[C@H]1C[C@@H](/C=C/c2c(C3CC3)cc3ncsc3c2-c2ccc(F)cc2)OC(C)(C)O1 ; O=C(O)C[C@H](O)C[C@H](O)/C=C/c1c(C2CC2)cc2ncsc2c1-c1ccc(F)cc1]

Step 7: To a solution of compound 7 (117 mg, 223.43 μmol, 1 eq) in MeCN (1 mL) was added HCl (1 M, 446.86 μL, 2 eq). The mixture was stirred at 45 °C for 2 hours. TLC showed the starting material was consumed completely. Then NaOH (1 M) was added to the mixture to make pH=10 at 25 °C. The mixture was stirred at 25 °C for 0.5 hour. The residue was purified by prep-HPLC (column: CD07-Daisogel SP-100-8-ODS-PK 150*25*10 μm; mobile phase: [water (NH₄HCO₃)-ACN]; gradient: 13%-43% B over 12 min) to give (3R,5S,E)-7-(5-cyclopropyl-7-(4-fluorophenyl)benzo[d]thiazol-6-yl)-3,5-dihydroxyhept-6-enoic acid sodium salt (16.1 mg, 16.01% yield) as a yellow solid. LCMS (ESI+): m/z=428.0 (M+1), RT: 1.181 min. LC/MS: Kinetex® XBridge C18 3.0*50 mm, 5 μm. Mobile phase: Ramp from 5% ACN in water (0.025% NH₃·H₂O) to 95% ACN in 3.00 min, Flow rate is set at 0.9 mL/min; then hold at 95% ACN for 0.70 minutes Flow rate is set at 0.9 mL/min; return back to 5% ACN in water and hold for 0.30 min. Flow rate is set at 1.2 mL/min. Column temperature at 40 °C and detector wavelength from 210 nm to 265 nm. ¹H NMR: (400 MHz, DMSO-d₆) δ 0.74-0.87 (m, 2H), 0.95-1.07 (m, 2H), 1.11-1.46 (m, 2H), 1.94-2.08 (m, 1H), 2.09-2.19 (m, 2H), 3.66-3.70 (m, 1H), 4.08-4.15 (m, 1H), 5.43-5.52 (m, 1H), 6.59 (d, J=16.0 Hz, 1H), 7.25-7.33 (m, 2H), 7.35-7.45 (m, 2H), 7.68 (s, 1H), 9.26 (s, 1H).

### Example 19. Synthesis of (3R,5S,E)-7-(4-(2-chloro-4-fluorophenyl)-2,6-dicyclopropylpyridin-3-yl)-3,5-dihydroxyhept-6-enoic acid sodium salt

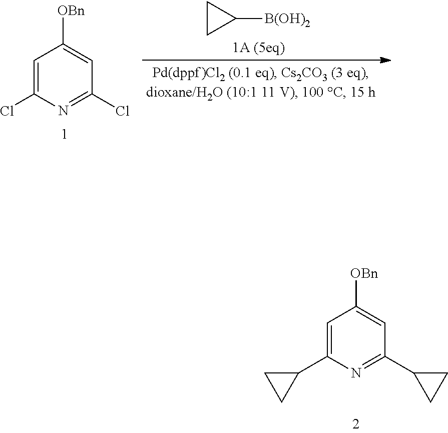 [SMILES: Clc1cc(OCc2ccccc2)cc(Cl)n1 ; OB(O)C1CC1 ; c1ccc(COc2cc(C3CC3)nc(C3CC3)c2)cc1]

Step 1: A mixture of compound 1 (10 g, 39.35 mmol, 1 eq), compound 1A (16.90 g, 196.76 mmol, 5 eq), Cs₂CO₃ (38.47 g, 118.06 mmol, 3 eq), Pd(dppf)Cl₂ (1.44 g, 1.97 mmol, 0.05 eq) and H₂O (10 mL) in dioxane (100 mL) was degassed and purged with N₂ for 3 times, and then the mixture was stirred at 100 °C for 15 hours under N₂ atmosphere. LCMS showed the starting material was consumed completely and desired MS was observed. After cooling to room temperature, the reaction mixture was diluted with H₂O (150 mL) and extracted with EtOAc (150 mL×3). The combined organic layers were washed with brine (150 mL×3), dried over Na₂SO₄, filtered and concentrated under reduced pressure. The residue was purified by flash column chromatography on silica gel (0-15% EtOAc in PE) to afford compound 2 (7 g, 67.04% yield) as a yellow solid. LCMS (ESI+): m/z=266.2 (M+1), RT: 0.331 min. LC/MS: Kinetex® EVO C18 2.1×30 mm 5 μm. Detection methods are photo-diode array (PDA &ELSD). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

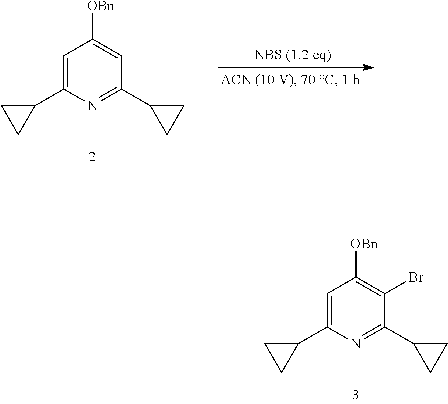 [SMILES: c1ccc(COc2cc(C3CC3)nc(C3CC3)c2)cc1 ; Brc1c(OCc2ccccc2)cc(C2CC2)nc1C1CC1]

Step 2: To a solution of compound 2 (5.0 g, 18.84 mmol, 1 eq) in ACN (50 mL) was added NBS (4.02 g, 22.61 mmol, 1.2 eq). The mixture was stirred at 70 °C for 1 hour to give a yellow solution. LCMS showed the starting material was consumed completely and desired MS was observed. After cooling, the reaction mixture was concentrated under reduced pressure. The residue was purified by flash column chromatography on silica gel (0-15% EtOAc in PE) to afford compound 3 (6.13 g, 94.50% yield) as an off-white solid. LCMS (ESI+): m/z=344.9 (M+1), RT: 0.486 min. LC/MS: Kinetex® EVO C18 2.1×30 mm 5 μm. Detection methods are photo-diode array (PDA &ELSD). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

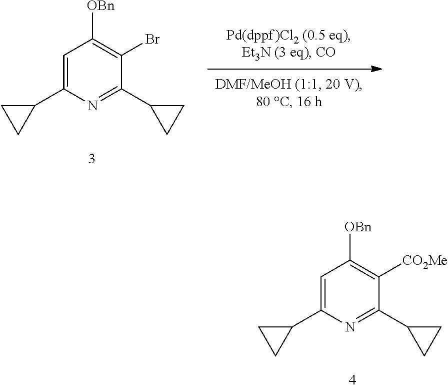 [SMILES: Brc1c(OCc2ccccc2)cc(C2CC2)nc1C1CC1 ; COC(=O)c1c(OCc2ccccc2)cc(C2CC2)nc1C1CC1]

Step 3: To a solution of compound 3 (5 g, 14.52 mmol, 1 eq) in DMF (50 mL) and MeOH (50 mL) was added Et₃N (2.94 g, 29.05 mmol, 2 eq) and Pd(dppf)Cl₂ (5.31 g, 7.26 mmol, 0.5 eq). The suspension was degassed and purged with CO for 3 times. The mixture was stirred under CO (45 Psi) at 80 °C for 16 hours. LCMS showed desired MS was observed. The reaction mixture was concentrated under reduced pressure. The reaction mixture was diluted by H₂O (100 mL) and extracted with EtOAc (100 mL×3). The combined organic layers were washed with brine (100 mL×3), dried over Na₂SO₄, filtered and concentrated under reduced pressure. The residue was purified by flash column chromatography on silica gel (0 to 10% EtOAc in PE) to afford compound 4 (2.7 g, 57.48% yield) as a white solid. LCMS (ESI+): m/z=324.0 (M+1), RT: 0.398 min. LC/MS: Kinetex® EVO C18 2.1×30 mm 5 μm. Detection methods are photo-diode array (PDA &ELSD). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

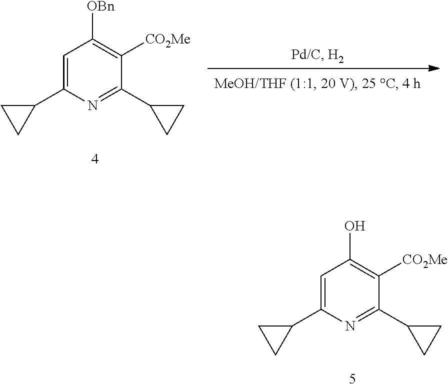 [SMILES: COC(=O)c1c(OCc2ccccc2)cc(C2CC2)nc1C1CC1 ; COC(=O)c1c(O)cc(C2CC2)nc1C1CC1]

Step 4: To a solution of compound 4 (2.4 g, 7.42 mmol, 1 eq) in MeOH (30 mL) and THF (30 mL) was added Pd/C (259.46 mg, 243.81 μmol, 10% purity) under N₂ atmosphere. The suspension was degassed and purged with H₂ for 3 times. The mixture was stirred under H₂ (45 Psi) at 25 °C for 4 hours. LCMS showed the starting material was remained and desired MS was observed. The reaction mixture was filtered and the filtrate was concentrated under reduced pressure to afford compound 5 (1.4 g, crude) as a gray solid. LCMS (ESI+): m/z=234.1 (M+1), RT: 0.247 min. LC/MS: Kinetex® EVO C18 2.1×30 mm 5 μm. Detection methods are photo-diode array (PDA &ELSD). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

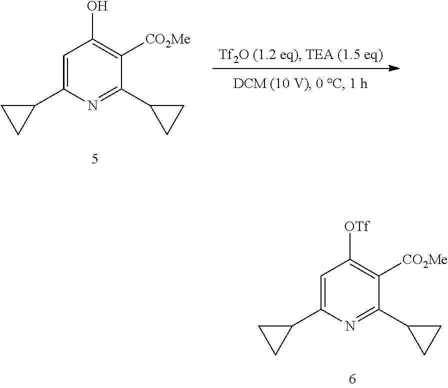 [SMILES: COC(=O)c1c(O)cc(C2CC2)nc1C1CC1 ; COC(=O)c1c(OS(=O)(=O)C(F)(F)F)cc(C2CC2)nc1C1CC1]

Step 5: To a solution compound 5 (700 mg, 3.00 mmol, 1 eq) in DCM (7 mL) was added TEA (455.49 mg, 4.50 mmol, 1.5 eq) and Tf₂O (1.02 g, 3.60 mmol, 1.2 eq). The mixture was stirred at 0 °C for 1 hour under N₂ atmosphere. LCMS showed the starting material was consumed completely and desired MS was observed. The reaction mixture was diluted by H₂O (10 mL), and extracted with DCM (10 mL×3). The combined organic layers were washed with brine (10 mL×3), dried over Na₂SO₄, filtered and concentrated under reduced pressure to afford compound 6 (1.9 g, crude) as colorless liquid. LCMS (ESI+): m/z=365.9 (M+1), RT: 0.553 min. LC/MS: Kinetex® EVO C18 2.1×30 mm 5 μm. Detection methods are photo-diode array (PDA &ELSD). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min.

Flow rate is set at 2.0 mL/min.

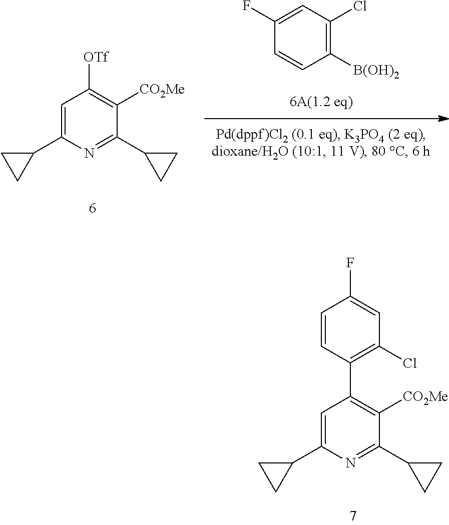 [SMILES: COC(=O)c1c(OS(=O)(=O)C(F)(F)F)cc(C2CC2)nc1C1CC1 ; OB(O)c1ccc(F)cc1Cl ; COC(=O)c1c(-c2ccc(F)cc2Cl)cc(C2CC2)nc1C1CC1]

Step 6: A mixture of compound 6 (1 g, 2.74 mmol, 1 eq), compound 6A (572.75 mg, 3.28 mmol, 1.2 eq), K₃PO₄ (1.16 g, 5.47 mmol, 2 eq), and Pd(dppf)Cl₂ (223.54 mg, 273.73 μmol, 0.1 eq) in dioxane (10 mL) and H₂O (1 mL) was degassed and purged with N₂ for 3 times and the mixture was stirred at 80 °C for 6 hours under N₂. LCMS showed the starting material was consumed completely and desired MS was observed. The reaction was concentrated under reduced pressure. The residue was purified by flash column chromatography on silica gel (0 to 5% EtOAc in PE) to afford compound 7 (680 mg, 70.50% yield) as yellow oil. LCMS (ESI+): m/z=346.1 (M+1), RT: 0.534 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

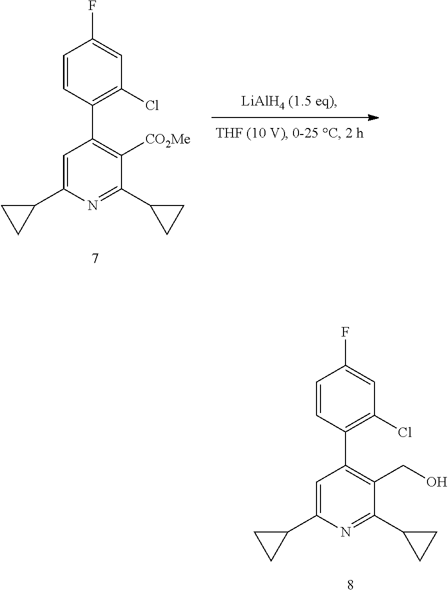 [SMILES: COC(=O)c1c(-c2ccc(F)cc2Cl)cc(C2CC2)nc1C1CC1 ; OCc1c(-c2ccc(F)cc2Cl)cc(C2CC2)nc1C1CC1]

Step 7: To a solution of compound 7 (680 mg, 1.97 mmol, 1 eq) in THF (8 mL) was added dropwise LAH (2.5 M, 1.02 mL, 1.3 eq) at 0 °C. The resulting mixture was stirred at 25 °C for 2 hours under N₂ atmosphere. LCMS showed the starting material was consumed completely and desired MS was observed. The reaction was quenched with (H₂O: 15% NaOH: H₂O=0.5 mL: 0.5 mL: 1.5 mL) at 0 °C, and stirred for 5 minutes. Na₂SO₄ was added. The mixture was stirred for 15 minutes and filtered. The filtrate was concentrated under reduced pressure to afford compound 8 (680 mg, crude) as a yellow solid. LCMS (ESI+): m/z=317.9 (M+1), RT: 0.334 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

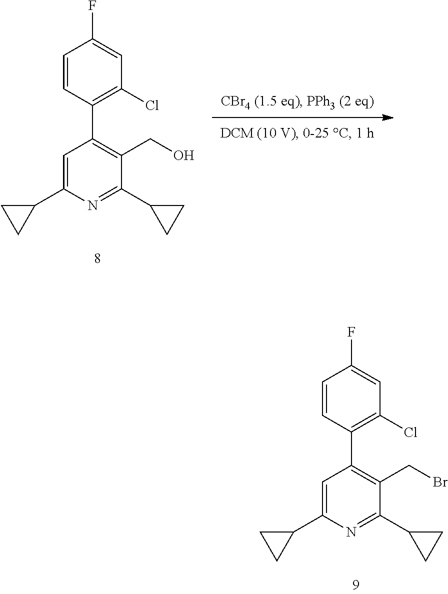 [SMILES: OCc1c(-c2ccc(F)cc2Cl)cc(C2CC2)nc1C1CC1 ; Fc1ccc(-c2cc(C3CC3)nc(C3CC3)c2CBr)c(Cl)c1]

Step 8: A mixture of compound 8 (550 mg, 1.73 mmol, 1 eq) in DCM (8 mL) was added CBr₄ (860.94 mg, 2.60 mmol, 1.5 eq) and PPh₃ (907.89 mg, 3.46 mmol, 2 eq) at 0 °C under N₂, then the mixture was stirred at 25 °C for 1 hour under N₂ atmosphere. LCMS showed desired product was observed. The mixture was concentrated under reduced pressure. The residue was purified by flash column chromatography on silica gel (0 to 10% EtOAc in PE) to afford compound 9 (460 mg, 69.82% yield) as yellow oil. LCMS (ESI+): m/z=381.9 (M+1), RT: 0.539 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

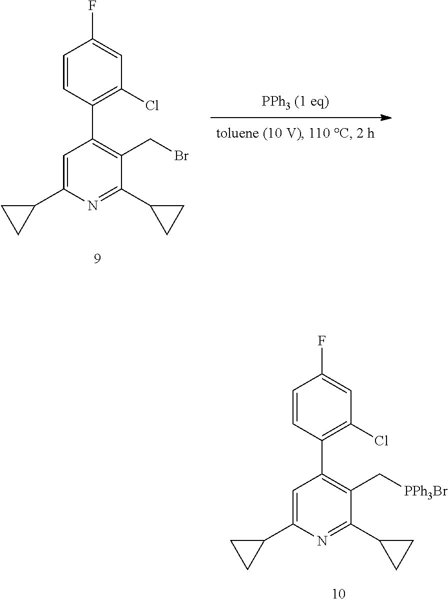

Step 9: To a solution of compound 9 (460 mg, 1.21 mmol, 1 eq) in toluene (6 mL) was added PPh₃ (316.94 mg, 1.21 mmol, 1 eq), then the mixture was stirred at 110 °C for 2 hours under N₂ atmosphere. LCMS showed desired product was observed. After cooling to room temperature, the reaction mixture was concentrated under reduced pressure to give compound 10 (790 mg, crude) as a white solid. LCMS (ESI+): m/z=562.4 (M-Br), RT: 0.451 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

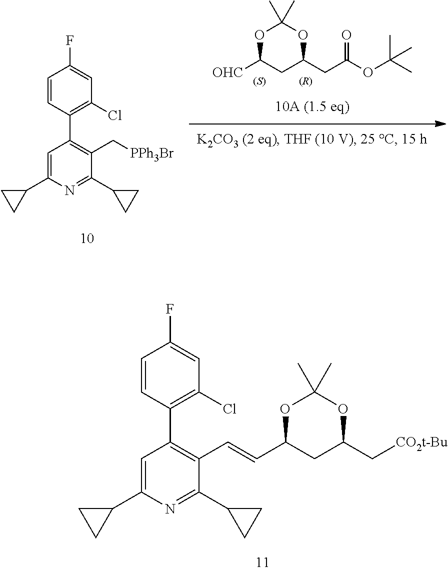

Step 10: To a solution of compound 10 (790 mg, 1.23 mmol, 1 eq) and compound 10A (476.07 mg, 1.84 mmol, 1.5 eq) in THF (10 mL) was added K₂CO₃ (339.62 mg, 2.46 mmol, 2 eq), then the mixture was stirred at 25 °C for 15 hours under N₂ atmosphere. LCMS showed the starting material was consumed completely and desired MS was observed. The reaction mixture was concentrated under reduced pressure. The residue was purified by flash column chromatography on silica gel (0 to 10% EtOAc in PE). The crude product was purified by prep-HPLC (column: Welch Xtimate C18 150*25 mm*5 μm; mobile phase: [water (TFA)-ACN]; gradient: 60%-80% B over 14 min) to afford compound 11 (110 mg, 16.52% yield) as colorless oil. ¹H NMR: (400 MHz, DMSO-d₆) δ 0.81-0.87 (m, 4H), 0.95-1.04 (m, 4H), 1.08-1.15 (m, 4H), 1.23 (s, 3H), 1.33 (s, 3H), 1.40 (s, 9H), 2.31 (dd, J=15.2, 5.2 Hz, 2H), 4.14-4.25 (m, 1H), 4.40-4.51 (m, 1H), 5.45 (dd, J=16.0, 5.6 Hz, 1H), 6.57 (d, J=16.0 Hz, 1H), 7.30-7.37 (m, 3H), 7.67 (d, J=6.0 Hz, 1H). LCMS (ESI+): m/z=542.3 (M+1), RT: 0.536 min. LC/MS: HALO C18 90A 2.7 μm 3.0×30 mm Detection methods are photo-diode array (DAD&ELSD). MS mode was positive electrospray ionization. MS range was 50-1050. Mobile phase: Ramp from 5% ACN (0.018% TFA) in water (0.037% TFA) to 95% ACN in water in 3.0 min, Flow rate is set at 1.0 mL/min; then hold at 95% ACN for 0.6 minutes Flow rate is set from 1.0 mL/min to 1.5 mL/min; return back to 5% ACN in water and hold for 0.4 min. Flow rate is set at 1.5 mL/min.

In some embodiments, compound 11 is the sodium salt of compound 11.

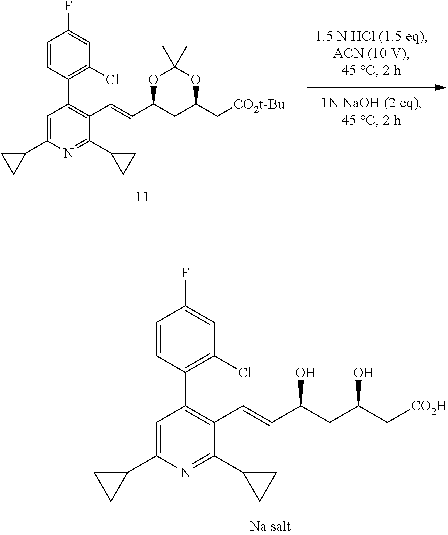 [SMILES: CC(C)(C)OC(=O)C[C@H]1C[C@@H](/C=C/c2c(-c3ccc(F)cc3Cl)cc(C3CC3)nc2C2CC2)OC(C)(C)O1 ; O=C(O)C[C@H](O)C[C@H](O)/C=C/c1c(-c2ccc(F)cc2Cl)cc(C2CC2)nc1C1CC1]

Step 11: To a solution of compound 11 (100 mg, 184.47 μmol, 1 eq) in ACN (1 mL) was added HCl (1.5 M, 184.47 μL, 1.5 eq) and the mixture was stirred at 45 °C for 2 hours under N₂ atmosphere. NaOH (1 M, 368.95 μL, 2 eq) was added and the mixture was stirred at 45 °C for 2 hours under N₂ atmosphere. LCMS showed the starting material was consumed completely and desired MS was observed. After cooling to room temperature, the reaction mixture was purified by prep-HPLC (column: Waters Xbridge 150*25 mm*5 μm; mobile phase: [water (NH₃·H₂O)-ACN]; gradient: 16%-36% B over 10 min) to afford (3R,5S,E)-7-(4-(2-chloro-4-fluorophenyl)-2,6-dicyclopropylpyridin-3-yl)-3,5-dihydroxyhept-6-enoic acid sodium salt (26.56 mg, 30.71% yield) as a white solid. ¹H NMR: (400 MHz, DMSO-d₆) δ 0.83-0.92 (m, 6H), 0.93-1.04 (m, 2H), 1.23-1.40 (m, 1H), 1.84 (dd, J=15.2, 7.6 Hz, 1H), 1.94-2.09 (m, 2H), 2.26 (dd, J=8.0, 4.4 Hz, 1H), 3.46-3.60 (m, 2H), 4.05 (q, J=6.4 Hz, 1H), 5.34 (dt, J=16.0, 6.0 Hz, 1H), 6.40 (dd, J=16.0, 8.4 Hz, 1H), 6.86 (s, 1H), 7.20-7.39 (m, 2H), 7.48 (td, J=9.2, 2.4 Hz, 1H). LCMS (ESI+): m/z=446.3 (M+1), RT: 1.759 min. LC/MS: HALO C18 90A 2.7 μm 3.0×30 mm Detection methods are photo-diode array (PDA&ELSD). MS mode was positive electrospray ionization. MS range was 50-1050. Mobile phase: Ramp from 5% ACN (0.018% TFA) in water (0.037% TFA) to 95% ACN in water in 3.0 min, Flow rate is set at 1.0 mL/min; then hold at 95% ACN for 0.6 minutes Flow rate is set from 1.0 mL/min to 1.5 mL/min; return back to 5% ACN in water and hold for 0.4 min. Flow rate is set at 1.5 mL/min.

### Example 20. Synthesis of (3R,5S,E)-7-(4-(2-chloro-4-fluorophenyl)-2,6-dicyclopropyl-5-(methoxymethyl)pyridin-3-yl)-3,5-dihydroxyhept-6-enoic acid sodium salt

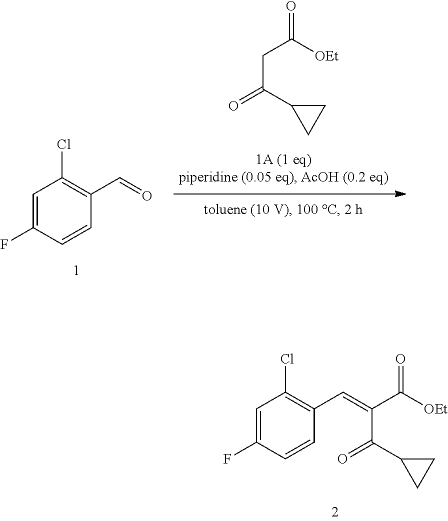 [SMILES: CCOC(=O)CC(=O)C1CC1 ; O=Cc1ccc(F)cc1Cl ; CCOC(=O)/C(=C/c1ccc(F)cc1Cl)C(=O)C1CC1]

Step 1: To a solution of compound 1A (10 g, 64.03 mmol, 1 eq), compound 1 (10.15 g, 64.03 mmol, 1 eq) in toluene (100 mL) was added piperidine (272.60 mg, 3.20 mmol, 316.16 μL, 0.05 eq) and HOAc (769.02 mg, 12.81 mmol, 733.10 μL, 0.2 eq) and the mixture was stirred at 100 °C for 2 hours. LCMS showed the reactant was consumed completely and 72% of desired product was observed. The mixture was added water (200 mL) and extracted with EtOAc (3× 200 mL). The combined organic layers were dried over anhydrous Na₂SO₄ and concentrated under reduced pressure. The crude product was purified by flash column chromatography on silica gel (0-10% EtOAc in PE) to give compound 2 (15.33 g, 80.69% yield) as colorless oil. LCMS (ESI+): m/z=297.1 (M+1), RT: 0.473 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

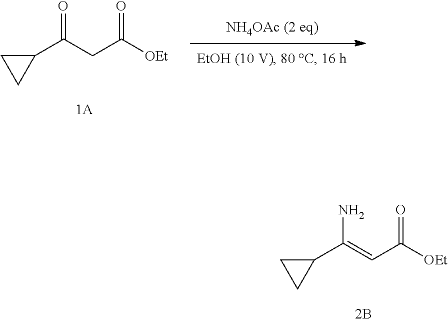 [SMILES: CCOC(=O)CC(=O)C1CC1 ; CCOC(=O)/C=C(\N)C1CC1]

Step 2: To a solution of ethyl compound 1A (5 g, 32.01 mmol, 1 eq) in EtOH (50 mL) was added ammonium acetate (4.94 g, 64.03 mmol, 2 eq) and the mixture was stirred at 80 °C for 16 hours. LCMS showed 28% of reactant remained and 49% of desired product was observed. The mixture was combined with a paralleled batch (5 g compound 1A was used) and concentrated under reduced pressure to give a crude product. The crude product was purified by flash column chromatography on silica gel (0-10% EtOAc in PE) to give compound 2B (3.18 g, 32.00% yield) as colorless oil. ¹H NMR: (400 MHz, CDCl₃) δ 0.75 (d, J=5.2 Hz, 2H), 0.87 (br d, J=6.4 Hz, 2H), 1.26 (t, J=7.2 Hz, 3H), 1.37-1.47 (m, 1H), 4.11 (q, J=7.2 Hz, 2H), 4.47 (s, 1H). LCMS (ESI+): m/z=156.2 (M+1), RT: 0.143 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

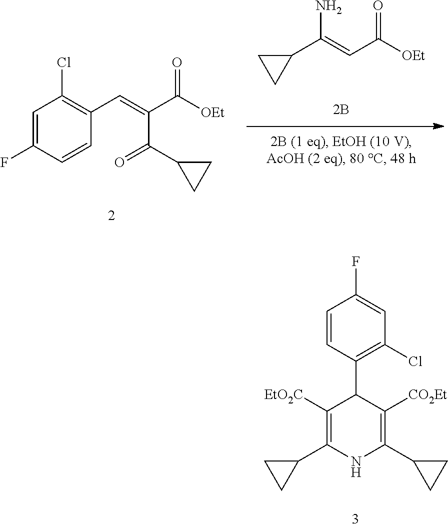 [SMILES: CCOC(=O)/C(=C/c1ccc(F)cc1Cl)C(=O)C1CC1 ; CCOC(=O)/C=C(\N)C1CC1 ; CCOC(=O)C1=C(C2CC2)NC(C2CC2)=C(C(=O)OCC)C1c1ccc(F)cc1Cl]

Step 3: A solution of compound 2 (6.5 g, 21.91 mmol, 1 eq), compound 2B (3.40 g, 21.91 mmol, 1 eq), AcOH (2.63 g, 43.81 mmol, 2.51 mL, 2 eq) in EtOH (50 mL) was stirred at 80 °C for 48 hours. LCMS showed the reactant was consumed completely and 55% of desired product was observed. The mixture was concentrated under reduced pressure. The crude product was purified by flash column chromatography on silica gel (0-5% EtOAc in PE) to give compound 3 (6.01 g, 63.23% yield) as yellow oil. LCMS (ESI+): m/z=388.1 (M+1), RT: 0.523 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

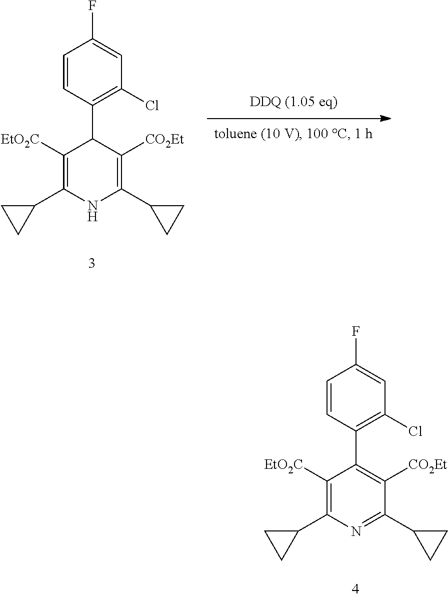 [SMILES: CCOC(=O)C1=C(C2CC2)NC(C2CC2)=C(C(=O)OCC)C1c1ccc(F)cc1Cl ; CCOC(=O)c1c(C2CC2)nc(C2CC2)c(C(=O)OCC)c1-c1ccc(F)cc1Cl]

Step 4: To a solution of compound 3 (6.01 g, 13.85 mmol, 1 eq) in toluene (60 mL) was added DDQ (3.30 g, 14.54 mmol, 1.05 eq) and the mixture was stirred at 100 °C for 1 hour. LCMS showed the reactant was consumed completely and 30% of desired product was observed. The reaction mixture was concentrated under reduced pressure. The crude product was purified by flash column chromatography on silica gel (0-5% EtOAc in PE) to give a compound 4 (5.3 g, 88.60% yield) as an off-white solid. LCMS (ESI+): m/z=432.1 (M+1), RT: 0.559 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

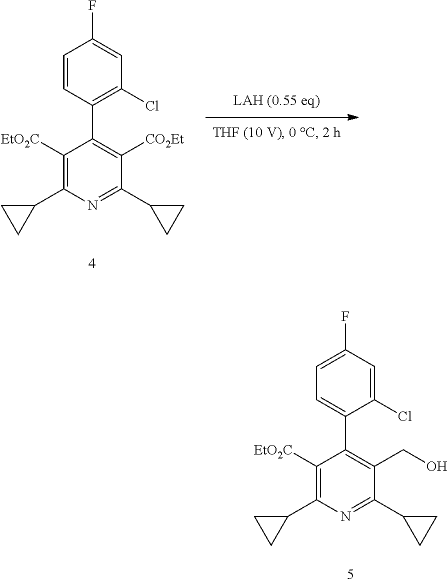 [SMILES: CCOC(=O)c1c(C2CC2)nc(C2CC2)c(C(=O)OCC)c1-c1ccc(F)cc1Cl ; CCOC(=O)c1c(C2CC2)nc(C2CC2)c(CO)c1-c1ccc(F)cc1Cl]

Step 5: To a solution of compound 4 (2.3 g, 5.33 mmol, 1 eq) in THF (20 mL) was added LiAlH₄ (1 M, 2.93 mL, 0.55 eq) and the mixture was stirred at 0 °C for 2 hours. LCMS showed 16% of reactant remained and 78% of desired product was observed. The mixture was quenched by adding EtOAc (10 mL), H₂O (1 mL) and 15% NaOH (1 mL) then dried over Na₂SO₄, filtered and the filtrate was concentrated under reduced pressure to give compound 5 (2.13 g, 77.97% yield) as yellow oil. LCMS (ESI+): m/z=390.1 (M+1), RT: 0.496 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

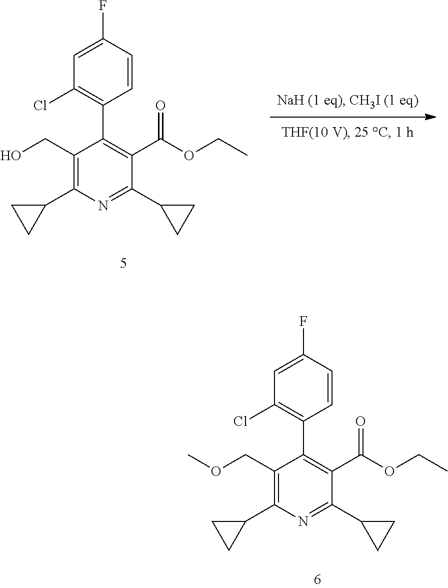 [SMILES: CCOC(=O)c1c(C2CC2)nc(C2CC2)c(CO)c1-c1ccc(F)cc1Cl ; CCOC(=O)c1c(C2CC2)nc(C2CC2)c(COC)c1-c1ccc(F)cc1Cl]

Step 6: A solution of compound 5 in THF (7 mL) was degassed with N₂ for three times then NaH (69.76 mg, 1.74 mmol, 60% purity, 1eq) was added at 0 °C and stirred for 15 min. Mel (247.58 mg, 1.74 mmol, 108.59 μL, 1eq) was added to the mixture and stirred at 25 °C for 1 hour. LCMS showed the desired mass was detected, the starting material was consumed. The reaction mixture was quenched with NH₄Cl (15 mL), extracted with ethyl acetate (20 mL×2) and washed with brine (20 mL×2), then the organic phase was dried over Na₂SO₄, filtered and concentrated under vacuum to give compound 6 (556 mg, 71.03% yield) as yellow oil. LCMS (ESI+): m/z=404.1 (M+1), RT: 0.553 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

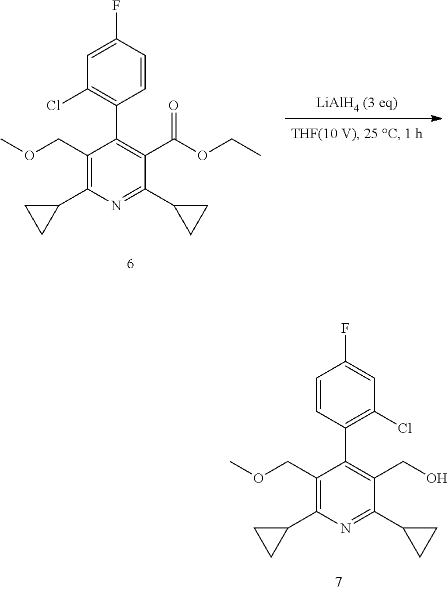 [SMILES: CCOC(=O)c1c(C2CC2)nc(C2CC2)c(COC)c1-c1ccc(F)cc1Cl ; COCc1c(C2CC2)nc(C2CC2)c(CO)c1-c1ccc(F)cc1Cl]

Step 7: A compound 6 in THF (4 mL) was degassed with N₂ three times, LiAlH₄ (128.56 mg, 3.39 mmol, 3 eq) was added to the mixture at 0 °C under N₂ atmosphere, the mixture was stirred at 25 °C for 1 hour. LCMS showed the desired mass was detected, the starting material was consumed. The reaction mixture was quenched with water (0.15 mL), NaOH (15%, 0.15 mL) and water (0.45 mL), then the mixture was filtered and the filtrate was dried over Na₂SO₄ and concentrated under vacuum to give compound 7 (500 mg, 97.91% yield) as a yellow solid. LCMS (ESI+): m/z=362.3 (M+1), RT: 0.449 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

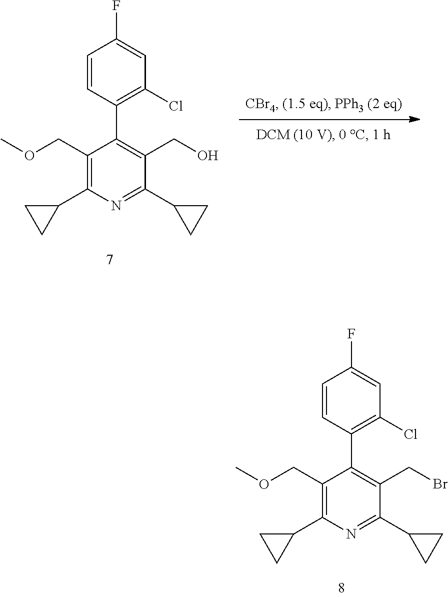 [SMILES: COCc1c(C2CC2)nc(C2CC2)c(CO)c1-c1ccc(F)cc1Cl ; COCc1c(C2CC2)nc(C2CC2)c(CBr)c1-c1ccc(F)cc1Cl]

Step 8: To a mixture of compound 7 (500 mg, 1.38 mmol, 1 eq) in DCM (5 mL) was added PPh₃ (724.87 mg, 2.76 mmol, 2 eq) and CBr₄ (687.38 mg, 2.07 mmol, 1.5 eq) at 0 °C, the mixture was warmed to 25 °C and stirred for 1 hour under N₂. LCMS showed the desired mass was detected, the starting material was consumed. The reaction mixture was concentrated under vacuum. The residue was purified by flash column chromatography on silica gel (0-10% EtOAc in PE) to give compound 8 (380 mg, 60.86% yield) as yellow oil. LCMS (ESI+): m/z=424.0 (M+1), RT: 0.579 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

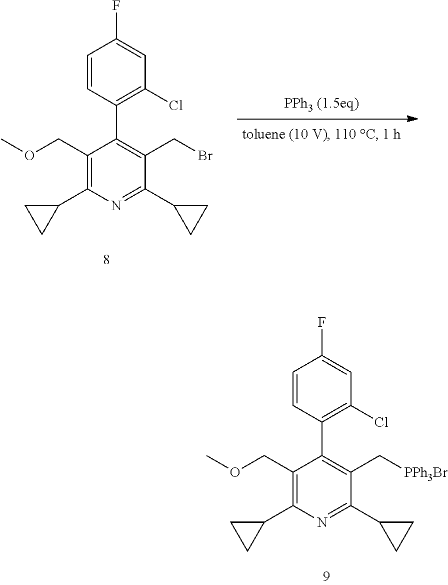

Step 9: To a mixture of compound 8 (380 mg, 894.68 μmol, 1eq) in toluene (4 mL) was degassed under vacuum and purged with N₂ for three times. PPh₃ (351.99 mg, 1.34 mmol, 1.5 eq) was added to the mixture, the mixture was heated to 110 °C and stirred for 1 hour under N₂. LCMS showed the desired mass was detected, the starting material was consumed. The reaction mixture was concentrated under vacuum to give compound 9 (614 mg, 99.89% yield) as a white solid which was used directly in the next step. LCMS (ESI+): m/z=606.2 (M−79), RT: 0.458 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

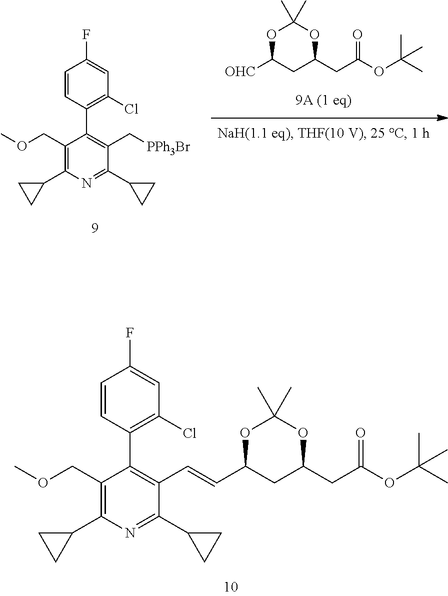

Step 10: To a mixture of compound 9 (560 mg, 815.12 μmol, 1eq) in THF (5 mL) was degassed under vacuum and purged with N₂ three times. NaH (35.86 mg, 896.63 μmol, 60% purity, 1.1 eq) was added to the mixture at 0 °C and stirred for 15 min. compound 9A (210.55 mg, 815.12 μmol, 1eq) was added to the reaction mixture, then the mixture was warmed to 25 °C and stirred for 1 hour under N₂. LCMS showed the desired mass was detected, the starting material was consumed. The reaction mixture was quenched with NH₄Cl (15 mL), extracted with ethyl acetate (24 mL×2) and washed with brine (24 mL×2), then the organic phase was dried over Na₂SO₄, filtered and concentrated under vacuum. The residue was purified by flash column chromatography on silica gel (0-10% EtOAc in PE) to give the crude product. The crude product was purified by prep-TLC (DCM/PE=3:1) to give the product. The crude product was purified by (column: Welch Ultimate C18 150*25 mm*5 μm; mobile phase: [water (TFA)-ACN]; gradient: 70%-100% B over 10 min) to give compound 10 (84 mg, 16.35% yield) as yellow oil. LCMS (ESI+): m/z=586.2 (M+1), RT: 3.275 min. LC/MS: Kinetex® EVO C18 2.1×30 mm 5 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in water in 0.60 min, Flow rate is set at 2.0 mL/min; then hold at 95% ACN for 0.18 minutes Flow rate is set at 2.0 mL/min; return back to 5% ACN in water and hold for 0.02 min. Flow rate is set at 2.0 mL/min.

In some embodiments, compound 10 is the sodium salt of compound 10.

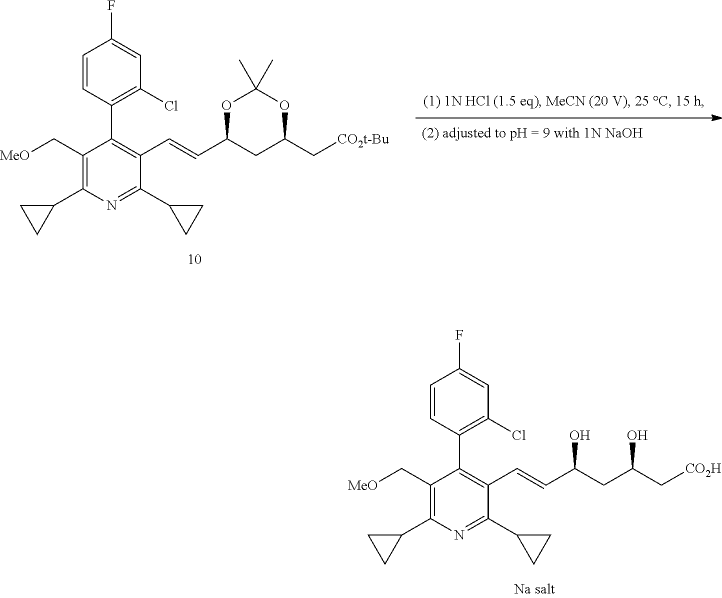 [SMILES: COCc1c(C2CC2)nc(C2CC2)c(/C=C/[C@@H]2C[C@H](CC(=O)OC(C)(C)C)OC(C)(C)O2)c1-c1ccc(F)cc1Cl ; COCc1c(C2CC2)nc(C2CC2)c(/C=C/[C@@H](O)C[C@@H](O)CC(=O)O)c1-c1ccc(F)cc1Cl]

Step 11: To a mixture of compound 10 (84 mg, 143.31 μmol, 1 eq) in MeCN (0.8 mL) was added HCl (1 M, 102.37 μL, 2 eq), the mixture was heated to 45 °C for 12 hours. NaOH (45.71 mg, 171.44 μmol, 15% purity, 1eq) was added, the mixture was stirred at 25 °C for 0.5 hour. LCMS showed the desired mass was detected, the starting material was consumed. The mixture was concentrated under vacuum. The crude product was purified with by prep-HPLC (column: Waters Xbridge 150*25 mm*5 μm; mobile phase: [water (NH₃·H₂O)-ACN]; gradient: 15%-45% B over 12 min) and lyophilized directly to give (3R,5S,E)-7-(4-(2-chloro-4-fluorophenyl)-2,6-dicyclopropyl-5-(methoxymethyl)pyridin-3-yl)-3,5-dihydroxyhept-6-enoic acid sodium salt (16.91 mg, 19.07% yield) as a white solid. ¹H NMR: (400 MHz, CDCl₃) δ 7.20-7.00 (m, 2H), 6.93 (t, J=8.0 Hz, 1H), 6.34-6.15 (m, 1H), 5.50-5.31 (m, 1H), 4.37 (d, J=10.4 Hz, 1H), 4.06 (d, J=1.6 Hz, 1H), 4.01-3.84 (m, 2H), 3.14 (s, 3H), 2.33-2.18 (m, 2H), 2.17-2.00 (m, 2H), 1.43-1.20 (m, 1H), 1.20-0.92 (m, 5H), 0.91-0.64 (m, 4H). LCMS (ESI+): m/z=490.0.0 (M+H), RT: 1.459 min. LC/MS: Kinetex® EVO C18 3.0×50 mm 2.6 μm. Detection methods are photo-diode array (PDA). MS mode was positive electrospray ionization. MS range was 50-1050. Mobile phase: Ramp from 5% ACN (0.01875% TFA) in water (0.0375% TFA) to 95% ACN in 3.40 min, Flow rate is set at 0.9 mL/min; then hold at 95% ACN for 0.30 minutes, Flow rate is set at, 0.9 mL/min; return back to 5% ACN in water and hold for 0.30 min. Flow rate is set at 1.2 mL/min.

### Example 21. Biological Activity of the Compounds of the Present Disclosure

The biological activity of the compounds of the present disclosure is determined utilizing the assay described herein.

### Cell Culture and Transfection

Neuroblastoma cells (ATCC) were cultured in DMEM/F12 (Gibco) supplemented with 10% fetal-bovine serum (Omega) and 1% penicillin-streptomycin (Gibco) at 37 °C with 5% CO₂. For knockdown experiments, cells were transfected with SMARTpool ON-TARGETplus siRNA or control siRNA pool (D001810-10) (GE Dharmacon) at a final concentration of 50 nM, for 96 h in two doses (0, 24 h), after complexing with Lipofectamin RNAiMAX (Invitrogen) in Opti-MEM (Gibco) for 10 min.

Generation of STMN2-NLuc SH-SY5Y lines

WT and TDP-mut (N352S) SH-SY5Y cells were edited using CRISPR-Cas9 to express Nanoluciferase at the C-terminus of both endogenous STMN2 alleles. A single guided RNA targeting STMN2 C-terminus was designed (Benchling webtool) and cloned into pSpCas9-2A-green fluorescent protein (GFP) plasmid (px458-Addgene) using the BbsI restriction site. The sequence for guided RNA targeting STMN2 was: TGTCTGGCTGAAGCAAGGGA. For the repair template, overlapping fragments of 800 bp STMN2 homology arms flanking Nanoluciferase (NLuc, Promega) were generated and ligated into pUC57 backbone. A 42 bp GS-linker was also included 5′ to NLuc to prevent steric hindrance of the reporter to the final STMN2 exon. Fragments were ligated using Gibson Assembly and nucleofected simultaneously with pSpCas9-2A-GFP-gRNA plasmid using the Amaxa Nucleofector (assay A-023). Forty-eight hours following electroporation, cells were collected, and GFP-positive cells sorted and single-cell seeded into 96-well plates using the SH800S Sony cell sorter. Individual clones were expanded and DNA was extracted for PCR amplification of the STMN2 Ex5 genomic locus using primers within NLuc and outside the STMN2 5′ homology arm to confirm presence of the insert. The entire STMN2 coding region and all exon-intron junctions (>500-bp length of each intronic region) were then sequenced to verify the absence of any additional DNA alteration. qPCR analysis was performed with a wild-type isogenic SH-SY5Y cell line that had undergone the mutagenesis attempt but not acquired the insertion mutation during the original screening process.

### Small-Molecule Screening and Validation Using Nanoluciferase Assay

TDP-mut STMN2-NLuc SH-SY5Y cells were used in small-molecule screening at ICCB-Longwood. 3000 cells/well were plated in white 384-well plates using a 50 ul sample volume. 24 hours after plating, compounds were added using 100 nl pin transfer. 24 hours later, 25 ul NLuc assay was added to each well according to manufacturer instructions (Promega, Cat #). Plates were shaken for 1 min prior to luminescence readings using EnVision2 machine. DMSO-only and WT STMN2-NLuc cells were included as negative and positive controls respectively on each plate, producing an average Z′score of ~0.7 across the screen. Each library plate was conducted in duplicate, and Z-scores calculated for each compound. For subsequent validation experiments, cell numbers in each well were quantified by hoescht staining at 1 μg/ml for 15 mins at 37C in black, clear bottom plates, prior to imaging at 4× using automated microscopy (ImageXpressMicro). Nuclei were then counted using a custom algorithm, and luciferase assay run as above. Luminescence results were then normalised against cell counts as RLU/cell.

### Neurite Extension Assay in SH-SY5Y Cells

WT SH-SY5Y cells were cultured in 96-well, black, glass-bottom plates. Compounds were added, and 24 hours later nuclei counted using hoescht staining and automated imaging as above. Neurite outgrowth staining kit (ThermoFisher, A₁₅₀₀₁) was then applied according to manufacturer instructions and fluorescence measured using a plate reader. Fluorescence was then normalised against cell count as RFU/cell.

### Immunoblotting

Total-cells extracts were collected in radioimmunoprecipitation lysis buffer (RIPA buffer). Proteins concentrations were determined by BCA assay (Bio-Rad) and equal amounts of total protein were boiled in SDS sample buffer for 10 min before running in 4-20% acrylamide gel. Proteins were transferred to PVDF, and blocked in 5% milk solution in tris-buffered saline and 0.1% Tween-20 (TBST) for 1 h before overnight incubation with the following primary antibodies: anti-TDP-43 (1:1,000) (ProteinTech, 10782-2-AP), anti-stathmin-2 (1:10,000) (MAB6930, R&D Systems), anti-GAPDH (1:5,000) (ab8245, Abcam). Immunoblots were washed in TBST and probed with horseradish peroxidase-conjugated secondary antibodies diluted 1:5,000 for 1 h at room temperature (Abcam), before being imaged using Chemidoc MP (Biorad). Target engagement within the mevalonate pathway was assessed using electromobility-shift assay (EMSA) as previously described (ref). Lysates were run as above and then blots stained using anti-HDJ-2 antibody (MA5-12748, ThermoFisher), followed by secondary staining using IRDye800-conjugated anti-mouse secondary.

### RNA Extraction, Complementary DNA Synthesis and RT-qPCR

For RNA extraction from cultured cells and neurons, direct lysis was applied using Buffer RLT (RNeasy Mini Kit, Qiagen) and RNA purified according to the manufacturer's instructions. For cDNA synthesis, 1 μg total RNA was reverse transcribed using the high-capacity reverse transcription kit (ABI) according to the manufacturers' instructions. RT-PCR reactions were performed using Q5 High-Fidelity DNA polymerase (NEB) in a T100 thermocycler PCR machine (Bio-Rad). For splicing analyses, RT-PCR products were separated on 2% polyacrylamide gels and then incubated with SYBR gold (Invitrogen) for imaging and analysis. Quantitative real-time PCR was carried out in triplicates, using iTaq Universal SYBR green (Bio-Rad) in a CFX384 real-time PCR machine. mRNA expression of glyceraldehyde-3-phosphate dehydrogenase (GAPDH) was used as endogenous control genes, as indicated.

### RNA Sequencing

Total RNA was extracted from SH-SY5Y cells as indicated above. Subsequently, lug of total RNA was used for mRNA libraries preparation using the TruSeq RNA kit (Illumina). Five biological replicates were sequenced per each condition. Western blotting validation was performed with at least three replicates. RNA sequencing was carried out on an Illumina HiSeq 4000 platform with a median of 25M reads per sample. Fifty-base-pair single-end FASTQ files were obtained using the Illumina demultiplexing pipeline. STAR and RSEM were used to align the reads to the human reference sequence HG38 and to calculate the raw counts and transcripts per million values for genes, respectively. Genes differentially expressed between sample groups were identified by DESeq2.

### Statistics

Statistical tests were performed using GraphPad Prism. Student's t-tests (two-tailed or one-tailed), one-way or two-way ANOVA were used as indicated in the text.

### Results

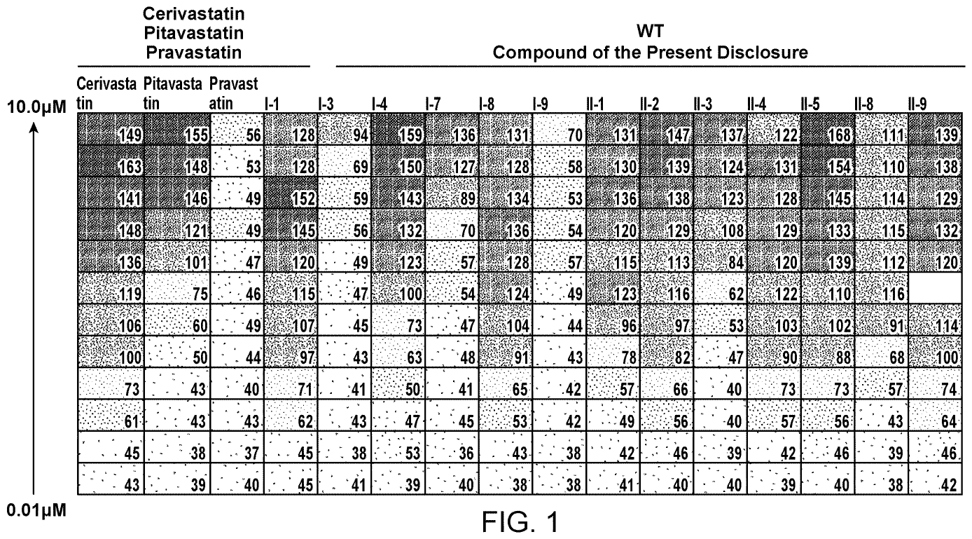

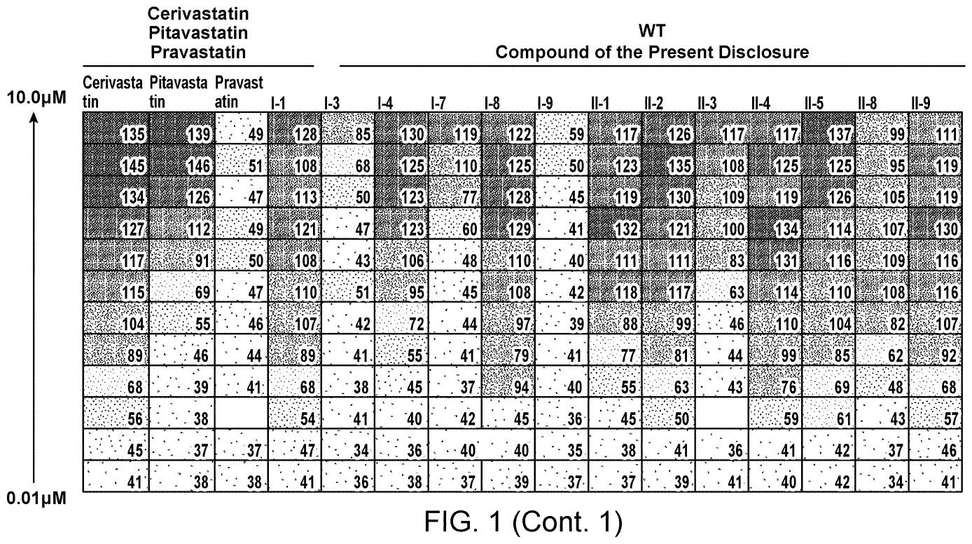

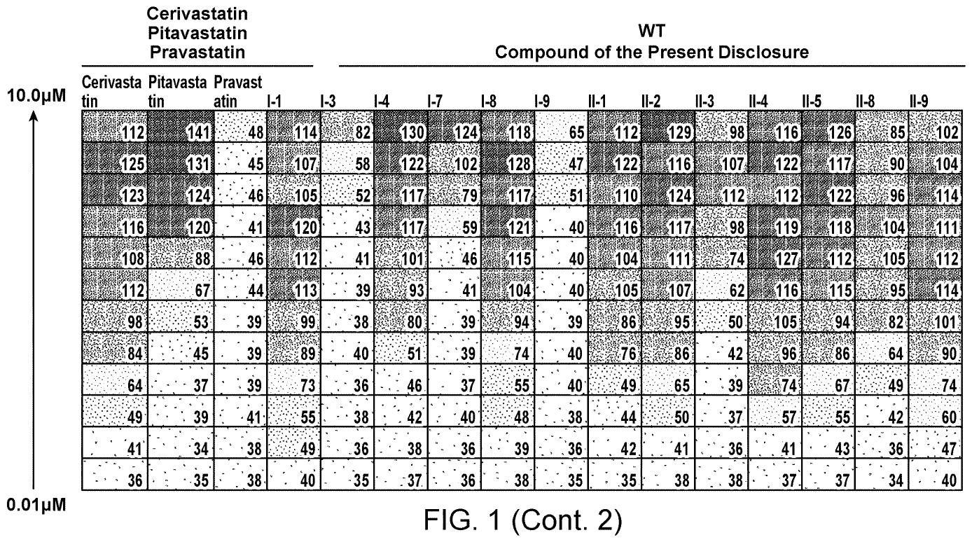

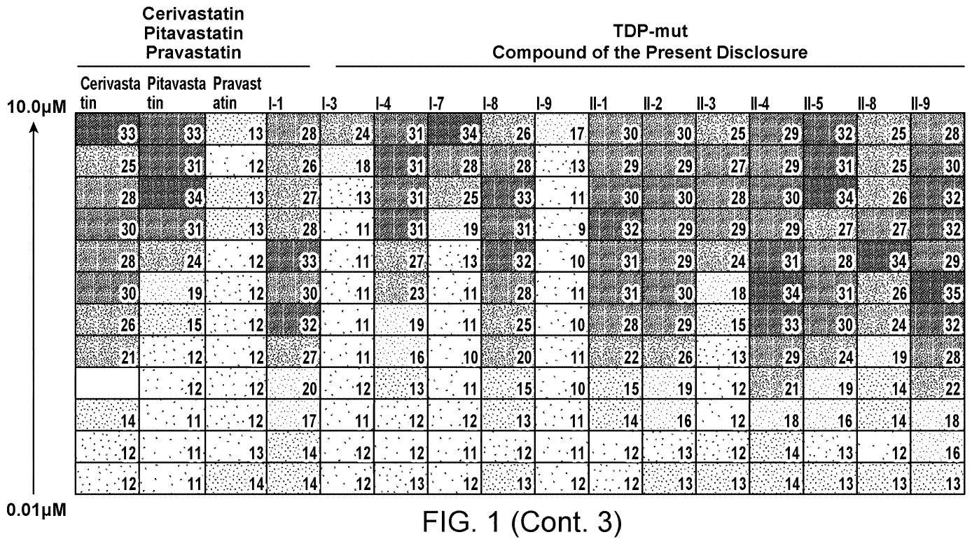

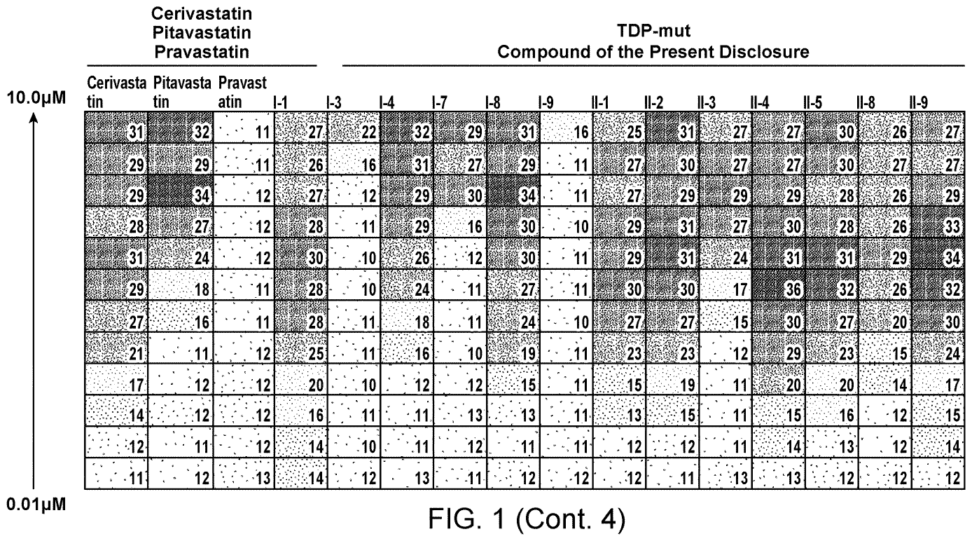

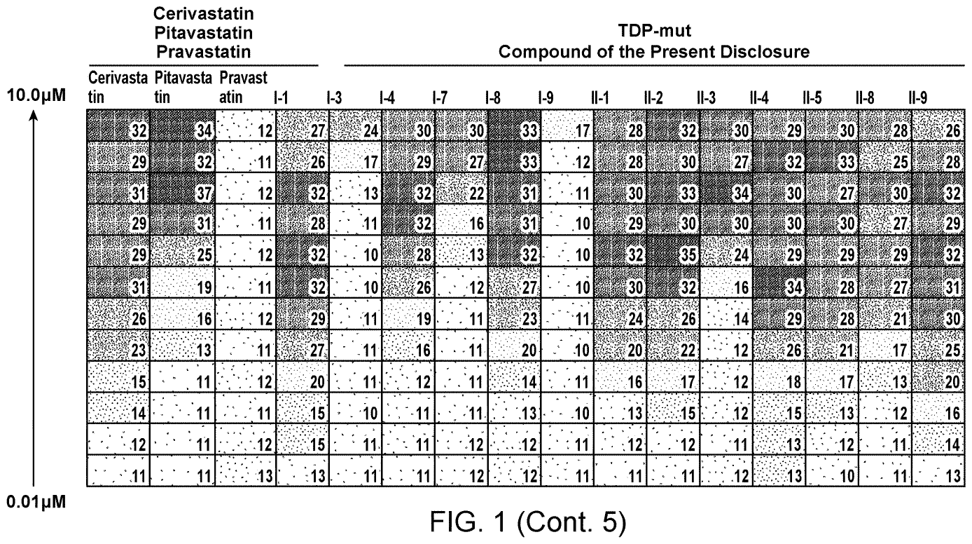

FIG. 1 depicts biological activity of compounds of the present disclosure tested tested in both WT (left) and TDP-43 mutant (right) SH-SY5Y cells, wherein TDP-43 mutant cells exhibit lower levels of endogenous STMN2. Each compound was tested in triplicate in log doses ranging 0.01 μM−10 μM. The values are represented in luminescence normalized to the number of cells in the well, wherein darker grey correlates to higher RLU per cell and higher STMN2 protein expression.

Table A provides EC₅₀ for compounds of the present disclosure in WT TDP-32 and mutant TDP-43 cell lines (“A” means≤0.1 μM; “B” means≥0.1 μM and <0.5 μM; “C” means≥0.5 and <1.0 μM; “D” means≥1.0 and <5.0 μM; “E” means≥5.0 and <10 μM; “F” means≥10 UM)

TABLE A

| | WT TDP-32 (μM) | mut TDP-43 (μM) |
|---|---|---|
| Cerivastatin | B | A |
| Pitavastatin | C | C |
| Pravastatin | F | F |
| I-1 | A | A |
| I-3 | F | E |
| I-4 | B | B |
| I-7 | D | D |
| I-8 | B | B |
| I-9 | F | E |
| II-1 | B | B |
| II-2 | B | A |
| II-3 | C | C |
| II-4 | A | A |
| II-5 | A | A |
| II-8 | B | B |
| II-9 | A | A |

Table B provides EC₅₀ and Emax for compounds of the present disclosure in WT TDP-32 and mutant TDP-43 cell lines. For EC₅₀, “A” means≤10 nM; “B” means≥10 nM and <50 nM; “C” means≥50 nM and <100 nM; “D” means≥100 nM and <500 nM; “E” means≥500 nM. For Emax, “*” means≥1 and <1.25; “**” means≥1.25 and <1.5; “****” means≥1.5 and <1.75; “****” means≥1.75.

TABLE B

| | | HD | | HD |
|---|---|---|---|---|
| | mut HD | Luciferase | WT HD | Luciferase |
| Compound | Luciferase | (mut, | Luciferase | (WT, |
| No. | (nM) | Emax) | (nM) | Emax) |
| I-1 | A | *** | B | **** |
| I-2 | E | * | E | * |
| I-3 | E | * | E | * |
| I-4 | B | *** | B | *** |
| I-5a | E | * | E | * |
| I-5b | C | ** | D | ** |
| I-5c | D | *** | D | ** |
| I-6 | B | *** | B | ** |
| I-7 | D | ** | D | * |
| I-8 | B | *** | B | ** |
| I-9 | E | * | E | * |
| I-10 | B | *** | B | *** |
| I-11 | B | ** | B | *** |
| I-13 | B | *** | B | *** |
| I-15 | B | *** | B | *** |
| I-25 | B | *** | B | **** |
| I-26 | E | * | E | * |
| I-33 | D | ** | D | *** |
| I-34 | C | *** | D | **** |
| II-1 | B | **** | B | *** |
| II-2 | A | *** | A | *** |
| II-3 | D | ** | D | ** |
| II-4 | A | *** | A | *** |
| II-5 | A | *** | A | *** |
| II-6 | B | *** | B | *** |
| II-7 | C | *** | B | *** |
| II-8 | B | *** | B | *** |
| II-9 | A | *** | A | **** |
| II-10 | A | *** | A | *** |
| II-12 | A | *** | A | *** |
| II-13 | B | *** | B | *** |
| II-14 | D | *** | D | *** |
| II-16 | D | ** | C | *** |
| II-17 | A | *** | A | **** |
| II-18 | B | *** | B | *** |
| II-19 | B | *** | B | *** |
| II-20 | B | *** | B | ** |
| II-21 | A | *** | A | **** |
| II-22 | B | *** | B | *** |
| II-23 | B | *** | B | *** |
| II-24 | B | ** | B | ** |
| II-25 | B | *** | B | *** |
| II-26 | E | * | E | * |
| II-27 | E | * | E | * |
| III-1 | D | ** | D | *** |

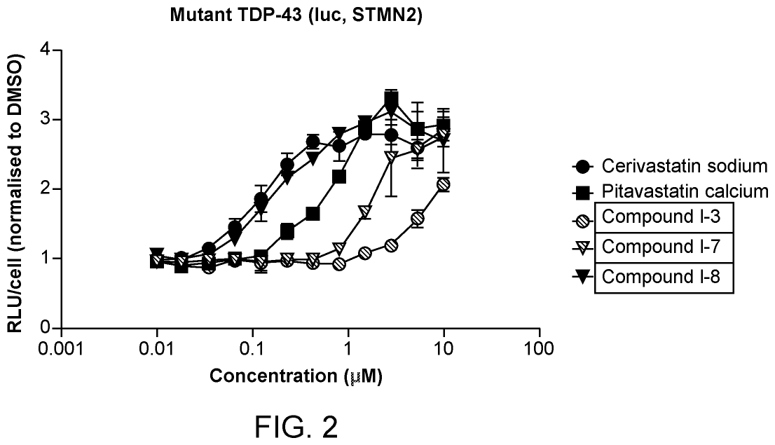

FIG. 2 depicts biological activity of compounds of the present disclosure wherein an increase in STMN2 protein expression is exhibited. Potency of the compounds of the present disclosure are provided in comparison to known statins cerivastatin and pitavastatin.

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A compound of Formula (I′): 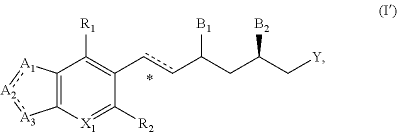
or a pharmaceutically acceptable salt thereof, wherein: [symbol]
is a single or double bond, as valency allows; [symbol]
is a single or double bond, wherein the double bond is the (E) isomer;
X₁ is CH or N;
A₁ is CR_{A1}, N, O, or S;
A₂ is CR_{A2}, N, O, or S;
A₃ is CR_{A3}, N, O, or S, wherein at least one A₁, A₂, or A₃ is S;
R₁ is C₆-C₁₀ aryl or 5- to 10-membered heteroaryl, wherein the aryl or heteroaryl is optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl;
R₂ is C₃-C₁₀ cycloalkyl;
B₁ is H or —OH;
B₂ is H or —OH;
Y is H, —C(O)OR₃, —C(O)N(R₃)₂,
each R₃ independently is H, C₁-C₆ alkyl, C₂-C₆ alkenyl, C₂-C₆ alkynyl, C₁-C₆ alkoxyl, or C₁-C₆ haloalkyl;
R_{A1} is H, C₁-C₆ alkyl, C₂-C₆ alkenyl, C₂-C₆ alkynyl, C₁-C₆ alkoxyl, C₁-C₆ haloalkyl, or C₃-C₁₀ cycloalkyl;
R_{A2} is H, C₁-C₆ alkyl, C₂-C₆ alkenyl, C₂-C₆ alkynyl, C₁-C₆ alkoxyl, C₁-C₆ haloalkyl, C₃-C₁₀ cycloalkyl, C₆-C₁₀ aryl, or 5- to 10-membered heteroaryl; and
R_{A3} is H, C₁-C₆ alkyl, C₂-C₆ alkenyl, C₂-C₆ alkynyl, C₁-C₆ alkoxyl, C₁-C₆ haloalkyl, C₃-C₁₀ cycloalkyl, 3- to 10-membered heterocyclyl optionally substituted with one or more C₁-C₆ alkyl, C₆-C₁₀ aryl, or 5- to 10-membered heteroaryl optionally substituted with one or more C₁-C₆ alkyl,
provided that:
(a) when R₂ is cyclopropyl, X₁ is N, A₁ is CR_{A1}, and A₂ is CR_{A2}, then at least one of R_{A1} and R_{A2} is not H; and
(b) when R₂ is cyclopropyl, X₁ is N, A₁ is CR_{A1}, and A₂ is CH, then R_{A1} is not ethyl.

2. The compound of claim 1, wherein [symbol]
is a single or double bond, as valency allows;
X₁ is CH or N;
A₁ is CR_{A1}, N, O, or S;
A₂ is CR_{A2}, N, O, or S;
A₃ is CR_{A3}, N, O, or S, wherein at least one A₁, A₂, or A₃ is S;
R₁ is C₆-C₁₀ aryl or 5- to 10-membered heteroaryl, wherein the aryl or heteroaryl is optionally substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl;
R₂ is C₃-C₁₀ cycloalkyl;
R₃ is H, C₁-C₆ alkyl, C₂-C₆ alkenyl, C₂-C₆ alkynyl, C₁-C₆ alkoxyl, or C₁-C₆ haloalkyl;
R_{A1} is H, C₁-C₆ alkyl, C₂-C₆ alkenyl, C₂-C₆ alkynyl, C₁-C₆ alkoxyl, or C₁-C₆ haloalkyl;
R_{A2} is H, C₁-C₆ alkyl, C₂-C₆ alkenyl, C₂-C₆ alkynyl, C₁-C₆ alkoxyl, or C₁-C₆ haloalkyl; and
R_{A3} is H, C₁-C₆ alkyl, C₂-C₆ alkenyl, C₂-C₆ alkynyl, C₁-C₆ alkoxyl, or C₁-C₆ haloalkyl,
provided that:
(a) when R₂ is cyclopropyl, X₁ is N, A₁ is CR_{A1}, and A₂ is CR_{A2}, then at least one of R_{A1} and R_{A2} is not H; and
(b) when R₂ is cyclopropyl, X₁ is N, A₁ is CR_{A1}, and A₂ is CH, then R_{A1} is not ethyl.

3. The compound of claim 1 or claim 2, wherein X₁ is N.

4. The compound of any one of the preceding claims, wherein A₁ is CR_{A1}, N, or S.

5. The compound of any one of the preceding claims, wherein A₂ is CR_{A2}.

6. The compound of any one of the preceding claims, wherein A₃ is CR_{A3}, N, or S.

7. The compound of any one of the preceding claims, wherein R₁ is Co aryl substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl.

8. The compound of any one of the preceding claims, wherein R₁ is

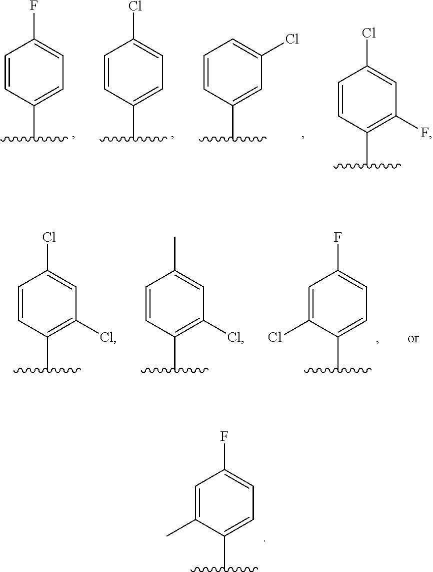

9. The compound of any one of the preceding claims, wherein R₂ is

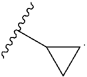 [SMILES: *C1CC1]

10. The compound of any one of the preceding claims, wherein R₃ is H or methyl.

11. The compound of any one of the preceding claims, wherein R_{A1} is H, methyl, or cyclopropyl.

12. The compound of any one of the preceding claims, wherein R_{A2} is H, methyl, or cyclopropyl.

13. The compound of any one of the preceding claims, wherein R_{A3} is H, methyl, phenyl, cyclopropyl, cyclobutyl, cyclohexyl.

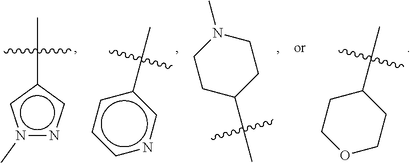

14. The compound of any one of the preceding claims, wherein the compound of Formula (I′) is of Formula (I′): 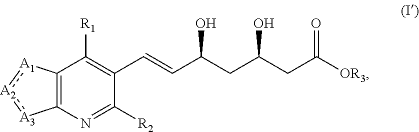
or a pharmaceutically acceptable salt thereof.

15. The compound of any one of the preceding claims, wherein the compound of Formula (I′) is of Formula (I-a), (I-b), (I-c), or (I-d): 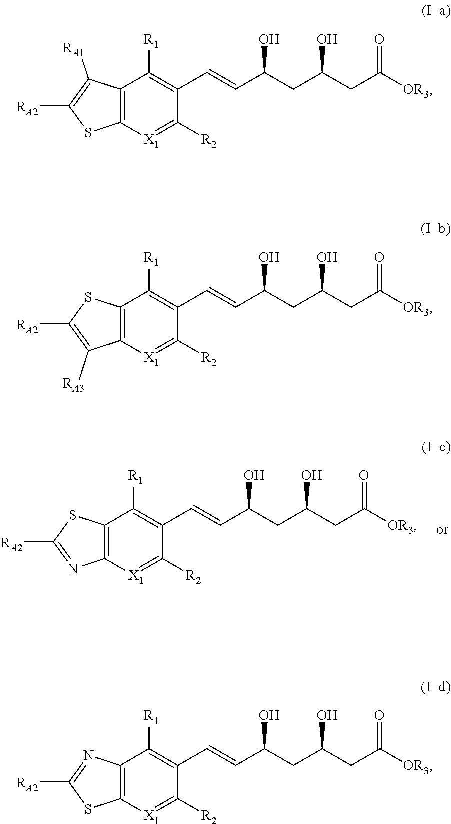
or a pharmaceutically acceptable salt thereof.

16. The compound of any one of the preceding claims, wherein the compound of Formula (I′) is of Formula (I′-a), (I′-b), (I′-c), or (I′-d): 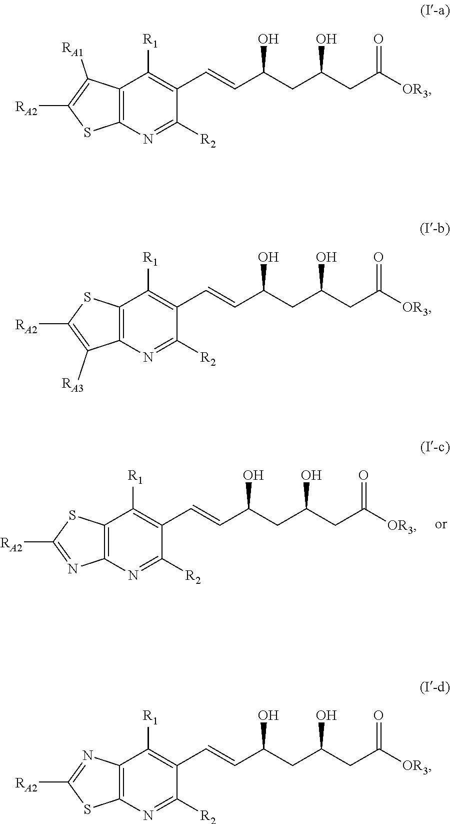
or a pharmaceutically acceptable salt thereof.

17. The compound of any one of claims 1-15, wherein the compound of Formula (I′) is of Formula (I-d), (I-e), (I-f), or (I-g): 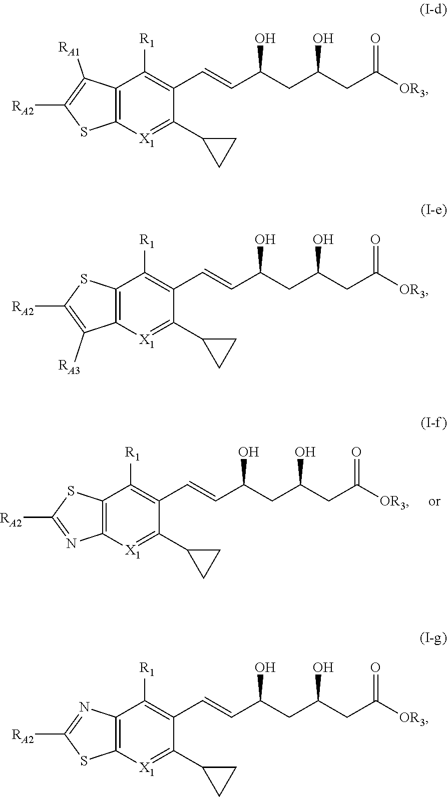
or a pharmaceutically acceptable salt thereof.

18. The compound of any one of the preceding claims, wherein the compound of Formula (I′) is of Formula (I′-d), (I′-e), (I′-f), or (I′-g): 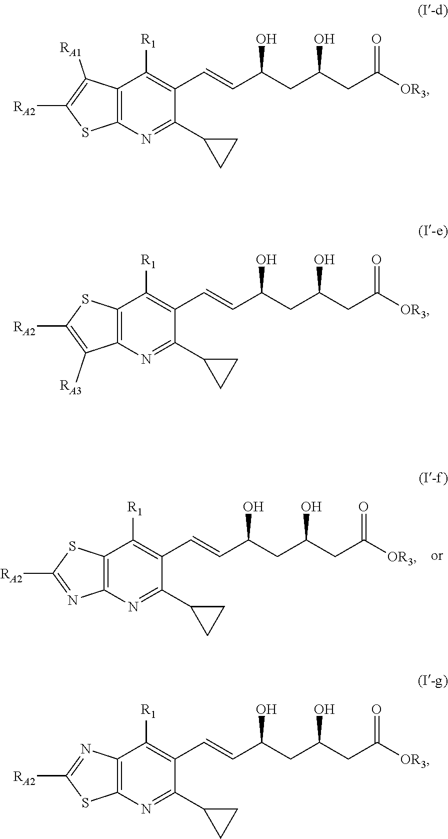
or a pharmaceutically acceptable salt thereof.

19. The compound of any one of claims 1-15, wherein the compound of Formula (I′) is of Formula (I-h), (I-i), (I-j), or (I-k): 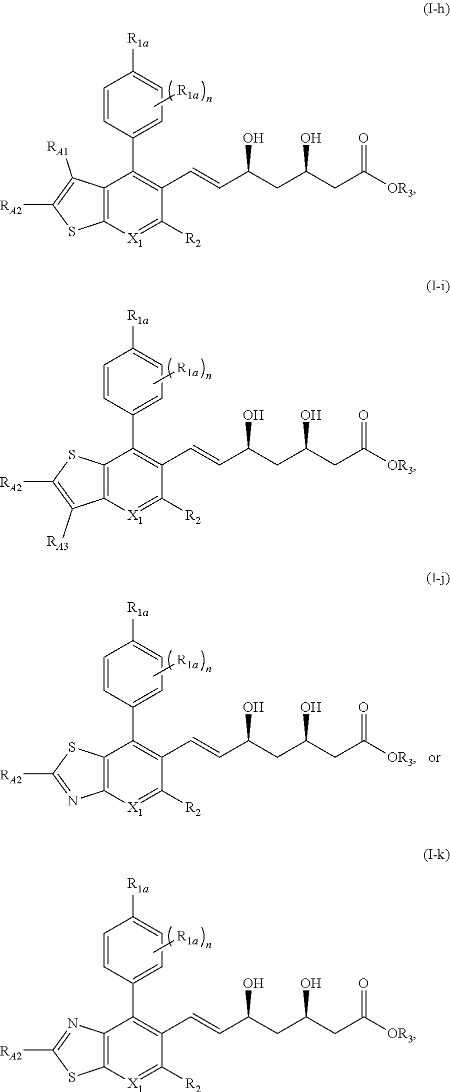
or a pharmaceutically acceptable salt thereof, wherein R₁ₐ is halo, —CN, —OH, —NH₂, C₁-C₆ alkyl, C₂-C₆ alkenyl, C₂-C₆ alkynyl, C₁-C₆ alkoxyl, or C₁-C₆ haloalkyl, and n is 0, 1, 2, 3, or 4.

20. The compound of any one of the preceding claims, wherein the compound of Formula (I′) is of Formula (I′-h), (I′-i), (I′-j), or (I′-k): 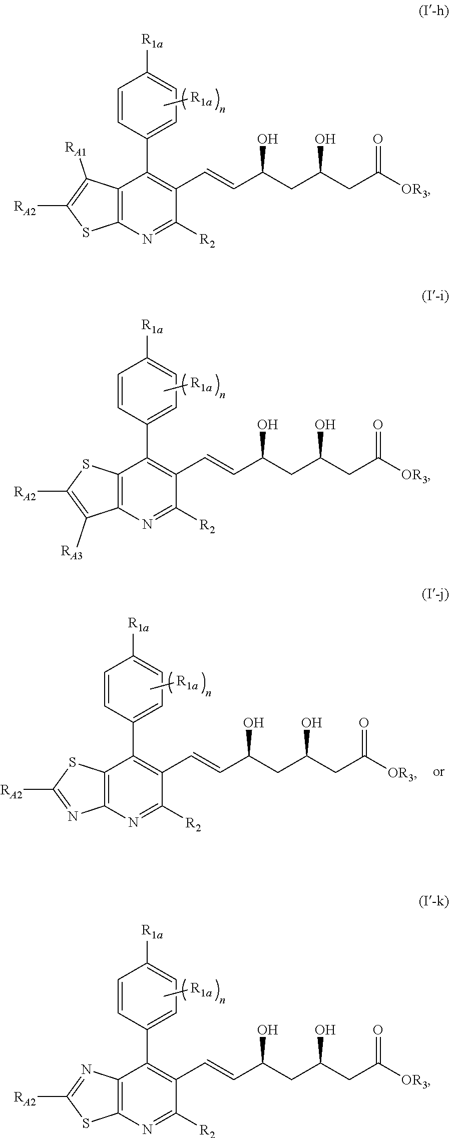
or a pharmaceutically acceptable salt thereof, wherein R₁ₐ is halo, —CN, —OH, —NH₂, C₁-C₆ alkyl, C₂-C₆ alkenyl, C₂-C₆ alkynyl, C₁-C₆ alkoxyl, or C₁-C₆ haloalkyl, and n is 0, 1, 2, 3, or 4.

21. The compound of any one of claims 1-15, wherein the compound of Formula (I′) is of Formula (I-1), (I-m), (I-n), or (I-o): 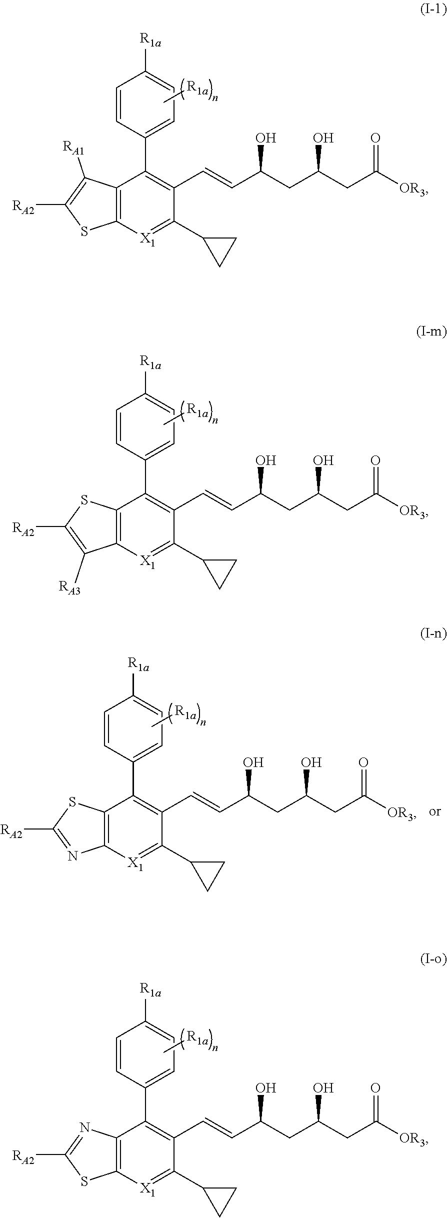
or a pharmaceutically acceptable salt thereof, wherein R₁ₐ is halo, —CN, —OH, —NH₂, C₁-C₆ alkyl, C₂-C₆ alkenyl, C₂-C₆ alkynyl, C₁-C₆ alkoxyl, or C₁-C₆ haloalkyl, and n is 0, 1, 2, 3, or 4.

22. The compound of any one of the preceding claims, wherein the compound of Formula (I′) is of Formula (I′-1), (I′-m), (I′-n), or (I′-o): 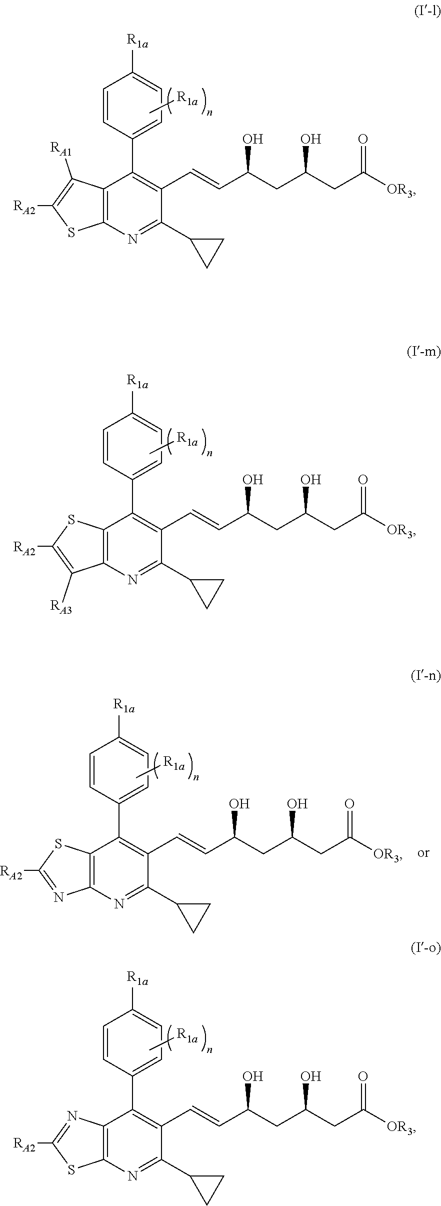
or a pharmaceutically acceptable salt thereof, wherein R₁ₐ is halo, —CN, —OH, —NH₂, C₁-C₆ alkyl, C₂-C₆ alkenyl, C₂-C₆ alkynyl, C₁-C₆ alkoxyl, or C₁-C₆ haloalkyl, and n is 0, 1, 2, 3, or 4.

23. A compound of Formula (II′): 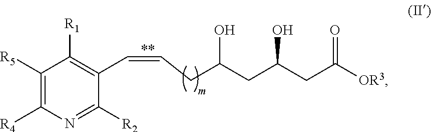
or a pharmaceutically acceptable salt thereof, wherein: [symbol]
is a double bond, wherein the double bond is the (E) or (Z) isomer;
R₁ is C₆-C₁₀ aryl or 5- to 10-membered heteroaryl, wherein the aryl or heteroaryl is optionally substituted with one or more R₁ₐ;
each R₁ₐ independently is halo, —CN, —OH, —NH₂, C₁-C₆ alkyl, C₂-C₆ alkenyl, C₂-C₆ alkynyl, C₁-C₆ alkoxyl, or C₁-C₆ haloalkyl;
R₂ is C₃-C₁₀ cycloalkyl or methyl;
R₃ is H, C₁-C₆ alkyl, C₂-C₆ alkenyl, C₂-C₆ alkynyl, C₁-C₆ alkoxyl, or C₁-C₆ haloalkyl;
R₄ is H, C₃-C₁₀ cycloalkyl, 3- to 10-membered heterocyclyl, C₁-C₆ alkyl, C₂-C₆ alkenyl, C₂-C₆ alkynyl, C₁-C₆ alkoxyl, or C₁-C₆ haloalkyl;
R₅ is H, C₁-C₆ alkyl, C₂-C₆ alkenyl, C₂-C₆ alkynyl, C₁-C₆ alkoxyl, C₁-C₆ haloalkyl, or C₃-C₁₀ cycloalkyl, wherein the alkyl, alkenyl, alkynyl, alkoxyl, haloalkyl or C₃-C₁₀ cycloalkyl is optionally substituted with one or more C₁-C₆ alkoxy or —O(C₃-C₁₀ cycloalkyl), or R₅ and one R₁ₐ, together with the intervening atoms, form 3- to 10-membered heterocyclyl; and
m is 0 or 1,
provided that:
(a) when R₂ is cyclopropyl, and R₅ is C₁ alkyl substituted by C₁ alkoxy, then R₄ is not isopropyl; and
(b) when R₁ is monosubstituted C₆ aryl substituted by one fluoro, R₂ is cyclopropyl, and R₅ is C₁ alkyl substituted by C₁ alkoxy, then R₄ is not cyclopropyl; and
(c) when R₂ is methyl, then R₄ is not C₁-C₆ alkyl.

24. The compound of claim 23, wherein R₁ is C₆ aryl substituted with one or more halo, —CN, —OH, —NH₂, C₁-C₆ alkyl, C₂-C₆ alkenyl, C₂-C₆ alkynyl, C₁-C₆ alkoxyl, or C₁-C₆ haloalkyl.

25. The compound of claim 23 or claim 24, wherein R₁ is

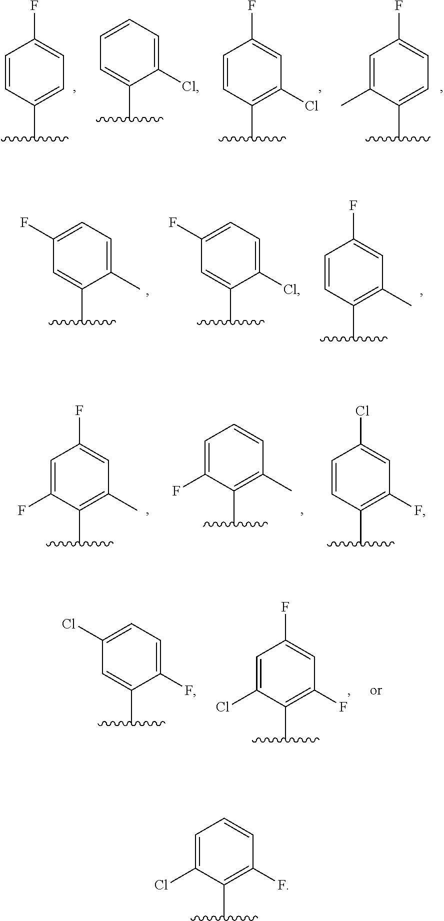

26. The compound of any one of claims 23-25, wherein R₂ is methyl,

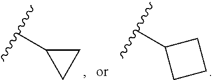

27. The compound of any one of claims 23-26, wherein R₃ is H.

28. The compound of any one of claims 23-27, wherein R₄ is H,

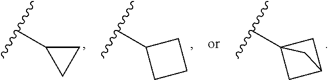

29. The compound of any one of claims 23-28, wherein R₅ is —(CH₂)—OCH₃, —(CH₂)—O-cyclopropyl, cyclopropyl, or H, or R₅ and one R₁ₐ, together with the intervening atoms, form tetrahydropyran.

30. The compound of any one of claims 23-29, wherein the compound of Formula (II′) is of Formula (II-a) or (II-b): 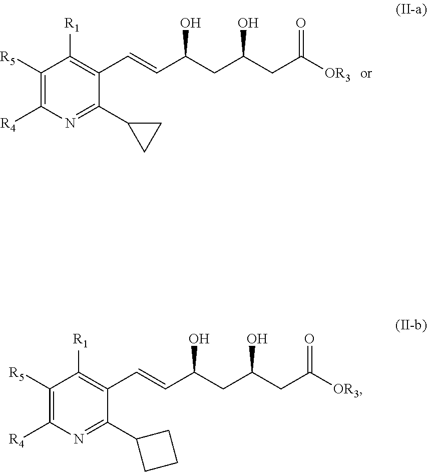
or a pharmaceutically acceptable salt thereof.

31. The compound of any one of claims 23-30, wherein the compound of Formula (II′) is of Formula (II-c): 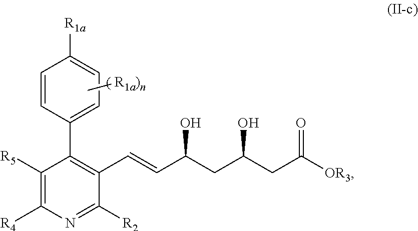
or a pharmaceutically acceptable salt thereof, wherein R₁ₐ is halo, —CN, —OH, —NH₂, C₁-C₆ alkyl, C₂-C₆ alkenyl, C₂-C₆ alkynyl, C₁-C₆ alkoxyl, or C₁-C₆ haloalkyl, and n is 0, 1, 2, 3, or 4.

32. The compound of any one of claims 23-31, wherein the compound of Formula (II′) is of Formula (II-d) or (II-e): 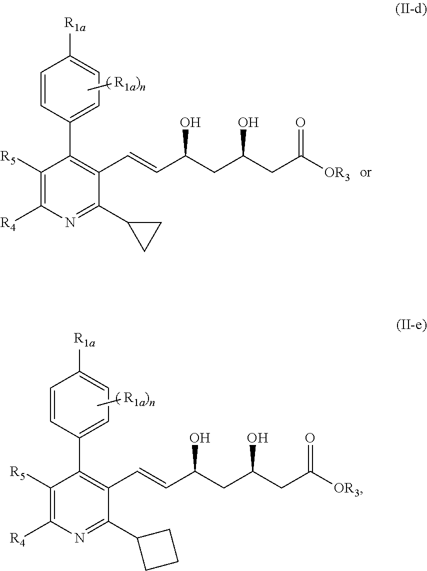
or a pharmaceutically acceptable salt thereof, wherein R₁ₐ is halo, —CN, —OH, —NH₂, C₁-C₆ alkyl, C₂-C₆ alkenyl, C₂-C₆ alkynyl, C₁-C₆ alkoxyl, or C₁-C₆ haloalkyl, and n is 0, 1, 2, 3, or 4.

33. The compound of any one of claims 23-32, wherein the compound of Formula (II′) is of Formula (II-f) or (II-g): 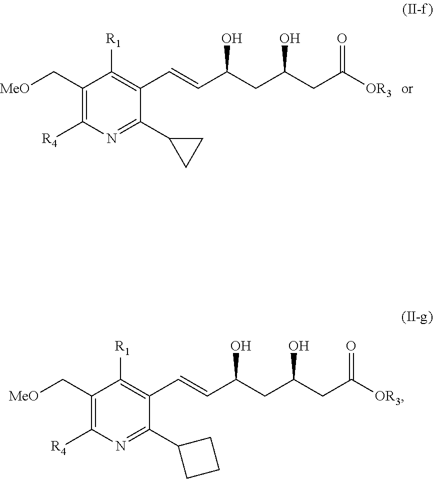
or a pharmaceutically acceptable salt thereof.

34. The compound of any one of claims 23-33, In some embodiments, the compound of Formula (II′) is of Formula (II-h): 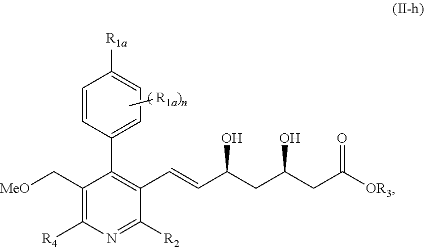
or a pharmaceutically acceptable salt thereof, wherein R₁ₐ is halo, —CN, —OH, —NH₂, C₁-C₆ alkyl, C₂-C₆ alkenyl, C₂-C₆ alkynyl, C₁-C₆ alkoxyl, or C₁-C₆ haloalkyl, and n is 0, 1, 2, 3, or 4.

35. The compound of any one of claims 23-34, In some embodiments, the compound of Formula (II′) is of Formula (II-i) or (II-j): 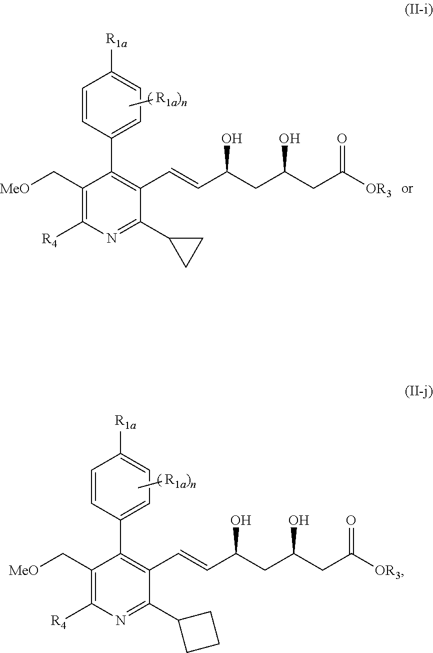
or a pharmaceutically acceptable salt thereof, wherein R₁ₐ is halo, —CN, —OH, —NH₂, C₁-C₆ alkyl, C₂-C₆ alkenyl, C₂-C₆ alkynyl, C₁-C₆ alkoxyl, or C₁-C₆ haloalkyl, and n is 0, 1, 2, 3, or 4.

36. The compound of any one of the preceding claims, wherein the compound is selected from a compound described in Table 1 or Table 1A, or a pharmaceutically acceptable salt thereof.

37. The compound of any one of the preceding claims, wherein the compound is selected from a compound described in Table 2 or Table 2A, or a pharmaceutically acceptable salt thereof.

38. The compound of any one of the preceding claims, wherein the compound is selected from a compound described in Table 3, or a pharmaceutically acceptable salt thereof.

39. A compound obtainable by, or obtained by, a method described herein; optionally, the method comprises one or more steps described in Schemes 1-7.

40. A pharmaceutical composition comprising the compound of any one of the preceding claims or a pharmaceutically acceptable salt thereof, and a pharmaceutically acceptable diluent or carrier.

41. The pharmaceutical composition of any one of claims 1-34, wherein the compound is selected from a compound described in Table 1, Table 1A, Table 2, Table 2A, or Table 3.

42. A method of modulating STMN2 activity with a compound of any one of claims 1-39 or a pharmaceutical composition of claim 40 or claim 41.

43. The compound of any one of claims 1-39 or pharmaceutical composition of claim 40 or claim 41 for use in modulating STMN2 activity.

44. Use of the compound of any one of claims 1-39 in the manufacture of a medicament for modulating STMN2 activity.

45. A method of treating or preventing a disease or disorder in a subject in need thereof, comprising administering to the subject a compound of any one of claims 1-39 or pharmaceutical composition of claim 40 or claim 41.

46. The compound of any one of claims 1-39 or pharmaceutical composition of claim 40 or claim 41 for use in treating or preventing a disease or disorder.

47. Use of the compound of any one of claims 1-39 in the manufacture of a medicament for treating or preventing a disease or disorder.

48. The method, compound, pharmaceutical composition, or use of any one of claims 45-47, wherein the disease or disorder is associated with an implicated STMN2 activity.

49. The method, compound, pharmaceutical composition, or use of any one of claims 45-48, wherein the disease or disorder is a neurodegenerative disease or disorder.

50. The method, compound, pharmaceutical composition, or use of any one of claims 45-48, wherein the disease or disorder is axonopathy.

51. The method, compound, pharmaceutical composition, or use of claim 49, wherein the neurodegenerative disease or disorder is Amyotrophic Lateral Sclerosis (ALS), Parkinson's Disease (PD), Alzheimer's Disease (AD), Frontotemporal Dementia (FTD), Inclusion Body Myopathies (IBM), Rett Syndrome, Alexander Syndrome, Perry Syndrome, Limbic-predominant Age-related TDP-43 Encephalopathy Neuropathologic Change (LATE-NC), Lewy Body Dementia (LBD), Peripheral neuropathies (chemotherapy induced neuropathy, injury induced), and Autism spectrum disorder.

52. The method, compound, pharmaceutical composition, or use of any one of claims 42-51, wherein the subject is human.
